SEQUENCE LISTING (86 sequences)

SEQ ID NO: 1 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - protein, 183 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTGQFPMVPSGLTPQPAQDWYLLDDDPRLLTLSASSKVPVTLAAV

SEQ ID NO: 3 - protein, 331 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 4 - protein, 1283 aa, Homo sapiens
MGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 5 - protein, 699 aa, Homo sapiens
MALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 6 - protein, 1323 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 7 - protein, 164 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTGQFPMVPSGLTPQPAQDWYLLDDDPRLLTLSASSKVPVTLAAV

SEQ ID NO: 8 - protein, 312 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 9 - protein, 624 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLT

SEQ ID NO: 10 - protein, 21 aa, Homo sapiens
MALTVIAGLVVIFMMLGGTFL

SEQ ID NO: 11 - protein, 678 aa, Homo sapiens
YWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 12 - protein, 44 aa, Homo sapiens
YWRGRRIQNKRAMRRYLERGESIEPLDPSEKANKVLARIFKETE

SEQ ID NO: 13 - protein, 258 aa, Homo sapiens
LRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFT

SEQ ID NO: 14 - protein, 376 aa, Homo sapiens
RMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 15 - protein, 164 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSC

SEQ ID NO: 16 - protein, 146 aa, Homo sapiens
PPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPK

SEQ ID NO: 17 - protein, 166 aa, Homo sapiens
ACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVA

SEQ ID NO: 18 - protein, 148 aa, Homo sapiens
EGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLT

SEQ ID NO: 19 - protein, 17 aa, Homo sapiens
QPLVYNKLTFQLEPNPH

SEQ ID NO: 20 - protein, 1283 aa, Macaca mulatta
MGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWKDIVRDQDAEIVVKDNGRSCPLCHEVCKGRCWGPGPEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMYNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKIIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGEAFQESAVSGSSEWCPRPVSLHPMPRGCLASESSEGHVTGSEAELQEKVSTCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPRPPRPSSLEELGYEYMDVGSDLSASLGSTQSCPLHPVPVMPTAGTTPDEDYEYMNRQRGGSGPGGDYAAMGACPASEQGYEEMRAFQGPGHQAPHVHYAHLKTLRSLEATDSAFDNPDYWHSRLFPKANAQRT

SEQ ID NO: 21 - protein, 120 aa, synthetic construct
DVQLQESGPDLVKPSQSLSLTCTVTGYSITSGYSWHWIRQFPGNKLEWMGSIHYSGGTNYNPSLKSRISITRDTSKNQFFLQLNSVTTEDTATYFCARMTTAPRYPFDYWGQGTTLTVSS

SEQ ID NO: 22 - protein, 120 aa, synthetic construct
DVQLQEWGAGLLKPSETLSLTCAVTGYSITSGYSWHWIRQFPGNGLEWIGSIHYSGGTNYNPTLKSRITISRDTSKNQFSLKLSSVTAADTAVYFCARMTTAPRYPFDYWGQGTLVTVSS

SEQ ID NO: 23 - protein, 120 aa, synthetic construct
DVQLQEWGAGLLKPSETLSLTCAVTGYSITSGYSWHWIRQFPGNGLEWIGSIHYSGGTNYNPSLKSRITISRDTSKNQFSLKLSSVTAADTAVYFCARMTTAPRYPFDYWGQGTLVTVSS

SEQ ID NO: 24 - protein, 120 aa, synthetic construct; identical to SEQ ID NO: 23

SEQ ID NO: 25 - protein, 120 aa, synthetic construct; identical to SEQ ID NO: 23

SEQ ID NO: 26 - protein, 120 aa, synthetic construct
DVQLQEWGAGLLKPSETLSLTCAVTGYSITSGYSWHWIRQFPGKGLEWIGSIHYSGGTNYNPSLKSRITISRDTSKNQFSLKLSSVTAADTAVYFCARMTTAPRYPFDYWGQGTLVTVSS

SEQ ID NO: 27 - protein, 120 aa, synthetic construct
DVQLQEWGAGLLKPSETLSLTCAVYGYSITSGYSWHWIRQPPGKGLEWIGSIHYSGGTNYNPSLKSRVTISRDTSKNQFSLKLSSVTAADTAVYYCARMTTAPRYPFDYWGQGTLVTVSS

SEQ ID NO: 28 - protein, 120 aa, synthetic construct
DVQLQEWGAGLLKPSETLSLTCAVTGYSITSGYSWHWIRQFPGNGLEWIGSIRYSGGTNYNPSLKSRITISRDTSKNQFSLKLGSVTAADTAVYFCARMTTAPRYPFDYWGQGTLVTVSS

SEQ ID NO: 29 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARMTTAPRYPFDYWGQGTLVTVSS

SEQ ID NO: 30 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARETTAPRYPFDYWGQGTLVTVSS

SEQ ID NO: 31 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARGTTAPRYPFDYWGQGTLVTVSS

SEQ ID NO: 32 - protein, 120 aa, synthetic construct
DVQLQEWGAGLLKPSETLSLTCAVTGYSITSGYSWHWIRQFPGNGLEWIGSIHYSGGTNYNPSLKSRITISRDTSKNQFSLRLSSVTAADTAVYFCARMTTAPRYPFDYWGQGTLVTVSS

SEQ ID NO: 33 - protein, 120 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSS

SEQ ID NO: 34 - protein, 120 aa, synthetic construct
QVQLQESGPGLVKPSQTLFLTCTVSGYSITSGYSWHWIRQHPGKGLEWIGSIRYSGGTDYNPSLKSLVTISVDTSKNQFSLKLSSVTAADTAVYYCARMTTAPWYPFDYWGQGTTVTVSS

SEQ ID NO: 35 - protein, 120 aa, synthetic construct
QVQLQESGPGLVKPSQTLSLTCTVSGYYITSGYSWHWIRQHPGKGLEWIGSIRYSGGTDYNPSLKSLATISADTSKNQFSLKLSSVTAADTAVYYCARMTTAPWYPFDYWGQGTAVTVSS

SEQ ID NO: 36 - protein, 120 aa, synthetic construct; identical to SEQ ID NO: 22

SEQ ID NO: 37 - protein, 120 aa, synthetic construct; identical to SEQ ID NO: 32

SEQ ID NO: 38 - protein, 9 aa, synthetic construct
GYSITSGYS

SEQ ID NO: 39 - protein, 9 aa, synthetic construct
GYYITSGYS

SEQ ID NO: 40 - protein, 9 aa, synthetic construct
  VARIANT 3: note = S can be replaced by Y
GYSITSGYS

SEQ ID NO: 41 - protein, 7 aa, synthetic construct
IHYSGGT

SEQ ID NO: 42 - protein, 7 aa, synthetic construct
IRYSGGT

SEQ ID NO: 43 - protein, 7 aa, synthetic construct
  VARIANT 2: note = H can be replaced by R
IHYSGGT

SEQ ID NO: 44 - protein, 13 aa, synthetic construct
ARMTTAPRYPFDY

SEQ ID NO: 45 - protein, 13 aa, synthetic construct
ARMTTAPWYPFDY

SEQ ID NO: 46 - protein, 13 aa, synthetic construct
ARETTAPRYPFDY

SEQ ID NO: 47 - protein, 13 aa, synthetic construct
ARGTTAPRYPFDY

SEQ ID NO: 48 - protein, 13 aa, synthetic construct
  VARIANT 3: note = M can be replaced by E or G
  VARIANT 8: note = R can be replaced by W
ARMTTAPRYPFDY

SEQ ID NO: 49 - protein, 107 aa, synthetic construct
DIVMTQSQKFMSTSVGDRVSVTCKASQIVGSNVAWYQQKPGQSPKPLIYSASYRYSGVPDRFTASGSGTDFTLTITNVQSEDLAEYFCQQYSSHPLTFGAGTKLELK

SEQ ID NO: 50 - protein, 107 aa, synthetic construct
DIVMTQSPSSLSASVGDLVTITCKASQIVGSNVAWYQMKPGKSPKPLIYSASYLYFGVPSRFSGSGSGTDFTLTISSLQPEDVAEYFCQQYSSHPLTFGPGTKVEIK

SEQ ID NO: 51 - protein, 107 aa, synthetic construct
DIVMTQSPSSLSASGGDRVTITCKASQIVGYNVAWYQQKPGKSPKPLIYSASYLYSDVPSRFSASGSGTDFTLTISSLQPEDVAEYFCQQYSSHPLTFGPGTKVEIK

SEQ ID NO: 52 - protein, 107 aa, synthetic construct
VIVMTQSPSSLSASVGDRVTITCKASQIVGPNVAWYQQKPGKSPKPLIYSASYGYSDVPSRFSGSGSGTDFTLTISSLQPEDVAEYFCQQYSTHPLTFGPGTKVEIK

SEQ ID NO: 53 - protein, 107 aa, synthetic construct
DIVMTQSPSSLSASVGDRVTITCKASQIVGSNVAWYQQKPGKSPKPLIYSASYGYSDVPSRFSGSGSGTDFTLTISSLRPEDVATYYCQQYSSHPLTFGPGTKVEIK

SEQ ID NO: 54 - protein, 107 aa, synthetic construct
DIVMTQSPSSLSASVGDRVTITCKASQIVGSNVAWYQQKPGKSPKPLIYSARYQYSGVPFRFSGSGSGTDFTLTISSLQPEDVATYYCQQYSSHPLTFGPGTKVEIK

SEQ ID NO: 55 - protein, 107 aa, synthetic construct; identical to SEQ ID NO: 54

SEQ ID NO: 56 - protein, 107 aa, synthetic construct; identical to SEQ ID NO: 54

SEQ ID NO: 57 - protein, 107 aa, synthetic construct
DIVMTQSPSSLSASVGDRVTITCKASQIVGSNVAWYQQMPGKSPEPLIYSASYLYSDVPSRFSGSGSGTDFTMTISSLQPEDVATYYCQQYSSHPLTFGPGTKVEIK

SEQ ID NO: 58 - protein, 107 aa, synthetic construct
DIQMTQSPSSVSASVGDRVTITCKASQIVGSNVAWYQQKPGKAPEPLIYSASYLYSGVPSRFSGSGSGTDFTLTISSLQPEDFATYYCQQYSSHPLTFGQGTKLEIK

SEQ ID NO: 59 - protein, 107 aa, synthetic construct
DIQMTQSPSSVSASVGDRVTFTCKASQIVGSNVAWYQQKPGKAPEPLIYSASYLYSSVPSRFSGSGSGTEFTMTISSLEPEDFATYYCQQYTTHPLTFGPGTKVEIK

SEQ ID NO: 60 - protein, 107 aa, synthetic construct
DIQMTQSPSSVSASVGDRVTITCKASQIVGSNVAWYQQKPGKAPMPLIYSASYGYSGVPSRFSGSGSGTDFTLTISSLQPEDFATYYCQQYSSHPLTFGQGTKLEIK

SEQ ID NO: 61 - protein, 107 aa, synthetic construct
DIVMTQSPSSLSASVGDLVTITCKASQIVGSNVAWYQMKLGKSPKPLIYSASYLYFGVPSRFSGSGSGTDFTLTISSLQPEDVAEYFCQQYFSHPLTFGPGTKVEIK

SEQ ID NO: 62 - protein, 107 aa, synthetic construct
DIVMTQSPSSLSASVGDRVTITCKASQIVGSNVAWYQQKPGKSPKPLIYSASYLYSDVPSRFSGSGSGTDFTMTISSLQPEDVATYYCQQYSSHPLTFGPGTKVEIK

SEQ ID NO: 63 - protein, 6 aa, synthetic construct
QIVGSN

SEQ ID NO: 64 - protein, 6 aa, synthetic construct
QIVGYN

SEQ ID NO: 65 - protein, 6 aa, synthetic construct
QIVGPN

SEQ ID NO: 66 - protein, 6 aa, synthetic construct
  VARIANT 5: note = S can be replaced by Y or P
QIVGSN

SEQ ID NO: 67-69 - skipped (3 entries)

SEQ ID NO: 70 - protein, 9 aa, synthetic construct
QQYSSHPLT

SEQ ID NO: 71 - protein, 9 aa, synthetic construct
QQYSTHPLT

SEQ ID NO: 72 - protein, 9 aa, synthetic construct
QQYTTHPLT

SEQ ID NO: 73 - protein, 9 aa, synthetic construct
QQYFSHPLT

SEQ ID NO: 74 - protein, 9 aa, synthetic construct
  VARIANT 5: note = S can be replaced by T
  VARIANT 4: note = S can be replaced by T or F
QQYSSHPLT

SEQ ID NO: 75 - protein, 450 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 214 aa, synthetic construct
DIQMTQSPSSVSASVGDRVTITCKASQIVGSNVAWYQQKPGKAPEPLIYSASYLYSGVPSRFSGSGSGTDFTLTISSLQPEDFATYYCQQYSSHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 78 - protein, 8 aa, synthetic construct
GFSLTNYG

SEQ ID NO: 79 - protein, 7 aa, synthetic construct
IWSGGNT

SEQ ID NO: 80 - protein, 13 aa, synthetic construct
ARALTYYDYEFAY

SEQ ID NO: 81 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 82 - protein, 6 aa, synthetic construct
QSIGTN

SEQ ID NO: 83 - skipped

SEQ ID NO: 84 - protein, 9 aa, synthetic construct
QQNNNWPTT

SEQ ID NO: 85 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0286006 A1
(43) Pub. Date: Sep. 24, 2026

(54) TREATMENT AND PREVENTION OF CANCER USING HER3 ANTIGEN-BINDING MOLECULES

(71) Applicant: Hummingbird Bioscience Pte. Ltd., Singapore (SG)

(72) Inventors: Piers Ingram, Singapore (SG); Jerome Boyd-Kirkup, Singapore (SG); Konrad Paszkiewicz, Singapore (SG); Dipti Thakkar, Singapore (SG); Namita Gandhi, Singapore (SG); Khang Luu, Singapore (SG); Kevin Fongching Chau, Singapore (SG); Weiyi Toy, Singapore (SG)

(21) Appl. No.: 19/472,479

(22) PCT Filed: Apr. 3, 2024
(86) PCT No.: PCT/EP2024/059029

§ 371 Date: Oct. 3, 2025

Related U.S. Application Data
(60) Provisional application No. 63/457,514, filed on Apr. 6, 2023.

(51) Int. Cl. C07K 16/32; A61K 39/00; A61P 35/00; C07K 16/28; C12Q 1/6886

(52) U.S. Cl. CPC C07K 16/32; A61P 35/00; C07K 16/2863; C12Q 1/6886; A61K 2039/505; C07K 2317/565; C07K 2317/76; C12Q 2600/106; C12Q 2600/156

(57) ABSTRACT

The present disclosure provides the use of an antigen-binding molecule that binds to HER3 for the treatment or prevention of a HER3-associated cancer, wherein the HER3-associated cancer does not comprise genetic variation resulting in an increase in the expression of, or an increase in the activity of a gene product of, MET.

This application claims priority from U.S. 63/457,514 filed 6 Apr. 2023, the contents and elements of which are herein incorporated by reference for all purposes.

### TECHNICAL FIELD

The present disclosure relates to the fields of molecular biology, more specifically antibody technology and methods of medical treatment and prophylaxis.

### BACKGROUND

A number of transmembrane protein kinases have been associated with oncogenesis (Roskoski Jr 2004). The human epidermal growth factor receptor 3 (HER3) has been identified as a key signaling hub in activating key growth factor signaling pathways such as the MAPK/ERK and PI3K/AKT/mTOR pathways, by forming heterodimers with either epidermal growth factor receptor (EGFR) or human epidermal growth factor receptor 2 (HER2) (Gala and Chandarlapaty, Clinical Cancer Research (2014) 6:1410-1416) (Haikala and Jänne, Clinical Cancer Research (2021) 27(13):3528-3539). HER3 activation has emerged as an important mechanism for both tumor progression and acquired resistance to standard of care therapies in multiple indications. HER3 targeting approaches to date have not shown the expected clinical efficacy. Suboptimal inhibition of HER3-mediated signalling is one possible explanation.

WO 2023/017151 A1 discloses the treatment and prevention of cancers characterised by the presence/absence of mutations in genes encoding factors involved in HER3-mediated signalling.

### SUMMARY

In a first aspect, the present disclosure provides an antigen-binding molecule that binds to HER3 for use in a method of treating or preventing a HER3-associated cancer in a subject, wherein the HER3-associated cancer does not comprise genetic variation resulting in an increase in the expression of, or an increase in the activity of a gene product of, MET.

The present disclosure also provides the use of an antigen-binding molecule that binds to HER3 in the manufacture of a medicament for use in treating or preventing a HER3-associated cancer in a subject, wherein the HER3-associated cancer does not comprise genetic variation resulting in an increase in the expression of, or an increase in the activity of a gene product of, MET.

The present disclosure also provides a method of treating or preventing a HER3-associated cancer in a subject, comprising administering to the subject a therapeutically- or prophylactically-effective amount of an antigen-binding molecule that binds to HER3, wherein the HER3-associated cancer does not comprise genetic variation resulting in an increase in the expression of, or an increase in the activity of a gene product of, MET.

In accordance with various aspects of the present disclosure, in some embodiments the HER3-associated cancer: (i) does not comprise genetic variation resulting in an increase in the expression of, or an increase in the activity of a gene product of, KRAS; or (ii) does not comprise genetic variation resulting in an increase in the expression of, or an increase in the activity of a gene product of PIK3CA (e.g. does not comprise an activating mutation to PIK3CA); or (iii) does not comprise genetic variation resulting in an increase in the expression of, or an increase in the activity of a gene product of, BRAF; or (iv) does not comprise genetic variation resulting in a decrease in the expression of, or a decrease in the activity of a gene product of, PTEN.

In some embodiments, the HER3-associated cancer: (i) does not comprise genetic variation resulting in an increase in the expression of, or an increase in the activity of a gene product of, KRAS; and (ii) does not comprise genetic variation resulting in an increase in the expression of, or an increase in the activity of a gene product of PIK3CA (e.g. does not comprise an activating mutation to PIK3CA); and (iii) does not comprise genetic variation resulting in an increase in the expression of, or an increase in the activity of a gene product of, BRAF; and (iv) does not comprise genetic variation resulting in a decrease in the expression of, or a decrease in the activity of a gene product of, PTEN.

In some embodiments, the HER3-associated cancer: (i) does not comprise amplification of MET, and (ii) does not comprise an activating mutation to KRAS, and (iii) does not comprise an activating mutation to PIK3CA, and (iv) does not comprise an activating mutation to BRAF, and (iv) does not comprise deletion of PTEN.

In some embodiments, the HER3-associated cancer: (i) lacks amplification of MET, and (ii) is homozygous for the wildtype allele of KRAS, and (iii) is homozygous for the wildtype allele of PIK3CA, and (iv) is homozygous for the wildtype allele of BRAF, and (iv) is homozygous for the wildtype allele of PTEN.

In some embodiments, the HER3-associated cancer comprises amplification of one or more genes located on chromosome 3q. In some embodiments, the HER3-associated cancer comprises amplification of one or more genes located within chromosome 3q26-3q28. In some embodiments, the HER3-associated cancer comprises amplification of one or more genes selected from: TP63, SOX2 and PIK3CA.

In some embodiments, the HER3-associated cancer comprises amplification of one or more genes located on chromosome 7p. In some embodiments, the HER3-associated cancer comprises amplification of one or more genes located within chromosome 7p11. In some embodiments, the HER3-associated cancer comprises amplification of EGFR.

In some embodiments, the HER3-associated cancer comprises deletion of one or more genes located on chromosome 3p. In some embodiments, the HER3-associated cancer comprises deletion of one or more genes located within chromosome 3p21. In some embodiments, the HER3-associated cancer comprises deletion of TUSC2.

In some embodiments, the HER3-associated cancer comprises genetic variation resulting in increased expression of a ligand for HER3.

In some embodiments, the HER3-associated cancer comprises an NRG gene fusion, an NRG1 gene fusion, or an NRG2 gene fusion.

In some embodiments, the HER3-associated cancer comprises an NRG gene fusion selected from: CLU-NRG1, CD74-NRG1, DOC4-NRG1, SLC3A2-NRG1, RBPMS-NRG1, WRN-NRG1, SDC4-NRG1, RAB21L1-NRG1, VAMP2-NRG1, KIF13B-NRG1, THAP7-NRG1, SMAD4-NRG1, MDK-NRG1, TNC-NRG1, DIP2B-NRG1, MRPL13-NRG1, PARP8-NRG1, ROCK1-NRG1, DPYSL2-NRG1, ATP1B1-NRG1, CDH6-NRG1, APP-NRG1, AKAP13-NRG1, THBS1-NRG1, FOXA1-NRG1, PDE7A-NRG1, RAB31L1-NRG1, CDK1-NRG1, BMPRIB-NRG1, TNFRSF10B-NRG1, MCPH1-NRG1, and SLC12A2-NRG2.

The present disclosure also provides an antigen-binding molecule that binds to HER3 for use in a method of treating or preventing a HER3-associated cancer in a subject, wherein the HER3-associated cancer comprises amplification of one or more genes located on chromosome 3q. The present disclosure also provides the use of an antigen-binding molecule that binds to HER3 in the manufacture of a medicament for use in treating or preventing a HER3-associated cancer in a subject, wherein the HER3-associated cancer comprises amplification of one or more of genes located on chromosome 3q. The present disclosure also provides a method of treating or preventing a HER3-associated cancer in a subject, comprising administering to the subject a therapeutically- or prophylactically-effective amount of an antigen-binding molecule that binds to HER3, wherein the HER3-associated cancer comprises amplification of one or more of genes located on chromosome 3q. In some embodiments, the one or more genes located on chromosome 3q are located within chromosome 3q26-3q28. In some embodiments, the one or more of genes are selected from: TP63, SOX2 and PIK3CA.

The present disclosure also provides an antigen-binding molecule that binds to HER3 for use in a method of treating or preventing a HER3-associated cancer in a subject, wherein the HER3-associated cancer comprises amplification of one or more genes located on chromosome 7p. The present disclosure also provides the use of an antigen-binding molecule that binds to HER3 in the manufacture of a medicament for use in treating or preventing a HER3-associated cancer in a subject, wherein the HER3-associated cancer comprises amplification of one or more of genes located on chromosome 7p. The present disclosure also provides a method of treating or preventing a HER3-associated cancer in a subject, comprising administering to the subject a therapeutically- or prophylactically-effective amount of an antigen-binding molecule that binds to HER3, wherein the HER3-associated cancer comprises amplification of one or more of genes located on chromosome 7p. In some embodiments, the one or more genes located on chromosome 7p are located within chromosome 7p11. In some embodiments, the gene is EGFR.

The present disclosure also provides an antigen-binding molecule that binds to HER3 for use in a method of treating or preventing a HER3-associated cancer in a subject, wherein the HER3-associated cancer comprises deletion of one or more genes located on chromosome 3p. The present disclosure also provides the use of an antigen-binding molecule that binds to HER3 in the manufacture of a medicament for use in treating or preventing a HER3-associated cancer in a subject, wherein the HER3-associated cancer comprises deletion of one or more of genes located on chromosome 3p. The present disclosure also provides a method of treating or preventing a HER3-associated cancer in a subject, comprising administering to the subject a therapeutically- or prophylactically-effective amount of an antigen-binding molecule that binds to HER3, wherein the HER3-associated cancer comprises deletion of one or more of genes located on chromosome 3p. In some embodiments, the one or more genes located on chromosome 3p are located within chromosome 3p21. In some embodiments, the gene is TUSC2.

In accordance with various aspects of the present disclosure, in some embodiments the HER3-associated cancer comprises amplification of one or more of genes selected from: TP63, SOX2 and PIK3CA.

In some embodiments, the HER3-associated cancer comprises amplification of TP63, and comprises amplification of SOX2, and comprises amplification of PIK3CA.

The present disclosure also provides an antigen-binding molecule that binds to HER3 for use in a method of treating or preventing a HER3-associated cancer in a subject, wherein the HER3-associated cancer comprises genetic variation resulting in an increase in the expression of, or an increase in the activity of a gene product of, MET, and wherein the method further comprises administering an antagonist of HER3-mediated signalling.

The present disclosure also provides the use of an antigen-binding molecule that binds to HER3 for use in the manufacture of a medicament for use in treating or preventing a HER3-associated cancer in a subject, wherein the HER3-associated cancer comprises genetic variation resulting in an increase in the expression of, or an increase in the activity of a gene product of, MET, and wherein the method further comprises administering an antagonist of HER3-mediated signalling.

The present disclosure also provides a method of treating or preventing a HER3-associated cancer in a subject, comprising administering to the subject a therapeutically- or prophylactically-effective amount of an antigen-binding molecule that binds to HER3, wherein the HER3-associated cancer comprises genetic variation resulting in an increase in the expression of, or an increase in the activity of a gene product of, MET, and wherein the method further comprises administering an antagonist of HER3-mediated signalling.

In accordance with various aspects of the present disclosure, the HER3-associated cancer: (i) comprises genetic variation resulting in an increase in the expression of, or an increase in the activity of a gene product of, KRAS; or (ii) comprises genetic variation resulting in an increase in the expression of, or an increase in the activity of a gene product of, PIK3CA; or (iii) comprises genetic variation resulting in an increase in the expression of, or an increase in the activity of a gene product of, BRAF; or (iv) comprises genetic variation resulting in a decrease in the expression of, or a decrease in the activity of a gene product of, PTEN.

In some embodiments, the HER3-associated cancer: (i) comprises genetic variation resulting in an increase in the expression of, or an increase in the activity of a gene product of, KRAS; and (ii) comprises genetic variation resulting in an increase in the expression of, or an increase in the activity of a gene product of, PIK3CA; and (iii) comprises genetic variation resulting in an increase in the expression of, or an increase in the activity of a gene product of, BRAF; and (iv) comprises genetic variation resulting in a decrease in the expression of, or a decrease in the activity of a gene product of, PTEN.

The present disclosure also provides a method of selecting a subject for treatment with an antigen-binding molecule that binds to HER3, comprising:
(a) analysing a subject's cancer to determine whether the cancer comprises genetic variation resulting in an increase in the expression of, or an increase in the activity of a gene product of, MET; and
(b) selecting a subject for treatment with an antigen-binding molecule that binds to HER3 where the subject's cancer is determined in step (a) not to comprise such genetic variation.

In some embodiments, the method comprises:
(a)
(i) analysing a subject's cancer to determine whether the cancer comprises genetic variation resulting in an increase in the expression of, or an increase in the activity of a gene product of, KRAS; or
(ii) analysing a subject's cancer to determine whether the cancer comprises an activating mutation to PIK3CA; or
(iii) analysing a subject's cancer to determine whether the cancer comprises genetic variation resulting in an increase in the expression of, or an increase in the activity of a gene product of, BRAF; or
(iv) analysing a subject's cancer to determine whether the cancer comprises genetic variation resulting in a decrease in the expression of, or a decrease in the activity of a gene product of, PTEN; and
(b) selecting a subject for treatment with an antigen-binding molecule that binds to HER3 where the subject's cancer is determined in step (a) not to comprise such genetic variation/mutation.

In some embodiments, the method comprises:
(a)
(i) analysing a subject's cancer to determine whether the cancer comprises genetic variation resulting in an increase in the expression of, or an increase in the activity of a gene product of, KRAS; and
(ii) analysing a subject's cancer to determine whether the cancer comprises an activating mutation to PIK3CA; and
(iii) analysing a subject's cancer to determine whether the cancer comprises genetic variation resulting in an increase in the expression of, or an increase in the activity of a gene product of, BRAF; and
(iv) analysing a subject's cancer to determine whether the cancer comprises genetic variation resulting in a decrease in the expression of, or a decrease in the activity of a gene product of, PTEN; and
(b) selecting a subject for treatment with an antigen-binding molecule that binds to HER3 where the subject's cancer is determined in step (a) not to comprise such genetic variation/mutation.

In some embodiments, the method further comprises:
(c) administering an antigen-binding molecule that binds to HER3 to a subject selected for treatment in step (b).

The present disclosure also provides a method of selecting a subject for treatment with (i) an antagonist of HER3-mediated signalling and (ii) an antigen-binding molecule that binds to HER3, comprising:
(a) analysing a subject's cancer to determine whether the cancer comprises genetic variation resulting in an increase in the expression of, or an increase in the activity of a gene product of, MET; and
(b) selecting a subject for treatment with (i) an antagonist of HER3-mediated signalling and (ii) an antigen-binding molecule that binds to HER3 where the subject's cancer is determined in step (a) to comprise such genetic variation.

In some embodiments, the method comprises:
(a)
(i) analysing a subject's cancer to determine whether the cancer comprises genetic variation resulting in an increase in the expression of, or an increase in the activity of a gene product of, KRAS; or
(ii) analysing a subject's cancer to determine whether the cancer comprises an activating mutation to PIK3CA; or
(iii) analysing a subject's cancer to determine whether the cancer comprises genetic variation resulting in an increase in the expression of, or an increase in the activity of a gene product of, BRAF; or
(iv) analysing a subject's cancer to determine whether the cancer comprises genetic variation resulting in a decrease in the expression of, or a decrease in the activity of a gene product of, PTEN; and
(b) selecting a subject for treatment with (i) an antagonist of HER3-mediated signalling and (ii) an antigen-binding molecule that binds to HER3 where the subject's cancer is determined in step (a) to comprise such genetic variation/mutation.

In some embodiments, the method comprises:
(a)
(i) analysing a subject's cancer to determine whether the cancer comprises genetic variation resulting in an increase in the expression of, or an increase in the activity of a gene product of, KRAS; and
(ii) analysing a subject's cancer to determine whether the cancer comprises an activating mutation to PIK3CA; and
(iii) analysing a subject's cancer to determine whether the cancer comprises genetic variation resulting in an increase in the expression of, or an increase in the activity of a gene product of, BRAF; and
(iv) analysing a subject's cancer to determine whether the cancer comprises genetic variation resulting in a decrease in the expression of, or a decrease in the activity of a gene product of, PTEN; and
(b) selecting a subject for treatment with (i) an antagonist of HER3-mediated signalling and (ii) an antigen-binding molecule that binds to HER3 where the subject's cancer is determined in step (a) to comprise such genetic variation/mutation.

In some embodiments, the method further comprises:
(c) administering (i) an antagonist of HER3-mediated signalling and (ii) an antigen-binding molecule that binds to HER3 to a subject selected for treatment in step (b).

In accordance with various aspects of the present disclosure, in some embodiments the HER3-associated cancer is selected from: a solid tumor, breast cancer, breast carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck, lung cancer, non-small cell lung cancer, lung adenocarcinoma, squamous cell lung carcinoma, ovarian cancer, ovarian carcinoma, ovarian serous adenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, uterine cancer, uterine corpus endometrial carcinoma, thyroid cancer, thyroid carcinoma, pheochromocytoma, paraganglioma, bladder cancer, bladder urothelial carcinoma, prostate cancer, prostate adenocarcinoma, sarcoma and thymoma.

In some embodiments, the antigen-binding molecule that binds to HER3 is selected from: 10D1F, seribantumab, elgemtumab, patritumab, GSK2849330, lumretuzumab, CDX-3379, AV-203, barecetamab, TK-A3, TK-A4, MP-EV20, 1A5-3D4, 9F7-F11, 16D3-C1, NG33, A5, F4, huHER3-8, REGN1400 and zenocutuzumab.

In some embodiments, the antigen-binding molecule that binds to HER3 comprises:
(i) a heavy chain variable (VH) region incorporating the following CDRs:
   HC-CDR1 having the amino acid sequence of SEQ ID NO:40
   HC-CDR2 having the amino acid sequence of SEQ ID NO:43
   HC-CDR3 having the amino acid sequence of SEQ ID NO:48; and
(ii) a light chain variable (VL) region incorporating the following CDRs:
   LC-CDR1 having the amino acid sequence of SEQ ID NO:66
   LC-CDR2 having the amino acid sequence of SEQ ID NO:69
   LC-CDR3 having the amino acid sequence of SEQ ID NO:74.

In some embodiments, the antigen-binding molecule that binds to HER3 comprises:
(i) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45; and
(ii) a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70.

In some embodiments, antigen-binding molecule comprises:
a VH region comprising an amino acid sequence having at least 70% sequence identity to the amino acid sequence of SEQ ID NO:33; and
a VL region comprising an amino acid sequence having at least 70% sequence identity to the amino acid sequence of SEQ ID NO:58.

In some embodiments, the antigen-binding molecule comprises:
a polypeptide comprising, or consisting of, an amino acid sequence having at least 70% sequence identity to the amino acid sequence of SEQ ID NO:75; and
a polypeptide comprising, or consisting of, an amino acid sequence having at least 70% sequence identity to the amino acid sequence of SEQ ID NO:76.

In accordance with the various different aspects of the present disclosure, in some embodiments the method of treating or preventing the HER3-associated cancer further comprises administering an antagonist of EGFR to the subject.

In some embodiments, the antagonist of EGFR is antigen-binding molecule that binds to EGFR.

In some embodiments, the antigen-binding molecule that binds to EGFR comprises:
(i) a VH region incorporating the following CDRs:
   HC-CDR1 having the amino acid sequence of SEQ ID NO:78
   HC-CDR2 having the amino acid sequence of SEQ ID NO:79
   HC-CDR3 having the amino acid sequence of SEQ ID NO:80; and
(ii) a VL region incorporating the following CDRs:
   LC-CDR1 having the amino acid sequence of SEQ ID NO:82
   LC-CDR2 having the amino acid sequence of SEQ ID NO:83
   LC-CDR3 having the amino acid sequence of SEQ ID NO:84.

In some embodiments, the antigen-binding molecule that binds to EGFR comprises:
a VH region comprising an amino acid sequence having at least 70% sequence identity to the amino acid sequence of SEQ ID NO:77; and
a VL region comprising an amino acid sequence having at least 70% sequence identity to the amino acid sequence of SEQ ID NO:81.

In some embodiments, the antigen-binding molecule that binds to EGFR comprises:
a polypeptide comprising, or consisting of, an amino acid sequence having at least 70% sequence identity to the amino acid sequence of SEQ ID NO:85; and
a polypeptide comprising, or consisting of, an amino acid sequence having at least 70% sequence identity to the amino acid sequence of SEQ ID NO:86.

### DESCRIPTION

The present disclosure is based in part on the inventors' determination that cancers comprising genetic variation resulting in an increase in the expression of MET (in particular, amplification of MET) are less likely to respond well to anti-HER3 antibody therapy. Cancers lacking such genetic variation are identified as cancers that are responsive to treatment with anti-HER3 antibodies. The inventors determined that in addition to evaluating the mutation status of PTEN, KRAS, PIK3CA and BRAF as described in WO 2023/017151 A1, further determining the amplification status of MET provides for the prediction of positive response to treatment with anti-HER3 antibody therapy with greater sensitivity (see Example 4).

The present disclosure is also based on the inventors' determination that cancers comprising genetic variation resulting in an increase in the expression of genes located within chromosome 3q26-3q28 (in particular, amplification of such genes) are more likely to respond well to anti-HER3 antibody therapy. Cancers comprising such genetic variation are identified as cancers that are responsive to treatment with anti-HER3 antibodies.

The present disclosure is also based on the inventors' determination that cancers comprising genetic variation resulting in an increase in the expression of genes located within chromosome 3q26-3q28 (in particular, amplification of such genes) are more likely to respond well to anti-HER3 antibody therapy. Cancers comprising such genetic variation are identified as cancers that are responsive to treatment with anti-HER3 antibodies.

### HER3 and HER3-Mediated Signalling

HER3 (also known e.g. as ERBB3, LCCS2, MDA-BF-1) is the protein identified by UniProt P21860.

The structure and function of HER3 is described e.g. in Cho and Leahy Science (2002) 297 (5585):1330-1333, Singer et al., Journal of Biological Chemistry (2001) 276, 44266-44274, Roskoski et al., Pharmacol. Res. (2014) 79: 34-74, Bazley and Gullick Endocrine-Related Cancer (2005) S17-S27 and Mujoo et al., Oncotarget (2014) 5(21):10222-10236, each of which are hereby incorporated by reference in their entirety. HER3 is a single-pass transmembrane ErbB receptor tyrosine kinase having an N-terminal extracellular region (SEQ ID NO:9) comprising two leucine-rich subdomains (domains I and Ill, shown in SEQ ID NOs:15 and 17, respectively) and two cysteine-rich subdomains (domains II and IV, shown in SEQ ID NOs:16 and 18, respectively). Domain II comprises a p hairpin dimerisation loop (SEQ ID NO:19) which is involved in intermolecular interactions with other HER receptor molecules. The extracellular region is linked via a transmembrane region (SEQ ID NO:10) to a cytoplasmic region (SEQ ID NO:11). The cytoplasmic region comprises a juxtamembrane segment (SEQ ID NO:12), a protein kinase domain (SEQ ID NO:13), and a C-terminal segment (SEQ ID NO:14).

In this specification ‘HER3’ refers to HER3 from any species and includes HER3 isoforms, fragments, variants (including mutants) or homologues from any species.

As used herein, a ‘fragment’, ‘variant’ or ‘homologue’ of a protein may optionally be characterised as having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference protein (e.g. a reference isoform). In some embodiments, fragments, variants, isoforms and homologues of a reference protein may be characterised by ability to perform a function performed by the reference protein.

A ‘fragment’ generally refers to a fraction of the reference protein. A ‘variant’ generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An ‘isoform’ generally refers to a variant of the reference protein expressed by the same species as the species of the reference protein (e.g. human HER3 isoforms 1 to 5 are all isoforms of one another). A ‘homologue’ generally refers to a variant of the reference protein produced by a different species as compared to the species of the reference protein. For example, human HER3 isoform 1 (P21860-1, v1; SEQ ID NO:1) and Rhesus macaque HER3 (UniProt: F7HEH3-1, v2; SEQ ID NO:20) are homologues of one another. Homologues include orthologues.

A ‘fragment’ of a reference protein may be of any length (by number of amino acids), although may optionally be at least 20% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein.

A fragment of HER3 may have a minimum length of one of 10, 20, 30, 40, 50, 100, 150, 200, 250, 300, 350, 400, 450, 500, 600, 700, 800, 900, 1000, 1100, 1200 amino acids, and may have a maximum length of one of 20, 30, 40, 50, 100, 150, 200, 250, 300, 350, 400, 450, 500, 600, 700, 800, 900, 1000, 1100, 1200, or 1300 amino acids.

In some embodiments, the HER3 is HER3 from a mammal (e.g. a primate (rhesus, cynomolgous, non-human primate or human) and/or a rodent (e.g. rat or murine) HER3). Isoforms, fragments, variants or homologues of HER3 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature HER3 isoform from a given species, e.g. human. Isoforms, fragments, variants or homologues may optionally be functional isoforms, fragments, variants or homologues, e.g. having a functional property/activity of the reference HER3 (e.g. human HER3 isoform 1), as determined by analysis by a suitable assay for the functional property/activity. For example, an isoform, fragment, variant or homologue of HER3 may display association with one or more of: HER2, NRG1 (type I, II, III, IV, V or VI) or NRG2 (a or p).

In some embodiments, the HER3 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs:1 to 8.

In some embodiments, a fragment of HER3 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs:9 to 19, e.g. one of 9, 16 or 19.

Signalling through HER3 involves receptor heteromultimerization (i.e. with other ErBB receptors, e.g. HER2, EGFR) and consequent autophosphorylation by the protein kinase domain of tyrosine residues of the cytoplasmic region. HER3 lacks kinase activity and does not form stable homodimers. Therefore, HER3 must be transphosphorylated by binding to a kinase-active heterodimer partner (e.g. EGFR or HER2) for signal transduction to take place (Berger M B et al., FEBS Lett 2004; 569:332-6; Kim H H et al., Biochem J 1998; 334:189-95.).

Multimerization (e.g. dimerization) of HER receptor family members is required for activating cell growth signaling pathways, and HER3 can dimerize with other HER family members in both a ligand-dependent and ligand-independent manner. The HER3 extracellular domain (ECD) exists in a reversible equilibrium between a ‘closed’ inactive conformation and an ‘open’ active conformation, in which the dimerization arm within domain II is exposed to allow dimerization along the domain II dimerization interface, and in particular through the cysteine-rich CR1 region (Carraway, K. L., et al., Nature, 1997. 387(6632): 512-6; Riese, D. J., et al., Mol Cell Biol, 1995. 15(10): 5770-6; Harari, D., et al., Oncogene, 1999. 18(17): 2681-9; Zhang, D., et al., Proc Natl Acad Sci USA, 1997. 94(18): 9562-7; Meyer et al., Nature, 1995. 378(6555):386-90; Jura, N., et al., Proc Natl Acad Sci USA, 2009. 106(51): 21608-13; Fornaro, L., et al., Nat Rev Gastroenterol Hepatol, 2011. 8(7):369-83; Mota et al., Oncotarget (2015) 5:89284-306). HER3 is ‘activated’ when the equilibrium is shifted in favor of the open conformation, increasing the probability of forming active heterodimers. The conventional model for activation is ligand-dependent, that is, the equilibrium shifts when HER3 in the open conformation is stabilized by binding of its ligand such as a neuregulin (NRG), e.g. NRG1 (also known as heregulin, HRG) or NRG2. In addition, the presence of any dimerization partner at a sufficient concentration will shift the equilibrium in favor of the open conformation, as they binds to and stabilize HER3 transiently in an open conformation. This is known as ligand-independent activation (Jura, N., et al., Proc Natl Acad Sci USA, 2009. 106(51): 21608-13; Fornaro, L., et al., Nat Rev Gastroenterol Hepatol, 2011. 8(7): p. 369-83; Mota et al., Oncotarget (2015) 5:89284-306).

Herein, ‘HER3-mediated signalling’ refers to signalling mediated by HER3 and/or multimeric ErBB family member receptor complexes comprising HER3. ‘Signalling’ refers to signal transduction and other cellular processes governing cellular activity. HER3-mediated signalling may be mediated by HER3 receptor-containing complexes, e.g. by heteromultimeric complexes comprising HER3 and other HER receptors (e.g. HER2, EGFR). HER3-mediated signalling may be ligand-dependent, e.g. triggered by binding of NRG (e.g. NRG1, NRG2), or may be ligand-independent.

HER3-mediated signalling progresses intracellularly through the MAPK/ERK and PI3K/AKT/mTOR pathways to promote cell survival and proliferation. HER3-mediated signalling is described e.g. in Gala and Chandarlapaty, Clin Cancer Res. (2014) 20(6): 1410-1416, Mishra et al., Oncol Rev. (2018) 12(1): 355, Baselga et al., Nat Rev Cancer (2009) 9:463-75, Yarden et al. Nat Rev Mol Cell Biol (2001) 2:35052073, Mota et al., Oncotarget (2015) 5:89284-306 and Haikala and Jsnne, Clin. Cancer Res. (2021) 27:3528-39, all of which are hereby incorporated by reference in their entirety.

Phosphorylated tyrosine residues in the protein kinase domains of HER3-containing receptor complexes recruit adaptor/effector proteins GRB2, via interaction with its SH2 domain. Upon ligand stimulation, the activated receptor (EGFR/HER2) undergoes autophosphorylation and provides phospho-tyrosine residues for recruiting GRB2. GRB2 binds via its SH3 domains to the guanine nucleotide exchange factor SOS. Activated SOS in GRB2-SOS complexes promotes removal of GDP from, and thereby activation of, Ras family GTPases such as H-Ras, N-Ras and K-Ras. Activated Ras GTPases in turn activate RAF kinases such as A-Raf, B-Raf and C-Raf. RAF kinases in turn phosphorylate and activate MEK1 and MEK2, which then phosphorylate and activate MAPKs (also known as ERKs). Activated MAPKs are able to directly regulate the activity of transcription factors such as c-Myc. Activated MAPKs also upregulate translation of mRNA into protein via phosphorylation of RSK, and consequent phosphorylation and activation of 40S ribosomal protein S6. Activated MAPKs also phosphorylate and activate MNK, which in turn phosphorylates and activates the transcription factor CREB.

Phosphorylated tyrosine residues in the protein kinase domain of HER3 also recruit the p85 subunit of P13K, through its SH2 domain. Association of p85 causes allosteric activation of the lipid kinase p100α subunit of P13K. Activated P13K results in conversion of PIP2 to PIP3, which recruits AKT to be phosphorylated and activated by mTORC2 and PDK1. Phosphorylated AKT has a number of activities, including activating CREB and mTOR. PTEN antagonises signalling through the PI3K/AKT/mTOR pathway by dephosphorylating PIP3 to PIP2, and PP2A inhibits the PI3K/AKT/mTOR pathway by dephosphorylating AKT.

Oncogenic Src homology region 2 protein tyrosine phosphatase 2 (SHP2) promotes tumor progression and serves as a pivotal hub to connect multiple oncogenic signaling pathways, such as PI3K/Akt, Ras/Raf/MAPK (Dong et al., Front. Cell Dev. Biol., 11 Mar. 2021). GAB2 binds to GRB2 and becomes phosphorylated at multiple tyrosine residues, capable of binding to the SH2 domains of SHP2 and p85 (Adams et al., Mol Cancer Res. 2012 October; 10(10):1265-70; (Liu et al., Proc. Natl. Acad. Sci. U.S.A. (2016) 113, 984-989). The interactions induce conformation changes, relieving the auto-inhibition of the SHP2 catalytic site (Neel et al., Trends Biochem Sci. 2003 June; 28(6):284-93) and relieving the inhibition of p85 on the p110 catalytic subunit of P13K (Cuevas et al., J Biol Chem. 2001 Jul. 20; 276(29):27455-6), respectively. SHP2 has been shown to activate RAS by direct dephosphorylation of RAS (Bunda et al., Nat Commun. 2015 Nov. 30; 60:8859), inhibition of RASGAP (RAS GTPase activating protein) (Neel et al., Trends Biochem Sci. 2003 June; 28(6):284-93) and SPRY (Hanafusa et al., J Biol Chem. 2004 May 28; 279(22):22992-5). SHP2 overexpression has been shown to enhance tumor invasion by activating the PI3K/Akt axis (Hu et al., Onco Targets Ther. (2017) 10, 3881-3891) while SHP2 knockdown inhibits cell migration and the tumor-promoting effect of SHP2 is partially related to Akt signaling (Cao et al., Pathol. Res. Pract. (2019) 215:152621).

STAT3 and 5 proteins are transcription factors that enhance the expression of p85a, p110a and AKT1 and thereby augment signaling through the PI3K/AKT signal transduction cascade (Radler et al., Mol Cell Endocrinol. 2017 Aug. 15; 451: 31-39). HER3-mediated activation of STAT3 results in the upregulation of SOX2 expression, promoting survival. Upon activation by JAK2, phosphorylated STAT5 binds to the SH2 domain of the p85a regulatory subunit of P13K in a PRL signaling-dependent manner suggesting that STAT5 may also directly participate in the signaling of P13K complexes. Another kinase that phosphorylates EGFR is the cytokine-regulated tyrosine kinase Jak2, thus allowing MAPK activation even by a kinase-defective mutant of EGFR (Mishra et al., Oncol Rev. (2018) 12(1): 355, Baselga et al., Nat Rev Cancer (2009) 9:463-75). The collective observations in genetic models overexpressing or lacking active STAT5 and AKT or expressing mutant PTEN supports the notion that STAT5 functions as a survival factor during normal mammary gland development and as an oncogene during mammary carcinogenesis are mediated by the PI3K/AKT pathway (Radler et al., Mol Cell Endocrinol. 2017 Aug. 15; 451: 31-39).

The proto-oncogene MET has been shown to phosphorylate and activate HER3. See Frazier et al., Oncogene (2019) 38(11):1936-1950. Elevated expression of MET has been shown to upregulate phosphorylation of HER3. HER3 preferentially interacts with MET during its maturation along the secretory pathway, resulting in accumulation of phosphorylated HER3 in the Golgi.

The PI3K/AKT-signaling pathway is commonly altered by gene amplification and/or mutations in certain cancers. The frequently amplified 3q26 to 3q28 chromosomal region, in which P/K3CA is located, also resides the TP63 and SOX2 cell lineage genes. TP63 is a member of the TP53 gene family and is expressed in the basal compartment of the skin, esophagus, lung airways and larynx during development and homeostasis. TP63 is used as a diagnostic marker of squamous versus adenocarcinoma forms of lung and esophageal cancer. Preclinical data suggests that TP63 regulates NRG1 expression in SCC (see e.g. Hedge et al., eLife. (2019) 8:e46551), suggesting that the HER3 signaling pathway is active in TP63-amplified squamous cell cancers.

### Genetic Variation, Alleles and Genotypes

Aspects and embodiments of the present disclosure are concerned with therapeutic and prophylactic intervention for the treatment/prevention of cancers characterised by the absence or presence of certain genetic variation with respect to genes encoding factors involved in HER3-mediated signalling.

In particular, the present disclosure pertains to cancers characterised by the absence or presence of genetic variation resulting in an increase/decrease in the gene and/or protein expression of genes encoding factors involved in HER3-mediated signalling, or genetic variation resulting in an increase/decrease in the activity of a gene product of, genes encoding factors involved in HER3-mediated signalling.

Genetic variation contemplated in accordance with the present disclosure includes mutation, gene amplification, and gene deletion.

As used herein, a ‘mutation’ refers to a difference relative to a reference nucleotide sequence, e.g. the most common nucleotide sequence of a given gene (which may be referred to as the ‘wildtype’ nucleotide sequence). In some embodiments, a mutation may be or comprise one or more of the following relative to the reference sequence (e.g. the wildtype sequence): a nucleotide polymorphism (e.g. a single nucleotide polymorphism (SNP) or a multiple nucleotide polymorphism (MNP)), an insertion, a deletion, a frame-shift mutation, a missense mutation or a translocation.

A nucleotide polymorphism may comprise substitution of a nucleotide of the reference sequence with another, non-identical nucleotide (e.g. in the case of a SNP), or may comprise substitution of two or more nucleotides of the reference sequence with non-identical nucleotides (e.g. in the case of a MNP). An insertion refers to introduction of one or more nucleotides into the nucleotide sequence of the reference sequence. A deletion refers to removal/excision of one or more nucleotides from the nucleotide sequence of the reference sequence. A frame-shift mutation refers to an insertion or deletion mutation that changes the reading frame for translation of from RNA encoded by the mutant nucleotide sequence. Such frameshift mutations comprise insertion/deletion of a number of nucleotides that is not divisible by three. A missense mutation refers to a substitution (or insertion or deletion) that does not alter the amino acid sequence of a peptide/polypeptide translated from RNA encoded by the mutant nucleotide sequence. Such mutations may result in the replacement of one or more nucleotides with (a) non-identical nucleotide(s), wherein the codon comprising the non-substituted nucleotide(s) and the codon comprising the substituted nucleotide(s) encode the same amino acid (as a consequence of degeneracy of the genetic code). Other such missense mutations may be provided in a non-protein encoding region of the nucleotide sequence, e.g. an intron. A translocation refers large-scale replacement of a nucleotide, or sequence of nucleotides, within the reference sequence with a non-identical nucleotide/sequence of nucleotides. Translocation occurs as a consequence of recombination between nucleotide sequences, e.g. provided on different chromosomes.

As used herein, an ‘activating mutation’ refers to a mutation that is known, or that is predicted, to result in an increase in the level of (gene or protein) expression of the relevant gene, and/or a mutation that is known, or that is predicted, to result in an increase in an activity of a product of the gene. An activating mutation may give rise to one or more of the following in a cell comprising one or more alleles of a gene comprising the activating mutation: increased transcription of the gene; an increase the level of RNA encoded by the gene; decreased degradation of RNA encoded by the gene; an increase in the level of a product of the gene; an increase in the level of a peptide/polypeptide encoded by the gene; an increase in the level of normal splicing of pre-mRNA encoded by the gene; increased translation of mRNA encoding a peptide/polypeptide encoded by the gene; an increase in the level of normal post-translational processing of a peptide/polypeptide encoded by the gene; an increase in the level of normal trafficking of a peptide/polypeptide encoded by the gene; decreased degradation of a peptide/polypeptide encoded by the gene; an increase in the level of one or more functional properties displayed by a product of the gene; an increase in the level of one or more functional properties displayed by a peptide/polypeptide encoded by the gene; a novel functional property displayed by a product of the gene; and/or a novel functional property displayed by a peptide/polypeptide encoded by the gene. In some embodiments, an activating mutation according to the present disclosure is not, or does not comprise, amplification of the relevant gene.

Conversely, an ‘inactivating mutation’ refers to a mutation resulting in a decrease in the level of expression of a given gene, and/or a mutation resulting in a decrease in an activity of a product of the gene. An inactivating mutation may give rise to one or more of the following in a cell comprising one or more alleles of a gene comprising the inactivating mutation: decreased transcription of the gene; a decrease the level of RNA encoded by the gene; increased degradation of RNA encoded by the gene; a decrease in the level of a product of the gene; a decrease in the level of a peptide/polypeptide encoded by the gene; a decrease in the level of normal splicing of pre-mRNA encoded by the gene; decreased translation of mRNA encoding a peptide/polypeptide encoded by the gene; a decrease in the level of normal post-translational processing of a peptide/polypeptide encoded by the gene; a decrease in the level of normal trafficking of a peptide/polypeptide encoded by the gene; increased degradation of a peptide/polypeptide encoded by the gene; a decrease in the level of one or more functional properties displayed by a product of the gene; and/or a decrease in the level of one or more functional properties displayed by a peptide/polypeptide encoded by the gene. In some embodiments, an inactivating mutation according to the present disclosure is not, or does not comprise, deletion of the relevant gene.

As used herein, ‘amplification’ refers to an increase in the number of copies of a given gene or a fragment thereof. Thus, ‘amplification’ of a given reference gene refers to the provision of one or more additional copies of the given gene, or additional copies of a fragment of the given gene, relative to the number of copies of the gene/fragment present in a healthy (e.g. non-cancerous) diploid cell. Accordingly, a cell comprising amplification of a given reference gene comprises one or more additional copies of the given gene, or additional copies of a fragment of the given gene, relative to the number of copies of the gene/fragment present in a healthy (e.g. non-cancerous) diploid cell. In some embodiments, a cell comprising amplification of a given reference gene/fragment thereof comprises more than two copies of the relevant gene/fragment. In some embodiments, the cell comprises one of 3, 4, 5, 6, 7, 8, 9, 10 or more copies of the relevant gene/fragment.

Amplification according to the present disclosure encompasses ‘gain’, which is sometimes used in the art to describe a relatively small increase in the number of copies of a given gene or a fragment thereof (e.g. to 3 or 4 copies of the relevant gene/fragment).

As used herein, ‘gene deletion’ refers to decrease in the number of copies of a given gene or a fragment thereof. Thus, ‘deletion’ of a given reference gene refers to a reduction in the number of copies of the given gene, or a reduction in the number of copies of a fragment of the given gene, relative to the number of copies of the gene/fragment present in a healthy (e.g. non-cancerous) diploid cell. Accordingly, a cell comprising deletion of a given reference gene have a reduction in the number of copies of the given gene, or a reduction in the number of copies of a fragment of the given gene, relative to the number of copies of the gene/fragment present in a healthy (e.g. non-cancerous) diploid cell. In some embodiments, a cell comprising deletion of a given reference gene/fragment thereof comprises fewer than two copies of the relevant gene/fragment. In some embodiments, the cell comprises 0 or 1 copies of the relevant gene/fragment.

Gene deletions according to the present disclosure encompass ‘shallow’ and ‘deep’ deletions. Shallow deletions may be heterozygous deletions, i.e. where only one copy of the gene/fragment is deleted. Deep deletions may be homozygous deletions, i.e. where both copies of the gene/fragment are deleted.

A gene fragment in accordance with present disclosure may comprise a contiguous sequence of nucleotides of the reference gene constituting at least 2%, e.g. constituting one of at least 5%, at least 10%, at least 15%, at least 20%, at least 25%, at least 30%, at least 40% or at least 50% of the complete nucleotide sequence of the reference gene.

Gene amplification and deletion may be evaluated using copy number analysis algorithms such as Genomic Identification of Significant Targets in Cancer (GISTIC) 2.0, which is described e.g. in Mermel et al., Genome Biology, 12:R41.

Genetic variation according to the present disclosure may result in an increase or decrease in the expression of a given gene. ‘Expression’ may be gene expression and/or protein expression. Gene expression can be evaluated using techniques providing for the detection and/or quantification of RNA transcribed from the relevant gene and/or RNA encoding a gene product encoded by the relevant gene. Such techniques include, e.g., quantitative real-time PCR (qRT-PCR). Protein expression can be evaluated using techniques providing for the detection and/or quantification of a peptide/polypeptide encoded by the relevant gene. Such techniques include, e.g., antibody-based methods, such as western blot, immunohistochemistry, immunocytochemistry, flow cytometry, etc.

It will be appreciated that protein expression of a given gene may also be referred to as expression of the relevant protein encoded by the gene. By way of illustration, ‘protein expression of MET’ may alternatively be referred to as ‘expression of c-Met’.

Genetic variation according to the present disclosure may result in an increase or decrease in the activity of a gene product of a given gene. As used herein, a ‘gene product’ refers to a molecule produced by gene expression from a given gene. A gene product may be an RNA encoded by the gene, e.g. an RNA transcribed from the gene, or an RNA produced by post-transcriptional processing of RNA transcribed from the gene. A gene product may be a peptide/polypeptide encoded by the gene, e.g. a peptide/polypeptide translated from RNA transcribed from the gene (e.g. after post-transcriptional processing), or peptide/polypeptide produced by post-translational processing of protein translated from RNA transcribed from the gene. In some embodiments, the gene product is a peptide/polypeptide encoded by the gene.

An activity of a gene product (e.g. a peptide/polypeptide encoded by the gene) may be any functional property displayed by the gene product. In some embodiments, the activity of a gene product is a functional property displayed by a gene product encoded by the wildtype allele of the relevant gene. By way of illustration, an activity of a gene product of MET may be a functional property displayed by c-Met, e.g. tyrosine kinase activity.

Genetic variation resulting in an increase in the (gene and/or protein) expression of a given gene may be known, or may be predicted, to give rise to one or more of the following in a cell comprising such genetic variation: an increase in the number of copies of the gene (e.g. to more than two copies); increased transcription of the gene; an increase the level of RNA encoded by the gene; decreased degradation of RNA encoded by the gene; an increase in the level of a product of the gene; an increase in the level of a peptide/polypeptide encoded by the gene; an increase in the level of normal splicing of pre-mRNA encoded by the gene; increased translation of mRNA encoding a peptide/polypeptide encoded by the gene; an increase in the level of normal post-translational processing of a peptide/polypeptide encoded by the gene; an increase in the level of normal trafficking of a peptide/polypeptide encoded by the gene; and/or decreased degradation of a peptide/polypeptide encoded by the gene. Genetic variation resulting in an increase in the activity of product of a given gene may be known, or may be predicted, to give rise to one or more of the following in a cell comprising such genetic variation: an increase in the level of one or more functional properties displayed by a product of the gene; an increase in the level of one or more functional properties displayed by a peptide/polypeptide encoded by the gene; a novel functional property displayed by a product of the gene; and/or a novel functional property displayed by a peptide/polypeptide encoded by the gene.

Genetic variation resulting in a decrease in the (gene and/or protein) expression of a given gene may be known, or may be predicted, to give rise to one or more of the following in a cell comprising such genetic variation: a decrease in the number of copies of the gene (e.g. to fewer than two copies); decreased transcription of the gene; a decrease the level of RNA encoded by the gene; increased degradation of RNA encoded by the gene; a decrease in the level of a product of the gene; a decrease in the level of a peptide/polypeptide encoded by the gene; a decrease in the level of normal splicing of pre-mRNA encoded by the gene; decreased translation of mRNA encoding a peptide/polypeptide encoded by the gene; a decrease in the level of normal post-translational processing of a peptide/polypeptide encoded by the gene; a decrease in the level of normal trafficking of a peptide/polypeptide encoded by the gene; and/or increased degradation of a peptide/polypeptide encoded by the gene. Genetic variation resulting in a decrease in the activity of product of a given gene may be known, or may be predicted, to give rise to one or more of the following in a cell comprising such genetic variation: a decrease in the level of one or more functional properties displayed by a product of the gene; and/or a decrease in the level of one or more functional properties displayed by a peptide/polypeptide encoded by the gene.

The most common version of the nucleotide sequence of a given gene may be referred to as the wildtype allele of the gene. A version of the nucleotide sequence of a given gene comprising genetic variation may be referred to as a ‘variant’ or ‘mutant’ allele of the gene. It will be appreciated that the nucleotide sequence of a variant/mutant allele of a given gene has a nucleotide sequence which is non-identical to the nucleotide sequence of the wildtype allele.

Herein, a cancer comprising cells having specified characteristics may be referred to herein simply as a cancer having those characteristics. It will be appreciated that in embodiments herein, cancers comprising cells having specified characteristics may be, or may comprise, one or more tumors comprising cells having those characteristics. That is, where a cancer is described as having a given genetic variation, mutation status, allele or genotype it will be appreciated that cells of the cancer have the relevant genetic variation, mutation status, allele or genotype.

By way of illustration, where a cancer is described as comprising a given mutation, the cancer comprises cells comprising the mutation. Similarly, where a cancer is described as being homozygous for a given genetic variation/allele, the cancer comprises cells which are homozygous for the genetic variation/allele. Similarly, where a cancer is described as being heterozygous for a given genetic variation/allele, the cancer comprises cells which are heterozygous for the genetic variation/allele.

Where a cancer is described as having a given genetic variation, mutation status, allele or genotype, one or more cells of the cancer have the relevant genetic variation, mutation status, allele or genotype. In some embodiments, where a cancer is described as having a given genetic variation, mutation status, allele or genotype the majority (i.e. >50%) of cells of the cancer have the relevant genetic variation, mutation status, allele or genotype. In some embodiments, one of ≥60%, ≥65%, ≥70%, ≥75%, ≥80%, ≥85%, ≥90%, ≥95% or 100% of the cells of the cancer possess the relevant genetic variation, mutation status, allele or genotype. In some embodiments, a cancer comprising a given genetic variation/mutation/allele/genotype may be a cancer in which >10% (e.g. one of ≥20%, ≥50%, ≥40%, ≥50%, ≥60%, ≥65%, ≥70%, ≥75%, ≥80%, ≥85%, ≥90%, ≥95% or 100%) of cells of the cancer comprise the genetic variation/mutation/allele/genotype.

In some embodiments, a cancer not comprising (i.e. lacking) a given genetic variation/mutation/allele/genotype may be a cancer in which <25% (e.g. one of ≤20%, ≤15%, ≤10%, ≤5%, 51% or none) of the cells of the cancer comprise the genetic variation/mutation/allele/genotype.

Herein, where a cell is described as comprising a given genetic variation/mutation, it will be appreciated that one or more alleles of the relevant gene comprise such genetic variation/mutation. In some embodiments, a cell comprising a given genetic variation/mutation may be heterozygous for an allele comprising the genetic variation/mutation. In some embodiments, a cell comprising a given genetic variation/mutation may be homozygous for an allele comprising the genetic variation/mutation.

Where a cell is described as not comprising a given genetic variation/mutation, it will be appreciated that none of the alleles of the relevant gene comprise such genetic variation/mutation (i.e. the cell is not homozygous or heterozygous for the mutation/mutant allele).

Aspects and embodiments according to the present disclosure concern genetic variation resulting in an increase in the expression of, or an increase in the activity of a gene product of, MET.

Genetic variation known or predicted to increase the expression and/or activity of MET/c-Met is described e.g. in Tovar and Graveel (2017) 5(10):205, which is hereby incorporated by reference in its entirety.

In some embodiments, genetic variation resulting in an increase in the expression of, or an increase in the activity of a gene product of, MET is an activating mutation to MET. Activating mutations to MET include mutation to E34 (e.g. E34K), H150 (e.g. H150Y), E168 (e.g. E168D), L269 (e.g. L269V), L299 (e.g. L299F), S323 (e.g. S323G), M362 (e.g. M362T), N375 (e.g. N375S), C358 (e.g. C358Y), R970 (e.g. R970C), R988 (e.g. R988C), P1009 (e.g. P1009S), T1010 (e.g. T10101), S1058 (e.g. S1058P), exon 14 skipping mutations, A1108 (e.g. A1108S), V1110 (e.g. V11101), H1112 (e.g. H1112R, H1112L, H1112I), H1124 (e.g. H1124D), G1137 (e.g. G1137V), M1149 (e.g. M1149T), T1191 (e.g. T11911), V1206 (e.g. V1206L), L1213 (e.g. L1213V), D1228 (e.g. D1228V), Y1230 (e.g. Y1230C, Y1230H, Y1230D), Y1235 (e.g. Y1235D), V1238 (e.g. V12381), D1246 (e.g. D1246N), Y1248 (e.g. Y1248C, Y1248D, Y1248H), K1262 (e.g. K1262R), M1268 (e.g. M1268T, M12681) and V1312 (e.g. V13121).

Accordingly, in some embodiments, an activating mutation to MET is selected from mutation to: E34 (e.g. E34K), H150 (e.g. H150Y), E168 (e.g. E168D), L269 (e.g. L269V), L299 (e.g. L299F), S323 (e.g. S323G), M362 (e.g. M362T), N375 (e.g. N375S), C358 (e.g. C358Y), R970 (e.g. R970C), R988 (e.g. R988C), P1009 (e.g. P1009S), T1010 (e.g. T10101), S1058 (e.g. S1058P), exon 14 skipping mutations, A1108 (e.g. A1108S), V1110 (e.g. V11101), H1112 (e.g. H1112R, H1112L, H1112I), H1124 (e.g. H1124D), G1137 (e.g. G1137V), M1149 (e.g. M1149T), T1191 (e.g. T11911), V1206 (e.g. V1206L), L1213 (e.g. L1213V), D1228 (e.g. D1228V), Y1230 (e.g. Y1230C, Y1230H, Y1230D), Y1235 (e.g. Y1235D), V1238 (e.g. V12381), D1246 (e.g. D1246N), Y1248 (e.g. Y1248C, Y1248D, Y1248H), K1262 (e.g. K1262R), M1268 (e.g. M1268T, M12681) or V1312 (e.g. V13121).

In some embodiments, genetic variation resulting in an increase in the expression of, or an increase in the activity of a gene product of, MET is or comprises amplification of MET. MET amplification can be identified using techniques well known in the art, such as in situ hybridization. For example, MET amplification can be evaluated by fluorescence in situ hybridization, e.g. as described in Awad et al., J. Clin. Oncol. (2016) 34(7):721-730 or Yeung et al., J Thorac Oncol (2015) 10:1292-300. MET amplification can be evaluated using Next-Generation Sequencing technology, e.g. as described in Schubart et al. Cancers (Basel). (2021) 13(19): 5023.

In some embodiments, a cell comprising MET amplification comprises >2, e.g. one of 3, 4, 5, 6, 7, 8, 9, 10 or more copies of MET, or >2, e.g. one of 3, 4, 5, 6, 7, 8, 9, 10 or more copies of a fragment of MET. MET amplification may arise as a consequence of gene multiplication or polysomy. In some embodiments, a MET-amplified cancer may comprise a ratio of MET to chromosome 7≥1 (e.g. as determined by ISH), e.g. ≥1.5, ≥2, ≥2.5. In some embodiments, a MET-amplified cancer may comprise a ratio of MET to centromere 7 (CEN7)≥1 (e.g. as determined by ISH), e.g. ≥1.5, ≥2, ≥2.5. In some embodiments, cells of a MET-amplified cancer may comprise a MET gene copy number (GCN)≥2, ≥3, ≥4, ≥5, ≥6, ≥7, ≥8, ≥9 or ≥10 (e.g. as determined by NGS).

In some embodiments, genetic variation resulting in an increase in the expression of, or an increase in the activity of a gene product of, MET is not or does not comprise mutation of MET. In some embodiments, genetic variation resulting in an increase in the expression of, or an increase in the activity of a gene product of, MET is genetic variation other than mutation of MET (e.g. is or comprises amplification of MET, e.g. as described hereinabove).

Aspects and embodiments according to the present disclosure concern genetic variation resulting in an increase in the expression of, or an increase in the activity of a gene product of, KRAS.

Genetic variation known or predicted to increase the expression and/or activity of KRAS/K-Ras is described e.g. in Hobbs and Der, Cancer Discov. (2019) 9(6):696-698, which is hereby incorporated by reference in its entirety. Activating mutations to KRAS include mutation to G12 (e.g. G12A, G12D, G12D, G12R, G12C, G12S and G12V), mutation to G13 (e.g. G13D, G13C), mutation to Q61 (e.g. Q61H, Q61L, Q61K, Q61R), mutation to A146 (e.g. A146T, A146V) and mutation to K117 (e.g. K117N). In some embodiments, an activating mutation to KRAS according to the present disclosure is G12C.

In some embodiments, genetic variation resulting in an increase in the expression of, or an increase in the activity of a gene product of, KRAS is or comprises amplification of KRAS. KRAS amplification can be identified using techniques well known in the art, such as in situ hybridization and NGS. For example, KRAS amplification can be evaluated by fluorescence in situ hybridization, e.g. as described in Valtorta et al., Int J Cancer. (2013) 133(5):1259-65.

In some embodiments, a cell comprising KRAS amplification comprises >2, e.g. one of 3, 4, 5, 6, 7, 8, 9, 10 or more copies of KRAS, or >2, e.g. one of 3, 4, 5, 6, 7, 8, 9, 10 or more copies of a fragment of KRAS. KRAS amplification may arise as a consequence of gene multiplication or polysomy. In some embodiments, a KRAS-amplified cancer may comprise a ratio of KRAS to chromosome 12≥1 (e.g. as determined by ISH), e.g. ≥1.5, ≥2, ≥2.5. In some embodiments, a KRAS-amplified cancer may comprise a ratio of KRAS to centromere 12 (CEN12)≥1 (e.g. as determined by ISH), e.g. 1.5, ≥2, ≥2.5. In some embodiments, cells of a KRAS-amplified cancer may comprise a KRAS gene copy number (GCN)>2, ≥3, ≥4, ≥5, ≥6, ≥7, ≥8, ≥9 or ≥10 (e.g. as determined by NGS).

In some embodiments, genetic variation resulting in an increase in the expression of, or an increase in the activity of a gene product of, KRAS is not or does not comprise amplification of KRAS. In some embodiments, genetic variation resulting in an increase in the expression of, or an increase in the activity of a gene product of, KRAS is genetic variation other than amplification of KRAS (e.g. is or comprises an activating mutation to KRAS, e.g. as described hereinabove).

Aspects and embodiments according to the present disclosure concern genetic variation resulting in an increase in the expression of, or an increase in the activity of a gene product of, PIK3CA.

Genetic variation known or predicted to increase the expression and/or activity of PIK3CA/P13K is described e.g. in Ligresti et al., Cell Cycle. (2009) 8(9): 1352-1358, which is hereby incorporated by reference in its entirety. Activating mutations to PIK3CA include mutation to H1047 (e.g. H1047R, H1047L), mutation to E542 (e.g. E542K, E542Q), mutation to E545 (e.g. E545K), mutation to P449 (e.g. P449T) and mutation to Q546 (e.g. Q546R). In some embodiments, an activating mutation to PIK3CA according to the present disclosure is Q546R or P449T.

In some embodiments, genetic variation resulting in an increase in the expression of, or an increase in the activity of a gene product of, PIK3CA is or comprises amplification of PIK3CA. PIK3CA amplification can be identified using techniques well known in the art, such as in situ hybridization and NGS. For example, PIK3CA amplification can be evaluated by fluorescence in situ hybridization, e.g. as described in Holst et al., Clin Cancer Res. (2019) 25(1):334-345.

In some embodiments, a cell comprising PIK3CA amplification comprises >2, e.g. one of 3, 4, 5, 6, 7, 8, 9, 10 or more copies of PIK3CA, or >2, e.g. one of 3, 4, 5, 6, 7, 8, 9, 10 or more copies of a fragment of PIK3CA. PIK3CA amplification may arise as a consequence of gene multiplication or polysomy. In some embodiments, a PIK3CA-amplified cancer may comprise a ratio of PIK3CA to chromosome 3≥1 (e.g. as determined by ISH), e.g. ≥1.5, ≥2, ≥2.5. In some embodiments, a PIK3CA-amplified cancer may comprise a ratio of PIK3CA to centromere 3 (CEN3)≥1 (e.g. as determined by ISH), e.g. ≥1.5, ≥2, ≥2.5. In some embodiments, cells of a PIK3CA-amplified cancer may comprise a PIK3CA gene copy number (GCN)>2, ≥3, ≥4, ≥5, ≥6, ≥7, ≥8, ≥9 or ≥10 (e.g. as determined by NGS).

In some embodiments, genetic variation resulting in an increase in the expression of, or an increase in the activity of a gene product of, PIK3CA is not or does not comprise amplification of PIK3CA. In some embodiments, genetic variation resulting in an increase in the expression of, or an increase in the activity of a gene product of, PIK3CA is genetic variation other than amplification of PIK3CA (e.g. is or comprises an activating mutation to PIK3CA, e.g. as described hereinabove).

Aspects and embodiments according to the present disclosure concern genetic variation resulting in an increase in the expression of, or an increase in the activity of a gene product of, BRAF.

Genetic variation known or predicted to increase the expression and/or activity of BRAF/B-Raf is described e.g. in Van Cutsem et al., Journal of Clinical Immunology (2011) 29(15): 2011-2019, which is hereby incorporated by reference in its entirety. Activating mutations to BRAF include mutations to V600 (e.g. V600E or V600K), T119 (e.g. T119S) and L597 (e.g. L597R). In some embodiments, an activating mutation to according to the present disclosure is V600E, V600K, T119S, or L597R.

In some embodiments, genetic variation resulting in an increase in the expression of, or an increase in the activity of a gene product of, BRAF is or comprises amplification of BRAF. BRAF amplification can be identified using techniques well known in the art, such as in situ hybridization and NGS. For example, BRAF amplification can be evaluated by fluorescence in situ hybridization, e.g. as described in Corcoran et al., Sci Signal. (2010) 3(149):ra84.

In some embodiments, a cell comprising BRAF amplification comprises >2, e.g. one of 3, 4, 5, 6, 7, 8, 9, 10 or more copies of BRAF, or >2, e.g. one of 3, 4, 5, 6, 7, 8, 9, 10 or more copies of a fragment of BRAF. BRAF amplification may arise as a consequence of gene multiplication or polysomy. In some embodiments, a BRAF-amplified cancer may comprise a ratio of BRAF to chromosome 7≥1 (e.g. as determined by ISH), e.g. ≥1.5, ≥2, ≥2.5. In some embodiments, a BRAF-amplified cancer may comprise a ratio of BRAF to centromere 7 (CEN7)≥1 (e.g. as determined by ISH), e.g. 1.5, ≥2, ≥2.5. In some embodiments, cells of a BRAF-amplified cancer may comprise a BRAF gene copy number (GCN)≥2, ≥3, ≥4, ≥5, ≥6, ≥7, ≥8, ≥9 or ≥10 (e.g. as determined by NGS).

In some embodiments, genetic variation resulting in an increase in the expression of, or an increase in the activity of a gene product of, BRAF is not or does not comprise amplification of BRAF. In some embodiments, genetic variation resulting in an increase in the expression of, or an increase in the activity of a gene product of, BRAF is genetic variation other than amplification of BRAF (e.g. is or comprises an activating mutation to BRAF, e.g. as described hereinabove).

Aspects and embodiments according to the present disclosure concern genetic variation resulting in a decrease in the expression of, or decrease in the activity of a gene product of, PTEN.

Genetic variation known or predicted to decrease the expression and/or activity of PTEN/PTEN is described e.g. in Chang et al., Biomolecules. (2019) 9(11):713, which is hereby incorporated by reference in its entirety. Inactivating mutations to PTEN include mutation to R130, mutation to R173, mutation to R233, mutation to K267, and mutation to N323.

In some embodiments, genetic variation resulting in a decrease in the expression of, or a decrease in the activity of a gene product of, PTEN is or comprises deletion of PTEN. PTEN deletion can be identified using techniques well known in the art, such as in situ hybridization and NGS. For example, PTEN deletion can be evaluated by fluorescence in situ hybridization, e.g. as described in Wang et al., Neoplasia. (2018) 20(6): 574-593.

In some embodiments, a cell comprising PTEN deletion comprises <2, e.g. one of 1 or 0 copies of PTEN. In some embodiments, a cancer comprising PTEN deletion may comprise a ratio of PTEN to chromosome 17≤1 (e.g. as determined by ISH). In some embodiments, a cancer comprising PTEN deletion may comprise a ratio of PTEN to centromere 17 (CEN17)≤1 (e.g. as determined by ISH). In some embodiments, cells of a cancer comprising PTEN deletion may comprise a PTEN gene copy number (GCN)≤2, e.g. ≤1.5, ≤1 or ≤0.5 (e.g. as determined by NGS).

In some embodiments, genetic variation resulting in a decrease in the expression of, or a decrease in the activity of a gene product of, PTEN is not or does not comprise mutation of PTEN. In some embodiments, genetic variation resulting in a decrease in the expression of, or a decrease in the activity of a gene product of, PTEN is genetic variation other than mutation of PTEN (e.g. is or comprises deletion of PTEN, e.g. as described hereinabove).

Aspects and embodiments according to the present disclosure concern genetic variation resulting in an increase in the expression of, or an increase in the activity of a gene product of, EGFR.

Genetic variation known or predicted to increase the expression and/or activity of EGFR/EGFR is described e.g. in Gazdar, Oncogene. (2009) 28(Suppl 1): S24-S31, which is hereby incorporated by reference in its entirety. Activating mutations to EGFR include mutations to L858 (e.g. L858R), G719 (e.g. G719S, G719A or G719C), T790 (e.g. T790M), V765 (e.g. V765A), T783 (e.g. T783A), and deletion mutations in exon 19 (e.g. deletions including L747 to E749).

In some embodiments, genetic variation resulting in an increase in the expression of, or an increase in the activity of a gene product of, EGFR is or comprises amplification of EGFR. EGFR amplification can be identified using techniques well known in the art, such as in situ hybridization and NGS. For example, EGFR amplification can be evaluated by fluorescence in situ hybridization, e.g. as described in French et al., Neuro-Oncology (2019) 21(10): 1263-1272.

In some embodiments, a cell comprising EGFR amplification comprises >2, e.g. one of 3, 4, 5, 6, 7, 8, 9, 10 or more copies of EGFR, or >2, e.g. one of 3, 4, 5, 6, 7, 8, 9, 10 or more copies of a fragment of EGFR. EGFR amplification may arise as a consequence of gene multiplication or polysomy. In some embodiments, a EGFR-amplified cancer may comprise a ratio of EGFR to chromosome 7≥1 (e.g. as determined by ISH), e.g. ≥1.5, ≥2, ≥2.5. In some embodiments, a EGFR-amplified cancer may comprise a ratio of EGFR to centromere 7 (CEN7)≥1 (e.g. as determined by ISH), e.g. 1.5, ≥2, ≥2.5. In some embodiments, cells of a EGFR-amplified cancer may comprise a EGFR gene copy number (GCN)>2, ≥3, ≥4, ≥5, ≥6, ≥7, ≥8, ≥9 or ≥10 (e.g. as determined by NGS).

Aspects and embodiments according to the present disclosure concern genetic variation resulting in an increase in the expression of, or an increase in the activity of a gene product of, TP63.

In some embodiments, genetic variation resulting in an increase in the expression of, or an increase in the activity of a gene product of, TP63 is or comprises amplification of TP63. TP63 amplification can be identified using techniques well known in the art, such as in situ hybridization and NGS. For example, TP63 amplification can be evaluated by fluorescence in situ hybridization, e.g. as described in Massion et al., Cancer Res. (2003) 63(21):7113-21.

In some embodiments, a cell comprising TP63 amplification comprises >2, e.g. one of 3, 4, 5, 6, 7, 8, 9, 10 or more copies of TP63, or >2, e.g. one of 3, 4, 5, 6, 7, 8, 9, 10 or more copies of a fragment of TP63. TP63 amplification may arise as a consequence of gene multiplication or polysomy. In some embodiments, a TP63-amplified cancer may comprise a ratio of TP63 to chromosome 3≥1 (e.g. as determined by ISH), e.g. ≥1.5, ≥2, ≥2.5. In some embodiments, a TP63-amplified cancer may comprise a ratio of TP63 to centromere 3 (CEN3)≥1 (e.g. as determined by ISH), e.g. ≥1.5, ≥2, ≥2.5. In some embodiments, cells of a TP63-amplified cancer may comprise a TP63 gene copy number (GCN)>2, ≥3, ≥4, ≥5, ≥6, ≥7, ≥8, ≥9 or ≥10 (e.g. as determined by NGS).

Aspects and embodiments according to the present disclosure concern genetic variation resulting in an increase in the expression of, or an increase in the activity of a gene product of, SOX2.

In some embodiments, genetic variation resulting in an increase in the expression of, or an increase in the activity of a gene product of, SOX2 is or comprises amplification of SOX2. SOX2 amplification can be identified using techniques well known in the art, such as in situ hybridization and NGS. For example, SOX2 amplification can be evaluated by fluorescence in situ hybridization, e.g. as described in Wilbertz et al., Modern Pathology (2011) 24: 944-953.

In some embodiments, a cell comprising SOX2 amplification comprises >2, e.g. one of 3, 4, 5, 6, 7, 8, 9, 10 or more copies of SOX2, or >2, e.g. one of 3, 4, 5, 6, 7, 8, 9, 10 or more copies of a fragment of SOX2. SOX2 amplification may arise as a consequence of gene multiplication or polysomy. In some embodiments, a SOX2-amplified cancer may comprise a ratio of SOX2 to chromosome 3≥1 (e.g. as determined by ISH), e.g. ≥1.5, ≥2, ≥2.5. In some embodiments, a SOX2-amplified cancer may comprise a ratio of SOX2 to centromere 3 (CEN3)≥1 (e.g. as determined by ISH), e.g. ≥1.5, ≥2, ≥2.5. In some embodiments, cells of a SOX2-amplified cancer may comprise a SOX2 gene copy number (GCN)>2, ≥3, ≥4, ≥5, ≥6, ≥7, ≥8, ≥9 or ≥10 (e.g. as determined by NGS).

Aspects and embodiments according to the present disclosure concern genetic variation resulting in an increase in the expression of, or an increase in the activity of a gene product of, one or more genes located on chromosome 3q. In some embodiments, genetic variation resulting in an increase in the expression of, or an increase in the activity of a gene product of, one or more genes located on chromosome 3q is or comprises amplification of the relevant gene(s). Amplification of one or more genes located on chromosome 3q can be identified using techniques well known in the art, such as in situ hybridization and NGS.

In some embodiments, a cell comprising amplification of one or more genes located on chromosome 3q comprises >2, e.g. one of 3, 4, 5, 6, 7, 8, 9, 10 or more copies of the relevant gene(s), or >2, e.g. one of 3, 4, 5, 6, 7, 8, 9, 10 or more copies of a fragment of the relevant gene(s). Amplification of one or more genes located on chromosome 3q may arise as a consequence of gene multiplication or polysomy. In some embodiments, a cancer comprising amplification of one or more genes located on chromosome 3q may comprise a ratio of the relevant gene(s) to chromosome 3≥1 (e.g. as determined by ISH), e.g. ≥1.5, ≥2, ≥2.5. In some embodiments, a cancer comprising amplification of one or more genes located on chromosome 3q may comprise a ratio of the relevant gene(s) to centromere 3 (CEN3)≥1 (e.g. as determined by ISH), e.g. ≥1.5, ≥2, ≥2.5. In some embodiments, cells of cancer comprising amplification of one or more genes located on chromosome 3q may comprise a gene copy number (GCN) for the relevant gene(s) that is >2, ≥3, ≥4, ≥5, ≥6, ≥7, ≥8, ≥9 or ≥10 (e.g. as determined by NGS).

Aspects and embodiments according to the present disclosure concern genetic variation resulting in an increase in the expression of, or an increase in the activity of a gene product of, one or more genes located within 3q26 to 3q28 (e.g. within 3q26, 3q27 or 3q28). In some embodiments, genetic variation resulting in an increase in the expression of, or an increase in the activity of a gene product of, one or more genes located within 3q26 to 3q28 (e.g. within 3q26, 3q27 or 3q28) is or comprises amplification of the relevant gene(s). Amplification of one or more genes located within 3q26 to 3q28 (e.g. within 3q26, 3q27 or 3q28) can be identified using techniques well known in the art, such as in situ hybridization and NGS.

In some embodiments, a cell comprising amplification of one or more genes located within 3q26 to 3q28 (e.g. within 3q26, 3q27 or 3q28) comprises >2, e.g. one of 3, 4, 5, 6, 7, 8, 9, 10 or more copies of the relevant gene(s), or >2, e.g. one of 3, 4, 5, 6, 7, 8, 9, 10 or more copies of a fragment of the relevant gene(s). Amplification of one or more genes located within 3q26 to 3q28 (e.g. within 3q26, 3q27 or 3q28) may arise as a consequence of gene multiplication or polysomy. In some embodiments, a cancer comprising amplification of one or more genes located within 3q26 to 3q28 (e.g. within 3q26, 3q27 or 3q28) may comprise a ratio of the relevant gene(s) to chromosome 3≥1 (e.g. as determined by ISH), e.g. ≥1.5, ≥2, ≥2.5. In some embodiments, a cancer comprising amplification of one or more genes located within 3q26 to 3q28 may comprise a ratio of the relevant gene(s) to centromere 3 (CEN3)≥1 (e.g. as determined by ISH), e.g. ≥1.5, ≥2, ≥2.5. In some embodiments, cells of cancer comprising amplification of one or more genes located within 3q26 to 3q28 (e.g. within 3q26, 3q27 or 3q28) may comprise a gene copy number (GCN) for the relevant gene(s) that is >2, ≥3, ≥4, ≥5, ≥6, ≥7, ≥8, ≥9 or ≥10 (e.g. as determined by NGS).

Genes located within 3q26 to 3q28 include TP63, PIK3CA and SOX2. Genes located within 3q28 include PIK3CA (3q26.32) and SOX2 (3q26.33). Genes located within 3q28 include TP63.

Aspects and embodiments according to the present disclosure concern genetic variation resulting in amplification of chromosome 3q. Amplification of chromosome 3q may arise as a consequence of polysomy.

Genes located on chromosome 3q include ADIPOQ, AMOTL2, ARHGAP31, TIMMDC1, C3orf70, CAMPD1, CCDC80, CD200R1, CHST13, CLDND1, CPN2, CPOX, DPPA2, DTX3L, DZIP3, EAF2, EFCC1, ETM1, ETV5, FAM3D, FAM43A, FAM162A, FBXO40, FILIP1L, GYG1, HACD2, HGD, IFT122, KIAA1257, LINC01279, LNCR5, LMLN, LRRC15, LSG1, MB21D2, MCCC1, MORC1, MYLK, NEPRO, NFKBIZ, OTOL1, PARP14, PCCB, PDCD10, PIK3CA, PISRT1, PROSER1, RAB7, RASA2, RETNLB, RHO, RIOX2, SELT, SENP7, SERP1, SOX2, SOX2OT, SPG14, SRPRB, TEX55, TMEM44, TM4SF1, TMPRSS7, TP63, TRAT1, USH3A, ZBED2 and ZNF9. In some embodiments, one or more genes located on chromosome 3q are selected from TP63, PIK3CA and SOX2.

Aspects and embodiments according to the present disclosure concern genetic variation resulting in amplification of 3q26. Aspects and embodiments according to the present disclosure concern genetic variation resulting in amplification of 3q27. Aspects and embodiments according to the present disclosure concern genetic variation resulting in amplification of 3q28. Aspects and embodiments according to the present disclosure concern genetic variation resulting in amplification of 3q26 to 3q28. Such amplification may arise as a consequence of polysomy.

Aspects and embodiments according to the present disclosure concern genetic variation resulting in an increase in the expression of, or an increase in the activity of a gene product of, one or more genes located on chromosome 7p. In some embodiments, genetic variation resulting in an increase in the expression of, or an increase in the activity of a gene product of, one or more genes located on chromosome 7p is or comprises amplification of the relevant gene(s). Amplification of one or more genes located on chromosome 7p can be identified using techniques well known in the art, such as in situ hybridization and NGS.

In some embodiments, a cell comprising amplification of one or more genes located on chromosome 7p comprises ≥2, e.g. one of 3, 4, 5, 6, 7, 8, 9, 10 or more copies of the relevant gene(s), or >2, e.g. one of 3, 4, 5, 6, 7, 8, 9, 10 or more copies of a fragment of the relevant gene(s). Amplification of one or more genes located on chromosome 7p may arise as a consequence of gene multiplication or polysomy. In some embodiments, a cancer comprising amplification of one or more genes located on chromosome 7p may comprise a ratio of the relevant gene(s) to chromosome 7≥1 (e.g. as determined by ISH), e.g. ≥1.5, ≥2, ≥2.5. In some embodiments, a cancer comprising amplification of one or more genes located on chromosome 7p may comprise a ratio of the relevant gene(s) to centromere 7 (CEN7)≥1 (e.g. as determined by ISH), e.g. ≥1.5, ≥2, ≥2.5. In some embodiments, cells of cancer comprising amplification of one or more genes located on chromosome 7p may comprise a gene copy number (GCN) for the relevant gene(s) that is >2, ≥3, ≥4, ≥5, ≥6, ≥7, ≥8, ≥9 or ≥10 (e.g. as determined by NGS).

Aspects and embodiments according to the present disclosure concern genetic variation resulting in an increase in the expression of, or an increase in the activity of a gene product of, one or more genes located within 7p11. In some embodiments, genetic variation resulting in an increase in the expression of, or an increase in the activity of a gene product of, one or more genes located within 7p11 is or comprises amplification of the relevant gene(s). Amplification of one or more genes located within 7p11 can be identified using techniques well known in the art, such as in situ hybridization and NGS.

In some embodiments, a cell comprising amplification of one or more genes located within 7p11 comprises >2, e.g. one of 3, 4, 5, 6, 7, 8, 9, 10 or more copies of the relevant gene(s), or >2, e.g. one of 3, 4, 5, 6, 7, 8, 9, 10 or more copies of a fragment of the relevant gene(s). Amplification of one or more genes located within 7p11 may arise as a consequence of gene multiplication or polysomy. In some embodiments, a cancer comprising amplification of one or more genes located within 7p11 may comprise a ratio of the relevant gene(s) to chromosome 3≥1 (e.g. as determined by ISH), e.g. ≥1.5, ≥2, ≥2.5. In some embodiments, a cancer comprising amplification of one or more genes located within 7p11 may comprise a ratio of the relevant gene(s) to centromere 7 (CEN7)≥1 (e.g. as determined by ISH), e.g. ≥1.5, ≥2, ≥2.5. In some embodiments, cells of cancer comprising amplification of one or more genes located within 7p11 may comprise a gene copy number (GCN) for the relevant gene(s) that is >2, ≥3, ≥4, ≥5, ≥6, ≥7, ≥8, ≥9 or 10 (e.g. as determined by NGS).

Genes located within 7p11 include EGFR (7p11.2).

Aspects and embodiments according to the present disclosure concern genetic variation resulting in amplification of chromosome 7p. Aspects and embodiments according to the present disclosure concern genetic variation resulting in amplification of 7p11. Such amplification may arise as a consequence of polysomy.

Aspects and embodiments according to the present disclosure concern genetic variation resulting in a decrease in the expression of, or a decrease in the activity of a gene product of, one or more genes located on chromosome 3p. In some embodiments, genetic variation resulting in a decrease in the expression of, or a decrease in the activity of a gene product of, one or more genes located on chromosome 3p is or comprises loss or deletion of the relevant gene(s). Loss or deletion of one or more genes located on chromosome 3p can be identified using techniques well known in the art, such as in situ hybridization and NGS.

In some embodiments, a cell comprising deletion of one or more genes located on chromosome 3p comprises <2, e.g. one of 1 or 0 copies of the relevant gene(s), or <2, e.g. one of 1 or 0 copies of a fragment of the relevant gene(s). In some embodiments, a cancer comprising deletion of one or more genes located on chromosome 3p may comprise a ratio of the relevant gene(s) to chromosome 3≥1 (e.g. as determined by ISH). In some embodiments, a cancer comprising deletion of one or more genes located on chromosome 3p may comprise a ratio of the relevant gene(s) to centromere 3 (CEN3)≤1 (e.g. as determined by ISH). In some embodiments, cells of cancer comprising deletion of one or more genes located on chromosome 3p may comprise a gene copy number (GCN) for the relevant gene(s) that is <2, e.g. ≤1.5, ≤1 or ≤0.5 (e.g. as determined by NGS).

Aspects and embodiments according to the present disclosure concern genetic variation resulting in a decrease in the expression of, or a decrease in the activity of a gene product of, one or more genes located within 3p21 (e.g. within 3p21.3). In some embodiments, genetic variation resulting in a decrease in the expression of, or a decrease in the activity of a gene product of, one or more genes located within 3p21 (e.g. within 3p21.3) is or comprises deletion of the relevant gene(s). Deletion of one or more genes located within 3p21 (e.g. within 3p21.3) can be identified using techniques well known in the art, such as in situ hybridization and NGS.

In some embodiments, a cell comprising deletion of one or more genes located within 3p21 (e.g. within 3p21.3) comprises <2, e.g. one of 1 or 0 copies of the relevant gene(s), or <2, e.g. one of 1 or 0 copies of a fragment of the relevant gene(s). In some embodiments, a cancer comprising deletion of one or more genes located within 3p21 (e.g. within 3p21.3) may comprise a ratio of the relevant gene(s) to chromosome 3≤1 (e.g. as determined by ISH). In some embodiments, a cancer comprising deletion of one or more genes located within 3p21 may comprise a ratio of the relevant gene(s) to centromere 3 (CEN3)≤1 (e.g. as determined by ISH). In some embodiments, cells of cancer comprising deletion of one or more genes located within 3p21 (e.g. within 3p21.3) may comprise a gene copy number (GCN) for the relevant gene(s) that is ≤2, e.g. ≤1.5, ≤1 or ≤0.5 (e.g. as determined by NGS).

Genes located within 3p21 (e.g. within 3p21.3) include TUSC2.

Aspects and embodiments according to the present disclosure concern genetic variation resulting in deletion of chromosome 3p. Aspects and embodiments according to the present disclosure concern genetic variation resulting in deletion of 3p21.

The present disclosure contemplates genetic variation in genes encoding factors of relevance to HER3-mediated signalling.

Aspects and embodiments according to the present disclosure contemplate genetic variation resulting in an increase in the expression of, or an increase in the activity of a gene product of, a positive regulator of HER3-mediated signalling. Aspects and embodiments according to the present disclosure contemplate genetic variation resulting in an increase in the expression of, or an increase in the activity of a gene product of, a positive regulator of signalling through the MAPK/ERK pathway. Aspects and embodiments according to the present disclosure contemplate genetic variation resulting in an increase in the expression of, or an increase in the activity of a gene product of, a positive regulator of signalling through the PI3K/AKT/mTOR pathway.

Aspects and embodiments according to the present disclosure contemplate genetic variation resulting in a decrease in the expression of, or a decrease in the activity of a gene product of, a negative regulator of HER3-mediated signalling. Aspects and embodiments according to the present disclosure contemplate genetic variation resulting in a decrease in the expression of, or a decrease in the activity of a gene product of, a negative regulator of signalling through the MAPK/ERK pathway. Aspects and embodiments according to the present disclosure contemplate genetic variation resulting in a decrease in the expression of, or a decrease in the activity of a gene product of, a negative regulator of signalling through the PI3K/AKT/mTOR pathway.

HER3-mediated signalling can be analysed e.g. using an assay of a correlate of HER3-mediated signalling, e.g. cell proliferation, and/or phosphorylation of one or more signal transduction molecules of the PI3K/AKT/mTOR and/or MAPK/ERK signal transduction pathways. For example, the level of PI3K/AKT/mTOR and/or MAPK/ERK signalling may be analysed by detection and quantification of the level of phosphorylation of one or more of the components of the PI3K/AKT/mTOR and/or MAPK/ERK pathways. Such analysis may be performed in vitro in a cell-based assay of HER3-mediated signalling, e.g. as described in Example 4.3 of 8.9 of WO 2019/185878 A1.

As used herein, a ‘positive regulator’ of signalling through a given pathway (e.g. a positive regulator of HER3-mediated signalling, a positive regulator of signalling through the MAPK/ERK pathway, or a positive regulator of signalling through the PI3K/AKT/mTOR pathway) refers to a factor whose expression/activity generally contributes positively to (i.e. potentiates, enhances) signalling through the relevant pathway. An increase in the level of expression and/or activity of a positive regulator may result in an increase in the level of signalling through the relevant pathway (e.g. as determined by analysis of a correlate of such signalling). A decrease in the level of expression and/or activity of a positive regulator may result in a decrease in the level of signalling through the relevant pathway.

A ‘negative regulator’ of signalling through a given pathway (e.g. a positive regulator of HER3-mediated signalling, a positive regulator of signalling through the MAPK/ERK pathway, or a positive regulator of signalling through the PI3K/AKT/mTOR pathway) refers to a factor whose expression/activity generally contributes negatively to (i.e. inhibits, antagonises) signalling through the relevant pathway. An increase in the level of expression and/or activity of a negative regulator may result in an decrease in the level of signalling through the relevant pathway (e.g. as determined by analysis of a correlate of such signalling). A decrease in the level of expression and/or activity of a negative regulator may result in an increase in the level of signalling through the relevant pathway.

In some embodiments, a gene encoding a positive regulator of HER3-mediated signalling may be selected from: KRAS, PIK3CA, PIK3CB, PIK3CD, ERBB3, ERBB2, ERBB4, EGFR, IGF1R, NRG1, NRG2, EGF, IRS2,GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3, STAT5, BRAF and MET. In some embodiments, a gene encoding a positive regulator of HER3-mediated signalling may be selected from: KRAS, PIK3CA, PIK3CB, BRAF and MET. In some embodiments, a gene encoding a positive regulator of HER3-mediated signalling is KRAS. In some embodiments, a gene encoding a positive regulator of HER3-mediated signalling is PIK3CA. In some embodiments, a gene encoding a positive regulator of HER3-mediated signalling is PIK3CB. In some embodiments, a gene encoding a positive regulator of HER3-mediated signalling is BRAF. In some embodiments, a gene encoding a positive regulator of HER3-mediated signalling is MET.

In some embodiments, a gene encoding a positive regulator of HER3-mediated signalling may be selected from: PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5.

In some embodiments, a gene encoding a positive regulator of signalling through the MAPK/ERK pathway may be selected from: ERBB3, ERBB2, ERBB4, EGFR, IGF1R, NRG1, NRG2, EGF, IRS2, GAB2, GRB2, PTPN11, SHP2, SOS1, HRAS, KRAS, NRAS, RAF1, MAP2K1, MAP2K2, MAPK1, MYC, RPS6KA1, RPS6, MKNK1, CREB1, and BRAF. In some embodiments, a gene encoding a positive regulator of signalling through the MAPK/ERK pathway is KRAS. In some embodiments, a gene encoding a positive regulator of signalling through the MAPK/ERK pathway is BRAF.

In some embodiments, a gene encoding a positive regulator of signalling through the MAPK/ERK pathway may be selected from: ERBB3, ERBB2, ERBB4, EGFR, IGF1R, NRG1, NRG2, EGF, IRS2, GAB2, GRB2, PTPN11, SHP2, SOS1, HRAS, KRAS, NRAS, RAF1, MAP2K1, MAP2K2, MAPK1, MYC, RPS6KA1, RPS6, MKNK1 and CREB1.

In some embodiments, a gene encoding a positive regulator of signalling through the PI3K/AKT/mTOR pathway may be selected from: ERBB3, ERBB2, ERBB4, EGFR, IGF1R, NRG1, NRG2, EGF, IRS2, GAB2, SHP2, CREB1, PIK3CA, PIK3CB, PIK3CD, MTOR, PDK1, AKT1, AKT2, AKT3, JAK2, STAT3 and STAT5. In some embodiments, a gene encoding a positive regulator of signalling through the PI3K/AKT/mTOR pathway is PIK3CA. In some embodiments, a gene encoding a positive regulator of signalling through the PI3K/AKT/mTOR pathway is PIK3CB.

In some embodiments, a gene encoding a positive regulator of signalling through the PI3K/AKT/mTOR pathway may be selected from: ERBB3, ERBB2, ERBB4, EGFR, IGF1R, NRG1, NRG2, EGF, IRS2, GAB2, SHP2, CREB1, PIK3CB, PIK3CD, MTOR, PDK1, AKT1, AKT2, AKT3, JAK2, STAT3 and STAT5.

In some embodiments, a gene encoding a negative regulator of HER3-mediated signalling may be selected from: PTEN, PPP2CA, PIK3R1, PIK3R2, NF1, BAD and PHLPP1. In some embodiments, a gene encoding a negative regulator of signalling through the PI3K/AKT/mTOR pathway may be selected from: PTEN, PPP2CA, PIK3R1, PIK3R2, BAD and PHLPP1. In some embodiments, a gene encoding a negative regulator of signalling through the MAPK/ERK pathway may be NF1. In some embodiments, a gene encoding a negative regulator of HER3-mediated signalling is PTEN. In some embodiments, a gene encoding a negative regulator of signalling through the PI3K/AKT/mTOR pathway is PTEN.

In some embodiments, a gene encoding a negative regulator of HER3-mediated signalling may be selected from: PPP2CA, PIK3R1, PIK3R2, NF1, BAD and PHLPP1. In some embodiments, a gene encoding a negative regulator of signalling through the PI3K/AKT/mTOR pathway may be selected from: PPP2CA, PIK3R1, PIK3R2, BAD and PHLPP1.

In some embodiments, the cancer to be treated/prevented comprises cells expressing a ligand for HER3 (e.g. NRG1 and/or NRG2). In some embodiments, the cancer to be treated/prevented comprises cells expressing a level of expression of NRG1 and/or NRG2 which is greater than the level of expression by equivalent non-cancerous cells/non-tumor tissue. The cancer may be described as comprising cells that overexpress NRG1 and/or NRG2.

HER3-binding antigen-binding molecules described herein bind to HER3 with extremely high affinity when HER3 is bound by NRG (i.e. when HER3 is provided in the ‘open’ conformation), and also when HER3 is not bound by NRG (i.e. when HER3 is provided in the ‘closed’ conformation). Thus, they are particularly useful for the treatment/prevention of cancers characterized by HER3 ligand expression/overexpression, for example cancers/tumors comprising cells expressing/overexpressing a ligand for HER3.

Aspects and embodiments according to the present disclosure contemplate genetic variation (e.g. mutation or gene amplification) resulting in increased (gene and/or protein) expression of a ligand for HER3.

Genetic variation which causes increased expression of a ligand for HER3 may result in gene or protein expression of a ligand for HER3 which is not expressed by, and/or not encoded by genomic nucleic acid of, an equivalent cell not harbouring the genetic variation. That is, the ligand for HER3 may be a neoantigen arising as a result of the genetic variation, and thus ‘increased expression’ may be from no expression. By way of illustration, a cell comprising CD74-NRG1 gene fusion displays increased expression of the CD74-NRG1 fusion polypeptide encoded by the gene fusion relative to cells lacking the CD74-NRG1 gene fusion.

Genetic variation which causes increased expression of a ligand for HER3 may result in increased gene or protein expression of a ligand for HER3 which is expressed by, and/or which is encoded by genomic nucleic acid of, an equivalent cell not comprising the genetic variation. By way of illustration, a cell may comprise genetic variation resulting in an increase in the level of transcription of nucleic acid encoding NRG1 relative to the level of transcription of nucleic acid encoding NRG1 by an equivalent cell not comprising the genetic variation.

In some embodiments, genetic variation which causes increased expression of a ligand for HER3 may cause an increase in gene expression of a ligand for HER3 relative to an equivalent cell not comprising the genetic variation. In some embodiments, genetic variation which causes increased expression of a ligand for HER3 may cause an increase in protein expression of a ligand for HER3 relative to an equivalent cell not comprising the genetic variation.

In some embodiments, genetic variation which causes increased expression of a ligand for HER3 may cause an increase in the level of a ligand for HER3 on or at the cell surface of a cell comprising the genetic variation, relative to an equivalent cell not comprising the genetic variation. In some embodiments, genetic variation which causes increased expression of a ligand for HER3 may cause an increase in the level of a secretion of a ligand for HER3 from a cell comprising the genetic variation, relative to an equivalent cell not comprising the genetic variation.

Cells having increased expression of a ligand for HER3 relative to the level of expression of the ligand for HER3 by a reference cell (e.g. as a result of mutation) may be described as ‘overexpressing’ the ligand for HER3, or having ‘upregulated expression’ of the ligand for HER3. For example, a cancer comprising cells harbouring genetic variation resulting in increased expression of a ligand for HER3 relative to equivalent cells lacking the genetic variation may be described as a cancer comprising cells displaying overexpression/upregulated expression of the ligand for HER3. In some embodiments, the reference cell lacking the genetic variation may be a non-cancerous cell (e.g. of equivalent cell type) or a cancerous cell (e.g. of equivalent cancer type).

Herein, a ‘ligand for HER3’ is generally intended to refer to a molecule capable of binding to HER3 through the ligand binding region of HER3 formed by domains I and III of HER3. In some embodiments, a ligand for HER3 binds to HER3 via interaction with domains I and/or III of HER3. Exemplary ligands for HER3 include neuregulins such as NRG1 and NRG2, which bind to HER3 via interaction between their EGF-like domains and the ligand binding region of HER3.

The HER3 ligand is preferably able to bind and trigger signaling through the HER3 receptor and/or receptor complexes comprising HER3. As will be clear from the present disclosure, receptor complexes comprising HER3 may further comprise an interaction partner for HER3 as described herein, e.g. HER3, HER2, EGFR, HER4, HGFR, IGF1R and/or cMet.

In some embodiments the ligand for HER3 is able to bind to HER3 receptor/receptor complex expressed by a cell other than the cell having increased expression of the HER3 ligand. For example, in some embodiments the ligand for HER3 is able to bind to a HER3-expressing cancer cell.

In some embodiments the ligand for HER3 is able to bind to HER3 receptor/receptor complex expressed by the cell having increased expression of the HER3 ligand.

In some embodiments the cancer to be treated/prevented comprises (i) cells expressing HER3, and (ii) cells expressing a ligand for HER3 (e.g. having increased expression of a ligand for HER3, e.g. as a consequence of genetic variation resulting in increased expression of a ligand for HER3).

In some embodiments the cancer to be treated/prevented comprises cells which (i) express HER3 and (ii) which also express a ligand for HER3 (e.g. which have increased expression of a ligand for HER3, e.g. as a consequence of genetic variation resulting in increased expression of a ligand for HER3).

In some embodiments, the ligand for HER3 comprises, or consists of, the amino acid sequence of a HER3-binding region of a ligand for HER3, or an amino acid sequence derived from a HER3-binding region of a ligand for HER3. An amino acid sequence which is derived from a HER3-binding region of a ligand for HER3 may comprise at least 60% (e.g. 70%, 75%, 80%, 85%, 90%, 91%, 92%, 93%, 94%, 95%, 96%, 97%, 98%, 99% or 100%) amino acid sequence identity to the amino acid sequence from which it is derived.

In some embodiments, the ligand for HER3 comprises an EGF-like domain capable of binding to HER3, or a HER3-binding fragment thereof. In some embodiments, a HER3-binding EGF-like domain/fragment is, or is derived from, an EGF family member (e.g. heparin-binding EGF-like growth factor (HB-EGF), transforming growth factor-α (TGF-α), amphiregulin (AR), epiregulin (EPR), epigen, betacellulin (BTC), NRG1, NRG2, NRG3 or NRG4).

Exemplary ligands for HER3 include neuregulins (NRGs). Neuregulins include NRG1 (including alpha, alpha2b, and alpha3 isoforms thereof), NRG2, NRG3 and NRG4. In some embodiments, an NRG is selected from NRG1, NRG2, NRG3 and NRG4. In some embodiments, an NRG is selected from NRG1 and NRG2.

The EGF-like domain of human NRG1, through which it binds to HER3, is formed by positions 178-222 of UniProt:Q02297-1. The EGF-like domain of human NRG2 is formed by positions 341-382 of UniProt:014511-1. The EGF-like domain of human NRG3 is formed by positions 286-329 of UniProt:B9EGV5-1. The EGF-like domain of human NRG4 is formed by positions 5-46 of UniProt:Q8WWG1-1. In some embodiments, an EGF-like domain/fragment comprises, or consists of, an amino acid sequence having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of an NRG (NRG1, NRG2, NRG3 or NRG4).

In some embodiments a ligand for HER3 is not an EGFR family protein (e.g. HER3, HER2, EGFR, HER4, HGFR, IGF1R, cMet).

In some embodiments, the genetic variation resulting in increased expression of a ligand for HER3 is an NRG gene fusion. In some embodiments, the ligand for HER3 is the product of (i.e. a polypeptide encoded by) an NRG gene fusion. In some embodiments the cancer comprises cells having an NRG gene fusion. As used herein, an ‘NRG gene fusion’ refers to a genetic variant encoding a polypeptide comprising (i) an amino acid sequence of an NRG protein (e.g. NRG1, NRG2, NRG3 or NRG4; e.g. NRG1 or NRG2), and (ii) an amino acid sequence of a protein other than the NRG protein.

NRG gene fusions may be detected and characterised using appropriate molecular assays, which are well known to the skilled person.

NRG gene fusions according to the present disclosure are preferably correctly orientated (i.e. with the nucleotide sequence encoding the NRG at the 3′ end of the transcript), and encode a fusion polypeptide comprising an EGF-like domain capable of binding to HER3.

It will be appreciated that an NRG gene fusion preferably encodes a HER3 ligand as described herein. In some embodiments, an NRG gene fusion encodes a polypeptide comprising a HER3-binding region of an NRG protein. In some embodiments, an NRG gene fusion encodes a polypeptide comprising the EGF-like domain of an NRG protein, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of an NRG protein.

In some embodiments, an NRG gene fusion encodes a fusion polypeptide comprising a transmembrane domain. In some embodiments, an NRG gene fusion encodes a fusion polypeptide comprising the transmembrane domain of a protein other than the NRG protein.

In some embodiments, an NRG gene fusion is an NRG1 gene fusion. In some embodiments, the NRG1 gene fusion encodes a polypeptide comprising the EGF-like domain of NRG1, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of NRG1.

NRG1 gene fusions represent an actionable fusion in tissue-agnostic cancers that can be targeted through HER3 inhibition. They are formed by inter-chromosomal translocation with a diverse set of genes leading to an overproduction of NRG1 ligands for HER3-binding, and aberrant activation leading to tumorigenesis. Studies have demonstrated a causal link between HER3 pathway activation and NRG1 gene fusions. NRG1 fusions are enriched among subjects having mucinous non-small cell lung cancer and pancreatic ductal adenocarcinoma; 8-32% of subjects with mucinous NSCLC have NRG1 gene fusions, while 10-20% (up to 70% in small studies) of subjects having KRAS wildtype PDAC have detectable NRG1 gene fusions. The therapeutic utility of monoclonal anti-HER3 antibody therapy targeting NRG1 gene fusions is demonstrated e.g. in WO 2021/048274 A1.

NRG1 gene fusions are described e.g. in WO 2021/048274 A1, WO 2018/182422 A1, WO 2019/051155 A1, Dhanasekaran et al., Nat Commun. (2014) 5: 5893, Drilon et al., Cancer Discov. (2018) 8(6):686-695, Nagasaka et al., Journal of Thoracic Oncology (2019) 14(8):1354-1359 and Jonna et al., Clin Cancer Res. (2019) 25(16):4966-4972, all of which are hereby incorporated by reference in their entirety. The diversity of NRG1 gene fusions may result from NRG1 being located on chromosome 8, which is particularly susceptible to genomic translocation events (Adelaide et al., Genes Chromosomes Cancer. (2003) 37(4):333-45).

In some embodiments, an NRG1 gene fusion is selected from CLU-NRG1, CD74-NRG1, DOC4-NRG1, SLC3A2-NRG1, RBPMS-NRG1, WRN-NRG1, SDC4-NRG1, RAB2!L1-NRG1, VAMP2-NRG1, KIF13B-NRG1, THAP7-NRG1, SMAD4-NRG1, MDK-NRG1, TNC-NRG1, DIP2B-NRG1, MRPL13-NRG1, PARP8-NRG1, ROCK1-NRG1, DPYSL2-NRG1, ATP1B1-NRG1, CDH6-NRG1, APP-NRG1, AKAP13-NRG1, THBS1-NRG1, FOXA1-NRG1, PDE7A-NRG1, RAB31L1-NRG1, CDK1-NRG1, BMPRIB-NRG1, TNFRSF10B-NRG1, and MCPH1-NRG1. In some embodiments, an NRG1 gene fusion is CLU-NRG1.

CD74-NRG1 gene fusion is described e.g. in Fernandez-Cuesta et al. Cancer Discov. (2014) 4:415-22 and Nakaoku et al., Clin Cancer Res (2014) 20:3087-93. DOC4-NRG1 gene fusion is described e.g. in Liu et al., Oncogene. (1999) 18(50):7110-4 and Wang et al., Oncogene. (1999) 18(41):5718-21. SLC3A2-NRG1 gene fusion is described e.g. in Nakaoku et al., Clin Cancer Res (2014) 20:3087-93, Shin et al., Oncotarget (2016) 7:69450-65 and Shin et al., Mol Cancer Ther. (2018) 17(9):2024-2033. RBPMS-NRG1, WRN-NRG1, RAB21L1-NRG1 and SDC4-NRG1 gene fusions are described e.g. in Dhanasekaran et al., Nat Commun. (2014) 5: 5893. VAMP2-NRG1 gene fusion is described e.g. in Jung et al., J Thorac Oncol. (2015) 10(7):1107-11 and Shim et al., J Thorac Oncol. (2015) 10(8):1156-62. KIF13B-NRG1 gene fusion is described e.g. in Xia et al., Int J Surg Pathol. (2017) 25(3):238-240. SMAD4-NRG1, AKAP13-NRG1, THBS1-NRG1, FOXA1-NRG1, PDE7A-NRG1, RAB31L1-NRG1 and THAP7-NRG1 gene fusions are described e.g. in Drilon et al., Cancer Discov. (2018) 8(6):686-695. MDK-NRG1, TNC-NRG1, DIP2B-NRG1, MRPL13-NRG1, PARP8-NRG1, ROCK1-NRG1 and DPYSL2-NRG1 gene fusions are described e.g. in Jonna et al., Clin Cancer Res. (2019) 25(16):4966-4972. ATP1B1-NRG1 gene fusion is described e.g. in Drilon et al., Cancer Discov. (2018) 8(6):686-695 and Jones et al., Annals of Oncology (2017) 28:3092-3097. CLU-NRG1 gene fusion is described e.g. in Drilon et al., Cancer Discov. (2018) 8(6):686-695 and Nagasaka et al., Journal of Thoracic Oncology (2019) 14(8):1354-1359.

In some embodiments, an NRG gene fusion is an NRG2 gene fusion. In some embodiments, the NRG2 gene fusion encodes a polypeptide comprising the EGF-like domain of NRG2, or an amino acid sequence which is capable of binding to HER3 and having at least 60% (e.g. 70%, 75%, 80%, 85%, 90%, 91%, 92%, 93%, 94%, 95%, 96%, 97%, 98%, 99% or 100%) amino acid sequence identity to the EGF-like domain of NRG2.

NRG2 gene fusions include SLC12A2-NRG2 described e.g. in WO 2021/048274 A1, WO 2015/093557 A1, and ZNF208-NRG2 described in Dupain et al., Mol Ther. (2019) 27(1):200-218.

### Cancers

The present disclosure relates to the treatment and prevention of cancers.

A cancer in accordance with the present disclosure may be any unwanted cell proliferation (or any disease manifesting itself by unwanted cell proliferation), neoplasm or tumor. The cancer may be benign or malignant. The cancer may be primary or secondary (e.g. metastatic). A neoplasm or tumor may be any abnormal growth or proliferation of cells, and may be located in (and/or derived from cells of) any organ/tissue.

A cancer may be of cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

A cancer may be, or may comprise, one or more tumors. A cancer may be a glioma, medulloblastoma, meningioma, neurofibroma, ependymoma, Schwannoma, neurofibrosarcoma, astrocytoma and oligodendroglioma, melanoma, mesothelioma, myeloma, lymphoma, Non-Hodgkin's lymphoma (NHL), Hodgkin's lymphoma, cutaneous T-cell lymphoma (CTCL), leukemia, chronic myelogenous leukemia (CML), acute myeloid leukemia (AML), chronic lymphocytic leukemia (CLL), myelodysplastic syndrome (MDS), hepatoma, epidermoid carcinoma, prostate cancer, breast cancer, lung cancer, NSCLC, colon cancer, ovarian cancer, pancreatic cancer, thymic cancer, hematologic cancer or sarcoma.

In some embodiments, a cancer according to the present disclosure is selected from: a solid tumor, breast cancer, breast carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck (SCCHN), lung cancer, non-small cell lung cancer, lung adenocarcinoma, squamous cell lung carcinoma, ovarian cancer, ovarian carcinoma, ovarian serous adenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, uterine cancer, uterine corpus endometrial carcinoma, thyroid cancer, thyroid carcinoma, pheochromocytoma, paraganglioma, bladder cancer, bladder urothelial carcinoma, prostate cancer, prostate adenocarcinoma, sarcoma and thymoma.

In some embodiments, a cancer is selected from: a cancer comprising cells expressing/overexpressing an EGFR family member (e.g. HER3, EGFR, HER2 or HER4), a cancer comprising cells expressing/overexpressing HER3, a cancer comprising cells expressing/overexpressing EGFR, a cancer comprising cells expressing/overexpressing HER3 and EGFR, a cancer comprising cells having a mutation resulting in increased expression of a ligand for HER3, a cancer comprising cells having a mutation resulting in increased expression of a ligand for EGFR, a cancer comprising cells having an NRG gene fusion, a cancer comprising cells having an NRG1 gene fusion, or a cancer comprising cells having an NRG2 gene fusion, a solid tumor, a hematological cancer, a squamous cell cancer, an EGFR-amplified squamous cell carcinoma, breast cancer, breast carcinoma, breast invasive carcinoma, ductal carcinoma, metastatic breast cancer, triple-negative breast cancer, HER2-positive breast cancer, gastric cancer, gastric carcinoma, gastric adenocarcinoma, gastrointestinal adenocarcinoma, colorectal cancer, metastatic colorectal cancer, colon cancer, colorectal carcinoma, colorectal adenocarcinoma, colon adenocarcinoma, head and neck cancer, head and neck squamous cell carcinoma, lung cancer, non-small cell lung cancer, lung adenocarcinoma, invasive mucinous lung adenocarcinoma, squamous cell lung cancer, lung squamous cell carcinoma, squamous non-small cell lung cancer, advanced squamous non-small cell lung cancer, metastatic squamous non-small cell lung cancer, ovarian cancer, ovarian carcinoma, ovarian serous adenocarcinoma, ovarian serous cystadenocarcinoma, fallopian tube cancer, renal cancer, renal cell carcinoma, renal clear cell carcinoma, renal cell adenocarcinoma, renal papillary cell carcinoma, pancreatic cancer, an exocrine cancer, pancreatic adenocarcinoma, pancreatic ductal adenocarcinoma, advanced pancreatic ductal adenocarcinoma, metastatic pancreatic ductal adenocarcinoma, cervical cancer, cervical squamous cell carcinoma, skin cancer, melanoma, oral cavity cancer, oropharyngeal cancer, esophageal cancer, esophageal squamous cell carcinoma (ESCC), esophageal adenocarcinoma, liver cancer, hepatocellular carcinoma, cholangiocarcinoma, gallbladder cancer, uterine cancer, endometrial cancer, uterine corpus endometrial carcinoma, uterine carcinosarcoma, thyroid cancer, thyroid cancer, thyroid carcinoma, pheochromocytoma, paraganglioma, bladder cancer, bladder urothelial carcinoma, prostate cancer, prostate adenocarcinoma, retinoblastoma, sarcoma, soft tissue sarcoma, peritoneal cancer, thymoma, neuroendocrine tumor, neuroendocrine tumor of the nasopharynx, squamous cell carcinoma of the skin, astrocytoma, low grade astrocytoma, high grade astrocytoma, anaplastic astrocytoma and glioblastoma multiforme.

In some embodiments, the cancer to be treated/prevented comprises cells expressing an EGFR family member (e.g. HER3, EGFR, HER2 or HER4), and/or cells expressing a ligand for an EGFR family member. In some embodiments, the cancer to be treated/prevented is a cancer which is positive for an EGFR family member. In some embodiments, the cancer comprises cells that overexpress an EGFR family member and/or a ligand for an EGFR family member. Overexpression can be determined by detection of a level of expression which is greater than the level of expression by equivalent non-cancerous cells/non-tumor tissue.

Expression may be determined by any suitable means. Expression may be gene expression or protein expression. Gene expression can be determined e.g. by detection of mRNA encoding HER3, for example by quantitative real-time PCR (qRT-PCR). Protein expression can be determined e.g. by antibody-based methods, for example by western blot, immunohistochemistry, immunocytochemistry, flow cytometry, or ELISA.

In some embodiments the cancer is a cancer in which HER3 is pathologically-implicated. That is, in some embodiments the cancer is a cancer which is caused or exacerbated by the expression of HER3, a cancer for which expression of HER3 is a risk factor and/or a cancer for which expression of HER3 is positively associated with onset, development, progression, severity or metastasis of the cancer. The cancer may be characterized by expression of HER3, e.g. the cancer may comprise cells (e.g. cells of tumor tissue) expressing HER3. Such cancers may be referred to as being positive for HER3. A cancer which is ‘positive’ for HER3 may be a cancer comprising cells expressing HER3 (e.g. at the cell surface). A cancer which is ‘positive’ for HER3 may overexpress HER3.

In some embodiments, the cancer to be treated/prevented comprises cells harbouring genetic variation (e.g. a mutation) which causes increased (gene and/or protein) expression and/or activity of HER3 relative to comparable cells harbouring a reference allele not comprising the genetic variation (e.g. a non-mutated, or ‘wildtype’ allele).

In some embodiments, a mutation which causes increased expression of HER3 may cause an increase in the level of HER3 on or at the cell surface of a cell comprising the mutation, relative to an equivalent cell not comprising the mutation.

Cells having increased expression of HER3 relative to the level of expression of HER3 by a reference cell (e.g. as a result of mutation) may be described as ‘overexpressing’ HER3, or having ‘upregulated expression’ of HER3. For example, a cancer comprising cells harbouring a mutation resulting in increased expression of HER3 relative to equivalent cells lacking the mutation may be described as a cancer comprising cells displaying overexpression/upregulated expression of HER3. In some embodiments, the reference cell lacking the mutation may be a non-cancerous cell (e.g. of equivalent cell type) or a cancerous cell (e.g. of equivalent cancer type).

In some embodiments, a cancer to be treated/prevented in accordance with the present disclosure may be characterized by an increase in the expression and/or activity of HER3 (i.e. gene and/or protein expression) in an organ/tissue/subject affected by the disease/condition e.g. as compared to normal organ/tissue/subject (i.e. in the absence of the disease/condition). In some embodiments, cells and/or a tumor of a cancer to be treated/prevented may be characterized by an increase in the expression and/or activity of HER3, e.g. as compared to the level of expression and/or activity observed in equivalent non-cancerous cells/non-tumor tissue.

A HER3-overexpressing cancer may overexpress HER3 as a consequence of amplification of the HER3 gene. In some embodiments, a cancer to be treated/prevented in accordance with the present disclosure is a HER3-amplified cancer.

HER3 amplification can be identified using techniques well known in the art, such as in situ hybridization. For example, HER3 amplification can be evaluated by fluorescence in situ hybridization, e.g. as described in Chung et al., J Gynecol Oncol. (2019) 30(5): e75.

In some embodiments, a cell comprising HER3 amplification comprises >2, e.g. one of 3, 4, 5, 6, 7, 8, 9, 10 or more copies of HER3, or >2, e.g. one of 3, 4, 5, 6, 7, 8, 9, 10 or more copies of a fragment of HER3. HER3 amplification may arise as a consequence of gene multiplication or polysomy. In some embodiments, a HER3-amplified cancer may comprise a ratio of HER3 to chromosome 12≥1 (e.g. as determined by ISH), e.g. ≥1.5, ≥2, ≥2.5. In some embodiments, a HER3-amplified cancer may comprise a ratio of HER3 to centromere 12 (CEN12)≥1 (e.g. as determined by ISH), e.g. ≥1.5, ≥2, ≥2.5. In some embodiments, cells of a HER3-amplified cancer may comprise a HER3 gene copy number (GCN)>2, ≥3, ≥4, ≥5, ≥6, ≥7, ≥8, ≥9 or ≥10 (e.g. as determined by NGS).

HER3 and its association with and role in cancer is reviewed e.g. in Mishra, et al., Oncol Rev. (2018) 12(1): 355, Karachaliou et al., BioDrugs. (2017) 31(1):63-73 and Zhang et al., Acta Biochimica et Biophysica Sinica (2016) 48(1): 39-48, all of which are hereby incorporated by reference in their entirety. Mishra, et al., Oncol Rev. (2018) 12(1): 355 also describes intervention targeting HER3 for the treatment of cancer, including monoclonal anti-HER3 antibody therapy.

In some aspects and embodiments according to the present disclosure, a cancer according to the present disclosure is non-identical to a cancer described in WO 2023/017151 A1. That is, in some embodiments, the cancer according to the present disclosure is not a cancer described in WO 2023/017151 A1. In some aspects and embodiments, a cancer according to the present disclosure comprises one or more characteristics distinguishing the cancer from a cancer described in WO 2023/017151 A1.

By way of illustration, in some embodiments a cancer according to the present disclosure (e.g. a cancer for therapeutic/prophylactic intervention using an antigen-binding molecule that binds to HER3) does not comprise genetic variation resulting in an increase in the expression of, or an increase in the activity of a gene product of, MET (e.g. does not comprise MET amplification). By way of further illustration, in some embodiments a cancer according to the present disclosure (e.g. a cancer for therapeutic/prophylactic intervention using an antigen-binding molecule that binds to HER3) is a cancer comprising genetic variation resulting in an increase in the expression of, or an increase in the activity of a gene product of, one or more genes located within chromosome 3q26-3q28 (e.g. one or more genes selected from TP63, SOX2 and P/K3CA; e.g. comprises amplification of one or more of TP63, SOX2 and P/K3CA). In some embodiments, a cancer according to the present disclosure (e.g. a cancer for therapeutic/prophylactic intervention using an antigen-binding molecule that binds to HER3) is a cancer comprising genetic variation resulting in an increase in the expression of, or an increase in the activity of a gene product of, one or more genes located within chromosome 7p (e.g. one or more genes located within chromosome 7p11; e.g. EGFR; e.g. comprises amplification of EGFR). In some embodiments, a cancer according to the present disclosure (e.g. a cancer for therapeutic/prophylactic intervention using an antigen-binding molecule that binds to HER3) is a cancer comprising genetic variation resulting in a decrease in the expression of, or a decrease in the activity of a gene product of, one or more genes located within chromosome 3p (e.g. one or more genes located within chromosome 3p21; e.g. TUSC2; e.g. comprises deletion of TUSC2).

Conversely, in some embodiments a cancer according to the present disclosure (e.g. a cancer for therapeutic/prophylactic intervention using an antigen-binding molecule that binds to HER3 in combination with an antagonist of HER3-mediated signalling) comprises genetic variation resulting in an increase in the expression of, or an increase in the activity of a gene product of, MET (e.g. comprises MET amplification). By way of further illustration, in some embodiments a cancer according to the present disclosure (e.g. a cancer for therapeutic/prophylactic intervention using an antigen-binding molecule that binds to HER3 in combination with an antagonist of HER3-mediated signalling) does not comprise genetic variation resulting in an increase in the expression of, or an increase in the activity of a gene product of, one or more genes located within chromosome 3q26-3q28 (e.g. one or more genes selected from TP63, SOX2 and PIK3CA; e.g. does not comprise amplification of one or more of TP63, SOX2 and PIK3CA). In some embodiments, a cancer according to the present disclosure (e.g. a cancer for therapeutic/prophylactic intervention using an antigen-binding molecule that binds to HER3 in combination with an antagonist of HER3-mediated signalling) does not comprise genetic variation resulting in an increase in the expression of, or an increase in the activity of a gene product of, one or more genes located within chromosome 7p (e.g. one or more genes located within chromosome 7p11; e.g. EGFR; e.g. does not comprise amplification of EGFR). In some embodiments, a cancer according to the present disclosure (e.g. a cancer for therapeutic/prophylactic intervention using an antigen-binding molecule that binds to HER3 in combination with an antagonist of HER3-mediated signalling) does not comprise genetic variation resulting in a decrease in the expression of, or a decrease in the activity of a gene product of, one or more genes located within chromosome 3p (e.g. one or more genes located within chromosome 3p21; e.g. TUSC2; e.g. does not comprise deletion of TUSC2).

### Cancers Characterised by the Absence of Genetic Variation Resulting in an Increase in HER3-Mediated Signalling

Aspects and embodiments of the present disclosure relate to cancers lacking genetic variation resulting in an increase in HER3-mediated signalling. Such cancers may be considered as being more sensitive to/more susceptible to/less resistant to (and therefore more likely to respond well to) therapeutic/prophylactic intervention with an antigen-binding molecule that binds to HER3 (e.g. as a monotherapy). It will be appreciated that cancers described in this section may be further characterised according to the section above entitled ‘Cancers’.

In aspects and embodiments according to the present disclosure, a cancer to be treated/prevented with an antigen-binding molecule that binds to HER3 is characterised according to one or more of the following features:
(1a)(i) is not homozygous for an activating mutation to MET (e.g. an activating mutation to MET described herein).
(1a)(ii) is not heterozygous for an activating mutation to MET (e.g. an activating mutation to MET described herein).
(1a)(iii) does not comprise amplification of MET (e.g. amplification of MET as described herein).
(1a)(iv) does not comprise genetic variation resulting in an increase in the expression of, or an increase in the activity of a gene product of, MET.
(2a)(i) is not homozygous for an activating mutation to KRAS (e.g. an activating mutation to KRAS as described herein).
(2a)(ii) is not heterozygous for an activating mutation to KRAS (e.g. an activating mutation to KRAS as described herein).
(2a)(iii) does not comprise amplification of KRAS (e.g. amplification of KRAS as described herein).
(2a)(iv) does not comprise genetic variation resulting in an increase in the expression of, or an increase in the activity of a gene product of, KRAS.
(3a)(i) is not homozygous for an activating mutation to PIK3CA (e.g. an activating mutation to PIK3CA as described herein).
(3a)(ii) is not heterozygous for an activating mutation to PIK3CA (e.g. an activating mutation to PIK3CA as described herein).
(3a)(iii) does not comprise amplification of PIK3CA (e.g. amplification of PIK3CA as described herein).
(3a)(iv) does not comprise genetic variation resulting in an increase in the expression of, or an increase in the activity of a gene product of, PIK3CA.
(4a)(i) is not homozygous for an activating mutation to BRAF (e.g. an activating mutation to BRAF as described herein).
(4a)(ii) is not heterozygous for an activating mutation to BRAF (e.g. an activating mutation to BRAF as described herein).
(4a)(iii) does not comprise amplification of BRAF (e.g. amplification of BRAF as described herein).
(4a)(iv) does not comprise genetic variation resulting in an increase in the expression of, or an increase in the activity of a gene product of, BRAF.
(5a)(i) does not comprise genetic variation resulting in a decrease in the expression of, or a decrease in the activity of a gene product of, PTEN.
(5a)(ii) is not homozygous for an inactivating mutation to PTEN (e.g. an inactivating mutation to PTEN as described herein).
(5a)(iii) is not heterozygous for an inactivating mutation to PTEN (e.g. an inactivating mutation to PTEN as described herein).
(5a)(iv) does not comprise deletion of PTEN (e.g. deletion of PTEN as described herein).
(6a)(i) comprises genetic variation resulting in an increase in the expression of, or an increase in the activity of a gene product of, TP63.
(6a)(ii) comprises amplification of TP63 (e.g. amplification of TP63 as described herein).
(7a)(i) comprises genetic variation resulting in an increase in the expression of, or an increase in the activity of a gene product of, PIK3CA.
(7a)(ii) comprises amplification of PIK3CA (e.g. amplification of PIK3CA as described herein).
(8a)(i) comprises genetic variation resulting in an increase in the expression of, or an increase in the activity of a gene product of, SOX2.
(8a)(ii) comprises amplification of SOX2 (e.g. amplification of SOX2 as described herein).
(9a)(i) comprises genetic variation resulting in an increase in the expression of a ligand for HER3 (e.g. as described herein).
(9a)(ii) comprises an NRG gene fusion (e.g. an NRG gene fusion as described herein).
(9a)(iii) comprises an NRG1 gene fusion (e.g. an NRG1 gene fusion as described herein).
(10a)(i) comprises genetic variation resulting in an increase in the expression of, or an increase in the activity of a gene product of, EGFR.
(10a)(ii) comprises amplification of EGFR (e.g. amplification of EGFR as described herein).
(11a)(i) does not comprise genetic variation resulting in a decrease in the expression of, or a decrease in the activity of a gene product of, TUSC2.
(11a)(ii) does not comprise deletion of TUSC2 (e.g. deletion of TUSC2 as described herein).

In some embodiments, a cancer to be treated/prevented with an antigen-binding molecule that binds to HER3 is characterised by one of the following combinations of features (with reference to the preceding paragraph): (1a), (2a); (1a), (3a); (1a), (4a); (1a), (5a); (1a), (6a); (1a), (7a); (1a), (8a); (1a), (9a); (1a), (10a); (1a), (11a); (1a), (2a), (3a); (1a), (2a), (4a); (1a), (2a), (5a); (1a), (2a), (6a); (1a), (2a), (7a); (1a), (2a), (8a); (1a), (2a), (9a); (1a), (2a), (10a); (1a), (2a), (11a); (1a), (3a), (4a); (1a), (3a), (5a); (1a), (3a), (6a); (1a), (3a), (7a); (1a), (3a), (8a); (1a), (3a), (9a); (1a), (3a), (10a); (1a), (3a), (11a); (1a), (4a), (5a); (1a), (4a), (6a); (1a), (4a), (7a); (1a), (4a), (8a); (1a), (4a), (9a); (1a), (4a), (10a); (1a), (4a), (11a); (1a), (5a), (6a); (1a), (5a), (7a); (1a), (5a), (8a); (1a), (5a), (9a); (1a), (5a), (10a); (1a), (5a), (11a); (1a), (6a), (7a); (1a), (6a), (8a); (1a), (6a), (9a); (1a), (6a), (10a); (1a), (6a), (11a); (1a), (7a), (8a); (1a), (7a), (9a); (1a), (7a), (10a); (1a), (7a), (11a); (1a), (8a), (9a); (1a), (8a), (10a); (1a), (8a), (11a); (1a), (9a), (10a); (1a), (9a), (11a); (1a), (10a), (11a); (1a), (2a), (3a), (4a); (1a), (2a), (3a), (5a); (1a), (2a), (3a), (6a); (1a), (2a), (3a), (7a); (1a), (2a), (3a), (8a); (1a), (2a), (3a), (9a); (1a), (2a), (3a), (10a); (1a), (2a), (3a), (11a); (1a), (2a), (4a), (5a); (1a), (2a), (4a), (6a); (1a), (2a), (4a), (7a); (1a), (2a), (4a), (8a); (1a), (2a), (4a), (9a); (1a), (2a), (4a), (10a); (1a), (2a), (4a), (11a); (1a), (2a), (5a), (6a); (1a), (2a), (5a), (7a); (1a), (2a), (5a), (8a); (1a), (2a), (5a), (9a); (1a), (2a), (5a), (10a); (1a), (2a), (5a), (11a); (1a), (2a), (6a), (7a); (1a), (2a), (6a), (8a); (1a), (2a), (6a), (9a); (1a), (2a), (6a), (10a); (1a), (2a), (6a), (11a); (1a), (2a), (7a), (8a); (1a), (2a), (7a), (9a); (1a), (2a), (7a), (10a); (1a), (2a), (7a), (11a); (1a), (2a), (8a), (9a); (1a), (2a), (8a), (10a); (1a), (2a), (8a), (11a); (1a), (2a), (9a), (10a); (1a), (2a), (9a), (11a); (1a), (2a), (10a), (11a); (1a), (3a), (4a), (5a); (1a), (3a), (4a), (6a); (1a), (3a), (4a), (7a); (1a), (3a), (4a), (8a); (1a), (3a), (4a), (9a); (1a), (3a), (4a), (10a); (1a), (3a), (4a), (11a); (1a), (3a), (5a), (6a); (1a), (3a), (5a), (7a); (1a), (3a), (5a), (8a); (1a), (3a), (5a), (9a); (1a), (3a), (5a), (10a); (1a), (3a), (5a), (11a); (1a), (3a), (6a), (7a); (1a), (3a), (6a), (8a); (1a), (3a), (6a), (9a); (1a), (3a), (6a), (10a); (1a), (3a), (6a), (11a); (1a), (3a), (7a), (8a); (1a), (3a), (7a), (9a); (1a), (3a), (7a), (10a); (1a), (3a), (7a), (11a); (1a), (3a), (8a), (9a); (1a), (3a), (8a), (10a); (1a), (3a), (8a), (11a); (1a), (3a), (9a), (10a); (1a), (3a), (9a), (11a); (1a), (3a), (10a), (11a); (1a), (4a), (5a), (6a); (1a), (4a), (5a), (7a); (1a), (4a), (5a), (8a); (1a), (4a), (5a), (9a); (1a), (4a), (5a), (10a); (1a), (4a), (5a), (11a); (1a), (4a), (6a), (7a); (1a), (4a), (6a), (8a); (1a), (4a), (6a), (9a); (1a), (4a), (6a), (10a); (1a), (4a), (6a), (11a); (1a), (4a), (7a), (8a); (1a), (4a), (7a), (9a); (1a), (4a), (7a), (10a); (1a), (4a), (7a), (11a); (1a), (4a), (8a), (9a); (1a), (4a), (8a), (10a); (1a), (4a), (8a), (11a); (1a), (4a), (9a), (10a); (1a), (4a), (9a), (11a); (1a), (4a), (10a), (11a); (1a), (5a), (6a), (7a); (1a), (5a), (6a), (8a); (1a), (5a), (6a), (9a); (1a), (5a), (6a), (10a); (1a), (5a), (6a), (11a); (1a), (5a), (7a), (8a); (1a), (5a), (7a), (9a); (1a), (5a), (7a), (10a); (1a), (5a), (7a), (11a); (1a), (5a), (8a), (9a); (1a), (5a), (8a), (10a); (1a), (5a), (8a), (11a); (1a), (5a), (9a), (10a); (1a), (5a), (9a), (11a); (1a), (5a), (10a), (11a); (1a), (6a), (7a), (8a); (1a), (6a), (7a), (9a); (1a), (6a), (7a), (10a); (1a), (6a), (7a), (11a); (1a), (6a), (8a), (9a); (1a), (6a), (8a), (10a); (1a), (6a), (8a), (11a); (1a), (6a), (9a), (10a); (1a), (6a), (9a), (11a); (1a), (6a), (10a), (11a); (1a), (7a), (8a), (9a); (1a), (7a), (8a), (10a); (1a), (7a), (8a), (11a); (1a), (7a), (9a), (10a); (1a), (7a), (9a), (11a); (1a), (7a), (10a), (11a); (1a), (8a), (9a), (10a); (1a), (8a), (9a), (11a); (1a), (8a), (10a), (11a); (1a), (9a), (10a), (11a); (1a), (2a), (3a), (4a), (5a); (1a), (2a), (3a), (4a), (6a); (1a), (2a), (3a), (4a), (7a); (1a), (2a), (3a), (4a), (8a); (1a), (2a), (3a), (4a), (9a); (1a), (2a), (3a), (4a), (10a); (1a), (2a), (3a), (4a), (11a); (1a), (2a), (3a), (5a), (6a); (1a), (2a), (3a), (5a), (7a); (1a), (2a), (3a), (5a), (8a); (1a), (2a), (3a), (5a), (9a); (1a), (2a), (3a), (5a), (10a); (1a), (2a), (3a), (5a), (11a); (1a), (2a), (3a), (6a), (7a); (1a), (2a), (3a), (6a), (8a); (1a), (2a), (3a), (6a), (9a); (1a), (2a), (3a), (6a), (10a); (1a), (2a), (3a), (6a), (11a); (1a), (2a), (3a), (7a), (8a); (1a), (2a), (3a), (7a), (9a); (1a), (2a), (3a), (7a), (10a); (1a), (2a), (3a), (7a), (11a); (1a), (2a), (3a), (8a), (9a); (1a), (2a), (3a), (8a), (10a); (1a), (2a), (3a), (8a), (11a); (1a), (2a), (3a), (9a), (10a); (1a), (2a), (3a), (9a), (11a); (1a), (2a), (3a), (10a), (11a); (1a), (2a), (4a), (5a), (6a); (1a), (2a), (4a), (5a), (7a); (1a), (2a), (4a), (5a), (8a); (1a), (2a), (4a), (5a), (9a); (1a), (2a), (4a), (5a), (10a); (1a), (2a), (4a), (5a), (11a); (1a), (2a), (4a), (6a), (7a); (1a), (2a), (4a), (6a), (8a); (1a), (2a), (4a), (6a), (9a); (1a), (2a), (4a), (6a), (10a); (1a), (2a), (4a), (6a), (11a); (1a), (2a), (4a), (7a), (8a); (1a), (2a), (4a), (7a), (9a); (1a), (2a), (4a), (7a), (10a); (1a), (2a), (4a), (7a), (11a); (1a), (2a), (4a), (8a), (9a); (1a), (2a), (4a), (8a), (10a); (1a), (2a), (4a), (8a), (11a); (1a), (2a), (4a), (9a), (10a); (1a), (2a), (4a), (9a), (11a); (1a), (2a), (4a), (10a), (11a); (1a), (2a), (5a), (6a), (7a); (1a), (2a), (5a), (6a), (8a); (1a), (2a), (5a), (6a), (9a); (1a), (2a), (5a), (6a), (10a); (1a), (2a), (5a), (6a), (11a); (1a), (2a), (5a), (7a), (8a); (1a), (2a), (5a), (7a), (9a); (1a), (2a), (5a), (7a), (10a); (1a), (2a), (5a), (7a), (11a); (1a), (2a), (5a), (8a), (9a); (1a), (2a), (5a), (8a), (10a); (1a), (2a), (5a), (8a), (11a); (1a), (2a), (5a), (9a), (10a); (1a), (2a), (5a), (9a), (11a); (1a), (2a), (5a), (10a), (11a); (1a), (2a), (6a), (7a), (8a); (1a), (2a), (6a), (7a), (9a); (1a), (2a), (6a), (7a), (10a); (1a), (2a), (6a), (7a), (11a); (1a), (2a), (6a), (8a), (9a); (1a), (2a), (6a), (8a), (10a); (1a), (2a), (6a), (8a), (11a); (1a), (2a), (6a), (9a), (10a); (1a), (2a), (6a), (9a), (11a); (1a), (2a), (6a), (10a), (11a); (1a), (2a), (7a), (8a), (9a); (1a), (2a), (7a), (8a), (10a); (1a), (2a), (7a), (8a), (11a); (1a), (2a), (7a), (9a), (10a); (1a), (2a), (7a), (9a), (11a); (1a), (2a), (7a), (10a), (11a); (1a), (2a), (8a), (9a), (10a); (1a), (2a), (8a), (9a), (11a); (1a), (2a), (8a), (10a), (11a); (1a), (2a), (9a), (10a), (11a); (1a), (3a), (4a), (5a), (6a); (1a), (3a), (4a), (5a), (7a); (1a), (3a), (4a), (5a), (8a); (1a), (3a), (4a), (5a), (9a); (1a), (3a), (4a), (5a), (10a); (1a), (3a), (4a), (5a), (11a); (1a), (3a), (4a), (6a), (7a); (1a), (3a), (4a), (6a), (8a); (1a), (3a), (4a), (6a), (9a); (1a), (3a), (4a), (6a), (10a); (1a), (3a), (4a), (6a), (11a); (1a), (3a), (4a), (7a), (8a); (1a), (3a), (4a), (7a), (9a); (1a), (3a), (4a), (7a), (10a); (1a), (3a), (4a), (7a), (11a); (1a), (3a), (4a), (8a), (9a); (1a), (3a), (4a), (8a), (10a); (1a), (3a), (4a), (8a), (11a); (1a), (3a), (4a), (9a), (10a); (1a), (3a), (4a), (9a), (11a); (1a), (3a), (4a), (10a), (11a); (1a), (3a), (5a), (6a), (7a); (1a), (3a), (5a), (6a), (8a); (1a), (3a), (5a), (6a), (9a); (1a), (3a), (5a), (6a), (10a); (1a), (3a), (5a), (6a), (11a); (1a), (3a), (5a), (7a), (8a); (1a), (3a), (5a), (7a), (9a); (1a), (3a), (5a), (7a), (10a); (1a), (3a), (5a), (7a), (11a); (1a), (3a), (5a), (8a), (9a); (1a), (3a), (5a), (8a), (10a); (1a), (3a), (5a), (8a), (11a); (1a), (3a), (5a), (9a), (10a); (1a), (3a), (5a), (9a), (11a); (1a), (3a), (5a), (10a), (11a); (1a), (3a), (6a), (7a), (8a); (1a), (3a), (6a), (7a), (9a); (1a), (3a), (6a), (7a), (10a); (1a), (3a), (6a), (7a), (11a); (1a), (3a), (6a), (8a), (9a); (1a), (3a), (6a), (8a), (10a); (1a), (3a), (6a), (8a), (11a); (1a), (3a), (6a), (9a), (10a); (1a), (3a), (6a), (9a), (11a); (1a), (3a), (6a), (10a), (11a); (1a), (3a), (7a), (8a), (9a); (1a), (3a), (7a), (8a), (10a); (1a), (3a), (7a), (8a), (11a); (1a), (3a), (7a), (9a), (10a); (1a), (3a), (7a), (9a), (11a); (1a), (3a), (7a), (10a), (11a); (1a), (3a), (8a), (9a), (10a); (1a), (3a), (8a), (9a), (11a); (1a), (3a), (8a), (10a), (11a); (1a), (3a), (9a), (10a), (11a); (1a), (4a), (5a), (6a), (7a); (1a), (4a), (5a), (6a), (8a); (1a), (4a), (5a), (6a), (9a); (1a), (4a), (5a), (6a), (10a); (1a), (4a), (5a), (6a), (11a); (1a), (4a), (5a), (7a), (8a); (1a), (4a), (5a), (7a), (9a); (1a), (4a), (5a), (7a), (10a); (1a), (4a), (5a), (7a), (11a); (1a), (4a), (5a), (8a), (9a); (1a), (4a), (5a), (8a), (10a); (1a), (4a), (5a), (8a), (11a); (1a), (4a), (5a), (9a), (10a); (1a), (4a), (5a), (9a), (11a); (1a), (4a), (5a), (10a), (11a); (1a), (4a), (6a), (7a), (8a); (1a), (4a), (6a), (7a), (9a); (1a), (4a), (6a), (7a), (10a); (1a), (4a), (6a), (7a), (11a); (1a), (4a), (6a), (8a), (9a); (1a), (4a), (6a), (8a), (10a); (1a), (4a), (6a), (8a), (11a); (1a), (4a), (6a), (9a), (10a); (1a), (4a), (6a), (9a), (11a); (1a), (4a), (6a), (10a), (11a); (1a), (4a), (7a), (8a), (9a); (1a), (4a), (7a), (8a), (10a); (1a), (4a), (7a), (8a), (11a); (1a), (4a), (7a), (9a), (10a); (1a), (4a), (7a), (9a), (11a); (1a), (4a), (7a), (10a), (11a); (1a), (4a), (8a), (9a), (10a); (1a), (4a), (8a), (9a), (11a); (1a), (4a), (8a), (10a), (11a); (1a), (4a), (9a), (10a), (11a); (1a), (5a), (6a), (7a), (8a); (1a), (5a), (6a), (7a), (9a); (1a), (5a), (6a), (7a), (10a); (1a), (5a), (6a), (7a), (11a); (1a), (5a), (6a), (8a), (9a); (1a), (5a), (6a), (8a), (10a); (1a), (5a), (6a), (8a), (11a); (1a), (5a), (6a), (9a), (10a); (1a), (5a), (6a), (9a), (11a); (1a), (5a), (6a), (10a), (11a); (1a), (5a), (7a), (8a), (9a); (1a), (5a), (7a), (8a), (10a); (1a), (5a), (7a), (8a), (11a); (1a), (5a), (7a), (9a), (10a); (1a), (5a), (7a), (9a), (11a); (1a), (5a), (7a), (10a), (11a); (1a), (5a), (8a), (9a), (10a); (1a), (5a), (8a), (9a), (11a); (1a), (5a), (8a), (10a), (11a); (1a), (5a), (9a), (10a), (11a); (1a), (6a), (7a), (8a), (9a); (1a), (6a), (7a), (8a), (10a); (1a), (6a), (7a), (8a), (11a); (1a), (6a), (7a), (9a), (10a); (1a), (6a), (7a), (9a), (11a); (1a), (6a), (7a), (10a), (11a); (1a), (6a), (8a), (9a), (10a); (1a), (6a), (8a), (9a), (11a); (1a), (6a), (8a), (10a), (11a); (1a), (6a), (9a), (10a), (11a); (1a), (7a), (8a), (9a), (10a); (1a), (7a), (8a), (9a), (11a); (1a), (7a), (8a), (10a), (11a); (1a), (7a), (9a), (10a), (11a); (1a), (8a), (9a), (10a), (11a); (1a), (2a), (3a), (4a), (5a), (6a); (1a), (2a), (3a), (4a), (5a), (7a); (1a), (2a), (3a), (4a), (5a), (8a); (1a), (2a), (3a), (4a), (5a), (9a); (1a), (2a), (3a), (4a), (5a), (10a); (1a), (2a), (3a), (4a), (5a), (11a); (1a), (2a), (3a), (4a), (6a), (7a); (1a), (2a), (3a), (4a), (6a), (8a); (1a), (2a), (3a), (4a), (6a), (9a); (1a), (2a), (3a), (4a), (6a), (10a); (1a), (2a), (3a), (4a), (6a), (11a); (1a), (2a), (3a), (4a), (7a), (8a); (1a), (2a), (3a), (4a), (7a), (9a); (1a), (2a), (3a), (4a), (7a), (10a); (1a), (2a), (3a), (4a), (7a), (11a); (1a), (2a), (3a), (4a), (8a), (9a); (1a), (2a), (3a), (4a), (8a), (10a); (1a), (2a), (3a), (4a), (8a), (11a); (1a), (2a), (3a), (4a), (9a), (10a); (1a), (2a), (3a), (4a), (9a), (11a); (1a), (2a), (3a), (4a), (10a), (11a); (1a), (2a), (3a), (5a), (6a), (7a); (1a), (2a), (3a), (5a), (6a), (8a); (1a), (2a), (3a), (5a), (6a), (9a); (1a), (2a), (3a), (5a), (6a), (10a); (1a), (2a), (3a), (5a), (6a), (11a); (1a), (2a), (3a), (5a), (7a), (8a); (1a), (2a), (3a), (5a), (7a), (9a); (1a), (2a), (3a), (5a), (7a), (10a); (1a), (2a), (3a), (5a), (7a), (11a); (1a), (2a), (3a), (5a), (8a), (9a); (1a), (2a), (3a), (5a), (8a), (10a); (1a), (2a), (3a), (5a), (8a), (11a); (1a), (2a), (3a), (5a), (9a), (10a); (1a), (2a), (3a), (5a), (9a), (11a); (1a), (2a), (3a), (5a), (10a), (11a); (1a), (2a), (3a), (6a), (7a), (8a); (1a), (2a), (3a), (6a), (7a), (9a); (1a), (2a), (3a), (6a), (7a), (10a); (1a), (2a), (3a), (6a), (7a), (11a); (1a), (2a), (3a), (6a), (8a), (9a); (1a), (2a), (3a), (6a), (8a), (10a); (1a), (2a), (3a), (6a), (8a), (11a); (1a), (2a), (3a), (6a), (9a), (10a); (1a), (2a), (3a), (6a), (9a), (11a); (1a), (2a), (3a), (6a), (10a), (11a); (1a), (2a), (3a), (7a), (8a), (9a); (1a), (2a), (3a), (7a), (8a), (10a); (1a), (2a), (3a), (7a), (8a), (11a); (1a), (2a), (3a), (7a), (9a), (10a); (1a), (2a), (3a), (7a), (9a), (11a); (1a), (2a), (3a), (7a), (10a), (11a); (1a), (2a), (3a), (8a), (9a), (10a); (1a), (2a), (3a), (8a), (9a), (11a); (1a), (2a), (3a), (8a), (10a), (11a); (1a), (2a), (3a), (9a), (10a), (11a); (1a), (2a), (4a), (5a), (6a), (7a); (1a), (2a), (4a), (5a), (6a), (8a); (1a), (2a), (4a), (5a), (6a), (9a); (1a), (2a), (4a), (5a), (6a), (10a); (1a), (2a), (4a), (5a), (6a), (11a); (1a), (2a), (4a), (5a), (7a), (8a); (1a), (2a), (4a), (5a), (7a), (9a); (1a), (2a), (4a), (5a), (7a), (10a); (1a), (2a), (4a), (5a), (7a), (11a); (1a), (2a), (4a), (5a), (8a), (9a); (1a), (2a), (4a), (5a), (8a), (10a); (1a), (2a), (4a), (5a), (8a), (11a); (1a), (2a), (4a), (5a), (9a), (10a); (1a), (2a), (4a), (5a), (9a), (11a); (1a), (2a), (4a), (5a), (10a), (11a); (1a), (2a), (4a), (6a), (7a), (8a); (1a), (2a), (4a), (6a), (7a), (9a); (1a), (2a), (4a), (6a), (7a), (10a); (1a), (2a), (4a), (6a), (7a), (11a); (1a), (2a), (4a), (6a), (8a), (9a); (1a), (2a), (4a), (6a), (8a), (10a); (1a), (2a), (4a), (6a), (8a), (11a); (1a), (2a), (4a), (6a), (9a), (10a); (1a), (2a), (4a), (6a), (9a), (11a); (1a), (2a), (4a), (6a), (10a), (11a); (1a), (2a), (4a), (7a), (8a), (9a); (1a), (2a), (4a), (7a), (8a), (10a); (1a), (2a), (4a), (7a), (8a), (11a); (1a), (2a), (4a), (7a), (9a), (10a); (1a), (2a), (4a), (7a), (9a), (11a); (1a), (2a), (4a), (7a), (10a), (11a); (1a), (2a), (4a), (8a), (9a), (10a); (1a), (2a), (4a), (8a), (9a), (11a); (1a), (2a), (4a), (8a), (10a), (11a); (1a), (2a), (4a), (9a), (10a), (11a); (1a), (2a), (5a), (6a), (7a), (8a); (1a), (2a), (5a), (6a), (7a), (9a); (1a), (2a), (5a), (6a), (7a), (10a); (1a), (2a), (5a), (6a), (7a), (11a); (1a), (2a), (5a), (6a), (8a), (9a); (1a), (2a), (5a), (6a), (8a), (10a); (1a), (2a), (5a), (6a), (8a), (11a); (1a), (2a), (5a), (6a), (9a), (10a); (1a), (2a), (5a), (6a), (9a), (11a); (1a), (2a), (5a), (6a), (10a), (11a); (1a), (2a), (5a), (7a), (8a), (9a); (1a), (2a), (5a), (7a), (8a), (10a); (1a), (2a), (5a), (7a), (8a), (11a); (1a), (2a), (5a), (7a), (9a), (10a); (1a), (2a), (5a), (7a), (9a), (11a); (1a), (2a), (5a), (7a), (10a), (11a); (1a), (2a), (5a), (8a), (9a), (10a); (1a), (2a), (5a), (8a), (9a), (11a); (1a), (2a), (5a), (8a), (10a), (11a); (1a), (2a), (5a), (9a), (10a), (11a); (1a), (2a), (6a), (7a), (8a), (9a); (1a), (2a), (6a), (7a), (8a), (10a); (1a), (2a), (6a), (7a), (8a), (11a); (1a), (2a), (6a), (7a), (9a), (10a); (1a), (2a), (6a), (7a), (9a), (11a); (1a), (2a), (6a), (7a), (10a), (11a); (1a), (2a), (6a), (8a), (9a), (10a); (1a), (2a), (6a), (8a), (9a), (11a); (1a), (2a), (6a), (8a), (10a), (11a); (1a), (2a), (6a), (9a), (10a), (11a); (1a), (2a), (7a), (8a), (9a), (10a); (1a), (2a), (7a), (8a), (9a), (11a); (1a), (2a), (7a), (8a), (10a), (11a); (1a), (2a), (7a), (9a), (10a), (11a); (1a), (2a), (8a), (9a), (10a), (11a); (1a), (3a), (4a), (5a), (6a), (7a); (1a), (3a), (4a), (5a), (6a), (8a); (1a), (3a), (4a), (5a), (6a), (9a); (1a), (3a), (4a), (5a), (6a), (10a); (1a), (3a), (4a), (5a), (6a), (11a); (1a), (3a), (4a), (5a), (7a), (8a); (1a), (3a), (4a), (5a), (7a), (9a); (1a), (3a), (4a), (5a), (7a), (10a); (1a), (3a), (4a), (5a), (7a), (11a); (1a), (3a), (4a), (5a), (8a), (9a); (1a), (3a), (4a), (5a), (8a), (10a); (1a), (3a), (4a), (5a), (8a), (11a); (1a), (3a), (4a), (5a), (9a), (10a); (1a), (3a), (4a), (5a), (9a), (11a); (1a), (3a), (4a), (5a), (10a), (11a); (1a), (3a), (4a), (6a), (7a), (8a); (1a), (3a), (4a), (6a), (7a), (9a); (1a), (3a), (4a), (6a), (7a), (10a); (1a), (3a), (4a), (6a), (7a), (11a); (1a), (3a), (4a), (6a), (8a), (9a); (1a), (3a), (4a), (6a), (8a), (10a); (1a), (3a), (4a), (6a), (8a), (11a); (1a), (3a), (4a), (6a), (9a), (10a); (1a), (3a), (4a), (6a), (9a), (11a); (1a), (3a), (4a), (6a), (10a), (11a); (1a), (3a), (4a), (7a), (8a), (9a); (1a), (3a), (4a), (7a), (8a), (10a); (1a), (3a), (4a), (7a), (8a), (11a); (1a), (3a), (4a), (7a), (9a), (10a); (1a), (3a), (4a), (7a), (9a), (11a); (1a), (3a), (4a), (7a), (10a), (11a); (1a), (3a), (4a), (8a), (9a), (10a); (1a), (3a), (4a), (8a), (9a), (11a); (1a), (3a), (4a), (8a), (10a), (11a); (1a), (3a), (4a), (9a), (10a), (11a); (1a), (3a), (5a), (6a), (7a), (8a); (1a), (3a), (5a), (6a), (7a), (9a); (1a), (3a), (5a), (6a), (7a), (10a); (1a), (3a), (5a), (6a), (7a), (11a); (1a), (3a), (5a), (6a), (8a), (9a); (1a), (3a), (5a), (6a), (8a), (10a); (1a), (3a), (5a), (6a), (8a), (11a); (1a), (3a), (5a), (6a), (9a), (10a); (1a), (3a), (5a), (6a), (9a), (11a); (1a), (3a), (5a), (6a), (10a), (11a); (1a), (3a), (5a), (7a), (8a), (9a); (1a), (3a), (5a), (7a), (8a), (10a); (1a), (3a), (5a), (7a), (8a), (11a); (1a), (3a), (5a), (7a), (9a), (10a); (1a), (3a), (5a), (7a), (9a), (11a); (1a), (3a), (5a), (7a), (10a), (11a); (1a), (3a), (5a), (8a), (9a), (10a); (1a), (3a), (5a), (8a), (9a), (11a); (1a), (3a), (5a), (8a), (10a), (11a); (1a), (3a), (5a), (9a), (10a), (11a); (1a), (3a), (6a), (7a), (8a), (9a); (1a), (3a), (6a), (7a), (8a), (10a); (1a), (3a), (6a), (7a), (8a), (11a); (1a), (3a), (6a), (7a), (9a), (10a); (1a), (3a), (6a), (7a), (9a), (11a); (1a), (3a), (6a), (7a), (10a), (11a); (1a), (3a), (6a), (8a), (9a), (10a); (1a), (3a), (6a), (8a), (9a), (11a); (1a), (3a), (6a), (8a), (10a), (11a); (1a), (3a), (6a), (9a), (10a), (11a); (1a), (3a), (7a), (8a), (9a), (10a); (1a), (3a), (7a), (8a), (9a), (11a); (1a), (3a), (7a), (8a), (10a), (11a); (1a), (3a), (7a), (9a), (10a), (11a); (1a), (3a), (8a), (9a), (10a), (11a); (1a), (4a), (5a), (6a), (7a), (8a); (1a), (4a), (5a), (6a), (7a), (9a); (1a), (4a), (5a), (6a), (7a), (10a); (1a), (4a), (5a), (6a), (7a), (11a); (1a), (4a), (5a), (6a), (8a), (9a); (1a), (4a), (5a), (6a), (8a), (10a); (1a), (4a), (5a), (6a), (8a), (11a); (1a), (4a), (5a), (6a), (9a), (10a); (1a), (4a), (5a), (6a), (9a), (11a); (1a), (4a), (5a), (6a), (10a), (11a); (1a), (4a), (5a), (7a), (8a), (9a); (1a), (4a), (5a), (7a), (8a), (10a); (1a), (4a), (5a), (7a), (8a), (11a); (1a), (4a), (5a), (7a), (9a), (10a); (1a), (4a), (5a), (7a), (9a), (11a); (1a), (4a), (5a), (7a), (10a), (11a); (1a), (4a), (5a), (8a), (9a), (10a); (1a), (4a), (5a), (8a), (9a), (11a); (1a), (4a), (5a), (8a), (10a), (11a); (1a), (4a), (5a), (9a), (10a), (11a); (1a), (4a), (6a), (7a), (8a), (9a); (1a), (4a), (6a), (7a), (8a), (10a); (1a), (4a), (6a), (7a), (8a), (11a); (1a), (4a), (6a), (7a), (9a), (10a); (1a), (4a), (6a), (7a), (9a), (11a); (1a), (4a), (6a), (7a), (10a), (11a); (1a), (4a), (6a), (8a), (9a), (10a); (1a), (4a), (6a), (8a), (9a), (11a); (1a), (4a), (6a), (8a), (10a), (11a); (1a), (4a), (6a), (9a), (10a), (11a); (1a), (4a), (7a), (8a), (9a), (10a); (1a), (4a), (7a), (8a), (9a), (11a); (1a), (4a), (7a), (8a), (10a), (11a); (1a), (4a), (7a), (9a), (10a), (11a); (1a), (4a), (8a), (9a), (10a), (11a); (1a), (5a), (6a), (7a), (8a), (9a); (1a), (5a), (6a), (7a), (8a), (10a); (1a), (5a), (6a), (7a), (8a), (11a); (1a), (5a), (6a), (7a), (9a), (10a); (1a), (5a), (6a), (7a), (9a), (11a); (1a), (5a), (6a), (7a), (10a), (11a); (1a), (5a), (6a), (8a), (9a), (10a); (1a), (5a), (6a), (8a), (9a), (11a); (1a), (5a), (6a), (8a), (10a), (11a); (1a), (5a), (6a), (9a), (10a), (11a); (1a), (5a), (7a), (8a), (9a), (10a); (1a), (5a), (7a), (8a), (9a), (11a); (1a), (5a), (7a), (8a), (10a), (11a); (1a), (5a), (7a), (9a), (10a), (11a); (1a), (5a), (8a), (9a), (10a), (11a); (1a), (6a), (7a), (8a), (9a), (10a); (1a), (6a), (7a), (8a), (9a), (11a); (1a), (6a), (7a), (8a), (10a), (11a); (1a), (6a), (7a), (9a), (10a), (11a); (1a), (6a), (8a), (9a), (10a), (11a); (1a), (7a), (8a), (9a), (10a), (11a); (1a), (2a), (3a), (4a), (5a), (6a), (7a); (1a), (2a), (3a), (4a), (5a), (6a), (8a); (1a), (2a), (3a), (4a), (5a), (6a), (9a); (1a), (2a), (3a), (4a), (5a), (6a), (10a); (1a), (2a), (3a), (4a), (5a), (6a), (11a); (1a), (2a), (3a), (4a), (5a), (7a), (8a); (1a), (2a), (3a), (4a), (5a), (7a), (9a); (1a), (2a), (3a), (4a), (5a), (7a), (10a); (1a), (2a), (3a), (4a), (5a), (7a), (11a); (1a), (2a), (3a), (4a), (5a), (8a), (9a); (1a), (2a), (3a), (4a), (5a), (8a), (10a); (1a), (2a), (3a), (4a), (5a), (8a), (11a); (1a), (2a), (3a), (4a), (5a), (9a), (10a); (1a), (2a), (3a), (4a), (5a), (9a), (11a); (1a), (2a), (3a), (4a), (5a), (10a), (11a); (1a), (2a), (3a), (4a), (6a), (7a), (8a); (1a), (2a), (3a), (4a), (6a), (7a), (9a); (1a), (2a), (3a), (4a), (6a), (7a), (10a); (1a), (2a), (3a), (4a), (6a), (7a), (11a); (1a), (2a), (3a), (4a), (6a), (8a), (9a); (1a), (2a), (3a), (4a), (6a), (8a), (10a); (1a), (2a), (3a), (4a), (6a), (8a), (11a); (1a), (2a), (3a), (4a), (6a), (9a), (10a); (1a), (2a), (3a), (4a), (6a), (9a), (11a); (1a), (2a), (3a), (4a), (6a), (10a), (11a); (1a), (2a), (3a), (4a), (7a), (8a), (9a); (1a), (2a), (3a), (4a), (7a), (8a), (10a); (1a), (2a), (3a), (4a), (7a), (8a), (11a); (1a), (2a), (3a), (4a), (7a), (9a), (10a); (1a), (2a), (3a), (4a), (7a), (9a), (11a); (1a), (2a), (3a), (4a), (7a), (10a), (11a); (1a), (2a), (3a), (4a), (8a), (9a), (10a); (1a), (2a), (3a), (4a), (8a), (9a), (11a); (1a), (2a), (3a), (4a), (8a), (10a), (11a); (1a), (2a), (3a), (4a), (9a), (10a), (11a); (1a), (2a), (3a), (5a), (6a), (7a), (8a); (1a), (2a), (3a), (5a), (6a), (7a), (9a); (1a), (2a), (3a), (5a), (6a), (7a), (10a); (1a), (2a), (3a), (5a), (6a), (7a), (11a); (1a), (2a), (3a), (5a), (6a), (8a), (9a); (1a), (2a), (3a), (5a), (6a), (8a), (10a); (1a), (2a), (3a), (5a), (6a), (8a), (11a); (1a), (2a), (3a), (5a), (6a), (9a), (10a); (1a), (2a), (3a), (5a), (6a), (9a), (11a); (1a), (2a), (3a), (5a), (6a), (10a), (11a); (1a), (2a), (3a), (5a), (7a), (8a), (9a); (1a), (2a), (3a), (5a), (7a), (8a), (10a); (1a), (2a), (3a), (5a), (7a), (8a), (11a); (1a), (2a), (3a), (5a), (7a), (9a), (10a); (1a), (2a), (3a), (5a), (7a), (9a), (11a); (1a), (2a), (3a), (5a), (7a), (10a), (11a); (1a), (2a), (3a), (5a), (8a), (9a), (10a); (1a), (2a), (3a), (5a), (8a), (9a), (11a); (1a), (2a), (3a), (5a), (8a), (10a), (11a); (1a), (2a), (3a), (5a), (9a), (10a), (11a); (1a), (2a), (3a), (6a), (7a), (8a), (9a); (1a), (2a), (3a), (6a), (7a), (8a), (10a); (1a), (2a), (3a), (6a), (7a), (8a), (11a); (1a), (2a), (3a), (6a), (7a), (9a), (10a); (1a), (2a), (3a), (6a), (7a), (9a), (11a); (1a), (2a), (3a), (6a), (7a), (10a), (11a); (1a), (2a), (3a), (6a), (8a), (9a), (10a); (1a), (2a), (3a), (6a), (8a), (9a), (11a); (1a), (2a), (3a), (6a), (8a), (10a), (11a); (1a), (2a), (3a), (6a), (9a), (10a), (11a); (1a), (2a), (3a), (7a), (8a), (9a), (10a); (1a), (2a), (3a), (7a), (8a), (9a), (11a); (1a), (2a), (3a), (7a), (8a), (10a), (11a); (1a), (2a), (3a), (7a), (9a), (10a), (11a); (1a), (2a), (3a), (8a), (9a), (10a), (11a); (1a), (2a), (4a), (5a), (6a), (7a), (8a); (1a), (2a), (4a), (5a), (6a), (7a), (9a); (1a), (2a), (4a), (5a), (6a), (7a), (10a); (1a), (2a), (4a), (5a), (6a), (7a), (11a); (1a), (2a), (4a), (5a), (6a), (8a), (9a); (1a), (2a), (4a), (5a), (6a), (8a), (10a); (1a), (2a), (4a), (5a), (6a), (8a), (11a); (1a), (2a), (4a), (5a), (6a), (9a), (10a); (1a), (2a), (4a), (5a), (6a), (9a), (11a); (1a), (2a), (4a), (5a), (6a), (10a), (11a); (1a), (2a), (4a), (5a), (7a), (8a), (9a); (1a), (2a), (4a), (5a), (7a), (8a), (10a); (1a), (2a), (4a), (5a), (7a), (8a), (11a); (1a), (2a), (4a), (5a), (7a), (9a), (10a); (1a), (2a), (4a), (5a), (7a), (9a), (11a); (1a), (2a), (4a), (5a), (7a), (10a), (11a); (1a), (2a), (4a), (5a), (8a), (9a), (10a); (1a), (2a), (4a), (5a), (8a), (9a), (11a); (1a), (2a), (4a), (5a), (8a), (10a), (11a); (1a), (2a), (4a), (5a), (9a), (10a), (11a); (1a), (2a), (4a), (6a), (7a), (8a), (9a); (1a), (2a), (4a), (6a), (7a), (8a), (10a); (1a), (2a), (4a), (6a), (7a), (8a), (11a); (1a), (2a), (4a), (6a), (7a), (9a), (10a); (1a), (2a), (4a), (6a), (7a), (9a), (11a); (1a), (2a), (4a), (6a), (7a), (10a), (11a); (1a), (2a), (4a), (6a), (8a), (9a), (10a); (1a), (2a), (4a), (6a), (8a), (9a), (11a); (1a), (2a), (4a), (6a), (8a), (10a), (11a); (1a), (2a), (4a), (6a), (9a), (10a), (11a); (1a), (2a), (4a), (7a), (8a), (9a), (10a); (1a), (2a), (4a), (7a), (8a), (9a), (11a); (1a), (2a), (4a), (7a), (8a), (10a), (11a); (1a), (2a), (4a), (7a), (9a), (10a), (11a); (1a), (2a), (4a), (8a), (9a), (10a), (11a); (1a), (2a), (5a), (6a), (7a), (8a), (9a); (1a), (2a), (5a), (6a), (7a), (8a), (10a); (1a), (2a), (5a), (6a), (7a), (8a), (11a); (1a), (2a), (5a), (6a), (7a), (9a), (10a); (1a), (2a), (5a), (6a), (7a), (9a), (11a); (1a), (2a), (5a), (6a), (7a), (10a), (11a); (1a), (2a), (5a), (6a), (8a), (9a), (10a); (1a), (2a), (5a), (6a), (8a), (9a), (11a); (1a), (2a), (5a), (6a), (8a), (10a), (11a); (1a), (2a), (5a), (6a), (9a), (10a), (11a); (1a), (2a), (5a), (7a), (8a), (9a), (10a); (1a), (2a), (5a), (7a), (8a), (9a), (11a); (1a), (2a), (5a), (7a), (8a), (10a), (11a); (1a), (2a), (5a), (7a), (9a), (10a), (11a); (1a), (2a), (5a), (8a), (9a), (10a), (11a); (1a), (2a), (6a), (7a), (8a), (9a), (10a); (1a), (2a), (6a), (7a), (8a), (9a), (11a); (1a), (2a), (6a), (7a), (8a), (10a), (11a); (1a), (2a), (6a), (7a), (9a), (10a), (11a); (1a), (2a), (6a), (8a), (9a), (10a), (11a); (1a), (2a), (7a), (8a), (9a), (10a), (11a); (1a), (3a), (4a), (5a), (6a), (7a), (8a); (1a), (3a), (4a), (5a), (6a), (7a), (9a); (1a), (3a), (4a), (5a), (6a), (7a), (10a); (1a), (3a), (4a), (5a), (6a), (7a), (11a); (1a), (3a), (4a), (5a), (6a), (8a), (9a); (1a), (3a), (4a), (5a), (6a), (8a), (10a); (1a), (3a), (4a), (5a), (6a), (8a), (11a); (1a), (3a), (4a), (5a), (6a), (9a), (10a); (1a), (3a), (4a), (5a), (6a), (9a), (11a); (1a), (3a), (4a), (5a), (6a), (10a), (11a); (1a), (3a), (4a), (5a), (7a), (8a), (9a); (1a), (3a), (4a), (5a), (7a), (8a), (10a); (1a), (3a), (4a), (5a), (7a), (8a), (11a); (1a), (3a), (4a), (5a), (7a), (9a), (10a); (1a), (3a), (4a), (5a), (7a), (9a), (11a); (1a), (3a), (4a), (5a), (7a), (10a), (11a); (1a), (3a), (4a), (5a), (8a), (9a), (10a); (1a), (3a), (4a), (5a), (8a), (9a), (11a); (1a), (3a), (4a), (5a), (8a), (10a), (11a); (1a), (3a), (4a), (5a), (9a), (10a), (11a); (1a), (3a), (4a), (6a), (7a), (8a), (9a); (1a), (3a), (4a), (6a), (7a), (8a), (10a); (1a), (3a), (4a), (6a), (7a), (8a), (11a); (1a), (3a), (4a), (6a), (7a), (9a), (10a); (1a), (3a), (4a), (6a), (7a), (9a), (11a); (1a), (3a), (4a), (6a), (7a), (10a), (11a); (1a), (3a), (4a), (6a), (8a), (9a), (10a); (1a), (3a), (4a), (6a), (8a), (9a), (11a); (1a), (3a), (4a), (6a), (8a), (10a), (11a); (1a), (3a), (4a), (6a), (9a), (10a), (11a); (1a), (3a), (4a), (7a), (8a), (9a), (10a); (1a), (3a), (4a), (7a), (8a), (9a), (11a); (1a), (3a), (4a), (7a), (8a), (10a), (11a); (1a), (3a), (4a), (7a), (9a), (10a), (11a); (1a), (3a), (4a), (8a), (9a), (10a), (11a); (1a), (3a), (5a), (6a), (7a), (8a), (9a); (1a), (3a), (5a), (6a), (7a), (8a), (10a); (1a), (3a), (5a), (6a), (7a), (8a), (11a); (1a), (3a), (5a), (6a), (7a), (9a), (10a); (1a), (3a), (5a), (6a), (7a), (9a), (11a); (1a), (3a), (5a), (6a), (7a), (10a), (11a); (1a), (3a), (5a), (6a), (8a), (9a), (10a); (1a), (3a), (5a), (6a), (8a), (9a), (11a); (1a), (3a), (5a), (6a), (8a), (10a), (11a); (1a), (3a), (5a), (6a), (9a), (10a), (11a); (1a), (3a), (5a), (7a), (8a), (9a), (10a); (1a), (3a), (5a), (7a), (8a), (9a), (11a); (1a), (3a), (5a), (7a), (8a), (10a), (11a); (1a), (3a), (5a), (7a), (9a), (10a), (11a); (1a), (3a), (5a), (8a), (9a), (10a), (11a); (1a), (3a), (6a), (7a), (8a), (9a), (10a); (1a), (3a), (6a), (7a), (8a), (9a), (11a); (1a), (3a), (6a), (7a), (8a), (10a), (11a); (1a), (3a), (6a), (7a), (9a), (10a), (11a); (1a), (3a), (6a), (8a), (9a), (10a), (11a); (1a), (3a), (7a), (8a), (9a), (10a), (11a); (1a), (4a), (5a), (6a), (7a), (8a), (9a); (1a), (4a), (5a), (6a), (7a), (8a), (10a); (1a), (4a), (5a), (6a), (7a), (8a), (11a); (1a), (4a), (5a), (6a), (7a), (9a), (10a); (1a), (4a), (5a), (6a), (7a), (9a), (11a); (1a), (4a), (5a), (6a), (7a), (10a), (11a); (1a), (4a), (5a), (6a), (8a), (9a), (10a); (1a), (4a), (5a), (6a), (8a), (9a), (11a); (1a), (4a), (5a), (6a), (8a), (10a), (11a); (1a), (4a), (5a), (6a), (9a), (10a), (11a); (1a), (4a), (5a), (7a), (8a), (9a), (10a); (1a), (4a), (5a), (7a), (8a), (9a), (11a); (1a), (4a), (5a), (7a), (8a), (10a), (11a); (1a), (4a), (5a), (7a), (9a), (10a), (11a); (1a), (4a), (5a), (8a), (9a), (10a), (11a); (1a), (4a), (6a), (7a), (8a), (9a), (10a); (1a), (4a), (6a), (7a), (8a), (9a), (11a); (1a), (4a), (6a), (7a), (8a), (10a), (11a); (1a), (4a), (6a), (7a), (9a), (10a), (11a); (1a), (4a), (6a), (8a), (9a), (10a), (11a); (1a), (4a), (7a), (8a), (9a), (10a), (11a); (1a), (5a), (6a), (7a), (8a), (9a), (10a); (1a), (5a), (6a), (7a), (8a), (9a), (11a); (1a), (5a), (6a), (7a), (8a), (10a), (11a); (1a), (5a), (6a), (7a), (9a), (10a), (11a); (1a), (5a), (6a), (8a), (9a), (10a), (11a); (1a), (5a), (7a), (8a), (9a), (10a), (11a); (1a), (6a), (7a), (8a), (9a), (10a), (11a); (1a), (2a), (3a), (4a), (5a), (6a), (7a), (8a); (1a), (2a), (3a), (4a), (5a), (6a), (7a), (9a); (1a), (2a), (3a), (4a), (5a), (6a), (7a), (10a); (1a), (2a), (3a), (4a), (5a), (6a), (7a), (11a); (1a), (2a), (3a), (4a), (5a), (6a), (8a), (9a); (1a), (2a), (3a), (4a), (5a), (6a), (8a), (10a); (1a), (2a), (3a), (4a), (5a), (6a), (8a), (11a); (1a), (2a), (3a), (4a), (5a), (6a), (9a), (10a); (1a), (2a), (3a), (4a), (5a), (6a), (9a), (11a); (1a), (2a), (3a), (4a), (5a), (6a), (10a), (11a); (1a), (2a), (3a), (4a), (5a), (7a), (8a), (9a); (1a), (2a), (3a), (4a), (5a), (7a), (8a), (10a); (1a), (2a), (3a), (4a), (5a), (7a), (8a), (11a); (1a), (2a), (3a), (4a), (5a), (7a), (9a), (10a); (1a), (2a), (3a), (4a), (5a), (7a), (9a), (11a); (1a), (2a), (3a), (4a), (5a), (7a), (10a), (11a); (1a), (2a), (3a), (4a), (5a), (8a), (9a), (10a); (1a), (2a), (3a), (4a), (5a), (8a), (9a), (11a); (1a), (2a), (3a), (4a), (5a), (8a), (10a), (11a); (1a), (2a), (3a), (4a), (5a), (9a), (10a), (11a); (1a), (2a), (3a), (4a), (6a), (7a), (8a), (9a); (1a), (2a), (3a), (4a), (6a), (7a), (8a), (10a); (1a), (2a), (3a), (4a), (6a), (7a), (8a), (11a); (1a), (2a), (3a), (4a), (6a), (7a), (9a), (10a); (1a), (2a), (3a), (4a), (6a), (7a), (9a), (11a); (1a), (2a), (3a), (4a), (6a), (7a), (10a), (11a); (1a), (2a), (3a), (4a), (6a), (8a), (9a), (10a); (1a), (2a), (3a), (4a), (6a), (8a), (9a), (11a); (1a), (2a), (3a), (4a), (6a), (8a), (10a), (11a); (1a), (2a), (3a), (4a), (6a), (9a), (10a), (11a); (1a), (2a), (3a), (4a), (7a), (8a), (9a), (10a); (1a), (2a), (3a), (4a), (7a), (8a), (9a), (11a); (1a), (2a), (3a), (4a), (7a), (8a), (10a), (11a); (1a), (2a), (3a), (4a), (7a), (9a), (10a), (11a); (1a), (2a), (3a), (4a), (8a), (9a), (10a), (11a); (1a), (2a), (3a), (5a), (6a), (7a), (8a), (9a); (1a), (2a), (3a), (5a), (6a), (7a), (8a), (10a); (1a), (2a), (3a), (5a), (6a), (7a), (8a), (11a); (1a), (2a), (3a), (5a), (6a), (7a), (9a), (10a); (1a), (2a), (3a), (5a), (6a), (7a), (9a), (11a); (1a), (2a), (3a), (5a), (6a), (7a), (10a), (11a); (1a), (2a), (3a), (5a), (6a), (8a), (9a), (10a); (1a), (2a), (3a), (5a), (6a), (8a), (9a), (11a); (1a), (2a), (3a), (5a), (6a), (8a), (10a), (11a); (1a), (2a), (3a), (5a), (6a), (9a), (10a), (11a); (1a), (2a), (3a), (5a), (7a), (8a), (9a), (10a); (1a), (2a), (3a), (5a), (7a), (8a), (9a), (11a); (1a), (2a), (3a), (5a), (7a), (8a), (10a), (11a); (1a), (2a), (3a), (5a), (7a), (9a), (10a), (11a); (1a), (2a), (3a), (5a), (8a), (9a), (10a), (11a); (1a), (2a), (3a), (6a), (7a), (8a), (9a), (10a); (1a), (2a), (3a), (6a), (7a), (8a), (9a), (11a); (1a), (2a), (3a), (6a), (7a), (8a), (10a), (11a); (1a), (2a), (3a), (6a), (7a), (9a), (10a), (11a); (1a), (2a), (3a), (6a), (8a), (9a), (10a), (11a); (1a), (2a), (3a), (7a), (8a), (9a), (10a), (11a); (1a), (2a), (4a), (5a), (6a), (7a), (8a), (9a); (1a), (2a), (4a), (5a), (6a), (7a), (8a), (10a); (1a), (2a), (4a), (5a), (6a), (7a), (8a), (11a); (1a), (2a), (4a), (5a), (6a), (7a), (9a), (10a); (1a), (2a), (4a), (5a), (6a), (7a), (9a), (11a); (1a), (2a), (4a), (5a), (6a), (7a), (10a), (11a); (1a), (2a), (4a), (5a), (6a), (8a), (9a), (10a); (1a), (2a), (4a), (5a), (6a), (8a), (9a), (11a); (1a), (2a), (4a), (5a), (6a), (8a), (10a), (11a); (1a), (2a), (4a), (5a), (6a), (9a), (10a), (11a); (1a), (2a), (4a), (5a), (7a), (8a), (9a), (10a); (1a), (2a), (4a), (5a), (7a), (8a), (9a), (11a); (1a), (2a), (4a), (5a), (7a), (8a), (10a), (11a); (1a), (2a), (4a), (5a), (7a), (9a), (10a), (11a); (1a), (2a), (4a), (5a), (8a), (9a), (10a), (11a); (1a), (2a), (4a), (6a), (7a), (8a), (9a), (10a); (1a), (2a), (4a), (6a), (7a), (8a), (9a), (11a); (1a), (2a), (4a), (6a), (7a), (8a), (10a), (11a); (1a), (2a), (4a), (6a), (7a), (9a), (10a), (11a); (1a), (2a), (4a), (6a), (8a), (9a), (10a), (11a); (1a), (2a), (4a), (7a), (8a), (9a), (10a), (11a); (1a), (2a), (5a), (6a), (7a), (8a), (9a), (10a); (1a), (2a), (5a), (6a), (7a), (8a), (9a), (11a); (1a), (2a), (5a), (6a), (7a), (8a), (10a), (11a); (1a), (2a), (5a), (6a), (7a), (9a), (10a), (11a); (1a), (2a), (5a), (6a), (8a), (9a), (10a), (11a); (1a), (2a), (5a), (7a), (8a), (9a), (10a), (11a); (1a), (2a), (6a), (7a), (8a), (9a), (10a), (11a); (1a), (3a), (4a), (5a), (6a), (7a), (8a), (9a); (1a), (3a), (4a), (5a), (6a), (7a), (8a), (10a); (1a), (3a), (4a), (5a), (6a), (7a), (8a), (11a); (1a), (3a), (4a), (5a), (6a), (7a), (9a), (10a); (1a), (3a), (4a), (5a), (6a), (7a), (9a), (11a); (1a), (3a), (4a), (5a), (6a), (7a), (10a), (11a); (1a), (3a), (4a), (5a), (6a), (8a), (9a), (10a); (1a), (3a), (4a), (5a), (6a), (8a), (9a), (11a); (1a), (3a), (4a), (5a), (6a), (8a), (10a), (11a); (1a), (3a), (4a), (5a), (6a), (9a), (10a), (11a); (1a), (3a), (4a), (5a), (7a), (8a), (9a), (10a); (1a), (3a), (4a), (5a), (7a), (8a), (9a), (11a); (1a), (3a), (4a), (5a), (7a), (8a), (10a), (11a); (1a), (3a), (4a), (5a), (7a), (9a), (10a), (11a); (1a), (3a), (4a), (5a), (8a), (9a), (10a), (11a); (1a), (3a), (4a), (6a), (7a), (8a), (9a), (10a); (1a), (3a), (4a), (6a), (7a), (8a), (9a), (11a); (1a), (3a), (4a), (6a), (7a), (8a), (10a), (11a); (1a), (3a), (4a), (6a), (7a), (9a), (10a), (11a); (1a), (3a), (4a), (6a), (8a), (9a), (10a), (11a); (1a), (3a), (4a), (7a), (8a), (9a), (10a), (11a); (1a), (3a), (5a), (6a), (7a), (8a), (9a), (10a); (1a), (3a), (5a), (6a), (7a), (8a), (9a), (11a); (1a), (3a), (5a), (6a), (7a), (8a), (10a), (11a); (1a), (3a), (5a), (6a), (7a), (9a), (10a), (11a); (1a), (3a), (5a), (6a), (8a), (9a), (10a), (11a); (1a), (3a), (5a), (7a), (8a), (9a), (10a), (11a); (1a), (3a), (6a), (7a), (8a), (9a), (10a), (11a); (1a), (4a), (5a), (6a), (7a), (8a), (9a), (10a); (1a), (4a), (5a), (6a), (7a), (8a), (9a), (11a); (1a), (4a), (5a), (6a), (7a), (8a), (10a), (11a); (1a), (4a), (5a), (6a), (7a), (9a), (10a), (11a); (1a), (4a), (5a), (6a), (8a), (9a), (10a), (11a); (1a), (4a), (5a), (7a), (8a), (9a), (10a), (11a); (1a), (4a), (6a), (7a), (8a), (9a), (10a), (11a); (1a), (5a), (6a), (7a), (8a), (9a), (10a), (11a); (1a), (2a), (3a), (4a), (5a), (6a), (7a), (8a), (9a); (1a), (2a), (3a), (4a), (5a), (6a), (7a), (8a), (10a); (1a), (2a), (3a), (4a), (5a), (6a), (7a), (8a), (11a); (1a), (2a), (3a), (4a), (5a), (6a), (7a), (9a), (10a); (1a), (2a), (3a), (4a), (5a), (6a), (7a), (9a), (11a); (1a), (2a), (3a), (4a), (5a), (6a), (7a), (10a), (11a); (1a), (2a), (3a), (4a), (5a), (6a), (8a), (9a), (10a); (1a), (2a), (3a), (4a), (5a), (6a), (8a), (9a), (11a); (1a), (2a), (3a), (4a), (5a), (6a), (8a), (10a), (11a); (1a), (2a), (3a), (4a), (5a), (6a), (9a), (10a), (11a); (1a), (2a), (3a), (4a), (5a), (7a), (8a), (9a), (10a); (1a), (2a), (3a), (4a), (5a), (7a), (8a), (9a), (11a); (1a), (2a), (3a), (4a), (5a), (7a), (8a), (10a), (11a); (1a), (2a), (3a), (4a), (5a), (7a), (9a), (10a), (11a); (1a), (2a), (3a), (4a), (5a), (8a), (9a), (10a), (11a); (1a), (2a), (3a), (4a), (6a), (7a), (8a), (9a), (10a); (1a), (2a), (3a), (4a), (6a), (7a), (8a), (9a), (11a); (1a), (2a), (3a), (4a), (6a), (7a), (8a), (10a), (11a); (1a), (2a), (3a), (4a), (6a), (7a), (9a), (10a), (11a); (1a), (2a), (3a), (4a), (6a), (8a), (9a), (10a), (11a); (1a), (2a), (3a), (4a), (7a), (8a), (9a), (10a), (11a); (1a), (2a), (3a), (5a), (6a), (7a), (8a), (9a), (10a); (1a), (2a), (3a), (5a), (6a), (7a), (8a), (9a), (11a); (1a), (2a), (3a), (5a), (6a), (7a), (8a), (10a), (11a); (1a), (2a), (3a), (5a), (6a), (7a), (9a), (10a), (11a); (1a), (2a), (3a), (5a), (6a), (8a), (9a), (10a), (11a); (1a), (2a), (3a), (5a), (7a), (8a), (9a), (10a), (11a); (1a), (2a), (3a), (6a), (7a), (8a), (9a), (10a), (11a); (1a), (2a), (4a), (5a), (6a), (7a), (8a), (9a), (10a); (1a), (2a), (4a), (5a), (6a), (7a), (8a), (9a), (11a); (1a), (2a), (4a), (5a), (6a), (7a), (8a), (10a), (11a); (1a), (2a), (4a), (5a), (6a), (7a), (9a), (10a), (11a); (1a), (2a), (4a), (5a), (6a), (8a), (9a), (10a), (11a); (1a), (2a), (4a), (5a), (7a), (8a), (9a), (10a), (11a); (1a), (2a), (4a), (6a), (7a), (8a), (9a), (10a), (11a); (1a), (2a), (5a), (6a), (7a), (8a), (9a), (10a), (11a); (1a), (3a), (4a), (5a), (6a), (7a), (8a), (9a), (10a); (1a), (3a), (4a), (5a), (6a), (7a), (8a), (9a), (11a); (1a), (3a), (4a), (5a), (6a), (7a), (8a), (10a), (11a); (1a), (3a), (4a), (5a), (6a), (7a), (9a), (10a), (11a); (1a), (3a), (4a), (5a), (6a), (8a), (9a), (10a), (11a); (1a), (3a), (4a), (5a), (7a), (8a), (9a), (10a), (11a); (1a), (3a), (4a), (6a), (7a), (8a), (9a), (10a), (11a); (1a), (3a), (5a), (6a), (7a), (8a), (9a), (10a), (11a); (1a), (4a), (5a), (6a), (7a), (8a), (9a), (10a), (11a); (1a), (2a), (3a), (4a), (5a), (6a), (7a), (8a), (9a), (10a); (1a), (2a), (3a), (4a), (5a), (6a), (7a), (8a), (9a), (11a); (1a), (2a), (3a), (4a), (5a), (6a), (7a), (8a), (10a), (11a); (1a), (2a), (3a), (4a), (5a), (6a), (7a), (9a), (10a), (11a); (1a), (2a), (3a), (4a), (5a), (6a), (8a), (9a), (10a), (11a); (1a), (2a), (3a), (4a), (5a), (7a), (8a), (9a), (10a), (11a); (1a), (2a), (3a), (4a), (6a), (7a), (8a), (9a), (10a), (11a); (1a), (2a), (3a), (5a), (6a), (7a), (8a), (9a), (10a), (11a); (1a), (2a), (4a), (5a), (6a), (7a), (8a), (9a), (10a), (11a); (1a), (3a), (4a), (5a), (6a), (7a), (8a), (9a), (10a), (11a); (1a), (2a), (3a), (4a), (5a), (6a), (7a), (8a), (9a), (10a), (11a). In the combinations of the preceding paragraph, ‘(1a)’ is independently selected from (1a)(i), (1a)(ii), (1a)(iii) and (1a)(iv) above; and ‘(2a)’ is independently selected from (2a)(i), (2a)(ii), (2a)(iii) and (2a)(iv) above; and ‘(3a)’ is independently selected from (3a)(i), (3a)(ii), (3a)(iii) and (3a)(iv) above; and ‘(4a)’ is independently selected from (4a)(i), (4a)(ii), (4a)(iii) and (4a)(iv) above; and ‘(5a)’ is independently selected from (5a)(i), (5a)(ii), (5a)(iii) and (5a)(iv) above; and ‘(6a)’ is independently selected from (6a)(i) and (6a)(ii) above; and ‘(7a)’ is independently selected from (7a)(i) and (7a)(ii) above; and ‘(8a)’ is independently selected from (8a)(i) and (8a)(ii) above; and ‘(9a)’ is independently selected from (9a)(i), (9a)(ii) and (9a)(iii) above.

All possible combinations of such features in accordance with the above are expressly contemplated. Purely by way of illustration, a cancer comprising the combination of features “(1a), (2a)” specifically contemplates cancers comprising: (1a)(i) and (2a)(i); (1a)(i) and (2a)(ii); (1a)(i) and (2a)(iii); (1a)(i) and (2a)(iv); (1a)(ii) and (2a)(i); (1a)(ii) and (2a)(ii); (1a)(ii) and (2a)(iii); (1a)(ii) and (2a)(iv); (1a)(iii) and (2a)(i); (1a)(iii) and (2a)(ii); (1a)(iii) and (2a)(iii); (1a)(iii) and (2a)(iv); (1a)(iv) and (2a)(ii); (1a)(iv) and (2a)(iii); and (1a)(iv) and (2a)(iv).

In embodiments where a cancer is characterised according to (7a)(i) or (7a)(ii) above, it may not be characterised according to (3a)(iii) or (3a)(iv) above. Conversely, in embodiments where a cancer is characterised according to (3a)(iii) or (3a)(iv) above, it may not be characterised according to (7a)(i) or (7a)(ii) above. However, in some embodiments, a cancer characterised by (3a)(i) or (3a)(ii) above, it may be further be characterised by (7a)(i) or (7a)(ii) above.

In aspects and embodiments according to the present disclosure, a cancer to be treated/prevented with an antigen-binding molecule that binds to HER3 is not homozygous for an activating mutation to a gene encoding a positive regulator of HER3-mediated signalling selected from: PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5. In some embodiments, the cancer is not heterozygous for an activating mutation to a gene encoding a positive regulator of HER3-mediated signalling selected from: PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5. In some embodiments, the cancer does not comprise genetic variation resulting in an increase in the expression of, or an increase in the activity of a gene product of, a gene encoding a positive regulator of HER3-mediated signalling selected from: PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5.

Herein, it will be appreciated that reference to ‘a gene’ means ‘at least one gene’, and includes ‘one or more genes’. Thus, ‘a gene’ selected from a given list may be (depending on the number of genes recited in the list) 1 gene of the list, or may be one of 2, 3, 4, 5, 6, 7, 9, 10 or more, or all of the genes recited in the list.

In aspects and embodiments according to the present disclosure, a cancer to be treated/prevented with an antigen-binding molecule that binds to HER3 is not homozygous for an activating mutation to a gene encoding a positive regulator of signalling through the MAPK/ERK pathway selected from: ERBB3, ERBB2, ERBB4, EGFR, IGF1R, NRG1, NRG2, EGF, IRS2, GAB2, GRB2, PTPN11, SHP2, SOS1, HRAS, KRAS, NRAS, RAF1, MAP2K1, MAP2K2, MAPK1, MYC, RPS6KA1, RPS6, MKNK1 and CREB1.

In some embodiments, the cancer is not heterozygous for an activating mutation to a gene encoding a positive regulator of signalling through the MAPK/ERK pathway selected from: ERBB3, ERBB2, ERBB4, EGFR, IGF1R, NRG1, NRG2, EGF, IRS2, GAB2, GRB2, PTPN11, SHP2, SOS1, HRAS, KRAS, NRAS, RAF1, MAP2K1, MAP2K2, MAPK1, MYC, RPS6KA1, RPS6, MKNK1 and CREB1. In some embodiments, the cancer does not comprise genetic variation resulting in an increase in the expression of, or an increase in the activity of a gene product of, a gene encoding a positive regulator of signalling through the MAPK/ERK pathway selected from: ERBB3, ERBB2, ERBB4, EGFR, IGF1R, NRG1, NRG2, EGF, IRS2, GAB2, GRB2, PTPN11, SHP2, SOS1, HRAS, KRAS, NRAS, RAF1, MAP2K1, MAP2K2, MAPK1, MYC, RPS6KA1, RPS6, MKNK1 and CREB1.

In aspects and embodiments according to the present disclosure, a cancer to be treated/prevented with an antigen-binding molecule that binds to HER3 is not homozygous for an activating mutation to a gene encoding a positive regulator of signalling through the PI3K/AKT/mTOR pathway selected from: ERBB3, ERBB2, ERBB4, EGFR, IGF1R, NRG1, NRG2, EGF, IRS2, GAB2, SHP2, CREB1, PIK3CB, PIK3CD, MTOR, PDK1, AKT1, AKT2, AKT3, JAK2, STAT3 and STAT5. In some embodiments, the cancer is not heterozygous for an activating mutation to a gene encoding a positive regulator of signalling through the PI3K/AKT/mTOR pathway selected from: ERBB3, ERBB2, ERBB4, EGFR, IGF1R, NRG1, NRG2, EGF, IRS2, GAB2, SHP2, CREB1, PIK3CB, PIK3CD, MTOR, PDK1, AKT1, AKT2, AKT3, JAK2, STAT3 and STAT5. In some embodiments, the cancer does not comprise genetic variation resulting in an increase in the expression of, or an increase in the activity of a gene product of, a gene encoding a positive regulator of signalling through the PI3K/AKT/mTOR pathway selected from: ERBB3, ERBB2, ERBB4, EGFR, IGF1R, NRG1, NRG2, EGF, IRS2, GAB2, SHP2, CREB1, PIK3CB, PIK3CD, MTOR, PDK1, AKT1, AKT2, AKT3, JAK2, STAT3 and STAT5.

In aspects and embodiments according to the present disclosure, a cancer to be treated/prevented with an antigen-binding molecule that binds to HER3 is not homozygous for an inactivating mutation to a gene encoding a negative regulator of HER3-mediated signalling selected from: PPP2CA, PIK3R1, PIK3R2, NF1, BAD and PHLPP1. In some embodiments, the cancer is not heterozygous for an inactivating mutation to a gene encoding a negative regulator of HER3-mediated signalling selected from: PPP2CA, PIK3R1, PIK3R2, NF1, BAD and PHLPP1. In some embodiments, the cancer does not comprise genetic variation resulting in a decrease in the expression of, or a decrease in the activity of a gene product of, a gene encoding a negative regulator of HER3-mediated signalling selected from: PPP2CA, PIK3R1, PIK3R2, NF1, BAD and PHLPP1.

In aspects and embodiments according to the present disclosure, a cancer to be treated/prevented with an antigen-binding molecule that binds to HER3 is not homozygous for an inactivating mutation to NF1. In some embodiments, the cancer is not heterozygous for an inactivating mutation to is not homozygous for an inactivating mutation to NF1. In some embodiments, the cancer does not comprise genetic variation resulting in a decrease in the expression of, or a decrease in the activity of a gene product of, is not homozygous for an inactivating mutation to NF1.

In aspects and embodiments according to the present disclosure, a cancer to be treated/prevented with an antigen-binding molecule that binds to HER3 is not homozygous for an inactivating mutation to a gene encoding a negative regulator of signalling through the PI3K/AKT/mTOR pathway selected from: PPP2CA, PIK3R1, PIK3R2, BAD and PHLPP1. In some embodiments, the cancer is not heterozygous for an inactivating mutation to a gene encoding a negative regulator of signalling through the PI3K/AKT/mTOR pathway selected from: PPP2CA, PIK3R1, PIK3R2, BAD and PHLPP1. In some embodiments, the cancer does not comprise genetic variation resulting in a decrease in the expression of, or a decrease in the activity of a gene product of, a gene encoding a negative regulator of signalling through the PI3K/AKT/mTOR pathway selected from: PPP2CA, PIK3R1, PIK3R2, BAD and PHLPP1.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, KRAS. In some embodiments, the cancer (i) does not comprise amplification of MET, and (ii) does not comprise an activating mutation to KRAS.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, PIK3CA. In some embodiments, the cancer (i) does not comprise amplification of MET, and (ii) does not comprise an activating mutation to PIK3CA.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, BRAF. In some embodiments, the cancer (i) does not comprise amplification of MET, and (ii) does not comprise an activating mutation to BRAF.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in a decrease in the expression of, or a decrease in the activity of a gene product of, PTEN. In some embodiments, the cancer (i) does not comprise amplification of MET, and (ii) does not comprise an inactivating mutation to PTEN.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, KRAS, and (iii) does not comprise genetic variation resulting in an increase in the expression of, or an increase in the activity of a gene product of, PIK3CA. In some embodiments, the cancer (i) does not comprise amplification of MET, and (ii) does not comprise an activating mutation to KRAS, and (iii) does not comprise an activating mutation to PIK3CA.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, KRAS, and (iii) does not comprise genetic variation resulting in an increase in the expression of, or an increase in the activity of a gene product of, BRAF. In some embodiments, the cancer (i) does not comprise amplification of MET, and (ii) does not comprise an activating mutation to KRAS, and (iii) does not comprise an activating mutation to BRAF.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, KRAS, and (iii) does not comprise genetic variation resulting in a decrease in the expression of, or a decrease in the activity of a gene product of, PTEN. In some embodiments, the cancer (i) does not comprise amplification of MET, and (ii) does not comprise an activating mutation to KRAS, and (iii) does not comprise an inactivating mutation to PTEN.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, PIK3CA, and (iii) does not comprise genetic variation resulting in an increase in the expression of, or an increase in the activity of a gene product of, BRAF. In some embodiments, the cancer (i) does not comprise amplification of MET, and (ii) does not comprise an activating mutation to PIK3CA, and (iii) does not comprise an activating mutation to BRAF.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, PIK3CA, and (iii) does not comprise genetic variation resulting in a decrease in the expression of, or a decrease in the activity of a gene product of, PTEN. In some embodiments, the cancer (i) does not comprise amplification of MET, and (ii) does not comprise an activating mutation to PIK3CA, and (iii) does not comprise an inactivating mutation to PTEN.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, BRAF, and (iii) does not comprise genetic variation resulting in a decrease in the expression of, or a decrease in the activity of a gene product of, PTEN. In some embodiments, the cancer (i) does not comprise amplification of MET, and (ii) does not comprise an activating mutation to BRAF, and (iii) does not comprise an inactivating mutation to PTEN.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, KRAS, and (iii) does not comprise genetic variation resulting in an increase in the expression of, or an increase in the activity of a gene product of, PIK3CA, and (iv) does not comprise genetic variation resulting in an increase in the expression of, or an increase in the activity of a gene product of, BRAF. In some embodiments, the cancer (i) does not comprise amplification of MET, and (ii) does not comprise an activating mutation to KRAS, and (iii) does not comprise an activating mutation to PIK3CA, and (iv) does not comprise an activating mutation to BRAF.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, KRAS, and (iii) does not comprise genetic variation resulting in an increase in the expression of, or an increase in the activity of a gene product of, PIK3CA, and (iv) does not comprise genetic variation resulting in a decrease in the expression of, or a decrease in the activity of a gene product of, PTEN. In some embodiments, the cancer (i) does not comprise amplification of MET, and (ii) does not comprise an activating mutation to KRAS, and (iii) does not comprise an activating mutation to PIK3CA, and (iv) does not comprise an inactivating mutation to PTEN.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, KRAS, and (iii) does not comprise genetic variation resulting in an increase in the expression of, or an increase in the activity of a gene product of, BRAF, and (iv) does not comprise genetic variation resulting in a decrease in the expression of, or a decrease in the activity of a gene product of, PTEN. In some embodiments, the cancer (i) does not comprise amplification of MET, and (ii) does not comprise an activating mutation to KRAS, and (iii) does not comprise an activating mutation to BRAF, and (iv) does not comprise an inactivating mutation to PTEN.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, PIK3CA, and (iii) does not comprise genetic variation resulting in an increase in the expression of, or an increase in the activity of a gene product of, BRAF, and (iv) does not comprise genetic variation resulting in a decrease in the expression of, or a decrease in the activity of a gene product of, PTEN. In some embodiments, the cancer (i) does not comprise amplification of MET, and (ii) does not comprise an activating mutation to PIK3CA, and (iii) does not comprise an activating mutation to BRAF, and (iv) does not comprise an inactivating mutation to PTEN.

In aspects and embodiments according to the present disclosure, a cancer to be treated/prevented with an antigen-binding molecule that binds to HER3 (i) does not comprise genetic variation resulting in an increase in the expression of, or an increase in the activity of a gene product of, MET, and (ii) does not comprise genetic variation resulting in an increase in the expression of, or an increase in the activity of a gene product of, KRAS, and (iii) does not comprise genetic variation resulting in an increase in the expression of, or an increase in the activity of a gene product of, PIK3CA, and (iv) does not comprise genetic variation resulting in an increase in the expression of, or an increase in the activity of a gene product of, BRAF, and (v) does not comprise genetic variation resulting in a decrease in the expression of, or a decrease in the activity of a gene product of, PTEN. In some embodiments, the cancer (i) does not comprise amplification of MET, and (ii) does not comprise an activating mutation to KRAS, and (iii) does not comprise an activating mutation to PIK3CA, and (iv) does not comprise an activating mutation to BRAF, and (v) does not comprise an inactivating mutation to PTEN.

In aspects and embodiments according to the present disclosure, a cancer to be treated/prevented with an antigen-binding molecule that binds to HER3 (i) does not comprise amplification of MET, and (ii) does not comprise an activating mutation to KRAS, and (iii) does not comprise an activating mutation to PIK3CA, and (iv) does not comprise an activating mutation to BRAF, and (v) does not comprise deletion of PTEN.

Cancers characterised by the presence of Genetic variation resulting in an increase in HER3-mediated signalling Aspects and embodiments of the present disclosure relate to cancers comprising genetic variation resulting in an increase in HER3-mediated signalling. Such cancers may be considered as being less sensitive to/less susceptible to/more resistant to (and therefore less likely to respond well to) therapeutic/prophylactic intervention with an antigen-binding molecule that binds to HER3 as a monotherapy. Administration of an antagonist of HER3-mediated signalling may be effective to render the cancer sensitive/susceptible to (and therefore more likely to respond well to) therapeutic/prophylactic intervention with an antigen-binding molecule that binds to HER3. It will be appreciated that cancers described in this section may be further characterised according to the section above entitled ‘Cancers’.

In aspects and embodiments according to the present disclosure, a cancer to be treated/prevented with an antigen-binding molecule that binds to HER3 in combination with an antagonist of HER3-mediated signalling is characterised according to one or more of the following features:
(1 b)(i) is homozygous for an activating mutation to MET (e.g. an activating mutation to MET described herein).
(1 b)(ii) is heterozygous for an activating mutation to MET (e.g. an activating mutation to MET described herein).
(1 b)(iii) comprises amplification of MET (e.g. amplification of MET as described herein).
(1 b)(iv) comprises genetic variation resulting in an increase in the expression of, or an increase in the activity of a gene product of, MET.
(2b)(i) is homozygous for an activating mutation to KRAS (e.g. an activating mutation to KRAS as described herein).
(2b)(ii) is heterozygous for an activating mutation to KRAS (e.g. an activating mutation to KRAS as described herein).
(2b)(iii) comprises amplification of KRAS (e.g. amplification of KRAS as described herein).
(2b)(iv) comprises genetic variation resulting in an increase in the expression of, or an increase in the activity of a gene product of, KRAS.
(3b)(i) is homozygous for an activating mutation to PIK3CA (e.g. an activating mutation to PIK3CA as described herein).
(3b)(ii) is heterozygous for an activating mutation to PIK3CA (e.g. an activating mutation to PIK3CA as described herein).
(3b)(iii) comprises amplification of PIK3CA (e.g. amplification of PIK3CA as described herein).
(3b)(iv) comprises genetic variation resulting in an increase in the expression of, or an increase in the activity of a gene product of, PIK3CA.
(4b)(i) is homozygous for an activating mutation to BRAF (e.g. an activating mutation to BRAF as described herein).
(4b)(ii) is heterozygous for an activating mutation to BRAF (e.g. an activating mutation to BRAF as described herein).
(4b)(iii) comprises amplification of BRAF (e.g. amplification of BRAF as described herein).
(4b)(iv) comprises genetic variation resulting in an increase in the expression of, or an increase in the activity of a gene product of, BRAF.
(5b)(i) comprises genetic variation resulting in a decrease in the expression of, or a decrease in the activity of a gene product of, PTEN.
(5b)(ii) is homozygous for an inactivating mutation to PTEN (e.g. an inactivating mutation to PTEN as described herein).
(5b)(iii) is heterozygous for an inactivating mutation to PTEN (e.g. an inactivating mutation to PTEN as described herein).
(5b)(iv) comprises deletion of PTEN (e.g. deletion of PTEN as described herein).
(6b)(i) comprises genetic variation resulting in an increase in the expression of, or an increase in the activity of a gene product of, TP63.
(6b)(ii) comprises amplification of TP63 (e.g. amplification of TP63 as described herein).
(7b)(i) comprises genetic variation resulting in an increase in the expression of, or an increase in the activity of a gene product of, SOX2.
(7b)(ii) comprises amplification of SOX2 (e.g. amplification of SOX2 as described herein).
(8b)(i) comprises genetic variation resulting in an increase in the expression of a ligand for HER3 (e.g. as described herein).
(8b)(ii) comprises an NRG gene fusion (e.g. an NRG gene fusion as described herein).
(8b)(iii) comprises an NRG1 gene fusion (e.g. an NRG1 gene fusion as described herein).
(9b)(i) comprises genetic variation resulting in an increase in the expression of, or an increase in the activity of a gene product of, EGFR.
(9b)(ii) comprises amplification of EGFR (e.g. amplification of EGFR as described herein).
(10b)(i) does not comprise genetic variation resulting in a decrease in the expression of, or a decrease in the activity of a gene product of, TUSC2.
(10b)(ii) does not comprise deletion of TUSC2 (e.g. deletion of TUSC2 as described herein).

In some embodiments, a cancer to be treated/prevented with an antigen-binding molecule that binds to HER3 in combination with an antagonist of HER3-mediated signalling is characterised by one of the following combinations of features (with reference to the preceding paragraph): (1 b), (2b); (1 b), (3b); (1 b), (4b); (1b), (5b); (1b), (6b); (1b), (7b); (1b), (8b); (1b), (9b); (1b), (10b); (1b), (2b), (3b); (1b), (2b), (4b); (1b), (2b), (5b); (1b), (2b), (6b); (1b), (2b), (7b); (1 b), (2b), (8b); (1b), (2b), (9b); (1b), (2b), (10b); (1b), (3b), (4b); (1 b), (3b), (5b); (1 b), (3b), (6b); (1 b), (3b), (7b); (1 b), (3b), (8b); (1 b), (3b), (9b); (1 b), (3b), (10b); (1b), (4b), (5b); (1b), (4b), (6b); (1b), (4b), (7b); (1b), (4b), (8b); (1b), (4b), (9b); (1b), (4b), (10b); (1 b), (5b), (6b); (1 b), (5b), (7b); (1 b), (5b), (8b); (1 b), (5b), (9b); (1 b), (5b), (10b); (1 b), (6b), (7b); (1 b), (6b), (8b); (1b), (6b), (9b); (1b), (6b), (10b); (1 b), (7b), (8b); (1b), (7b), (9b); (1b), (7b), (10b); (1 b), (8b), (9b); (1 b), (8b), (10b); (1 b), (9b), (10b); (1 b), (2b), (3b), (4b); (1 b), (2b), (3b), (5b); (1 b), (2b), (3b), (6b); (1 b), (2b), (3b), (7b); (1 b), (2b), (3b), (8b); (1 b), (2b), (3b), (9b); (1 b), (2b), (3b), (10b); (1 b), (2b), (4b), (5b); (1 b), (2b), (4b), (6b); (1 b), (2b), (4b), (7b); (1 b), (2b), (4b), (8b); (1 b), (2b), (4b), (9b); (1 b), (2b), (4b), (10b); (1b), (2b), (5b), (6b); (1b), (2b), (5b), (7b); (1b), (2b), (5b), (8b); (1b), (2b), (5b), (9b); (1b), (2b), (5b), (10b); (1b), (2b), (6b), (7b); (1b), (2b), (6b), (8b); (1b), (2b), (6b), (9b); (1b), (2b), (6b), (10b); (1b), (2b), (7b), (8b); (1b), (2b), (7b), (9b); (1b), (2b), (7b), (10b); (1b), (2b), (8b), (9b); (1b), (2b), (8b), (10b); (1 b), (2b), (9b), (10b); (1 b), (3b), (4b), (5b); (1 b), (3b), (4b), (6b); (1 b), (3b), (4b), (7b); (1 b), (3b), (4b), (8b); (1b), (3b), (4b), (9b); (1b), (3b), (4b), (10b); (1b), (3b), (5b), (6b); (1b), (3b), (5b), (7b); (1b), (3b), (5b), (8b); (1b), (3b), (5b), (9b); (1b), (3b), (5b), (10b); (1b), (3b), (6b), (7b); (1b), (3b), (6b), (8b); (1b), (3b), (6b), (9b); (1b), (3b), (6b), (10b); (1b), (3b), (7b), (8b); (1b), (3b), (7b), (9b); (1b), (3b), (7b), (10b); (1 b), (3b), (8b), (9b); (1 b), (3b), (8b), (10b); (1 b), (3b), (9b), (10b); (1 b), (4b), (5b), (6b); (1 b), (4b), (5b), (7b); (1b), (4b), (5b), (8b); (1b), (4b), (5b), (9b); (1b), (4b), (5b), (10b); (1b), (4b), (6b), (7b); (1b), (4b), (6b), (8b); (1b), (4b), (6b), (9b); (1b), (4b), (6b), (10b); (1b), (4b), (7b), (8b); (1b), (4b), (7b), (9b); (1b), (4b), (7b), (10b); (1b), (4b), (8b), (9b); (1b), (4b), (8b), (10b); (1b), (4b), (9b), (10b); (1b), (5b), (6b), (7b); (1b), (5b), (6b), (8b); (1b), (5b), (6b), (9b); (1b), (5b), (6b), (10b); (1b), (5b), (7b), (8b); (1b), (5b), (7b), (9b); (1b), (5b), (7b), (10b); (1b), (5b), (8b), (9b); (1b), (5b), (8b), (10b); (1b), (5b), (9b), (10b); (1b), (6b), (7b), (8b); (1b), (6b), (7b), (9b); (1b), (6b), (7b), (10b); (1b), (6b), (8b), (9b); (1b), (6b), (8b), (10b); (1b), (6b), (9b), (10b); (1b), (7b), (8b), (9b); (1b), (7b), (8b), (10b); (1b), (7b), (9b), (10b); (1b), (8b), (9b), (10b); (1 b), (2b), (3b), (4b), (5b); (1 b), (2b), (3b), (4b), (6b); (1 b), (2b), (3b), (4b), (7b); (1 b), (2b), (3b), (4b), (8b); (1b), (2b), (3b), (4b), (9b); (1b), (2b), (3b), (4b), (10b); (1b), (2b), (3b), (5b), (6b); (1b), (2b), (3b), (5b), (7b); (1 b), (2b), (3b), (5b), (8b); (1 b), (2b), (3b), (5b), (9b); (1 b), (2b), (3b), (5b), (10b); (1 b), (2b), (3b), (6b), (7b); (1b), (2b), (3b), (6b), (8b); (1b), (2b), (3b), (6b), (9b); (1b), (2b), (3b), (6b), (10b); (1b), (2b), (3b), (7b), (8b); (1b), (2b), (3b), (7b), (9b); (1b), (2b), (3b), (7b), (10b); (1b), (2b), (3b), (8b), (9b); (1b), (2b), (3b), (8b), (10b); (1b), (2b), (3b), (9b), (10b); (1b), (2b), (4b), (5b), (6b); (1b), (2b), (4b), (5b), (7b); (1 b), (2b), (4b), (5b), (8b); (1 b), (2b), (4b), (5b), (9b); (1 b), (2b), (4b), (5b), (10b); (1 b), (2b), (4b), (6b), (7b); (1 b), (2b), (4b), (6b), (8b); (1 b), (2b), (4b), (6b), (9b); (1 b), (2b), (4b), (6b), (10b); (1 b), (2b), (4b), (7b), (8b); (1b), (2b), (4b), (7b), (9b); (1b), (2b), (4b), (7b), (10b); (1b), (2b), (4b), (8b), (9b); (1b), (2b), (4b), (8b), (10b); (1 b), (2b), (4b), (9b), (10b); (1 b), (2b), (5b), (6b), (7b); (1 b), (2b), (5b), (6b), (8b); (1 b), (2b), (5b), (6b), (9b); (1b), (2b), (5b), (6b), (10b); (1b), (2b), (5b), (7b), (8b); (1b), (2b), (5b), (7b), (9b); (1b), (2b), (5b), (7b), (10b); (1b), (2b), (5b), (8b), (9b); (1b), (2b), (5b), (8b), (10b); (1b), (2b), (5b), (9b), (10b); (1b), (2b), (6b), (7b), (8b); (1b), (2b), (6b), (7b), (9b); (1b), (2b), (6b), (7b), (10b); (1b), (2b), (6b), (8b), (9b); (1 b), (2b), (6b), (8b), (10b); (1 b), (2b), (6b), (9b), (10b); (1 b), (2b), (7b), (8b), (9b); (1 b), (2b), (7b), (8b), (10b); (1b), (2b), (7b), (9b), (10b); (1b), (2b), (8b), (9b), (10b); (1b), (3b), (4b), (5b), (6b); (1b), (3b), (4b), (5b), (7b); (1 b), (3b), (4b), (5b), (8b); (1 b), (3b), (4b), (5b), (9b); (1 b), (3b), (4b), (5b), (10b); (1 b), (3b), (4b), (6b), (7b); (1 b), (3b), (4b), (6b), (8b); (1 b), (3b), (4b), (6b), (9b); (1 b), (3b), (4b), (6b), (10b); (1b), (3b), (4b), (7b), (8b); (1b), (3b), (4b), (7b), (9b); (1b), (3b), (4b), (7b), (10b); (1b), (3b), (4b), (8b), (9b); (1 b), (3b), (4b), (8b), (10b); (1 b), (3b), (4b), (9b), (10b); (1 b), (3b), (5b), (6b), (7b); (1 b), (3b), (5b), (6b), (8b); (1b), (3b), (5b), (6b), (9b); (1b), (3b), (5b), (6b), (10b); (1b), (3b), (5b), (7b), (8b); (1b), (3b), (5b), (7b), (9b); (1b), (3b), (5b), (7b), (10b); (1b), (3b), (5b), (8b), (9b); (1b), (3b), (5b), (8b), (10b); (1 b), (3b), (5b), (9b), (10b); (1 b), (3b), (6b), (7b), (8b); (1 b), (3b), (6b), (7b), (9b); (1 b), (3b), (6b), (7b), (10b); (1b), (3b), (6b), (8b), (9b); (1b), (3b), (6b), (8b), (10b); (1b), (3b), (6b), (9b), (10b); (1b), (3b), (7b), (8b), (9b); (1 b), (3b), (7b), (8b), (10b); (1 b), (3b), (7b), (9b), (10b); (1 b), (3b), (8b), (9b), (10b); (1 b), (4b), (5b), (6b), (7b); (1 b), (4b), (5b), (6b), (8b); (1 b), (4b), (5b), (6b), (9b); (1 b), (4b), (5b), (6b), (10b); (1 b), (4b), (5b), (7b), (8b); (1 b), (4b), (5b), (7b), (9b); (1 b), (4b), (5b), (7b), (10b); (1 b), (4b), (5b), (8b), (9b); (1b), (4b), (5b), (8b), (10b); (1b), (4b), (5b), (9b), (10b); (1b), (4b), (6b), (7b), (8b); (1b), (4b), (6b), (7b), (9b); (1b), (4b), (6b), (7b), (10b); (1b), (4b), (6b), (8b), (9b); (1b), (4b), (6b), (8b), (10b); (1b), (4b), (6b), (9b), (10b); (1 b), (4b), (7b), (8b), (9b); (1 b), (4b), (7b), (8b), (10b); (1 b), (4b), (7b), (9b), (10b); (1 b), (4b), (8b), (9b), (10b); (1b), (5b), (6b), (7b), (8b); (1b), (5b), (6b), (7b), (9b); (1b), (5b), (6b), (7b), (10b); (1b), (5b), (6b), (8b), (9b); (1b), (5b), (6b), (8b), (10b); (1b), (5b), (6b), (9b), (10b); (1b), (5b), (7b), (8b), (9b); (1b), (5b), (7b), (8b), (10b); (1b), (5b), (7b), (9b), (10b); (1b), (5b), (8b), (9b), (10b); (1b), (6b), (7b), (8b), (9b); (1b), (6b), (7b), (8b), (10b); (1b), (6b), (7b), (9b), (10b); (1b), (6b), (8b), (9b), (10b); (1b), (7b), (8b), (9b), (10b); (1b), (2b), (3b), (4b), (5b), (6b); (1b), (2b), (3b), (4b), (5b), (7b); (1b), (2b), (3b), (4b), (5b), (8b); (1b), (2b), (3b), (4b), (5b), (9b); (1b), (2b), (3b), (4b), (5b), (10b); (1b), (2b), (3b), (4b), (6b), (7b); (1 b), (2b), (3b), (4b), (6b), (8b); (1 b), (2b), (3b), (4b), (6b), (9b); (1 b), (2b), (3b), (4b), (6b), (10b); (1 b), (2b), (3b), (4b), (7b), (8b); (1 b), (2b), (3b), (4b), (7b), (9b); (1 b), (2b), (3b), (4b), (7b), (10b); (1 b), (2b), (3b), (4b), (8b), (9b); (1b), (2b), (3b), (4b), (8b), (10b); (1b), (2b), (3b), (4b), (9b), (10b); (1b), (2b), (3b), (5b), (6b), (7b); (1 b), (2b), (3b), (5b), (6b), (8b); (1 b), (2b), (3b), (5b), (6b), (9b); (1 b), (2b), (3b), (5b), (6b), (10b); (1b), (2b), (3b), (5b), (7b), (8b); (1b), (2b), (3b), (5b), (7b), (9b); (1b), (2b), (3b), (5b), (7b), (10b); (1b), (2b), (3b), (5b), (8b), (9b); (1b), (2b), (3b), (5b), (8b), (10b); (1b), (2b), (3b), (5b), (9b), (10b); (1b), (2b), (3b), (6b), (7b), (8b); (1 b), (2b), (3b), (6b), (7b), (9b); (1 b), (2b), (3b), (6b), (7b), (10b); (1 b), (2b), (3b), (6b), (8b), (9b); (1b), (2b), (3b), (6b), (8b), (10b); (1b), (2b), (3b), (6b), (9b), (10b); (1b), (2b), (3b), (7b), (8b), (9b); (1b), (2b), (3b), (7b), (8b), (10b); (1b), (2b), (3b), (7b), (9b), (10b); (1b), (2b), (3b), (8b), (9b), (10b); (1b), (2b), (4b), (5b), (6b), (7b); (1b), (2b), (4b), (5b), (6b), (8b); (1b), (2b), (4b), (5b), (6b), (9b); (1b), (2b), (4b), (5b), (6b), (10b); (1b), (2b), (4b), (5b), (7b), (8b); (1b), (2b), (4b), (5b), (7b), (9b); (1b), (2b), (4b), (5b), (7b), (10b); (1b), (2b), (4b), (5b), (8b), (9b); (1b), (2b), (4b), (5b), (8b), (10b); (1b), (2b), (4b), (5b), (9b), (10b); (1 b), (2b), (4b), (6b), (7b), (8b); (1 b), (2b), (4b), (6b), (7b), (9b); (1 b), (2b), (4b), (6b), (7b), (10b); (1b), (2b), (4b), (6b), (8b), (9b); (1b), (2b), (4b), (6b), (8b), (10b); (1b), (2b), (4b), (6b), (9b), (10b); (1b), (2b), (4b), (7b), (8b), (9b); (1b), (2b), (4b), (7b), (8b), (10b); (1b), (2b), (4b), (7b), (9b), (10b); (1 b), (2b), (4b), (8b), (9b), (10b); (1 b), (2b), (5b), (6b), (7b), (8b); (1 b), (2b), (5b), (6b), (7b), (9b); (1b), (2b), (5b), (6b), (7b), (10b); (1b), (2b), (5b), (6b), (8b), (9b); (1b), (2b), (5b), (6b), (8b), (10b); (1b), (2b), (5b), (6b), (9b), (10b); (1b), (2b), (5b), (7b), (8b), (9b); (1b), (2b), (5b), (7b), (8b), (10b); (1b), (2b), (5b), (7b), (9b), (10b); (1b), (2b), (5b), (8b), (9b), (10b); (1b), (2b), (6b), (7b), (8b), (9b); (1b), (2b), (6b), (7b), (8b), (10b); (1b), (2b), (6b), (7b), (9b), (10b); (1b), (2b), (6b), (8b), (9b), (10b); (1b), (2b), (7b), (8b), (9b), (10b); (1b), (3b), (4b), (5b), (6b), (7b); (1b), (3b), (4b), (5b), (6b), (8b); (1b), (3b), (4b), (5b), (6b), (9b); (1b), (3b), (4b), (5b), (6b), (10b); (1b), (3b), (4b), (5b), (7b), (8b); (1b), (3b), (4b), (5b), (7b), (9b); (1b), (3b), (4b), (5b), (7b), (10b); (1b), (3b), (4b), (5b), (8b), (9b); (1b), (3b), (4b), (5b), (8b), (10b); (1b), (3b), (4b), (5b), (9b), (10b); (1b), (3b), (4b), (6b), (7b), (8b); (1b), (3b), (4b), (6b), (7b), (9b); (1b), (3b), (4b), (6b), (7b), (10b); (1b), (3b), (4b), (6b), (8b), (9b); (1b), (3b), (4b), (6b), (8b), (10b); (1b), (3b), (4b), (6b), (9b), (10b); (1b), (3b), (4b), (7b), (8b), (9b); (1b), (3b), (4b), (7b), (8b), (10b); (1b), (3b), (4b), (7b), (9b), (10b); (1b), (3b), (4b), (8b), (9b), (10b); (1b), (3b), (5b), (6b), (7b), (8b); (1b), (3b), (5b), (6b), (7b), (9b); (1b), (3b), (5b), (6b), (7b), (10b); (1b), (3b), (5b), (6b), (8b), (9b); (1b), (3b), (5b), (6b), (8b), (10b); (1b), (3b), (5b), (6b), (9b), (10b); (1b), (3b), (5b), (7b), (8b), (9b); (1b), (3b), (5b), (7b), (8b), (10b); (1 b), (3b), (5b), (7b), (9b), (10b); (1 b), (3b), (5b), (8b), (9b), (10b); (1 b), (3b), (6b), (7b), (8b), (9b); (1 b), (3b), (6b), (7b), (8b), (10b); (1b), (3b), (6b), (7b), (9b), (10b); (1b), (3b), (6b), (8b), (9b), (10b); (1b), (3b), (7b), (8b), (9b), (10b); (1 b), (4b), (5b), (6b), (7b), (8b); (1 b), (4b), (5b), (6b), (7b), (9b); (1 b), (4b), (5b), (6b), (7b), (10b); (1b), (4b), (5b), (6b), (8b), (9b); (1b), (4b), (5b), (6b), (8b), (10b); (1b), (4b), (5b), (6b), (9b), (10b); (1b), (4b), (5b), (7b), (8b), (9b); (1b), (4b), (5b), (7b), (8b), (10b); (1b), (4b), (5b), (7b), (9b), (10b); (1b), (4b), (5b), (8b), (9b), (10b); (1b), (4b), (6b), (7b), (8b), (9b); (1b), (4b), (6b), (7b), (8b), (10b); (1 b), (4b), (6b), (7b), (9b), (10b); (1 b), (4b), (6b), (8b), (9b), (10b); (1 b), (4b), (7b), (8b), (9b), (10b); (1 b), (5b), (6b), (7b), (8b), (9b); (1 b), (5b), (6b), (7b), (8b), (10b); (1 b), (5b), (6b), (7b), (9b), (10b); (1 b), (5b), (6b), (8b), (9b), (10b); (1b), (5b), (7b), (8b), (9b), (10b); (1b), (6b), (7b), (8b), (9b), (10b); (1b), (2b), (3b), (4b), (5b), (6b), (7b); (1 b), (2b), (3b), (4b), (5b), (6b), (8b); (1 b), (2b), (3b), (4b), (5b), (6b), (9b); (1 b), (2b), (3b), (4b), (5b), (6b), (10b); (1 b), (2b), (3b), (4b), (5b), (7b), (8b); (1 b), (2b), (3b), (4b), (5b), (7b), (9b); (1b), (2b), (3b), (4b), (5b), (7b), (10b); (1b), (2b), (3b), (4b), (5b), (8b), (9b); (1b), (2b), (3b), (4b), (5b), (8b), (10b); (1 b), (2b), (3b), (4b), (5b), (9b), (10b); (1 b), (2b), (3b), (4b), (6b), (7b), (8b); (1 b), (2b), (3b), (4b), (6b), (7b), (9b); (1b), (2b), (3b), (4b), (6b), (7b), (10b); (1b), (2b), (3b), (4b), (6b), (8b), (9b); (1b), (2b), (3b), (4b), (6b), (8b), (10b); (1b), (2b), (3b), (4b), (6b), (9b), (10b); (1b), (2b), (3b), (4b), (7b), (8b), (9b); (1 b), (2b), (3b), (4b), (7b), (8b), (10b); (1 b), (2b), (3b), (4b), (7b), (9b), (10b); (1 b), (2b), (3b), (4b), (8b), (9b), (10b); (1b), (2b), (3b), (5b), (6b), (7b), (8b); (1b), (2b), (3b), (5b), (6b), (7b), (9b); (1b), (2b), (3b), (5b), (6b), (7b), (10b); (1b), (2b), (3b), (5b), (6b), (8b), (9b); (1b), (2b), (3b), (5b), (6b), (8b), (10b); (1b), (2b), (3b), (5b), (6b), (9b), (10b); (1b), (2b), (3b), (5b), (7b), (8b), (9b); (1b), (2b), (3b), (5b), (7b), (8b), (10b); (1b), (2b), (3b), (5b), (7b), (9b), (10b); (1b), (2b), (3b), (5b), (8b), (9b), (10b); (1b), (2b), (3b), (6b), (7b), (8b), (9b); (1b), (2b), (3b), (6b), (7b), (8b), (10b); (1b), (2b), (3b), (6b), (7b), (9b), (10b); (1b), (2b), (3b), (6b), (8b), (9b), (10b); (1 b), (2b), (3b), (7b), (8b), (9b), (10b); (1 b), (2b), (4b), (5b), (6b), (7b), (8b); (1 b), (2b), (4b), (5b), (6b), (7b), (9b); (1 b), (2b), (4b), (5b), (6b), (7b), (10b); (1 b), (2b), (4b), (5b), (6b), (8b), (9b); (1b), (2b), (4b), (5b), (6b), (8b), (10b); (1b), (2b), (4b), (5b), (6b), (9b), (10b); (1b), (2b), (4b), (5b), (7b), (8b), (9b); (1b), (2b), (4b), (5b), (7b), (8b), (10b); (1b), (2b), (4b), (5b), (7b), (9b), (10b); (1b), (2b), (4b), (5b), (8b), (9b), (10b); (1b), (2b), (4b), (6b), (7b), (8b), (9b); (1b), (2b), (4b), (6b), (7b), (8b), (10b); (1b), (2b), (4b), (6b), (7b), (9b), (10b); (1b), (2b), (4b), (6b), (8b), (9b), (10b); (1b), (2b), (4b), (7b), (8b), (9b), (10b); (1b), (2b), (5b), (6b), (7b), (8b), (9b); (1b), (2b), (5b), (6b), (7b), (8b), (10b); (1b), (2b), (5b), (6b), (7b), (9b), (10b); (1b), (2b), (5b), (6b), (8b), (9b), (10b); (1b), (2b), (5b), (7b), (8b), (9b), (10b); (1 b), (2b), (6b), (7b), (8b), (9b), (10b); (1 b), (3b), (4b), (5b), (6b), (7b), (8b); (1 b), (3b), (4b), (5b), (6b), (7b), (9b); (1b), (3b), (4b), (5b), (6b), (7b), (10b); (1b), (3b), (4b), (5b), (6b), (8b), (9b); (1b), (3b), (4b), (5b), (6b), (8b), (10b); (1b), (3b), (4b), (5b), (6b), (9b), (10b); (1b), (3b), (4b), (5b), (7b), (8b), (9b); (1b), (3b), (4b), (5b), (7b), (8b), (10b); (1b), (3b), (4b), (5b), (7b), (9b), (10b); (1b), (3b), (4b), (5b), (8b), (9b), (10b); (1b), (3b), (4b), (6b), (7b), (8b), (9b); (1b), (3b), (4b), (6b), (7b), (8b), (10b); (1b), (3b), (4b), (6b), (7b), (9b), (10b); (1b), (3b), (4b), (6b), (8b), (9b), (10b); (1b), (3b), (4b), (7b), (8b), (9b), (10b); (1b), (3b), (5b), (6b), (7b), (8b), (9b); (1b), (3b), (5b), (6b), (7b), (8b), (10b); (1b), (3b), (5b), (6b), (7b), (9b), (10b); (1b), (3b), (5b), (6b), (8b), (9b), (10b); (1b), (3b), (5b), (7b), (8b), (9b), (10b); (1b), (3b), (6b), (7b), (8b), (9b), (10b); (1b), (4b), (5b), (6b), (7b), (8b), (9b); (1b), (4b), (5b), (6b), (7b), (8b), (10b); (1b), (4b), (5b), (6b), (7b), (9b), (10b); (1b), (4b), (5b), (6b), (8b), (9b), (10b); (1b), (4b), (5b), (7b), (8b), (9b), (10b); (1 b), (4b), (6b), (7b), (8b), (9b), (10b); (1 b), (5b), (6b), (7b), (8b), (9b), (10b); (1 b), (2b), (3b), (4b), (5b), (6b), (7b), (8b); (1 b), (2b), (3b), (4b), (5b), (6b), (7b), (9b); (1 b), (2b), (3b), (4b), (5b), (6b), (7b), (10b); (1 b), (2b), (3b), (4b), (5b), (6b), (8b), (9b); (1 b), (2b), (3b), (4b), (5b), (6b), (8b), (10b); (1 b), (2b), (3b), (4b), (5b), (6b), (9b), (10b); (1 b), (2b), (3b), (4b), (5b), (7b), (8b), (9b); (1 b), (2b), (3b), (4b), (5b), (7b), (8b), (10b); (1b), (2b), (3b), (4b), (5b), (7b), (9b), (10b); (1b), (2b), (3b), (4b), (5b), (8b), (9b), (10b); (1b), (2b), (3b), (4b), (6b), (7b), (8b), (9b); (1b), (2b), (3b), (4b), (6b), (7b), (8b), (10b); (1b), (2b), (3b), (4b), (6b), (7b), (9b), (10b); (1b), (2b), (3b), (4b), (6b), (8b), (9b), (10b); (1b), (2b), (3b), (4b), (7b), (8b), (9b), (10b); (1b), (2b), (3b), (5b), (6b), (7b), (8b), (9b); (1b), (2b), (3b), (5b), (6b), (7b), (8b), (10b); (1b), (2b), (3b), (5b), (6b), (7b), (9b), (10b); (1b), (2b), (3b), (5b), (6b), (8b), (9b), (10b); (1b), (2b), (3b), (5b), (7b), (8b), (9b), (10b); (1b), (2b), (3b), (6b), (7b), (8b), (9b), (10b); (1b), (2b), (4b), (5b), (6b), (7b), (8b), (9b); (1 b), (2b), (4b), (5b), (6b), (7b), (8b), (10b); (1 b), (2b), (4b), (5b), (6b), (7b), (9b), (10b); (1 b), (2b), (4b), (5b), (6b), (8b), (9b), (10b); (1b), (2b), (4b), (5b), (7b), (8b), (9b), (10b); (1b), (2b), (4b), (6b), (7b), (8b), (9b), (10b); (1b), (2b), (5b), (6b), (7b), (8b), (9b), (10b); (1b), (3b), (4b), (5b), (6b), (7b), (8b), (9b); (1b), (3b), (4b), (5b), (6b), (7b), (8b), (10b); (1 b), (3b), (4b), (5b), (6b), (7b), (9b), (10b); (1 b), (3b), (4b), (5b), (6b), (8b), (9b), (10b); (1b), (3b), (4b), (5b), (7b), (8b), (9b), (10b); (1b), (3b), (4b), (6b), (7b), (8b), (9b), (10b); (1b), (3b), (5b), (6b), (7b), (8b), (9b), (10b); (1b), (4b), (5b), (6b), (7b), (8b), (9b), (10b); (1b), (2b), (3b), (4b), (5b), (6b), (7b), (8b), (9b); (1 b), (2b), (3b), (4b), (5b), (6b), (7b), (8b), (10b); (1 b), (2b), (3b), (4b), (5b), (6b), (7b), (9b), (10b); (1b), (2b), (3b), (4b), (5b), (6b), (8b), (9b), (10b); (1b), (2b), (3b), (4b), (5b), (7b), (8b), (9b), (10b); (1b), (2b), (3b), (4b), (6b), (7b), (8b), (9b), (10b); (1b), (2b), (3b), (5b), (6b), (7b), (8b), (9b), (10b); (1b), (2b), (4b), (5b), (6b), (7b), (8b), (9b), (10b); (1b), (3b), (4b), (5b), (6b), (7b), (8b), (9b), (10b); (1b), (2b), (3b), (4b), (5b), (6b), (7b), (8b), (9b), (10b).

In the combinations of the preceding paragraph, ‘(1 b)’ is independently selected from (1 b)(i), (1 b)(ii), (1b)(iii) and (1b)(iv) above; and ‘(2b)’ is independently selected from (2b)(i), (2b)(ii), (2b)(iii) and (2b)(iv) above; and ‘(3b)’ is independently selected from (3b)(i), (3b)(ii), (3b)(iii) and (3b)(iv) above; and ‘(4b)’ is independently selected from (4b)(i), (4b)(ii), (4b)(iii) and (4b)(iv) above; and ‘(5b)’ is independently selected from (5b)(i), (5b)(ii), (5b)(iii) and (5b)(iv) above; and ‘(6b)’ is independently selected from (6b)(i) and (6b)(ii) above; and ‘(7b)’ is independently selected from (7b)(i) and (7b)(ii) above; and ‘(8b)’ is independently selected from (8b)(i) and (8b)(ii) above.

All possible combinations of such features in accordance with the above are expressly contemplated. Purely by way of illustration, a cancer comprising the combination of features “(1 b), (2b)” specifically contemplates cancers comprising: (1b)(i) and (2b)(i); (1b)(i) and (2b)(ii); (1b)(i) and (2b)(iii); (1b)(i) and (2b)(iv); (1b)(ii) and (2b)(i); (1b)(ii) and (2b)(ii); (1 b)(ii) and (2b)(iii); (1b)(ii) and (2b)(iv); (1b)(iii) and (2b)(i); (1 b)(iii) and (2b)(ii); (1 b)(iii) and (2b)(iii); (1 b)(iii) and (2b)(iv); (1 b)(iv) and (2b)(ii); (1 b)(iv) and (2b)(iii); and (1b)(iv) and (2b)(iv).

In aspects and embodiments according to the present disclosure, a cancer to be treated/prevented with an antigen-binding molecule that binds to HER3 in combination with an antagonist of HER3-mediated signalling is homozygous for an activating mutation to a gene encoding a positive regulator of HER3-mediated signalling selected from: PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5. In some embodiments, the cancer is heterozygous for an activating mutation to a gene encoding a positive regulator of HER3-mediated signalling selected from: PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5. In some embodiments, the cancer comprises genetic variation resulting in an increase in the expression of, or an increase in the activity of a gene product of, a gene encoding a positive regulator of HER3-mediated signalling selected from: PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5.

In aspects and embodiments according to the present disclosure, a cancer to be treated/prevented with an antigen-binding molecule that binds to HER3 in combination with an antagonist of HER3-mediated signalling is homozygous for an activating mutation to a gene encoding a positive regulator of signalling through the MAPK/ERK pathway selected from: ERBB3, ERBB2, ERBB4, EGFR, IGF1R, NRG1, NRG2, EGF, IRS2, GAB2, GRB2, PTPN11, SHP2, SOS1, HRAS, KRAS, NRAS, RAF1, MAP2K1, MAP2K2, MAPK1, MYC, RPS6KA1, RPS6, MKNK1 and CREB1. In some embodiments, the cancer is heterozygous for an activating mutation to a gene encoding a positive regulator of signalling through the MAPK/ERK pathway selected from: ERBB3, ERBB2, ERBB4, EGFR, IGF1R, NRG1, NRG2, EGF, IRS2, GAB2, GRB2, PTPN11, SHP2, SOS1, HRAS, KRAS, NRAS, RAF1, MAP2K1, MAP2K2, MAPK1, MYC, RPS6KA1, RPS6, MKNK1 and CREB1. In some embodiments, the cancer comprises genetic variation resulting in an increase in the expression of, or an increase in the activity of a gene product of, a gene encoding a positive regulator of signalling through the MAPK/ERK pathway selected from: ERBB3, ERBB2, ERBB4, EGFR, IGF1R, NRG1, NRG2, EGF, IRS2, GAB2, GRB2, PTPN11, SHP2, SOS1, HRAS, KRAS, NRAS, RAF1, MAP2K1, MAP2K2, MAPK1, MYC, RPS6KA1, RPS6, MKNK1 and CREB1.

In aspects and embodiments according to the present disclosure, a cancer to be treated/prevented with an antigen-binding molecule that binds to HER3 in combination with an antagonist of HER3-mediated signalling is homozygous for an activating mutation to a gene encoding a positive regulator of signalling through the PI3K/AKT/mTOR pathway selected from: ERBB3, ERBB2, ERBB4, EGFR, IGF1R, NRG1, NRG2, EGF, IRS2, GAB2, SHP2, CREB1, PIK3CB, PIK3CD, MTOR, PDK1, AKT1, AKT2, AKT3, JAK2, STAT3 and STAT5. In some embodiments, the cancer is heterozygous for an activating mutation to a gene encoding a positive regulator of signalling through the PI3K/AKT/mTOR pathway selected from: ERBB3, ERBB2, ERBB4, EGFR, IGF1R, NRG1, NRG2, EGF, IRS2, GAB2, SHP2, CREB1, PIK3CB, PIK3CD, MTOR, PDK1, AKT1, AKT2, AKT3, JAK2, STAT3 and STAT5. In some embodiments, the cancer comprises genetic variation resulting in an increase in the expression of, or an increase in the activity of a gene product of, a gene encoding a positive regulator of signalling through the PI3K/AKT/mTOR pathway selected from: ERBB3, ERBB2, ERBB4, EGFR, IGF1R, NRG1, NRG2, EGF, IRS2, GAB2, SHP2, CREB1, PIK3CB, PIK3CD, MTOR, PDK1, AKT1, AKT2, AKT3, JAK2, STAT3 and STAT5.

In aspects and embodiments according to the present disclosure, a cancer to be treated/prevented with an antigen-binding molecule that binds to HER3 in combination with an antagonist of HER3-mediated signalling is homozygous for an inactivating mutation to a gene encoding a negative regulator of HER3-mediated signalling selected from: PPP2CA, PIK3R1, PIK3R2, NF1, BAD and PHLPP1. In some embodiments, the cancer is heterozygous for an inactivating mutation to a gene encoding a negative regulator of HER3-mediated signalling selected from: PPP2CA, PIK3R1, PIK3R2, NF1, BAD and PHLPP1. In some embodiments, the cancer comprises genetic variation resulting in a decrease in the expression of, or a decrease in the activity of a gene product of, a gene encoding a negative regulator of HER3-mediated signalling selected from: PPP2CA, PIK3R1, PIK3R2, NF1, BAD and PHLPP1.

In aspects and embodiments according to the present disclosure, a cancer to be treated/prevented with an antigen-binding molecule that binds to HER3 in combination with an antagonist of HER3-mediated signalling is homozygous for an inactivating mutation to NF1. In some embodiments, the cancer is heterozygous for an inactivating mutation to is homozygous for an inactivating mutation to NF1. In some embodiments, the cancer comprises genetic variation resulting in a decrease in the expression of, or a decrease in the activity of a gene product of, is homozygous for an inactivating mutation to NF1.

In aspects and embodiments according to the present disclosure, a cancer to be treated/prevented with an antigen-binding molecule that binds to HER3 in combination with an antagonist of HER3-mediated signalling is homozygous for an inactivating mutation to a gene encoding a negative regulator of signalling through the PI3K/AKT/mTOR pathway selected from: PPP2CA, PIK3R1, PIK3R2, BAD and PHLPP1. In some embodiments, the cancer is heterozygous for an inactivating mutation to a gene encoding a negative regulator of signalling through the PI3K/AKT/mTOR pathway selected from: PPP2CA, PIK3R1, PIK3R2, BAD and PHLPP1. In some embodiments, the cancer comprises genetic variation resulting in a decrease in the expression of, or a decrease in the activity of a gene product of, a gene encoding a negative regulator of signalling through the PI3K/AKT/mTOR pathway selected from: PPP2CA, PIK3R1, PIK3R2, BAD and PHLPP1.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, KRAS. In some embodiments, the cancer (i) comprises amplification of MET, and (ii) comprises an activating mutation to KRAS.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, PIK3CA. In some embodiments, the cancer (i) comprises amplification of MET, and (ii) comprises an activating mutation to PIK3CA.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, BRAF. In some embodiments, the cancer (i) comprises amplification of MET, and (ii) comprises an activating mutation to BRAF.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in a decrease in the expression of, or a decrease in the activity of a gene product of, PTEN. In some embodiments, the cancer (i) comprises amplification of MET, and (ii) comprises an inactivating mutation to PTEN.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, KRAS, and (iii) comprises genetic variation resulting in an increase in the expression of, or an increase in the activity of a gene product of, PIK3CA. In some embodiments, the cancer (i) comprises amplification of MET, and (ii) comprises an activating mutation to KRAS, and (iii) comprises an activating mutation to PIK3CA.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, KRAS, and (iii) comprises genetic variation resulting in an increase in the expression of, or an increase in the activity of a gene product of, BRAF. In some embodiments, the cancer (i) comprises amplification of MET, and (ii) comprises an activating mutation to KRAS, and (iii) comprises an activating mutation to BRAF.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, KRAS, and (iii) comprises genetic variation resulting in a decrease in the expression of, or a decrease in the activity of a gene product of, PTEN. In some embodiments, the cancer (i) comprises amplification of MET, and (ii) comprises an activating mutation to KRAS, and (iii) comprises an inactivating mutation to PTEN.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, PIK3CA, and (iii) comprises genetic variation resulting in an increase in the expression of, or an increase in the activity of a gene product of, BRAF. In some embodiments, the cancer (i) comprises amplification of MET, and (ii) comprises an activating mutation to PIK3CA, and (iii) comprises an activating mutation to BRAF.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, PIK3CA, and (iii) comprises genetic variation resulting in a decrease in the expression of, or a decrease in the activity of a gene product of, PTEN. In some embodiments, the cancer (i) comprises amplification of MET, and (ii) comprises an activating mutation to PIK3CA, and (iii) comprises an inactivating mutation to PTEN.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, BRAF, and (iii) comprises genetic variation resulting in a decrease in the expression of, or a decrease in the activity of a gene product of, PTEN. In some embodiments, the cancer (i) comprises amplification of MET, and (ii) comprises an activating mutation to BRAF, and (iii) comprises an inactivating mutation to PTEN.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, KRAS, and (iii) comprises genetic variation resulting in an increase in the expression of, or an increase in the activity of a gene product of, PIK3CA, and (iv) comprises genetic variation resulting in an increase in the expression of, or an increase in the activity of a gene product of, BRAF. In some embodiments, the cancer (i) comprises amplification of MET, and (ii) comprises an activating mutation to KRAS, and (iii) comprises an activating mutation to PIK3CA, and (iv) comprises an activating mutation to BRAF.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, KRAS, and (iii) comprises genetic variation resulting in an increase in the expression of, or an increase in the activity of a gene product of, PIK3CA, and (iv) comprises genetic variation resulting in a decrease in the expression of, or a decrease in the activity of a gene product of, PTEN. In some embodiments, the cancer (i) comprises amplification of MET, and (ii) comprises an activating mutation to KRAS, and (iii) comprises an activating mutation to PIK3CA, and (iv) comprises an inactivating mutation to PTEN.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, KRAS, and (iii) comprises genetic variation resulting in an increase in the expression of, or an increase in the activity of a gene product of, BRAF, and (iv) comprises genetic variation resulting in a decrease in the expression of, or a decrease in the activity of a gene product of, PTEN. In some embodiments, the cancer (i) comprises amplification of MET, and (ii) comprises an activating mutation to KRAS, and (iii) comprises an activating mutation to BRAF, and (iv) comprises an inactivating mutation to PTEN.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, PIK3CA, and (iii) comprises genetic variation resulting in an increase in the expression of, or an increase in the activity of a gene product of, BRAF, and (iv) comprises genetic variation resulting in a decrease in the expression of, or a decrease in the activity of a gene product of, PTEN. In some embodiments, the cancer (i) comprises amplification of MET, and (ii) comprises an activating mutation to PIK3CA, and (iii) comprises an activating mutation to BRAF, and (iv) comprises an inactivating mutation to PTEN.

In aspects and embodiments according to the present disclosure, a cancer to be treated/prevented with an antigen-binding molecule that binds to HER3 in combination with an antagonist of HER3-mediated signalling (i) comprises genetic variation resulting in an increase in the expression of, or an increase in the activity of a gene product of, MET, and (ii) comprises genetic variation resulting in an increase in the expression of, or an increase in the activity of a gene product of, KRAS, and (iii) comprises genetic variation resulting in an increase in the expression of, or an increase in the activity of a gene product of, PIK3CA, and (iv) comprises genetic variation resulting in an increase in the expression of, or an increase in the activity of a gene product of, BRAF, and (v) comprises genetic variation resulting in a decrease in the expression of, or a decrease in the activity of a gene product of, PTEN. In some embodiments, the cancer (i) comprises amplification of MET, and (ii) comprises an activating mutation to KRAS, and (iii) comprises an activating mutation to PIK3CA, and (iv) comprises an activating mutation to BRAF, and (v) comprises an inactivating mutation to PTEN.

### Cancers Characterised by the Presence of Genetic Variation Resulting in an Increase in the Expression of, or an Increase in the Activity of a Gene Product of a Chromosome 3q Gene

Aspects and embodiments of the present disclosure relate to cancers comprising genetic variation resulting in an increase in the expression of, or an increase in the activity of a gene product of, a gene located on chromosome 3q. It will be appreciated that cancers described in this section may be further characterised according to the section above entitled ‘Cancers’.

Characteristics of early oncogenesis in squamous cell carcinomas include amplification of chromosome 3q (Chr3q), which is a well known genetic aberration associated with carcinogen exposure (Rooney et al. Oncologist (2013) 18(6):707-716). Chr3q amplifications leads to increased transcriptional activities of a number of oncogenes including TP63 (which directly promotes HER3 ligand NRG1 expression), SOX2 (which directly promotes expression of EGFR ligands), and PIK3CA genes (which enhances activation of the PI3K pathway) (Perez-Moreno et al., Clinical Cancer Research (2012) 18(9):2443-2451).

In aspects and embodiments according to the present disclosure, a cancer to be treated/prevented in accordance with the present disclosure is characterised according to one or more of the following features:
(6a)(i) comprises genetic variation resulting in an increase in the expression of, or an increase in the activity of a gene product of, TP63.
(6a)(ii) comprises amplification of TP63 (e.g. amplification of TP63 as described herein).
(7a)(i) comprises genetic variation resulting in an increase in the expression of, or an increase in the activity of a gene product of, PIK3CA.
(7a)(ii) comprises amplification of PIK3CA (e.g. amplification of PIK3CA as described herein).
(8a)(i) comprises genetic variation resulting in an increase in the expression of, or an increase in the activity of a gene product of, SOX2.
(8a)(ii) comprises amplification of SOX2 (e.g. amplification of SOX2 as described herein).

In some embodiments, a cancer to be treated/prevented in accordance with the present disclosure is characterised by one of the following combinations of features (with reference to the preceding paragraph): (6a); (7a); (8a); (6a), (7a); (6a), (8a); (7a), (8a); (6a), (7a), (8a).

In the combinations of the preceding paragraph, ‘(6a)’ is independently selected from (6a)(i) and (6a)(ii) above; and ‘(7a)’ is independently selected from (7a)(i) and (7a)(ii) above; and ‘(8a)’ is independently selected from (8a)(i) and (8a)(ii) above.

All possible combinations of such features in accordance with the above are expressly contemplated. Purely by way of illustration, a cancer comprising the combination of features “(6a), (7a)” specifically contemplates cancers comprising: (6a)(i) and (7a)(i); (6a)(i) and (7a)(ii); (6a)(ii) and (7a)(i); and (6a)(ii) and (7a)(ii).

In aspects and embodiments according to the present disclosure, a cancer to be treated/prevented in accordance with the present disclosure comprises amplification of chromosome 3q.

In aspects and embodiments according to the present disclosure, a cancer to be treated/prevented in accordance with the present disclosure comprises genetic variation resulting in an increase in the expression of, or an increase in the activity of a gene product of, a gene selected from ADIPOQ, AMOTL2, ARHGAP31, TIMMDC1, C3orf70, CAMPD1, CCDC80, CD200R1, CHST13, CLDND1, CPN2, CPOX, DPPA2, DTX3L, DZIP3, EAF2, EFCC1, ETM1, ETV5, FAM3D, FAM43A, FAM162A, FBXO40, FILIP1L, GYG1, HACD2, HGD, IFT122, KIAA1257, LINC01279, LNCR5, LMLN, LRRC15, LSG1, MB21D2, MCCC1, MORC1, MYLK, NEPRO, NFKBIZ, OTOL1, PARP14, PCCB, PDCD10, PIK3CA, PISRT1, PROSER1, RAB7, RASA2, RETNLB, RHO, RIOX2, SELT, SENP7, SERP1, SOX2, SOX2OT, SPG14, SRPRB, TEX55, TMEM44, TM4SF1, TMPRSS7, TP63, TRAT1, USH3A, ZBED2 and ZNF9. In some embodiments, the cancer comprises genetic variation resulting in an increase in the expression of, or an increase in the activity of a gene product of, a gene selected from: TP63, PIK3CA and SOX2.

In aspects and embodiments according to the present disclosure, a cancer to be treated/prevented in accordance with the present disclosure comprises amplification of a gene selected from ADIPOQ, AMOTL2, ARHGAP31, TIMMDC1, C3orf70, CAMPD1, CCDC80, CD200R1, CHST13, CLDND1, CPN2, CPOX, DPPA2, DTX3L, DZIP3, EAF2, EFCC1, ETM1, ETV5, FAM3D, FAM43A, FAM162A, FBXO40, FILIP1L, GYG1, HACD2, HGD, IFT122, KIAA1257, LINC01279, LNCR5, LMLN, LRRC15, LSG1, MB21D2, MCCC1, MORC1, MYLK, NEPRO, NFKBIZ, OTOL1, PARP14, PCCB, PDCD10, PIK3CA, PISRT1, PROSER1, RAB7, RASA2, RETNLB, RHO, RIOX2, SELT, SENP7, SERP1, SOX2, SOX2OT, SPG14, SRPRB, TEX55, TMEM44, TM4SF1, TMPRSS7, TP63, TRAT1, USH3A, ZBED2 and ZNF9. In some embodiments, the cancer comprises amplification of a gene selected from: TP63, PIK3CA and SOX2.

In aspects and embodiments according to the present disclosure, a cancer to be treated/prevented in accordance with the present disclosure comprises genetic variation resulting in an increase in the expression of, or an increase in the activity of a gene product of, TP63. In some embodiments, the cancer comprises amplification of TP63.

In aspects and embodiments according to the present disclosure, a cancer to be treated/prevented in accordance with the present disclosure comprises genetic variation resulting in an increase in the expression of, or an increase in the activity of a gene product of, PIK3CA. In some embodiments, the cancer comprises amplification of PIK3CA.

In aspects and embodiments according to the present disclosure, a cancer to be treated/prevented in accordance with the present disclosure comprises genetic variation resulting in an increase in the expression of, or an increase in the activity of a gene product of, SOX2. In some embodiments, the cancer comprises amplification of SOX2.

In aspects and embodiments according to the present disclosure, a cancer to be treated/prevented in accordance with the present disclosure (i) comprises genetic variation resulting in an increase in the expression of, or an increase in the activity of a gene product of, TP63, and (ii) comprises genetic variation resulting in an increase in the expression of, or an increase in the activity of a gene product of, PIK3CA. In some embodiments, the cancer (i) comprises amplification of TP63, and (ii) comprises amplification of PIK3CA.

In aspects and embodiments according to the present disclosure, a cancer to be treated/prevented in accordance with the present disclosure (i) comprises genetic variation resulting in an increase in the expression of, or an increase in the activity of a gene product of, TP63, and (ii) comprises genetic variation resulting in an increase in the expression of, or an increase in the activity of a gene product of, SOX2. In some embodiments, the cancer (i) comprises amplification of TP63, and (ii) comprises amplification of SOX2.

In aspects and embodiments according to the present disclosure, a cancer to be treated/prevented in accordance with the present disclosure (i) comprises genetic variation resulting in an increase in the expression of, or an increase in the activity of a gene product of, PIK3CA, and (ii) comprises genetic variation resulting in an increase in the expression of, or an increase in the activity of a gene product of, SOX2. In some embodiments, the cancer (i) comprises amplification of PIK3CA, and (ii) comprises amplification of SOX2.

In aspects and embodiments according to the present disclosure, a cancer to be treated/prevented in accordance with the present disclosure (i) comprises genetic variation resulting in an increase in the expression of, or an increase in the activity of a gene product of, TP63, and (ii) comprises genetic variation resulting in an increase in the expression of, or an increase in the activity of a gene product of, PIK3CA, and (iii) comprises genetic variation resulting in an increase in the expression of, or an increase in the activity of a gene product of, SOX2. In some embodiments, the cancer (i) comprises amplification of TP63, and (ii) comprises amplification of PIK3CA, and (iii) comprises amplification of SOX2.

### Cancers Characterised by the Presence of Genetic Variation Resulting in an Increase in the Expression of, or an Increase in the Activity of a Gene Product of a Chromosome 7p Gene

Aspects and embodiments of the present disclosure relate to cancers comprising genetic variation resulting in an increase in the expression of, or an increase in the activity of a gene product of, a gene located on chromosome 7p. It will be appreciated that cancers described in this section may be further characterised according to the section above entitled ‘Cancers’.

Chromosome 7p (Chr7p) encoding EGFR has been found to be frequently amplified in LUSC (Couceiro, et al. Revista Portuguesa de Pneumologia (2010) 16(3):453-462).

In aspects and embodiments according to the present disclosure, a cancer to be treated/prevented in accordance with the present disclosure is characterised according to one or more of the following features:
(10a)(i) comprises genetic variation resulting in an increase in the expression of, or an increase in the activity of a gene product of, EGFR.
(10a)(ii) comprises amplification of EGFR (e.g. amplification of EGFR as described herein). In aspects and embodiments according to the present disclosure, a cancer to be treated/prevented in accordance with the present disclosure comprises amplification of chromosome 7p.

In aspects and embodiments according to the present disclosure, a cancer to be treated/prevented in accordance with the present disclosure comprises genetic variation resulting in an increase in the expression of, or an increase in the activity of a gene product of, EGFR. In aspects and embodiments according to the present disclosure, a cancer to be treated/prevented in accordance with the present disclosure comprises amplification of EGFR.

### Cancers Characterised by the Presence of Genetic Variation Resulting in a Decrease in the Expression of, or a Decrease in the Activity of a Gene Product of a Chromosome 3p Gene

Aspects and embodiments of the present disclosure relate to cancers comprising genetic variation resulting in a decrease in the expression of, or a decrease in the activity of a gene product of, a gene located on chromosome 3p. It will be appreciated that cancers described in this section may be further characterised according to the section above entitled ‘Cancers’.

Characteristics of early oncogenesis in squamous cell carcinomas include loss of chromosome 3p (Chr3p), which is a well known genetic aberration associated with carcinogen exposure (Rooney et al. Oncologist (2013) 18(6):707-716). Chr3p loss leads to the deletion of several putative tumor suppressor proteins, including tumor suppressive candidate 2 (TUSC2), which inhibits EGFR (Perez-Moreno et al., Clinical Cancer Research (2012) 18(9):2443-2451). TUSC2 is found to be deleted in ~12-17% of squamous cancer patients (TCGA). Additionally, studies have shown that TUSC2 can negatively regulate EGFR signalling (Dai et al., PLoS One (2015) 10(6):e0123967; Cao et al., Sci Rep. (2016) 6:35741).

In aspects and embodiments according to the present disclosure, a cancer to be treated/prevented in accordance with the present disclosure is characterised according to one or more of the following features:
(11a)(i) does not comprise genetic variation resulting in a decrease in the expression of, or a decrease in the activity of a gene product of, TUSC2.
(11a)(ii) does not comprise deletion of TUSC2 (e.g. deletion of TUSC2 as described herein).

In aspects and embodiments according to the present disclosure, a cancer to be treated/prevented in accordance with the present disclosure comprises deletion of chromosome 3p.

In aspects and embodiments according to the present disclosure, a cancer to be treated/prevented in accordance with the present disclosure comprises genetic variation resulting in a decrease in the expression of, or a decrease in the activity of a gene product of, TUSC2. In aspects and embodiments according to the present disclosure, a cancer to be treated/prevented in accordance with the present disclosure comprises deletion of TUSC2.

### Antigen-Binding Molecules

The present disclosure relates to the therapeutic and prophylactic use of antigen-binding molecules that bind to HER3.

An ‘antigen-binding molecule’ refers to a molecule that binds to a given target antigen. Antigen-binding molecules include antibodies (i.e. immunoglobulins (Igs)) and antigen-binding fragments thereof. As used herein, ‘antibodies’ include monoclonal antibodies, polyclonal antibodies, monospecific and multispecific (e.g., bispecific, trispecific, etc.) antibodies, and antibody-derived antigen-binding molecules such as scFv, scFab, diabodies, triabodies, scFv-Fc, minibodies, single domain antibodies (e.g. VhH, etc.). Antigen-binding fragments of antibodies include e.g. Fv, Fab, F(ab′)₂ and F(ab′) fragments. In some embodiments, an antigen-binding molecule may be an antibody or an antigen-binding fragment thereof.

Antigen-binding molecules according to the present disclosure also include antibody-derived molecules, e.g. molecules comprising an antigen-binding region/domain derived from an antibody. Antibody-derived antigen-binding molecules may comprise an antigen-binding region/domain that comprises, or consists of, the antigen-binding region of an antibody (e.g. an antigen-binding fragment of an antibody). In some embodiments, the antigen-binding region/domain of an antibody-derived antigen-binding molecule may be or comprise the Fv (e.g. provided as an scFv) or the Fab region of an antibody, or the whole antibody. For example, antigen-binding molecules according to the present disclosure include antibody-drug conjugates (ADCs) comprising a (cytotoxic) drug moiety (e.g. as described hereinbelow). Antigen-binding molecules according to the present disclosure also include multispecific antigen-binding molecules such as immune cell engager molecules comprising a domain for recruiting (effector) immune cells (reviewed e.g. in Goebeler and Bargou, Nat. Rev. Clin. Oncol. (2020) 17: 418-434 and Ellerman, Methods (2019) 154:102-117, both of which are hereby incorporated by reference in their entirety), including BiTEs, BiKEs and TriKEs. Antigen-binding molecules according to the present disclosure also include chimeric antigen receptors (CARs), which are recombinant receptors providing both antigen-binding and T cell activating functions (CAR structure, function and engineering is reviewed e.g. in Dotti et al., Immunol Rev (2014) 257(1) and Jayaraman et al., EBioMedicine (2020) 58:102931, both of which are hereby incorporated by reference in their entirety).

The antigen-binding molecules of the present disclosure comprise a moiety or moieties capable of binding to a target antigen(s). In some embodiments, the moiety capable of binding to a target antigen comprises an antibody heavy chain variable region (VH) and an antibody light chain variable region (VL) of an antibody capable of specific binding to the target antigen. In some embodiments, the moiety capable of binding to a target antigen comprises or consists of an aptamer capable of binding to the target antigen, e.g. a nucleic acid aptamer (reviewed, for example, in Zhou and Rossi Nat Rev Drug Discov. 2017 16(3):181-202). In some embodiments, the moiety capable of binding to a target antigen comprises or consists of an antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

As used herein, a ‘peptide’ refers to a chain of two or more amino acid monomers linked by peptide bonds. A peptide typically has a length in the region of about 2 to 50 amino acids. A ‘polypeptide’ is a polymer chain of two or more peptides. Polypeptides typically have a length greater than about 50 amino acids.

The antigen-binding molecules of the present disclosure generally comprise an antigen-binding domain comprising a VH and a VL of an antibody capable of specific binding to the target antigen. The antigen-binding domain formed by a VH and a VL may also be referred to herein as an Fv region.

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. In some embodiments, the polypeptides form part of a larger polypeptide comprising the polypeptides (e.g. in the case of scFv comprising VH and VL, or in the case of scFab comprising VH-CH1 and VL-CL).

An antigen-binding molecule may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The antigen-binding molecules of the present disclosure may be designed and prepared using the sequences of monoclonal antibodies (mAbs) capable of binding to HER3. Antigen-binding regions of antibodies, such as single chain variable fragment (scFv), Fab and F(ab′)₂ fragments may also be used/provided. An ‘antigen-binding region’ is any fragment of an antibody that binds to the target for which the given antibody is specific.

Antibodies generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody that binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015) 43 (Database issue):D413-22), which uses the IMGT V-DOMAIN numbering rules as described in Lefranc et al., Dev. Comp. Immunol. (2003) 27:55-77. In preferred embodiments, the CDRs and FRs of antigen-binding molecules referred to herein are defined according to the IMGT information system.

The VH and VL region of an antigen-binding region of an antibody together constitute the Fv region. In some embodiments, an antigen-binding molecule according to the present disclosure comprises, or consists of, an Fv region that binds to HER3. In some embodiments, the VH and VL regions of the Fv are provided as single polypeptide joined by a linker sequence, i.e. a single chain Fv (scFv).

The VL and light chain constant (CL) region, and the VH region and heavy chain constant 1 (CH1) region of an antigen-binding region of an antibody together constitute the Fab region. In some embodiments, the antigen-binding molecule comprises a Fab region comprising a VH, a CH1, a VL and a CL (e.g. Cκ or Cλ). In some embodiments, the Fab region comprises a polypeptide comprising a VH and a CH1 (e.g. a VH-CH1 fusion polypeptide), and a polypeptide comprising a VL and a CL (e.g. a VL-CL fusion polypeptide). In some embodiments, the Fab region comprises a polypeptide comprising a VH and a CL (e.g. a VH-CL fusion polypeptide) and a polypeptide comprising a VL and a CH (e.g. a VL-CH1 fusion polypeptide); that is, in some embodiments, the Fab region is a CrossFab region. In some embodiments, the VH, CH1, VL and CL regions of the Fab or CrossFab are provided as single polypeptide joined by linker regions, i.e. as a single chain Fab (scFab) or a single chain CrossFab (scCrossFab).

In some embodiments, an antigen-binding molecule described herein comprises, or consists of, a whole antibody which binds to HER3. As used herein, ‘whole antibody’ refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are ~150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments, the antigen-binding molecule comprises, or consists of, an IgG (e.g. IgG1, IgG2, IgG3, IgG4), IgA (e.g. IgA1, IgA2), IgD, IgE, or IgM which binds to HER3.

In some embodiments, the antigen-binding molecule comprises the CDRs of an antigen-binding molecule which is capable of binding to HER3. In some embodiments, the antigen-binding molecule comprises the FRs of an antigen-binding molecule which is capable of binding to HER3. In some embodiments, the antigen-binding molecule comprises the CDRs and the FRs of an antigen-binding molecule which is capable of binding to HER3. That is, in some embodiments the antigen-binding molecule comprises the VH region and the VL region of an antigen-binding molecule which is capable of binding to HER3.

In some embodiments, an antigen-binding molecule which is capable of binding to HER3 according to the present disclosure may be selected from: any embodiment of an antigen-binding molecule described in WO 2019185878 A1 (which is hereby incorporated by reference in its entirety), 10D1F (described e.g. in WO 2019185878 A1), seribantumab (also known as MM-121, described e.g. in Schoeberl et al., Sci. Signal. (2009) 2(77): ra31), elgemtumab (also known as LJM-716, described e.g. in Garner et al., Cancer Res (2013) 73: 6024-6035), patritumab (also known as U-1287 and AMG-888, described e.g. in Shimizu et al. Cancer Chemother Pharmacol. (2017) 79(3):489-495), GSK2849330 (described e.g. in Clarke et al., Eur J Cancer. (2014) 50:98-9), lumretuzumab (also known as RG7116 and RO-5479599, described e.g. in Mirschberger et al. Cancer Research (2013) 73(16) 5183-5194), CDX-3379 (also known as KTN3379, described e.g. in Lee et al., Proc Natl Acad Sci USA. 2015 Oct. 27; 112(43):13225), AV-203 (also known as CAN-017, described e.g. in Meetze et al., Eur J Cancer 2012; 48:126), barecetamab (also known as ISU104, described e.g. in Kim et al., Cancer Res (2018) 78(13 Suppl):Abstract #830), TK-A3, TK-A4 (described e.g. in Malm et al., MAbs (2016) 8:1195-209), MP-EV20 (described e.g. in Sala et al., Transl. Oncol. (2013) 6:676-84), 1A5-3D4 (described e.g. in Wang et al., Cancer Lett (2016) 380:20-30), 9F7-F11, 16D3-C1 (described e.g. in Lazrek et al., Neoplasia (2013) 15:335-47), NG33, A5, F4 (described e.g. in Gaborit et al., PNAS USA (2015) 112:839-44), huHER3-8 (described e.g. in Kugel et al., Cancer Res. (2014) 74:4122-32), REGN1400 (described e.g. in Zhang et al., Mol Cancer Ther (2014) 13:1345-1355) and zenocutuzumab (also known as MCLA-128, described e.g. in de Vries Schultink et al., Clin Pharmacokinet. (2020) 59: 875-884).

In some embodiments, the antigen-binding molecule is selected from 10D1F and seribantumab. In some embodiments, the antigen-binding molecule is 10D1F.

In some embodiments, the antigen-binding molecule comprises the CDRs of, or comprises the VH and VL of, a HER3-binding antibody clone selected from 10D1_c89, 10D1, 10D1_c75, 10D1_c76, 10D1_c77, 10D1_c78v1, 10D1_c78v2, 10D1_11B, 10D1_c85v1, 10D1_c85v2, 10D1_c85o1, 10D1_c85o2, 10D1_c87, 10D1_c90, 10D1_c91, 10D1_c92 and 10D1_c93.

In some embodiments, the antigen-binding molecule comprises:
(1) a VH region incorporating the following CDRs:
   HC-CDR1 having the amino acid sequence of SEQ ID NO:40
   HC-CDR2 having the amino acid sequence of SEQ ID NO:43
   HC-CDR3 having the amino acid sequence of SEQ ID NO:48,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:66
   LC-CDR2 having the amino acid sequence of SEQ ID NO:69
   LC-CDR3 having the amino acid sequence of SEQ ID NO:74;
   or a variant thereof in which one or two or three amino acids in one or more of LC-CDR1, LC-CDR2 or LC-CDR3 are substituted with another amino acid.
(2) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(3) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:64
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(4) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:65
   LC-CDR2 having the amino acid sequence of SEQ ID NO:67
   LC-CDR3 having the amino acid sequence of SEQ ID NO:71;
   or a variant thereof in which one or two or three amino acids in one or more of LC-CDR1, LC-CDR2 or LC-CDR3 are substituted with another amino acid.
(5) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(6) a VH region incorporating the following CDRs:
   HC-CDR1 having the amino acid sequence of SEQ ID NO:39
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(7) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(8) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:46,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(9) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:47,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(10) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:72;
   or a variant thereof in which one or two or three amino acids in one or more of LC-CDR1, LC-CDR2 or LC-CDR3 are substituted with another amino acid.
(11) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:73;
   or a variant thereof in which one or two or three amino acids in one or more of LC-CDR1, LC-CDR2 or LC-CDR3 are substituted with another amino acid.

In some embodiments, the antigen-binding molecule comprises:
(12) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:21; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:49.
(13) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:50.
(14) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:51.
(15) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:52.
(16) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(17) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:26; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(18) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:27; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(19) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:28; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(20) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:29; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(21) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:30; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(22) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:31; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(23) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:32; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:57.
(24) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:33; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:58.
(25) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:34; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:59.
(26) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:35; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:60.
(27) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:61.
(28) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:32; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:62.

In some embodiments described herein, one or more amino acids of an amino acid sequence referred to herein (e.g. an amino acid sequence of an antigen-binding molecule, e.g. an amino acid sequence of a CDR or VH/VL region) are substituted with another amino acid. A substitution comprises substitution of an amino acid residue with a non-identical ‘replacement’ amino acid residue. A replacement amino acid residue of a substitution according to the present disclosure may be a naturally-occurring amino acid residue (i.e. encoded by the genetic code) which is non-identical to the amino acid residue at the relevant position of the equivalent, unsubstituted amino acid sequence, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (Ile): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid may be a non-naturally occurring amino acid residue—i.e. an amino acid residue other than those recited in the preceding sentence. Examples of non-naturally occurring amino acid residues include norleucine, ornithine, norvaline, homoserine, aib, and other amino acid residue analogues such as those described in Ellman, et al., Meth. Enzym. 202 (1991) 301-336.

In some embodiments, a substitution may be biochemically conservative. In some embodiments, where an amino acid to be substituted is provided in one of rows 1 to 5 of the table below, the replacement amino acid of the substitution is another, non-identical amino acid provided in the same row:

| Row | Shared property | Amino acids |
|---|---|---|
| 1 | Hydrophobic | Met, Ala, Val, Leu, Ile, |
| | | Trp, Tyr, Phe, Norleucine |
| 2 | Neutral hydrophilic | Cys, Ser, Thr, Asn, Gln |
| 3 | Acidic or negatively-charged | Asp, Glu |
| 4 | Basic or positively-charged | His, Lys, Arg |
| 5 | Orientation influencing | Gly, Pro |

By way of illustration, in some embodiments wherein substitution is of a Met residue, the replacement amino acid may be selected from Ala, Val, Leu, lie, Trp, Tyr, Phe and Norleucine.

In some embodiments, a replacement amino acid in a substitution may have the same side chain polarity as the amino acid residue it replaces. In some embodiments, a replacement amino acid in a substitution may have the same side chain charge (at pH 7.4) as the amino acid residue it replaces:

| | Side-chain | Side-chain charge |
|---|---|---|
| Amino Acid | polarity | (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another, non-identical nonpolar amino acid. In some embodiments, a polar amino acid is substituted with another, non-identical polar amino acid. In some embodiments, an acidic polar amino acid is substituted with another, non-identical acidic polar amino acid. In some embodiments, a basic polar amino acid is substituted with another, non-identical basic polar amino acid. In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally conservative. That is, in some embodiments, the substitution may not affect (or may not substantially affect) one or more functional properties (e.g. target binding) of the antigen-binding molecule comprising the substitution as compared to the equivalent unsubstituted molecule.

The VH and VL region of an antigen-binding region of an antibody together constitute the Fv region. In some embodiments, the antigen-binding molecule according to the present disclosure comprises, or consists of, an Fv region that binds to HER3. In some embodiments the VH and VL regions of the Fv are provided as single polypeptide joined by a linker region, i.e. a single chain Fv (scFv).

In some embodiments, the antigen-binding molecule of the present disclosure comprises one or more regions of an immunoglobulin heavy chain constant sequence. In some embodiments, the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM.

The VL and light chain constant (CL) region, and the VH region and heavy chain constant 1 (CH1) region of an antigen-binding region of an antibody together constitute the Fab region. In some embodiments, the antigen-binding molecule of the present disclosure comprises, or consists of, a Fab region that binds to HER3.

In some embodiments, the antigen-binding molecule described herein comprises, or consists of, a whole antibody that binds to HER3. As used herein, ‘whole antibody’ refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are ~150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments, the antigen-binding molecule comprises, or consists of, an IgG (e.g. IgG1, IgG2, IgG3, IgG4), IgA (e.g. IgA1, IgA2), IgD, IgE, or IgM that binds to HER3.

In some embodiments, the antigen-binding molecule comprises, or consists of:
(i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:75; and
(ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:76.

Antigen-binding molecules according to the present disclosure may be provided in the form of compositions comprising such antigen-binding molecules. The antigen-binding molecules, may be formulated as pharmaceutical compositions or medicaments for clinical use and may comprise a pharmaceutically acceptable carrier, diluent, excipient or adjuvant. Compositions may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration, which may include injection or infusion.

Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via cannula) to the blood, a tumor, or a selected region of the human or animal body. In some embodiments, compositions may be formulated for injection or infusion, e.g. into a blood vessel or tumor.

### Antagonists of HER3-Mediated Signalling

Aspects and embodiments of the present disclosure relate to therapeutic/prophylactic intervention with (i) an antagonist of HER3-mediated signalling, and (ii) an antigen-binding molecule that binds to HER3.

While HER3-binding antigen-binding molecules according to the present disclosure may behave as antagonists of HER3-mediated signalling, it will be appreciated that in accordance with aspects and embodiments of the present disclosure relating to combination treatment, components (i) and (ii) of the combination are preferably non-identical (i.e. they are different agents).

Treatment with an antagonist of HER3-mediated signalling in accordance with the present disclosure is contemplated, in particular, in connection with therapeutic/prophylactic intervention for the treatment/prevention of cancers described in the section entitled ‘cancers characterised by the presence of genetic variation resulting in an increase in HER3-mediated signalling’ hereinabove. In accordance with such embodiments, treatment with an antagonist of HER3-mediated signalling may be effective to restore the level of signalling to the level observed in the absence of mutation(s) (i.e. the level of signalling by equivalent cells harbouring only the wildtype allele(s)).

In this way, treatment with an antagonist of HER3-mediated signalling may be useful to condition a subject for treatment with a HER3-binding antigen-binding molecule described herein. That is, administration of an antagonist of HER3-mediated signalling is preferably effective to sensitize the cancer to (i.e. render the cancer susceptible to) treatment with a HER3-binding antigen-binding molecule, such that treatment of the subject's cancer with a HER3-binding antigen-binding molecule is more effective than it would have been in the absence of treatment with an antagonist of HER3-mediated signalling.

It will be appreciated that the particular antagonist of HER3-mediated signalling to be employed in a combination treatment according to the present disclosure may be selected in accordance with the mutation status/genotype of the cancer to be treated. By way of illustration, where a cancer comprises an activating mutation to KRAS, the antagonist may be an antagonist of signalling through the MAPK/ERK pathway. Similarly, where a cancer comprises an activating mutation to PIK3CA or an inactivating mutation to PTEN, the antagonist may be an antagonist of signalling through the PI3K/AKT/mTOR pathway.

In some embodiments, an antagonist of HER3-mediated signalling according to the present disclosure is a pan-ErbB inhibitor (e.g. sapitinib or Sym013). In some embodiments, an antagonist of HER3-mediated signalling is an inhibitor of signalling mediated by EGFR (e.g. cetuximab, panitumumab, gefitinib, erlotinib, lapatinib, afatinib, brigatinib, icotinib, osimertinib, zalutumumab, vandetanib, necitumumab, nimotuzumab, dacomitinib, duligotuzumab or matuzumab). In some embodiments, an antagonist of HER3-mediated signalling is an inhibitor of signalling mediated by HER2 (e.g. trastuzumab, pertuzumab, lapatinib, neratinib, afatinib, dacomitinib, MM-111, zenocutuzumab, MCLA-128 or margetuximab). In some embodiments, an antagonist of HER3-mediated signalling is an inhibitor of signalling mediated by HER3 (e.g. seribantumab, lumretuzumab, elgemtumab, KTN3379, AV-203, GSK2849330, REGN1400, MP-RM-1, EV20, pertuzumab, duligotuzumab, MM-111, zenocutuzumab, istiratumab, MCLA-128, patritumab, EZN-3920, RB200, U3-1402, TX2-121-2, EZN-3920 and miR-205). In some embodiments, an antagonist of HER3-mediated signalling is an inhibitor of signalling mediated by HER4 (e.g. lapatinib, ibrutinib, afatinib, dacomitinib or neratinib).

In some embodiments, an antagonist of HER3-mediated signalling inhibits a downstream effector of HER3 signalling. Downstream effectors of HER3-mediated signalling include e.g. PI3K, AKT, K-Ras, B-Raf, MEK/ERK and mTOR. In some embodiments, an antagonist of HER3-mediated signalling is an inhibitor of the MAPK/ERK pathway. In some embodiments, an antagonist of HER3-mediated signalling is an inhibitor of the PI3K/ATK/mTOR pathway.

In some embodiments, an antagonist of HER3-mediated signalling is a PI3K inhibitor (e.g. pictilisib, buparlisib, dactolisib, SAR245409, AZD8186, idelalisib, copanlisib or duvelisib). In some embodiments, an antagonist of HER3-mediated signalling is an AKT inhibitor (e.g. MK-2206, AZD5363, GSK690693, GSK2110183, ipatasertib, VQD-002, perifosine or miltefosine). In some embodiments, an antagonist of HER3-mediated signalling is an inhibitor of KRAS (e.g. sotorasib (also known as AMG 510), ARS-1620, adagrasib (also known as MRTX849), LY3499446, ARS-3248/JNJ-74699157, B12852 or RRSP chimeric toxin). In some embodiments, an antagonist of HER3-mediated signalling is a BRAF inhibitor (e.g. vemurafenib, dabrafenib, SB590885, XL281, RAF265, encorafenib, belvarafenib, PLX8394, LY3009120, LXH254, GDC-0879, PLX-4720, sorafenib, or LGX818). In some embodiments, an antagonist of HER3-mediated signalling is a MEK/ERK inhibitor (e.g. trametinib, cobimetinib, binimetinib, selumetinib, pimasertib, PD-325901, CI-1040, PD035901, or TAK-733). In some embodiments, an antagonist of HER3-mediated signalling is a mTOR inhibitor (e.g. rapamycin, deforolimus, temsirolimus, everolimus, ridaforolimus or sapanisertib).

### Therapeutic and Prophylactic Intervention

Aspects and embodiments of the present disclosure relate to therapeutic and prophylactic intervention for the treatment and prevention of the cancers described herein.

The present disclosure provides an antigen-binding molecule that binds to HER3 for use in a method of treating or preventing a cancer described herein in a subject. Also provided is the use of an antigen-binding molecule that binds to HER3 in the manufacture of a medicament for use in treating or preventing a cancer described herein in a subject. Also provided is a method of treating or preventing a cancer described herein in a subject, comprising administering to the subject a therapeutically- or prophylactically-effective amount of an antigen-binding molecule that binds to HER3.

The present disclosure provides an antigen-binding molecule that binds to HER3 for use in a method of treating or preventing a cancer described herein, wherein the method further comprises administering an antagonist of HER3-mediated signalling. Also provided is an antagonist of HER3-mediated signalling for use in a method of treating or preventing a cancer described herein, wherein the method further comprises administering an antigen-binding molecule that binds to HER3. Also provided is the use of an antigen-binding molecule that binds to HER3 in the manufacture of a medicament for use in a method of treating or preventing a cancer described herein, wherein the method further comprises administering an antagonist of HER3-mediated signalling. Also provided is the use of an antagonist of HER3-mediated signalling in the manufacture of a medicament for use in a method of treating or preventing a cancer described herein, wherein the method further comprises administering an antigen-binding molecule that binds to HER3. Further provided is a method of treating or preventing a cancer described herein, the method comprising administering a therapeutically- or prophylactically-effective amount of (i) an antigen-binding molecule that binds to HER3 and (ii) an antagonist of HER3-mediated signalling to a subject in need of treatment.

The present disclosure also provides an antigen-binding molecule that binds to HER3 and an antagonist of HER3-mediated signalling (e.g. in the form of a pharmaceutical combination or pharmaceutical composition comprising an antigen-binding molecule that binds to HER3 and an antagonist of HER3-mediated signalling) for use in a method of treating or preventing a cancer described herein. Also provided is the use of an antigen-binding molecule that binds to HER3 and an antagonist of HER3-mediated signalling (e.g. in the form of a pharmaceutical combination or pharmaceutical composition comprising an antigen-binding molecule that binds to HER3 and an antagonist of HER3-mediated signalling) in the manufacture of a medicament for use in a method of treating or preventing a cancer described herein. Also provided is a method of treating or preventing a cancer described herein, the method comprising administering a therapeutically- or prophylactically-effective amount of an antigen-binding molecule that binds to HER3 and an antagonist of HER3-mediated signalling (e.g. in the form of a pharmaceutical combination or pharmaceutical composition comprising an antigen-binding molecule that binds to HER3 and an antagonist of HER3-mediated signalling) to a subject in need of treatment.

In some aspects and embodiments, an antigen-binding molecule that binds to HER3 and an antagonist of HER3-mediated signalling may be provided as a combination therapy. In some embodiments, an antigen-binding molecule that binds to HER3 and an antagonist of HER3-mediated signalling may be administered simultaneously or sequentially.

Simultaneous administration refers to administration of the two or more agents together, for example as a pharmaceutical composition containing both agents (i.e. as a combined preparation), or immediately after one another (e.g. within 1, 4, 6, 8 or 12 hours), and optionally via the same route of administration, e.g. to the same artery, vein or other blood vessel.

Sequential administration refers to administration of one of the agents followed after a given time interval by separate administration of another agent. It is not required that the agents are administered by the same route, although this is the case in some embodiments. The time interval may be any time interval.

In some embodiments, therapeutic or prophylactic intervention according to the present disclosure comprises: (i) administering an antagonist of HER3-mediated signalling (e.g. an antagonist of HER3-mediated signalling described herein) to a subject having a cancer (e.g. a cancer characterised by the presence of genetic variation resulting in an increase in HER3-mediated signalling described herein), and (ii) administering to the subject an antigen-binding molecule that binds to HER3 (e.g. a HER3-binding antigen-binding molecule as described herein). In some embodiments, (i) and (ii) are performed simultaneously. In some embodiments, (i) and (ii) are performed sequentially (e.g. (i) may be followed by (ii), or (ii) may be followed by (i)).

Therapeutic or prophylactic intervention in accordance with the present disclosure may be effective to reduce the development or progression of the cancer, alleviate one or more symptoms of the cancer or reduce the pathology of cancer. The intervention may be effective to prevent progression of the cancer, e.g. to prevent worsening of, or to slow the rate of development of, the cancer. In some embodiments, the intervention may lead to an improvement in the cancer, e.g. a reduction in the symptoms of the cancer or a reduction in some other correlate of the severity/activity of the cancer. In some embodiments, the methods may prevent development of the cancer to a later stage (e.g. a more severe stage or metastasis).

In some embodiments, the therapeutic or prophylactic intervention may be aimed at one or more of: delaying/preventing the onset/progression of symptoms of the cancer, reducing the severity of symptoms of the cancer, reducing the survival/growth/invasion/metastasis of cells of the cancer, reducing the number of cells of the cancer and/or increasing survival of the subject.

In some embodiments, therapeutic or prophylactic intervention according to the present disclosure may be associated with one or more of: inhibition of the development/progression of the cancer, a delay to/prevention of onset of the cancer, a reduction in/delay to/prevention of tumor growth, a reduction in/delay to/prevention of tissue invasion, a reduction in/delay to/prevention of metastasis, a reduction in the severity of one or more symptoms of the cancer, a reduction in the number of cancer cells, a reduction in the cancer burden, a reduction in tumor size/volume, and/or an increase in survival of subjects having the cancer (e.g. progression free survival or overall survival).

Administration of the agents, pharmaceutical combinations and pharmaceutical compositions of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (Ed. A. Adejare), 23rd Edition (2020), Academic Press.

Administration of the articles of the present disclosure may be e.g. parenteral, systemic, topical, intracavitary, intravascular, intravenous, intra-arterial, intramuscular, intrathecal, intraocular, intraconjunctival, intratumoral, subcutaneous, intradermal, oral ortransdermal. Administration may be by injection, infusion or ingestion.

In some aspects and embodiments, articles of the present disclosure may be administered to a tissue/organ of interest (e.g. a tissue/organ affected by the disease/condition) affected by the condition (e.g. a tissue/organ in which symptoms of the disease/condition manifest). In some aspects and embodiments, articles of the present disclosure may be administered to the blood (i.e. intravenous/intra-arterial administration) by injection or infusion (e.g. via cannula), or may be administered subcutaneously or orally. In some aspects and embodiments, articles of the present disclosure may be administered to a tumor.

Multiple doses of the agents, pharmaceutical combinations and pharmaceutical compositions may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days: such as 4, 5, 6, 8, 9 or 10 days; 11, 12, 13, 15, 16 or 17 days; 18, 19, 20, 22, 23, or 24 days; or 25, 26, 27, 29, 30 or 31 days). That is, there may be one treatment event every 7 days, every 14 days, every 21 days, or every 28 days. There may be a treatment holiday between doses/administrations of about 7 days (plus or minus 3, 2, or 1 days), about 14 days (plus or minus 3, 2, or 1 days), about 21 days (plus or minus 3, 2, or 1 days), or about 28 days (plus or minus 3, 2, or 1 days), respectively.

In some embodiments, the intervention comprises additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy. In some embodiments, the therapeutic or prophylactic intervention comprises leukapheresis. In some embodiments, the therapeutic or prophylactic intervention comprises a stem cell transplant.

### Therapeutic/Prophylactic Intervention Employing an Antagonist of EGFR

Aspects and embodiments of therapeutic/prophylactic intervention according to the present disclosure employ antagonists of EGFR.

The present disclosure provides an antigen-binding molecule that binds to HER3 for use in a method of treating or preventing a cancer described herein, wherein the method further comprises administering an antagonist of EGFR. Also provided is an antagonist of EGFR for use in a method of treating or preventing a cancer described herein, wherein the method further comprises administering an antigen-binding molecule that binds to HER3.

Also provided is the use of an antigen-binding molecule that binds to HER3 in the manufacture of a medicament for use in a method of treating or preventing a cancer described herein, wherein the method further comprises administering an antagonist of EGFR. Also provided is the use of an antagonist of EGFR in the manufacture of a medicament for use in a method of treating or preventing a cancer described herein, wherein the method further comprises administering an antigen-binding molecule that binds to HER3.

Further provided is a method of treating or preventing a cancer described herein, the method comprising administering a therapeutically- or prophylactically-effective amount of (i) an antigen-binding molecule that binds to HER3 and (ii) an antagonist of EGFR to a subject in need of treatment.

The present disclosure also provides (i) an antigen-binding molecule that binds to HER3 and (ii) an antagonist of EGFR for use in a method of treating or preventing a cancer described herein in a subject. Also provided is the use of (i) an antigen-binding molecule that binds to HER3 and (ii) an antagonist of EGFR in the manufacture of a medicament for use in treating or preventing a cancer described herein in a subject. Also provided is a method of treating or preventing a cancer described herein in a subject, comprising administering to the subject a therapeutically- or prophylactically-effective amount of (i) an antigen-binding molecule that binds to HER3 and (ii) an antagonist of EGFR.

In embodiments in accordance with aspects of the preceding paragraph, provision of (i) and (ii) may be as a combination therapy. In some embodiments, (i) and (ii) may be provided simultaneously or sequentially.

In some embodiments, an antagonist of EGFR is an inhibitor of one or more function/activities of EGFR, or is an inhibitor of EGFR expression (i.e. gene and/or protein expression). Antagonists of EGFR include inhibitors of EGFR-mediated signalling.

In some embodiments, an antagonist of EGFR binds to EGFR. EGFR-binding antagonists of EGFR may inhibit one or more functions of EGFR. EGFR-binding antagonists of EGFR include e.g. EGFR-binding antibodies and antigen-binding fragments and derivatives thereof, EGFR-binding peptides/polypeptides, EGFR-binding nucleic acid aptamers, and EGFR-binding small molecule inhibitors of EGFR.

In some embodiments, an antagonist of EGFR is an agent that inhibits expression of EGFR. Such EGFR antagonists include small molecule inhibitors of EGFR expression and nucleic acid capable of preventing or reducing the expression of EGFR (e.g. antisense oligonucleotides).

In some embodiments, an antagonist of EGFR according to the present disclosure is a small molecule inhibitor of EGFR. In some embodiments, an antagonist of EGFR is a pan-ErbB inhibitor (e.g. sapitinib or Sym013). In some embodiments, an antagonist of EGFR is selected from gefitinib, erlotinib, lapatinib, afatinib, brigatinib, icotinib, osimertinib, vandetanib and dacomitinib.

In some embodiments, an antagonist of EGFR is an antigen-binding molecule that binds to EGFR, e.g. an EGFR-binding antibody or an antigen-binding fragment or derivatives thereof. In some embodiments, an antagonist of EGFR is selected from cetuximab, panitumumab, zalutumumab, duligotuzumab and matuzumab.

In some embodiments, an antigen-binding molecule that binds to EGFR comprises the CDRs of an antigen-binding molecule which is capable of binding to EGFR. In some embodiments, the antigen-binding molecule comprises the FRs of an antigen-binding molecule which is capable of binding to EGFR. In some embodiments, the antigen-binding molecule comprises the CDRs and the FRs of an antigen-binding molecule which is capable of binding to EGFR. That is, in some embodiments the antigen-binding molecule comprises the VH region and the VL region of an antigen-binding molecule which is capable of binding to EGFR.

In some embodiments, an antigen-binding molecule which is capable of binding to EGFR according to the present disclosure may be selected from: any embodiment of an antigen-binding molecule described in U.S. Pat. No. 6,217,866 B1 (which is hereby incorporated by reference in its entirety), cetuximab (described e.g. in U.S. Pat. No. 6,217,866 B1 and Wong et al., Clin Ther. (2005) 27(6):684-694; DrugBank Acc. No. DB00002), panitumumab (described e.g. in Foon et al., Int J Radiat Oncol Biol Phys. (2004) 58(3):984-990; DrugBank Acc. No. DB01269), zalutumumab (described e.g. in Bastholt et al., Radiother Oncol. (2007) 85(1):24-28; DrugBank Acc. No. DB12202), necitumumab (described e.g. in Kuenen et al., Clin Cancer Res. (2010) 16(6):1915-1923; DrugBank Acc. No. DB09559), nimotuzumab (described e.g. in Ramakrishnan et al., mAbs (2009) 1(1): 41-48; DrugBank Acc. No. DB06192), duligotuzumab (described e.g. in Fayette et al., Front Oncol. (2016) 6:232; DrugBank Acc. No. DB12142) and matuzumab (DrugBank Acc. No. DB05101). In some embodiments, the antigen-binding molecule is cetuximab.

In some embodiments the antigen-binding molecule is capable of binding the same region of EGFR, or an overlapping region of EGFR, to the region of EGFR which is bound by an antibody comprising the VH and VL sequences of cetuximab.

In some embodiments, the antigen-binding molecule comprises the CDRs of, or comprises the VH and VL of cetuximab.

In some embodiments, an antigen-binding molecule that binds to EGFR comprises:
a VH region incorporating the following CDRs:
   HC-CDR1 having the amino acid sequence of SEQ ID NO:78
   HC-CDR2 having the amino acid sequence of SEQ ID NO:79
   HC-CDR3 having the amino acid sequence of SEQ ID NO:80,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:82
   LC-CDR2 having the amino acid sequence of SEQ ID NO:83
   LC-CDR3 having the amino acid sequence of SEQ ID NO:84;
   or a variant thereof in which one or two or three amino acids in one or more of LC-CDR1, LC-CDR2 or LC-CDR3 are substituted with another amino acid.

In some embodiments, an antigen-binding molecule that binds to EGFR comprises:
a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:77; and
a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:81.

In some embodiments, an antigen-binding molecule that binds to EGFR comprises, or consists of:
(i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:85; and
(ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:86.

In some embodiments, an antigen-binding molecule that binds to EGFR is cetuximab.

### Diagnostic and Prognostic Methods, and Patient Selection

The present disclosure also provides diagnostic, prognostic and predictive methods in connection with the cancers described herein.

The methods may be performed in vitro on a sample obtained from a subject, or following processing of a sample obtained from a subject. Once the sample is collected, the subject is not required to be present for the in vitro method to be performed, and therefore the method may be one which is not practised on the human or animal body. However, in some embodiments the methods may be performed in vivo.

A sample may be taken from any tissue or bodily fluid. A sample may comprise or may be derived from: a quantity of blood; a quantity of serum derived from a subject's blood which may comprise the fluid portion of the blood obtained after removal of the fibrin clot and blood cells; a tissue sample or biopsy; pleural fluid; cerebrospinal fluid (CSF); or cells isolated from a subject. In some embodiments, the sample may be obtained or derived from a tissue or tissues which are affected by the disease/condition (e.g. tissue or tissues in which symptoms of the disease manifest, or which are involved in the pathogenesis of the disease/condition). In some embodiments, the sample may be obtained or derived from a cancer, tumor, or cells thereof.

The methods may be performed for the purpose of diagnosing a cancer (e.g. a cancer described herein). The methods may be performed for the purpose of prognosing/predicting the likely response of a subject to therapeutic/prophylactic intervention as described herein. The methods may be useful to predict the likely response to a given therapeutic/prophylactic intervention, e.g. in terms of efficacy, and may therefore by useful for supporting clinical decision-making. The methods may be performed for the purpose of identifying/selecting a subject for therapeutic/prophylactic intervention as described herein.

In some aspects and embodiments, the methods comprise analysing a subject's cancer to determine whether the cancer is a cancer described herein, e.g. a cancer according to an embodiment described in the section entitled ‘Cancers characterised by the absence of genetic variation resulting in an increase in HER3-mediated signalling’, or a cancer according to an embodiment described in the section entitled ‘Cancers characterised by the presence of genetic variation resulting in an increase in HER3-mediated signalling’, or a cancer according to an embodiment described in the section entitled ‘Cancers characterised by the presence of genetic variation resulting in an increase in the expression of, or an increase in the activity of a gene product of a chromosome 3q gene’, or a cancer according to an embodiment described in the section entitled ‘Cancers characterised by the presence of genetic variation resulting in an increase in the expression of, or an increase in the activity of a gene product of a chromosome 7p gene’, or a cancer according to an embodiment described in the section entitled ‘Cancers characterised by the presence of genetic variation resulting in a decrease in the expression of, or a decrease in the activity of a gene product of a chromosome 3p gene’.

In some embodiments, the methods comprise evaluating a cancer to determine whether it comprises genetic variation as described herein. In some embodiments, the methods comprise evaluating a cancer to determine whether it comprises cells comprising comprises genetic variation as described herein.

Aspects and embodiments of the present disclosure also comprise selecting a subject for therapeutic or prophylactic intervention in accordance with the present disclosure.

A subject having a cancer which is identified following such analysis to be a cancer described herein (e.g. a cancer according to an embodiment described in the section entitled ‘Cancers characterised by the absence of genetic variation resulting in an increase in HER3-mediated signalling’, or a cancer according to an embodiment described in the section entitled ‘Cancers characterised by the presence of genetic variation resulting in an increase in the expression of, or an increase in the activity of a gene product of a chromosome 3q gene’, or a cancer according to an embodiment described in the section entitled ‘Cancers characterised by the presence of genetic variation resulting in an increase in the expression of, or an increase in the activity of a gene product of a chromosome 7p gene’, or a cancer according to an embodiment described in the section entitled ‘Cancers characterised by the presence of genetic variation resulting in a decrease in the expression of, or a decrease in the activity of a gene product of a chromosome 3p gene’), may be selected for therapeutic/prophylactic intervention using an antigen-binding molecule that binds to HER3 according to the present disclosure.

A subject having a cancer which is identified following such analysis to be a cancer described herein (e.g. a cancer according to an embodiment described in the section entitled ‘Cancers characterised by the presence of genetic variation resulting in an increase in HER3-mediated signalling’), may be selected for therapeutic/prophylactic intervention using (i) an antagonist of HER3-mediated signalling according to the present disclosure, and (ii) an antigen-binding molecule that binds to HER3 according to the present disclosure.

In some embodiments, a subject is selected/not selected for therapeutic/prophylactic intervention in accordance with the present disclosure based on the result of analysis of their cancer to determine whether the cancer is a cancer described herein, e.g. a cancer according to an embodiment described in the section entitled ‘Cancers characterised by the absence of genetic variation resulting in an increase in HER3-mediated signalling’, or a cancer according to an embodiment described in the section entitled ‘Cancers characterised by the presence of genetic variation resulting in an increase in HER3-mediated signalling’, or a cancer according to an embodiment described in the section entitled ‘Cancers characterised by the presence of genetic variation resulting in an increase in the expression of, or an increase in the activity of a gene product of a chromosome 3q gene’, or a cancer according to an embodiment described in the section entitled ‘Cancers characterised by the presence of genetic variation resulting in an increase in the expression of, or an increase in the activity of a gene product of a chromosome 7p gene’, or a cancer according to an embodiment described in the section entitled ‘Cancers characterised by the presence of genetic variation resulting in a decrease in the expression of, or a decrease in the activity of a gene product of a chromosome 3p gene’.

In some aspects and embodiments, the methods comprise:
(a) analysing a subject's cancer to determine whether the cancer is a cancer according to an embodiment described in the section entitled ‘Cancers characterised by the absence of genetic variation resulting in an increase in HER3-mediated signalling’, or a cancer according to an embodiment described in the section entitled ‘Cancers characterised by the presence of genetic variation resulting in an increase in the expression of, or an increase in the activity of a gene product of a chromosome 3q gene’, or a cancer according to an embodiment described in the section entitled ‘Cancers characterised by the presence of genetic variation resulting in an increase in the expression of, or an increase in the activity of a gene product of a chromosome 7p gene’, or a cancer according to an embodiment described in the section entitled ‘Cancers characterised by the presence of genetic variation resulting in a decrease in the expression of, or a decrease in the activity of a gene product of a chromosome 3p gene’; and
(b) selecting a subject for treatment with an antigen-binding molecule that binds to HER3 according to the present disclosure where the subject's cancer is determined in step (a) to be a cancer according to an embodiment described in the section entitled ‘Cancers characterised by the absence of genetic variation resulting in an increase in HER3-mediated signalling’, or a cancer according to an embodiment described in the section entitled ‘Cancers characterised by the presence of genetic variation resulting in an increase in the expression of, or an increase in the activity of a gene product of a chromosome 3q gene’, or a cancer according to an embodiment described in the section entitled ‘Cancers characterised by the presence of genetic variation resulting in an increase in the expression of, or an increase in the activity of a gene product of a chromosome 7p gene’, or a cancer according to an embodiment described in the section entitled ‘Cancers characterised by the presence of genetic variation resulting in a decrease in the expression of, or a decrease in the activity of a gene product of a chromosome 3p gene’.

In some embodiments, the methods further comprise:
(c) administering an antigen-binding molecule that binds to HER3 according to the present disclosure to a subject selected for treatment in step (b).

In some aspects and embodiments, the methods comprise:
(a) analysing a subject's cancer to determine whether the cancer is a cancer according to an embodiment described in the section entitled ‘Cancers characterised by the presence of genetic variation resulting in an increase in HER3-mediated signalling’; and
(b) selecting a subject for treatment with (i) an antagonist of HER3-mediated signalling according to the present disclosure, and (ii) an antigen-binding molecule that binds to HER3 according to the present disclosure, where the subject's cancer is determined in step (a) to comprise or to be a cancer according to an embodiment described in the section entitled ‘Cancers characterised by the presence of genetic variation resulting in an increase in HER3-mediated signalling’.

In some embodiments, the methods further comprise:
(c) administering (i) an antagonist of HER3-mediated signalling according to the present disclosure, and (ii) an antigen-binding molecule that binds to HER3 according to the present disclosure, to a subject selected for treatment in step (b).

In some aspects and embodiments, the methods comprise:
(a) analysing a subject's cancer to determine whether the cancer is a cancer according to an embodiment described in the section entitled ‘Cancers characterised by the presence of genetic variation resulting in an increase in HER3-mediated signalling’; and
(b) selecting a subject for treatment with (i) an antagonist of HER3-mediated signalling according to the present disclosure, and (ii) an antigen-binding molecule that binds to HER3 according to the present disclosure, where the subject's cancer is determined in step (a) to comprise or to be a cancer according to an embodiment described in the section entitled ‘Cancers characterised by the presence of genetic variation resulting in an increase in HER3-mediated signalling’;
(c) administering an antagonist of HER3-mediated signalling according to the present disclosure to a subject selected for treatment in step (b); and
(d) administering an antigen-binding molecule that binds to HER3 according to the present disclosure to a subject selected for treatment in step (b).

It will be appreciated that steps (c) and (d) of the preceding paragraph may be performed simultaneously or sequentially.

In accordance with the various aspects described above, in some embodiments the methods further comprise:
(c) administering an antagonist of EGFR according to the present disclosure to the subject selected for treatment in step (b).

Analysis of a cancer may comprise analysing the nucleotide sequence(s) of gene(s)/gene region(s) described herein (e.g. in a nucleic acid-containing sample obtained from the cancer), to determine whether the cancer comprises genetic variation as described herein. Analysis may be of cells of a cancer. Analysis may be of a nucleic acid-containing sample/biopsy obtained from a cancer/cells thereof. Such analysis is preferably performed in vitro.

Methods for evaluating the nucleotide sequence(s) of genes of interest are well known in the art, and include e.g. sequencing by the classic chain termination method, and next generation sequencing (NGS) technologies, which are reviewed e.g. by Metzker, M. L., Nat Rev Genet (2010) 11(1): 31-46 (hereby incorporated by reference in its entirety). Further methods for evaluating the nucleotide sequence(s) of genes of interest for the presence/absence of genetic variation include restriction fragment length polymorphism identification (RFLPI) of genomic DNA, random amplified polymorphic detection (RAPD) of genomic DNA, amplified fragment length polymorphism detection (AFLPD), multiple locus variable number tandem repeat (VNTR) analysis (MLVA), SNP genotyping, multilocus sequence typing, allele specific oligonucleotide (ASO) probes, and oligonucleotide microarrays or beads. Other suitable methods are described e.g. in Edenberg H J and Liu Y, Cold Spring Harb Protoc; 2009; doi:10.1101/pdb.top62, and Tsuchihashi Z and Dracopoli N C, Pharmacogenomics J., 2002, 2:103-110.

In further aspects, the present disclosure provides methods for determining whether a subject is likely to respond to therapeutic or prophylactic intervention using an antigen-binding molecule that binds to HER3.

The response of a subject/cancer to a given therapeutic or prophylactic intervention may be evaluated in accordance with the response evaluation criteria in solid tumours (RECIST) criteria, e.g. the RECIST 1.1 criteria as described in Eisenhauer et al., Eur J Cancer. 2009 January; 45(2):228-47, which is hereby incorporated by reference in its entirety. In some embodiments, subjects may be evaluated in accordance with the Revised Criteria for Response Assessment: The Lugano Classification (described e.g. in Cheson et al., J Clin Oncol (2014) 32: 3059-3068, incorporated by reference hereinabove) in order to determine their response to treatment. In some embodiments, a subject/cancer is considered to ‘respond’ to a given intervention if one of the following is achieved: complete response, partial response, or stable disease. In some embodiments, a subject/cancer is considered to ‘respond’ to a given intervention if one of the following is achieved: complete response or partial response.

A complete response (CR) refers to a complete macroscopic disappearance of all target and/or non-target tumours. A CR may involve normalisation of tumour marker level. A partial response (PR) refers to a reduction of at least 30% in the sum of all target tumour diameters compared to baseline sum diameters calculated before treatment. Stable disease refers to neither partial response nor progressive disease compared to the tumour burden at the start of treatment. Progressive disease (PD) refers to an at least a 20% increase in the sum of diameters of target lesions taking as reference the smallest sum on study=nadir (this includes the baseline sum if that is the smallest on study). In addition to the relative increase of 20%, the sum must also demonstrate an absolute increase of at least 5 mm. Unequivocal progression of existing non target lesions or the appearance of one or more new lesions will also constitute progressive disease.

In some embodiments, the methods comprise: analysing a subject's cancer to determine whether the cancer is a cancer described herein, wherein a subject having determined to comprise a cancer according to an embodiment described in the section entitled ‘Cancers characterised by the absence of genetic variation resulting in an increase in HER3-mediated signalling’ is determined to be likely to respond to therapeutic or prophylactic intervention using an antigen-binding molecule that binds to HER3 (i.e. as a monotherapy).

In some embodiments, the methods comprise: analysing a subject's cancer to determine whether the cancer is a cancer described herein, wherein a subject having determined to comprise a cancer according to an embodiment described in the section entitled ‘Cancers characterised by the presence of genetic variation resulting in an increase in the expression of, or an increase in the activity of a gene product of a chromosome 3q gene’ is determined to be likely to respond to therapeutic or prophylactic intervention using an antigen-binding molecule that binds to HER3 (i.e. as a monotherapy).

In some embodiments, the methods comprise: analysing a subject's cancer to determine whether the cancer is a cancer described herein, wherein a subject having determined to comprise a cancer according to an embodiment described in the section entitled ‘Cancers characterised by the presence of genetic variation resulting in an increase in the expression of, or an increase in the activity of a gene product of a chromosome 7p gene’ is determined to be likely to respond to therapeutic or prophylactic intervention using an antigen-binding molecule that binds to HER3 (i.e. as a monotherapy).

In some embodiments, the methods comprise: analysing a subject's cancer to determine whether the cancer is a cancer described herein, wherein a subject having determined to comprise a cancer according to an embodiment described in the section entitled ‘Cancers characterised by the presence of genetic variation resulting in a decrease in the expression of, or a decrease in the activity of a gene product of a chromosome 3p gene’ is determined to be likely to respond to therapeutic or prophylactic intervention using an antigen-binding molecule that binds to HER3 (i.e. as a monotherapy).

In some embodiments, the methods comprise: analysing a subject's cancer to determine whether the cancer is a cancer described herein, wherein a subject having determined to comprise a cancer according to an embodiment described in the section entitled ‘Cancers characterised by the presence of genetic variation resulting in an increase in HER3-mediated signalling’ is determined to be likely not to respond to therapeutic or prophylactic intervention using an antigen-binding molecule that binds to HER3 (i.e. as a monotherapy).

### Subjects

The subject in accordance with aspects described herein may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer, e.g. a cancer described herein), may be suspected of having such a disease/condition, or may be at risk of developing/contracting such a disease/condition.

In some embodiments, the subject to be treated according to a therapeutic or prophylactic method of the present disclosure herein is a subject having, or at risk of developing, a cancer, e.g. a cancer described herein. In embodiments according to the present disclosure, a subject may be selected for treatment according to the methods based on characterization for certain markers of such disease/condition.

In some embodiments, a subject may be selected for treatment described herein based on the detection of a cancer described herein, e.g. in a sample obtained from the subject (e.g. a biopsy, e.g. of a tumor).

### Kits

The present disclosure also provides kits of parts. A kit according to the present disclosure may comprise components for performing a method described herein, in whole or in part.

In some embodiments, a kit may comprise means for evaluating a cancer to determine whether it comprises genetic variation as described herein. In some embodiments, a kit may further comprise an antigen-binding molecule that binds to HER3 as described herein. In some embodiments, a kit may further comprise an antagonist of HER3-mediated signalling as described herein. In some embodiments, a kit may further comprise an antagonist of EGFR as described herein.

Means for evaluating a cancer to determine whether it comprises genetic variation described herein may comprise e.g. one or more oligonucleotides having complementarity to the nucleotide sequence of one or more gene(s) described herein.

The kit may comprise instructions for evaluation of a cancer to determine whether it comprises genetic variation as described herein. The kit may comprise instructions for administering an antigen-binding molecule that binds to HER3 as described herein to a subject. The kit may further comprise instructions for administering an antagonist of HER3-mediated signalling as described herein to a subject. The kit may further comprise instructions for administering an antagonist of EGFR as described herein to a subject.

The kit may further comprise reagents, buffers and/or standards required for execution of a method according to the present disclosure. Kits according to the present disclosure may include instructions for use, e.g. in the form of an instruction booklet or leaflet. The instructions may include a protocol for performing any one or more of the methods described herein.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780) software. When using such software, the default parameters, e.g. for gap penalty and extension penalty, are preferably used.

Sequences

| SEQ | | |
|---|---|---|
| ID NO: | DESCRIPTION | SEQUENCE |
| 1 | Human HER3 | MRANDALQVLGLLFSLARGSEVGNSQAVCPGTLNGLSVTGDAENQYQTLYKLYERCEVVMGNLEIVLTGHN |
| | isoform 1 | ADLSFLQWIREVTGYVLVAMNEFSTLPLPNLRVVRGTQVYDGKFAIFVMLNYNTNSSHALRQLRLTQLTEILS |
| | (UniProt: | GGVYIEKNDKLCHMDTIDWRDIVRDRDAEIVVKDNGRSCPPCHEVCKGRCWGPGSEDCQTLTKTICAPQCN |
| | P21860-1, v1) | GHCFGPNPNQCCHDECAGGCSGPQDTDCFACRHFNDSGACVPRCPQPLVYNKLTFQLEPNPHTKYQYGG |
| | | VCVASCPHNFVVDQTSCVRACPPDKMEVDKNGLKMCEPCGGLCPKACEGTGSGSRFQTVDSSNIDGFVN |
| | | CTKILGNLDFLITGLNGDPWHKIPALDPEKLNVFRTVREITGYLNIQSWPPHMHNFSVFSNLTTIGGRSLYNRG |
| | | FSLLIMKNLNVTSLGFRSLKEISAGRIYISANRQLCYHHSLNWTKVLRGPTEERLDIKHNRPRRDCVAEGKVC |
| | | DPLCSSGGCWGPGPGQCLSCRNYSRGGVCVTHCNFLNGEPREFAHEAECFSCHPECQPMEGTATQNGS |
| | | GSDTCAQCAHFRDGPHCVSSCPHGVLGAKGPIYKYPDVQNECRPCHENCTQGCKGPELQDCLGQTLVLIG |
| | | KTHLTMALTVIAGLVVIFMMLGGTFLYWRGRRIQNKRAMRRYLERGESIEPLDPSEKANKVLARIFKETELRK |
| | | LKVLGSGVFGTVHKGVWIPEGESIKIPVCIKVIEDKSGRQSFQAVTDHMLAIGSLDHAHIVRLLGLCPGSSLQL |
| | | VTQYLPLGSLLDHVRQHRGALGPQLLLNWGVQIAKGMYYLEEHGMVHRNLAARNVLLKSPSQVQVADFGV |
| | | ADLLPPDDKQLLYSEAKTPIKWMALESIHFGKYTHQSDVWSYGVTVWELMTFGAEPYAGLRLAEVPDLLEK |
| | | GERLAQPQICTIDVYMVMVKCWMIDENIRPTFKELANEFTRMARDPPRYLVIKRESGPGIAPGPEPHGLTNK |
| | | KLEEVELEPELDLDLDLEAEEDNLATTTLGSALSLPVGTLNRPRGSQSLLSPSSGYMPMNQGNLGESCQES |
| | | AVSGSSERCPRPVSLHPMPRGCLASESSEGHVTGSEAELQEKVSMCRSRSRSRSPRPRGDSAYHSQRHS |
| | | LLTPVTPLSPPGLEEEDVNGYVMPDTHLKGTPSSREGTLSSVGLSSVLGTEEEDEDEEYEYMNRRRRHSPP |
| | | HPPRPSSLEELGYEYMDVGSDLSASLGSTQSCPLHPVPIMPTAGTTPDEDYEYMNRQRDGGGPGGDYAA |
| | | MGACPASEQGYEEMRAFQGPGHQAPHVHYARLKTLRSLEATDSAFDNPDYWHSRLFPKANAQRT |
| 2 | Human HER3 | MRANDALQVLGLLFSLARGSEVGNSQAVCPGTLNGLSVTGDAENQYQTLYKLYERCEVVMGNLEIVLTGHN |
| | isoform 2 | ADLSFLQWIREVTGYVLVAMNEFSTLPLPNLRVVRGTQVYDGKFAIFVMLNYNTNSSHALRQLRLTQLTGQF |
| | (UniProt: | PMVPSGLTPQPAQDWYLLDDDPRLLTLSASSKVPVTLAAV |
| | P21860-2) | |
| 3 | Human HER3 | MRANDALQVLGLLFSLARGSEVGNSQAVCPGTLNGLSVTGDAENQYQTLYKLYERCEVVMGNLEIVLTGHN |
| | isoform 3 | ADLSFLQWIREVTGYVLVAMNEFSTLPLPNLRVVRGTQVYDGKFAIFVMLNYNTNSSHALRQLRLTQLTEILS |
| | (UniProt: | GGVYIEKNDKLCHMDTIDWRDIVRDRDAEIVVKDNGRSCPPCHEVCKGRCWGPGSEDCQTLTKTICAPQCN |
| | P21860-3) | GHCFGPNPNQCCHDECAGGCSGPQDTDCFACRHFNDSGACVPRCPQPLVYNKLTFQLEPNPHTKYQYGG |
| | | VCVASCPHNFVVDQTSCVRACPPDKMEVDKNGLKMCEPCGGLCPKAF |
| 4 | Human HER3 | MGNLEIVLTGHNADLSFLQWIREVTGYVLVAMNEFSTLPLPNLRVVRGTQVYDGKFAIFVMLNYNTNSSHAL |
| | isoform 4 | RQLRLTQLTEILSGGVYIEKNDKLCHMDTIDWRDIVRDRDAEIVVKDNGRSCPPCHEVCKGRCWGPGSEDC |
| | (UniProt: | QTLTKTICAPQCNGHCFGPNPNQCCHDECAGGCSGPQDTDCFACRHFNDSGACVPRCPQPLVYNKLTFQL |
| | P21860-4) | EPNPHTKYQYGGVCVASCPHNFVVDQTSCVRACPPDKMEVDKNGLKMCEPCGGLCPKACEGTGSGSRFQ |
| | | TVDSSNIDGFVNCTKILGNLDFLITGLNGDPWHKIPALDPEKLNVFRTVREITGYLNIQSWPPHMHNFSVFSNL |
| | | TTIGGRSLYNRGFSLLIMKNLNVTSLGFRSLKEISAGRIYISANRQLCYHHSLNWTKVLRGPTEERLDIKHNRP |
| | | RRDCVAEGKVCDPLCSSGGCWGPGPGQCLSCRNYSRGGVCVTHCNFLNGEPREFAHEAECFSCHPECQ |
| | | PMEGTATCNGSGSDTCAQCAHFRDGPHCVSSCPHGVLGAKGPIYKYPDVQNECRPCHENCTQGCKGPEL |
| | | QDCLGQTLVLIGKTHLTMALTVIAGLVVIFMMLGGTFLYWRGRRIQNKRAMRRYLERGESIEPLDPSEKANKV |
| | | LARIFKETELRKLKVLGSGVFGTVHKGVWIPEGESIKIPVCIKVIEDKSGRQSFQAVTDHMLAIGSLDHAHIVRL |
| | | LGLCPGSSLQLVTQYLPLGSLLDHVRQHRGALGPQLLLNWGVQIAKGMYYLEEHGMVHRNLAARNVLLKSP |
| | | SQVQVADFGVADLLPPDDKQLLYSEAKTPIKWMALESIHFGKYTHQSDVWSYGVTVWELMTFGAEPYAGLR |
| | | LAEVPDLLEKGERLAQPQICTIDVYMVMVKCWMIDENIRPTFKELANEFTRMARDPPRYLVIKRESGPGIAPG |
| | | PEPHGLTNKKLEEVELEPELDLDLDLEAEEDNLATTTLGSALSLPVGTLNRPRGSQSLLSPSSGYMPMNQGN |
| | | LGESCQESAVSGSSERCPRPVSLHPMPRGCLASESSEGHVTGSEAELQEKVSMCRSRSRSRSPRPRGDS |
| | | AYHSQRHSLLTPVTPLSPPGLEEEDVNGYVMPDTHLKGTPSSREGTLSSVGLSSVLGTEEEDEDEEYEYMN |
| | | RRRRHSPPHPPRPSSLEELGYEYMDVGSDLSASLGSTQSCPLHPVPIMPTAGTTPDEDYEYMNRQRDGGG |
| | | PGGDYAAMGACPASEQGYEEMRAFQGPGHQAPHVHYARLKTLRSLEATDSAFDNPDYWHSRLFPKANAQ |
| | | RT |
| 5 | Human HER3 | MALTVIAGLVVIFMMLGGTFLYWRGRRIQNKRAMRRYLERGESIEPLDPSEKANKVLARIFKETELRKLKVLG |
| | isoform 5 | SGVFGTVHKGVWIPEGESIKIPVCIKVIEDKSGRQSFQAVTDHMLAIGSLDHAHIVRLLGLCPGSSLQLVTQYL |
| | (UniProt: | PLGSLLDHVRQHRGALGPQLLLNWGVQIAKGMYYLEEHGMVHRNLAARNVLLKSPSQVQVADFGVADLLP |
| | P21860-5) | PDDKQLLYSEAKTPIKWMALESIHFGKYTHQSDVWSYGVTVWELMTFGAEPYAGLRLAEVPDLLEKGERLA |
| | | QPQICTIDVYMVMVKCWMIDENIRPTFKELANEFTRMARDPPRYLVIKRESGPGIAPGPEPHGLTNKKLEEVE |
| | | LEPELDLDLDLEAEEDNLATTTLGSALSLPVGTLNRPRGSQSLLSPSSGYMPMNQGNLGESCQESAVSGSS |
| | | ERCPRPVSLHPMPRGCLASESSEGHVTGSEAELQEKVSMCRSRSRSRSPRPRGDSAYHSQRHSLLTPVTP |
| | | LSPPGLEEEDVNGYVMPDTHLKGTPSSREGTLSSVGLSSVLGTEEEDEDEEYEYMNRRRRHSPPHPPRPS |
| | | SLEELGYEYMDVGSDLSASLGSTQSCPLHPVPIMPTAGTTPDEDYEYMNRQRDGGGPGGDYAAMGACPA |
| | | SEQGYEEMRAFQGPGHQAPHVHYARLKTLRSLEATDSAFDNPDYWHSRLFPKANAQRT |
| 6 | Mature human | SEVGNSQAVCPGTLNGLSVTGDAENQYQTLYKLYERCEVVMGNLEIVLTGHNADLSFLQWIREVTGYVLVA |
| | HER3 isoform 1 | MNEFSTLPLPNLRVVRGTQVYDGKFAIFVMLNYNTNSSHALRQLRLTQLTEILSGGVYIEKNDKLCHMDTIDW |
| | (UniProt: | RDIVRDRDAEIVVKDNGRSCPPCHEVCKGRCWGPGSEDCQTLTKTICAPQCNGHCFGPNPNQCCHDECA |
| | P21860-1, v1 | GGCSGPQDTDCFACRHFNDSGACVPRCPQPLVYNKLTFQLEPNPHTKYQYGGVCVASCPHNFVVDQTSC |
| | positions 20 to | VRACPPDKMEVDKNGLKMCEPCGGLCPKACEGTGSGSRFQTVDSSNIDGFVNCTKILGNLDFLITGLNGDP |
| | 1342) | WHKIPALDPEKLNVFRTVREITGYLNIQSWPPHMHNFSVFSNLTTIGGRSLYNRGFSLLIMKNLNVTSLGFRS |
| | | LKEISAGRIYISANRQLCYHHSLNWTKVLRGPTEERLDIKHNRPRRDCVAEGKVCDPLCSSGGCWGPGPGQ |
| | | CLSCRNYSRGGVCVTHCNFLNGEPREFAHEAECFSCHPECQPMEGTATCNGSGSDTCAQCAHFRDGPHC |
| | | VSSCPHGVLGAKGPIYKYPDVQNECRPCHENCTQGCKGPELQDCLGQTLVLIGKTHLTMALTVIAGLVVIFM |
| | | MLGGTFLYWRGRRIQNKRAMRRYLERGESIEPLDPSEKANKVLARIFKETELRKLKVLGSGVFGTVHKGVWI |
| | | PEGESIKIPVCIKVIEDKSGRQSFQAVTDHMLAIGSLDHAHIVRLLGLCPGSSLQLVTQYLPLGSLLDHVRQHR |
| | | GALGPQLLLNWGVQIAKGMYYLEEHGMVHRNLAARNVLLKSPSQVQVADFGVADLLPPDDKQLLYSEAKTP |
| | | IKWMALESIHFGKYTHQSDVWSYGVTVWELMTFGAEPYAGLRLAEVPDLLEKGERLAQPQICTIDVYMVMV |
| | | KCWMIDENIRPTFKELANEFTRMARDPPRYLVIKRESGPGIAPGPEPHGLTNKKLEEVELEPELDLDLDLEAE |
| | | EDNLATTTLGSALSLPVGTLNRPRGSQSLLSPSSGYMPMNQGNLGESCQESAVSGSSERCPRPVSLHPMP |
| | | RGCLASESSEGHVTGSEAELQEKVSMCRSRSRSRSPRPRGDSAYHSQRHSLLTPVTPLSPPGLEEEDVNG |
| | | YVMPDTHLKGTPSSREGTLSSVGLSSVLGTEEEDEDEEYEYMNRRRRHSPPHPPRPSSLEELGYEYMDVG |
| | | SDLSASLGSTQSCPLHPVPIMPTAGTTPDEDYEYMNRQRDGGGPGGDYAAMGACPASEQGYEEMRAFQG |
| | | PGHQAPHVHYARLKTLRSLEATDSAFDNPDYWHSRLFPKANAQRT |
| 7 | Mature human | SEVGNSQAVCPGTLNGLSVTGDAENQYQTLYKLYERCEVVMGNLEIVLTGHNADLSFLQWIREVTGYVLVA |
| | HER3 isoform 2 | MNEFSTLPLPNLRVVRGTQVYDGKFAIFVMLNYNTNSSHALRQLRLTQLTGQFPMVPSGLTPQPAQDWYLL |
| | (UniProt: | DDDPRLLTLSASSKVPVTLAAV |
| | P21860-2 | |
| | positions 20 to | |
| | 183) | |
| 8 | Mature human | SEVGNSQAVCPGTLNGLSVTGDAENQYQTLYKLYERCEVVMGNLEIVLTGHNADLSFLQWIREVTGYVLVA |
| | HER3 isoform 3 | MNEFSTLPLPNLRVVRGTQVYDGKFAIFVMLNYNTNSSHALRQLRLTQLTEILSGGVYIEKNDKLCHMDTIDW |
| | (UniProt: | RDIVRDRDAEIVVKDNGRSCPPCHEVCKGRCWGPGSEDCQTLTKTICAPQCNGHCFGPNPNQCCHDECA |
| | P21860-3 | GGCSGPQDTDCFACRHFNDSGACVPRCPQPLVYNKLTFQLEPNPHTKYQYGGVCVASCPHNFVVDQTSC |
| | positions 20 to | VRACPPDKMEVDKNGLKMCEPCGGLCPKAF |
| | 331) | |
| 9 | Human HER3 | SEVGNSQAVCPGTLNGLSVTGDAENQYQTLYKLYERCEVVMGNLEIVLTGHNADLSFLQWIREVTGYVLVA |
| | isoform 1 | MNEFSTLPLPNLRVVRGTQVYDGKFAIFVMLNYNTNSSHALRQLRLTQLTEILSGGVYIEKNDKLCHMDTIDW |
| | extracellular | RDIVRDRDAEIVVKDNGRSCPPCHEVCKGRCWGPGSEDCQTLTKTICAPQCNGHCFGPNPNQCCHDECA |
| | region (UniProt: | GGCSGPQDTDCFACRHFNDSGACVPRCPQPLVYNKLTFQLEPNPHTKYQYGGVCVASCPHNFVVDQTSC |
| | P21860-1, v1 | VRACPPDKMEVDKNGLKMCEPCGGLCPKACEGTGSGSRFQTVDSSNIDGFVNCTKILGNLDFLITGLNGDP |
| | positions 20 to | WHKIPALDPEKLNVFRTVREITGYLNIQSWPPHMHNFSVFSNLTTIGGRSLYNRGFSLLIMKNLNVTSLGFRS |
| | 643) | LKEISAGRIYISANRQLCYHHSLNWTKVLRGPTEERLDIKHNRPRRDCVAEGKVCDPLCSSGGCWGPGPGQ |
| | | CLSCRNYSRGGVCVTHCNFLNGEPREFAHEAECFSCHPECQPMEGTATCNGSGSDTCAQCAHFRDGPHC |
| | | VSSCPHGVLGAKGPIYKYPDVQNECRPCHENCTQGCKGPELQDCLGQTLVLIGKTHLT |
| 10 | Human HER3 | MALTVIAGLVVIFMMLGGTFL |
| | isoform 1 | |
| | transmembrane | |
| | domain (UniProt: | |
| | P21860-1, v1 | |
| | positions 644 to | |
| | 664) | |
| 11 | Human HER3 | YWRGRRIQNKRAMRRYLERGESIEPLDPSEKANKVLARIFKETELRKLKVLGSGVFGTVHKGVWIPEGESIKI |
| | isoform 1 | PVCIKVIEDKSGRQSFQAVTDHMLAIGSLDHAHIVRLLGLCPGSSLQLVTQYLPLGSLLDHVRQHRGALGPQL |
| | cytoplasmic | LLNWGVQIAKGMYYLEEHGMVHRNLAARNVLLKSPSQVQVADFGVADLLPPDDKQLLYSEAKTPIKWMALE |
| | domain (UniProt: | SIHFGKYTHQSDVWSYGVTVWELMTFGAEPYAGLRLAEVPDLLEKGERLAQPQICTIDVYMVMVKCWMIDE |
| | P21860-1, v1 | NIRPTFKELANEFTRMARDPPRYLVIKRESGPGIAPGPEPHGLTNKKLEEVELEPELDLDLDLEAEEDNLATTT |
| | positions 665 to | LGSALSLPVGTLNRPRGSQSLLSPSSGYMPMNQGNLGESCQESAVSGSSERCPRPVSLHPMPRGCLASES |
| | 1342) | SEGHVTGSEAELQEKVSMCRSRSRSRSPRPRGDSAYHSQRHSLLTPVTPLSPPGLEEEDVNGYVMPDTHL |
| | | KGTPSSREGTLSSVGLSSVLGTEEEDEDEEYEYMNRRRRHSPPHPPRPSSLEELGYEYMDVGSDLSASLG |
| | | STQSCPLHPVPIMPTAGTTPDEDYEYMNRQRDGGGPGGDYAAMGACPASEQGYEEMRAFQGPGHQAPH |
| | | VHYARLKTLRSLEATDSAFDNPDYWHSRLFPKANAQRT |
| 12 | Human HER3 | YWRGRRIQNKRAMRRYLERGESIEPLDPSEKANKVLARIFKETE |
| | isoform 1 | |
| | juxtamembrane | |
| | segment | |
| | (UniProt: | |
| | P21860-1, v1 | |
| | positions 665 to | |
| | 708) | |
| 13 | Human HER3 | LRKLKVLGSGVFGTVHKGVWIPEGESIKIPVCIKVIEDKSGRQSFQAVTDHMLAIGSLDH |
| | isoform 1 protein | AHIVRLLGLCPGSSLQLVTQYLPLGSLLDHVRQHRGALGPQLLLNWGVQIAKGMYYLEEH |
| | kinase domain | GMVHRNLAARNVLLKSPSQVQVADFGVADLLPPDDKQLLYSEAKTPIKWMALESIHFGKY |
| | (UniProt: | THQSDVWSYGVTVWELMTFGAEPYAGLRLAEVPDLLEKGERLAQPQICTIDVYMVMVKCW |
| | P21860-1, v1 | MIDENIRPTFKELANEFT |
| | positions 709 to | |
| | 966) | |
| 14 | Human HER3 | RMARDPPRYLVIKRESGPGIAPGPEPHGLTNKKLEEVELEPELDLDLDLEAEEDNLATTTLGSALSLPVGTLN |
| | isoform 1 C | RPRGSQSLLSPSSGYMPMNQGNLGESCQESAVSGSSERCPRPVSLHPMPRGCLASESSEGHVTGSEAEL |
| | terminal | QEKVSMCRSRSRSRSPRPRGDSAYHSQRHSLLTPVTPLSPPGLEEEDVNGYVMPDTHLKGTPSSREGTLS |
| | segment | SVGLSSVLGTEEEDEDEEYEYMNRRRRHSPPHPPRPSSLEELGYEYMDVGSDLSASLGSTQSCPLHPVPIM |
| | (UniProt: | PTAGTTPDEDYEYMNRQRDGGGPGGDYAAMGACPASEQGYEEMRAFQGPGHQAPHVHYARLKTLRSLE |
| | P21860-1, v1 | ATDSAFDNPDYWHSRLFPKANAQRT |
| | positions 967 to | |
| | 1342) | |
| 15 | Human HER3 | SEVGNSQAVCPGTLNGLSVTGDAENQYQTLYKLYERCEVVMGNLEIVLTGHNADLSFLQWIREVTGYVLVA |
| | extracellular | MNEFSTLPLPNLRVVRGTQVYDGKFAIFVMLNYNTNSSHALRQLRLTQLTEILSGGVYIEKNDKLCHMDTIDW |
| | region | RDIVRDRDAEIVVKDNGRSC |
| | subdomain I | |
| | (UniProt: | |
| | P21860-1, v1 | |
| | positions 20 to | |
| | 183) | |
| 16 | Human HER3 | PPCHEVCKGRCWGPGSEDCQTLTKTICAPQCNGHCFGPNPNQCCHDECAGGCSGPQDTDCFACRHFND |
| | extracellular | SGACVPRCPQPLVYNKLTFQLEPNPHTKYQYGGVCVASCPHNFVVDQTSCVRACPPDKMEVDKNGLKMC |
| | region | EPCGGLCPK |
| | subdomain II | |
| | (UniProt: | |
| | P21860-1, v1 | |
| | positions 184 to | |
| | 329) | |
| 17 | Human HER3 | ACEGTGSGSRFQTVDSSNIDGFVNCTKILGNLDFLITGLNGDPWHKIPALDPEKLNVFRTVREITGYLNIQSW |
| | extracellular | PPHMHNFSVFSNLTTIGGRSLYNRGFSLLIMKNLNVTSLGFRSLKEISAGRIYISANRQLCYHHSLNWTKVLR |
| | region | GPTEERLDIKHNRPRRDCVA |
| | subdomain III | |
| | (UniProt: | |
| | P21860-1, v1 | |
| | positions 330 to | |
| | 495) | |
| 18 | Human HER3 | EGKVCDPLCSSGGCWGPGPGQCLSCRNYSRGGVCVTHCNFLNGEPREFAHEAECFSCHPECQPMEGTA |
| | extracellular | TCNGSGSDTCAQCAHFRDGPHCVSSCPHGVLGAKGPIYKYPDVQNECRPCHENCTQGCKGPELQDCLGQ |
| | region | TLVLIGKTHLT |
| | subdomain IV | |
| | (UniProt: | |
| | P21860-1, v1 | |
| | positions 496 to | |
| | 643) | |
| 19 | Human HER3 | QPLVYNKLTFQLEPNPH |
| | extracellular | |
| | region | |
| | subdomain II | |
| | dimerisation loop | |
| | (UniProt: | |
| | P21860-1, v1 | |
| | positions 261 to | |
| | 278) | |
| 20 | Rhesus | MGNLEIVLTGHNADLSFLQWIREVTGYVLVAMNEFSTLPLPNLRVVRGTQVYDGKFAIFVMLNYNTNSSHAL |
| | macaque HER3 | RQLRLTQLTEILSGGVYIEKNDKLCHMDTIDWKDIVRDQDAEIVVKDNGRSCPLCHEVCKGRCWGPGPEDC |
| | (UniProt: | QTLTKTICAPQCNGHCFGPNPNQCCHDECAGGCSGPQDTDCFACRHFNDSGACVPRCPQPLVYNKLTFQL |
| | F7HEH3-1, v2) | EPNPHTKYQYGGVCVASCPHNFVVDQTSCVRACPPDKMEVDKNGLKMCEPCGGLCPKACEGTGSGSRFQ |
| | | TVDSSNIDGFVNCTKILGNLDFLITGLNGDPWHKIPALDPEKLNVFRTVREITGYLNIQSWPPHMYNFSVFSNL |
| | | TTIGGRSLYNRGFSLLIMKNLNVTSLGFRSLKEISAGRIYISANRQLCYHHSLNWTKVLRGPTEERLDIKHNRP |
| | | RRDCVAEGKVCDPLCSSGGCWGPGPGQCLSCRNYSRGGVCVTHCNFLNGEPREFAHEAECFSCHPECQ |
| | | PMEGTATCNGSGSDTCAQCAHFRDGPHCVSSCPHGVLGAKGPIYKYPDVQNECRPCHENCTQGCKGPEL |
| | | QDCLGQTLVLIGKTHLTMALTVIAGLVVIFMMLGGTFLYWRGRRIQNKRAMRRYLERGESIEPLDPSEKANKV |
| | | LARIFKETELRKLKVLGSGVFGTVHKGVWIPEGESIKIPVCIKIIEDKSGRQSFQAVTDHMLAIGSLDHAHIVRLL |
| | | GLCPGSSLQLVTQYLPLGSLLDHVRQHRGALGPQLLLNWGVQIAKGMYYLEEHGMVHRNLAARNVLLKSPS |
| | | QVQVADFGVADLLPPDDKQLLYSEAKTPIKWMALESIHFGKYTHQSDVWSYGVTVWELMTFGAEPYAGLRL |
| | | AEVPDLLEKGERLAQPQICTIDVYMVMVKCWMIDENIRPTFKELANEFTRMARDPPRYLVIKRESGPGIAPGP |
| | | EPHGLTNKKLEEVELEPELDLDLDLEAEEDNLATTTLGSALSLPVGTLNRPRGSQSLLSPSSGYMPMNQGNL |
| | | GEAFQESAVSGSSEWCPRPVSLHPMPRGCLASESSEGHVTGSEAELQEKVSTCRSRSRSRSPRPRGDSA |
| | | YHSQRHSLLTPVTPLSPPGLEEEDVNGYVMPDTHLKGTPSSREGTLSSVGLSSVLGTEEEDEDEEYEYMNR |
| | | RRRHSPPRPPRPSSLEELGYEYMDVGSDLSASLGSTQSCPLHPVPVMPTAGTTPDEDYEYMNRQRGGSG |
| | | PGGDYAAMGACPASEQGYEEMRAFQGPGHQAPHVHYAHLKTLRSLEATDSAFDNPDYWHSRLFPKANAQ |
| | | RT |
| 21 | 10D1 heavy | DVQLQESGPDLVKPSQSLSLTCTVTGYSITSGYSWHWIRQFPGNKLEWMGSIHYSGGTNYNPSLKSRISITR |
| | chain variable | DTSKNQFFLQLNSVTTEDTATYFCARMTTAPRYPFDYWGQGTTLTVSS |
| | region | |
| 22 | 10D1_c75 heavy | DVQLQEWGAGLLKPSETLSLTCAVTGYSITSGYSWHWIRQFPGNGLEWIGSIHYSGGTNYNPTLKSRITISR |
| | chain variable | DTSKNQFSLKLSSVTAADTAVYFCARMTTAPRYPFDYWGQGTLVTVSS |
| | region | |
| 23 | 10D1_c76 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLKLSSVTAADTAVYFCARMTTAPRYPFDYWGQGTLVTVSS |
| | region | |
| 24 | 10D1_c77 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLKLSSVTAADTAVYFCARMTTAPRYPFDYWGQGTLVTVSS |
| | region | |
| 25 | 10D1_c78v1 | DVQLQEWGAGLLKPSETLSLTCAVTGYSITSGYSWHWIRQFPGNGLEWIGSIHYSGGTNYNPSLKSRITISR |
| | heavy chain | DTSKNQFSLKLSSVTAADTAVYFCARMTTAPRYPFDYWGQGTLVTVSS |
| | variable region | |
| 26 | 10D1_c78v2 | DVQLQEWGAGLLKPSETLSLTCAVTGYSITSGYSWHWIRQFPGKGLEWIGSIHYSGGTNYNPSLKSRITISRD |
| | heavy chain | TSKNQFSLKLSSVTAADTAVYFCARMTTAPRYPFDYWGQGTLVTVSS |
| | variable region | |
| 27 | 10D1_11B | DVQLQEWGAGLLKPSETLSLTCAVYGYSITSGYSWHWIRQPPGKGLEWIGSIHYSGGTNYNPSLKSRVTISR |
| | heavy chain | DTSKNQFSLKLSSVTAADTAVYYCARMTTAPRYPFDYWGQGTLVTVSS |
| | variable region | |
| 28 | 10D1_c85v1 | DVQLQEWGAGLLKPSETLSLTCAVTGYSITSGYSWHWIRQFPGNGLEWIGSIRYSGGTNYNPSLKSRITISR |
| | heavy chain | DTSKNQFSLKLGSVTAADTAVYFCARMTTAPRYPFDYWGQGTLVTVSS |
| | variable region | |
| 29 | 10D1_c85v2 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARMTTAPRYPFDYWGQGTLVTVSS |
| | variable region | |
| 30 | 10D1_c8501 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARETTAPRYPFDYWGQGTLVTVSS |
| | variable region | |
| 31 | 10D1_c85o2 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARGTTAPRYPFDYWGQGTLVTVSS |
| | variable region | |
| 32 | 10D1_c87 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLRLSSVTAADTAVYFCARMTTAPRYPFDYWGQGTLVTVSS |
| | region | |
| 33 | 10D1_c89 heavy | QVQLQESGPGLVKPSQTLSLTCTVSGYSITSGYSWHWIRQHPGKGLEWIGSIRYSGGTDYNPSLKSLVTISA |
| | chain variable | DTSKNQFSLKLSSVTAADTAVYYCARMTTAPWYPFDYWGQGTTVTVSS |
| | region | |
| 34 | 10D1_c90 heavy | QVQLQESGPGLVKPSQTLFLTCTVSGYSITSGYSWHWIRQHPGKGLEWIGSIRYSGGTDYNPSLKSLVTISV |
| | chain variable | DTSKNQFSLKLSSVTAADTAVYYCARMTTAPWYPFDYWGQGTTVTVSS |
| | region | |
| 35 | 10D1_c91 heavy | QVQLQESGPGLVKPSQTLSLTCTVSGYYITSGYSWHWIRQHPGKGLEWIGSIRYSGGTDYNPSLKSLATISA |
| | chain variable | DTSKNQFSLKLSSVTAADTAVYYCARMTTAPWYPFDYWGQGTAVTVSS |
| | region | |
| 36 | 10D1_c92 heavy | DVQLQEWGAGLLKPSETLSLTCAVTGYSITSGYSWHWIRQFPGNGLEWIGSIHYSGGTNYNPTLKSRITISR |
| | chain variable | DTSKNQFSLKLSSVTAADTAVYFCARMTTAPRYPFDYWGQGTLVTVSS |
| | region | |
| 37 | 10D1_c93 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLRLSSVTAADTAVYFCARMTTAPRYPFDYWGQGTLVTVSS |
| | region | |
| 38 | 10D1, | GYSITSGYS |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c85v1, | |
| | 10D1_c85v2, | |
| | 10D1_c8501, | |
| | 10D1_c85o2, | |
| | 10D1_c87, | |
| | 10D1_c89, | |
| | 10D1_c90, | |
| | 10D1_c92, | |
| | 10D1_c93 heavy | |
| | chain CDR1 | |
| 39 | 10D1_c91 heavy | GYYITSGYS |
| | chain CDR1 | |
| 40 | 10D1 derived | GYX₁ITSGYS |
| | consensus | wherein X₁ = S or Y |
| | heavy chain | |
| | CDR1 | |
| 41 | 10D1, | IHYSGGT |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c87, | |
| | 10D1_c92, | |
| | 10D1_c93 heavy | |
| | chain CDR2 | |
| 42 | 10D1_c85v1, | IRYSGGT |
| | 10D1_c85v2, | |
| | 10D1_c8501, | |
| | 10D1_c85o2, | |
| | 10D1_c89, | |
| | 10D1_c90, | |
| | 10D1_c91 heavy | |
| | chain CDR2 | |
| 43 | 10D1 derived | IX₂YSGGT |
| | consensus | wherein X₂ = H or R |
| | heavy chain | |
| | CDR2 | |
| 44 | 10D1, | ARMTTAPRYPFDY |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c85v1, | |
| | 10D1_c85v2, | |
| | 10D1_c87, | |
| | 10D1_c92, | |
| | 10D1_c93 heavy | |
| | chain CDR3 | |
| 45 | 10D1_c89, | ARMTTAPWYPFDY |
| | 10D1_c90, | |
| | 10D1_c91 heavy | |
| | chain CDR3 | |
| 46 | 10D1_c8501 | ARETTAPRYPFDY |
| | heavy chain | |
| | CDR3 | |
| 47 | 10D1_c85o2 | ARGTTAPRYPFDY |
| | heavy chain | |
| | CDR3 | |
| 48 | 10D1 derived | ARX₃TTAPX₄YPFDY |
| | consensus | wherein X₃ = M, E or G; X₄ = R or W |
| | heavy chain | |
| | CDR3 | |
| 49 | 10D1 light chain | DIVMTQSQKFMSTSVGDRVSVTCKASQIVGSNVAWYQQKPGQSPKPLIYSASYRYSGVPDRFTASGSGTDF |
| | variable region | TLTITNVQSEDLAEYFCQQYSSHPLTFGAGTKLELK |
| 50 | 10D1_c75 light | DIVMTQSPSSLSASVGDLVTITCKASQIVGSNVAWYQMKPGKSPKPLIYSASYLYFGVPSRFSGSGSGTDFT |
| | chain variable | LTISSLQPEDVAEYFCQQYSSHPLTFGPGTKVEIK |
| | region | |
| 51 | 10D1_c76 light | DIVMTQSPSSLSASGGDRVTITCKASQIVGYNVAWYQQKPGKSPKPLIYSASYLYSDVPSRFSASGSGTDFT |
| | chain variable | LTISSLQPEDVAEYFCQQYSSHPLTFGPGTKVEIK |
| | region | |
| 52 | 10D1_c77 light | VIVMTQSPSSLSASVGDRVTITCKASQIVGPNVAWYQQKPGKSPKPLIYSASYGYSDVPSRFSGSGSGTDFT |
| | chain variable | LTISSLQPEDVAEYFCQQYSTHPLTFGPGTKVEIK |
| | region | |
| 53 | 10D1_c78v1, | DIVMTQSPSSLSASVGDRVTITCKASQIVGSNVAWYQQKPGKSPKPLIYSASYGYSDVPSRFSGSGSGTDFT |
| | 10D1_c78v2, | LTISSLRPEDVATYYCQQYSSHPLTFGPGTKVEIK |
| | 10D1_11B light | |
| | chain variable | |
| | region | |
| 54 | 10D1_c85v1, | DIVMTQSPSSLSASVGDRVTITCKASQIVGSNVAWYQQKPGKSPKPLIYSARYQYSGVPFRFSGSGSGTDFT |
| | 10D1_c85v2 | LTISSLQPEDVATYYCQQYSSHPLTFGPGTKVEIK |
| | light chain | |
| | variable region | |
| 55 | 10D1_c8501 | DIVMTQSPSSLSASVGDRVTITCKASQIVGSNVAWYQQKPGKSPKPLIYSARYQYSGVPFRFSGSGSGTDFT |
| | light chain | LTISSLQPEDVATYYCQQYSSHPLTFGPGTKVEIK |
| | variable region | |
| 56 | 10D1_c85o2 | DIVMTQSPSSLSASVGDRVTITCKASQIVGSNVAWYQQKPGKSPKPLIYSARYQYSGVPFRFSGSGSGTDFT |
| | light chain | LTISSLQPEDVATYYCQQYSSHPLTFGPGTKVEIK |
| | variable region | |
| 57 | 10D1_c87 light | DIVMTQSPSSLSASVGDRVTITCKASQIVGSNVAWYQQMPGKSPEPLIYSASYLYSDVPSRFSGSGSGTDFT |
| | chain variable | MTISSLQPEDVATYYCQQYSSHPLTFGPGTKVEIK |
| | region | |
| 58 | 10D1_c89 light | DIQMTQSPSSVSASVGDRVTITCKASQIVGSNVAWYQQKPGKAPEPLIYSASYLYSGVPSRFSGSGSGTDFT |
| | chain variable | LTISSLQPEDFATYYCQQYSSHPLTFGQGTKLEIK |
| | region | |
| 59 | 10D1_c90 light | DIQMTQSPSSVSASVGDRVTFTCKASQIVGSNVAWYQQKPGKAPEPLIYSASYLYSSVPSRFSGSGSGTEF |
| | chain variable | TMTISSLEPEDFATYYCQQYTTHPLTFGPGTKVEIK |
| | region | |
| 60 | 10D1_c91 light | DIQMTQSPSSVSASVGDRVTITCKASQIVGSNVAWYQQKPGKAPMPLIYSASYGYSGVPSRFSGSGSGTDF |
| | chain variable | TLTISSLQPEDFATYYCQQYSSHPLTFGQGTKLEIK |
| | region | |
| 61 | 10D1_c92 light | DIVMTQSPSSLSASVGDLVTITCKASQIVGSNVAWYQMKLGKSPKPLIYSASYLYFGVPSRFSGSGSGTDFTL |
| | chain variable | TISSLQPEDVAEYFCQQYFSHPLTFGPGTKVEIK |
| | region | |
| 62 | 10D1_c93 light | DIVMTQSPSSLSASVGDRVTITCKASQIVGSNVAWYQQKPGKSPKPLIYSASYLYSDVPSRFSGSGSGTDFT |
| | chain variable | MTISSLQPEDVATYYCQQYSSHPLTFGPGTKVEIK |
| | region | |
| 63 | 10D1, | QIVGSN |
| | 10D1_c75, | |
| | 10D1_c78v1, | |
| | 10D1_c78v2, | |
| | 10D1_11B, | |
| | 10D1_c85v1, | |
| | 10D1_c85v2, | |
| | 10D1_c8501, | |
| | 10D1_c85o2, | |
| | 10D1_c87, | |
| | 10D1_c89, | |
| | 10D1_c90, | |
| | 10D1_c91, | |
| | 10D1_c92, | |
| | 10D1_c93 light | |
| | chain CDR1 | |
| 64 | 10D1_c76 light | QIVGYN |
| | chain CDR1 | |
| 65 | 10D1_c77 light | QIVGPN |
| | chain CDR1 | |
| 66 | 10D1 derived | QIVGX₅N |
| | consensus light | wherein X₅ = S, Y or P |
| | chain CDR1 | |
| 67 | 10D1, | SAS |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c87, | |
| | 10D1_c89, | |
| | 10D1_c90, | |
| | 10D1_c91, | |
| | 10D1_c92, | |
| | 10D1_c93 | |
| | light chain CDR2 | |
| 68 | 10D1_c85v1, | SAR |
| | 10D1_c85v2, | |
| | 10D1_c8501, | |
| | 10D1_c85o2 | |
| | light chain CDR2 | |
| 69 | 10D1 derived | SAX₆ |
| | consensus light | wherein X₆ = S or R |
| | chain CDR2 | |
| 70 | 10D1, | QQYSSHPLT |
| | 10D1_c75, | |
| | 10D1_c76, | |
| | 10D1_c78v1, | |
| | 10D1_c78v2, | |
| | 10D1_11B, | |
| | 10D1_c85v1, | |
| | 10D1_c85v2, | |
| | 10D1_c8501, | |
| | 10D1_c85o2, | |
| | 10D1_c87, | |
| | 10D1_c89, | |
| | 10D1_c91, | |
| | 10D1_c93 light | |
| | chain CDR3 | |
| 71 | 10D1_c77 light | QQYSTHPLT |
| | chain CDR3 | |
| 72 | 10D1_c90 light | QQYTTHPLT |
| | chain CDR3 | |
| 73 | 10D1_c92 light | QQYFSHPLT |
| | chain CDR3 | |
| 74 | 10D1 derived | QQYX₇X₈HPLT |
| | consensus light | wherein X₇ = S, T or F; X₈ = S or T |
| | chain CDR3 | |
| 75 | 10D1F hlgG1 | QVQLQESGPGLVKPSQTLSLTCTVSGYSITSGYSWHWIRQHPGKGLEWIGSIRYSGGTDYNPSLKSLVTISA |
| | HC | DTSKNQFSLKLSSVTAADTAVYYCARMTTAPWYPFDYWGQGTTVTVSSASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKSRWQQGNVFSCS |
| | | VMHEALHNHYTQKSLSLSPGK |
| 76 | 10D1F K LC | DIQMTQSPSSVSASVGDRVTITCKASQIVGSNVAWYQQKPGKAPEPLIYSASYLYSGVPSRFSGSGSGTDFT |
| | | LTISSLQPEDFATYYCQQYSSHPLTFGQGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVYACEVTHQGLSSPVTKSFNRGEC |
| 77 | cetuximab VH | QVQLKQSGPGLVQPSQSLSITCTVSGFSLTNYGVHWVRQSPGKGLEWLGVIWSGGNTDYNTPFTSRLSINK |
| | | DNSKSQVFFKMNSLQSNDTAIYYCARALTYYDYEFAYWGQGTLVTVSA |
| 78 | cetuximab HC- | GFSLTNYG |
| | CDR1 | |
| 79 | cetuximab HC- | IWSGGNT |
| | CDR2 | |
| 80 | cetuximab HC- | ARALTYYDYEFAY |
| | CDR3 | |
| 81 | cetuximab VL | DILLTQSPVILSVSPGERVSFSCRASQSIGTNIHWYQQRTNGSPRLLIKYASESISGIPSRFSGSGSGTDFTLSI |
| | | NSVESEDIADYYCQQNNNWPTTFGAGTKLELK |
| 82 | cetuximab LC- | QSIGTN |
| | CDR1 | |
| 83 | cetuximab LC- | YAS |
| | CDR2 | |
| 84 | cetuximab LC- | QQNNNWPTT |
| | CDR3 | |
| 85 | cetuximab HC | QVQLKQSGPGLVQPSQSLSITCTVSGFSLTNYGVHWVRQSPGKGLEWLGVIWSGGNTDYNTPFTSRLSINK |
| | | DNSKSQVFFKMNSLQSNDTAIYYCARALTYYDYEFAYWGQGTLVTVSAASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKSRWQQGNVFSCS |
| | | VMHEALHNHYTQKSLSLSPGK |
| 86 | cetuximab LC | DILLTQSPVILSVSPGERVSFSCRASQSIGTNIHWYQQRTNGSPRLLIKYASESISGIPSRFSGSGSGTDFTLSI |
| | | NSVESEDIADYYCQQNNNWPTTFGAGTKLELKRTVAAPSVFIFPPSDEQLKSGTASVVCLLNNFYPREAKVQ |
| | | WKVDNALQSGNSQESVTEQDSKDSTYSLSSTLTLSKADYEKHKVYACEVTHQGLSSPVTKSFNRGEC |

The present disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

It must be noted that, as used in the specification and the appended claims, the singular forms ‘a’, ‘an’, and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about’, it will be understood that the particular value forms another embodiment.

Where a nucleic acid sequence is disclosed or referred to herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

Values may be expressed herein as ‘about’ a particular value. Similarly, ranges may be expressed herein as from ‘about’ a particular value, and/or to ‘about’ another particular value. The term ‘about’ in relation to a numerical value is optional, and means for example+/−10%. By way of illustration, reference e.g. to ‘about 10%’ is to be construed as 9% to 11%. In instances herein where ‘about’ is recited, the value it precedes is also specifically contemplated. By way of illustration, reference e.g. to ‘about 10%’ also specifically contemplates 10%.

### BRIEF DESCRIPTION OF THE FIGURES

Embodiments and experiments illustrating the principles of the present disclosure will now be discussed with reference to the accompanying figures.

FIGS. 1A and 1B. Bar chart and graph showing comparison of anti-cancer efficacy of 10D1F/10D1 P for different cancer cell lines determined by analysis in in vitro and in vivo studies. (1A) shows the efficacy of 10D1F/10D1 P to inhibit growth of cells of the indicated cancer cell lines in vitro, or in vivo in cell-line derived xenograft models. (1B) shows a linear regression modelling the relationship between efficacy determined for treatment of the cell lines with 10D1F/10D1 P in vitro and in vivo.

FIGS. 2A and 2B. Graphs showing the response of cancer cell lines having different genotypes (see Example 2) to treatment with (2A) 10D1F/10D1 P, and (2B) seribantumab, as determined by analysis in in vitro and in vivo studies. ****P50.0001.

FIGS. 3A and 3B. Graph showing the response of cancer cell lines having different genotypes (see Example 3) to treatment with (3A) 10D1F/10D1 P, and (3B) seribantumab, as determined by analysis in in vitro and in vivo studies. ****P50.0001.

FIG. 4. Graphs showing the response of cancer cell lines having different genotypes (see Example 4) to treatment with 10D1F/10D1 P.

FIG. 5. Graphs showing the response of cancer cell lines having different genotypes (see Example 4) to treatment with 10D1F/10D1 P.

FIG. 6. Schematic representation of hypothetical etiology and tumorigenesis in squamous cell carcinoma and the influence of anti-HER3 antibody therapy

### EXAMPLES

In the present Examples the inventors demonstrate that extent of response to treatment with anti-HER3 antibodies is dependent on the mutational status of the cancer in genes encoding regulators of HER3-mediated signalling.

### Example 1: Analysis of Correlation Between Inhibition of Cell Proliferation In Vitro and Tumor Growth Inhibition In Vivo

The inventors investigated whether the results of analysis of the ability of anti-HER3 antibody 10D1F hIgG1 (comprising VH=SEQ ID NO:33, VL=SEQ ID NO:58) or 10D1 P hIgG1 (comprising VH=SEQ ID NO:21, VL=SEQ ID NO:49) to inhibit growth of cells of cancer cell lines in vitro is predictive of its ability to inhibit tumor growth of cell line-derived xenograft tumors in vivo.

The cell lines analysed were N87 (gastric cancer), FaDu (head and neck cancer), OvCAR8 (ovarian cancer), SNU16 (gastric cancer), A549 (lung cancer), HCC95 (lung cancer), AHCN (kidney cancer) and 22Rv1 (prostate cancer).

For analysis of inhibition of growth in vitro, cells of the different cell lines were treated with 10-point serially diluted concentrations of anti-HER3 antibody (3-fold dilations, starting from a maximum concentration of 1500 μg/ml), in triplicate. Cell viability was measured using CCK-8 assay (Dojindo) after 3-5 days. The percentage inhibition of growth of the cells was calculated by comparison to the CCK-8 assay signal for cells treated with buffer only (PBS). In vitro efficacy was determined to be the percentage inhibition observed at 1500 μg/ml.

For analysis of inhibition of growth in vivo, cell line-derived xenograft models were established by subcutaneous injection of cells into the right flank of female NCr nude mice (approximately 6-8 weeks old). Anti-HER3 antibody was administered by intraperitoneal injection, twice weekly at 25 mg/kg bodyweight per dose. Control treatment groups received an equal volume of PBS. Tumor volumes were measured 3 times a week using a digital caliper and calculated using the formula [L×W2/2]. Study End point was considered to have been reached once the tumors of the control arm measured>1.5 cm in length. In vivo efficacy was determined to be the tumor growth inhibition percentage at Study End.

Efficacy data from in vitro and in vivo studies were normalised to set the maximum inhibition as 100% and minimum inhibition as 0%, where the values were above or below this range. In vitro-in vivo efficacy correlation was studied by a simple linear regression and the correlation coefficient (R²) was calculated using GraphPad Prism software.

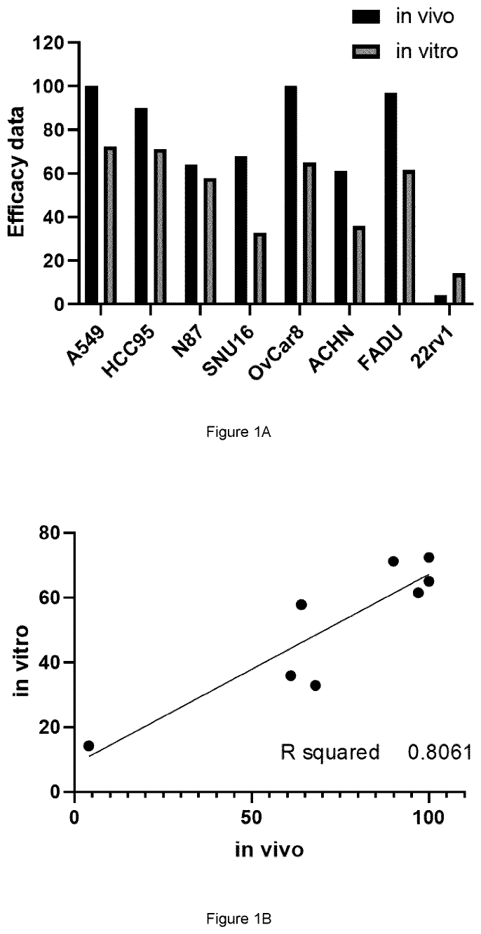

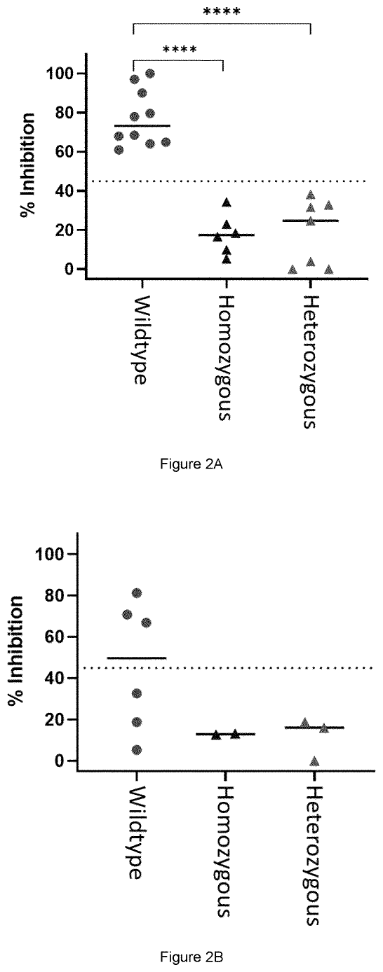

The results of the analysis are shown in FIGS. 1A and 1B. There was a strong correlation between results obtained in in vitro studies and in vivo studies (R²=0.8061). This indicates that the results of in vitro analyses of inhibition of cancer cell growth are predictive of efficacy of tumor growth inhibition in vivo (and vice versa).

### Example 2: Analysis of Efficacy of HER3-Binding Antigen-Binding Molecules to Treat Cancer Based on Mutation Status of Regulators of HER3-Mediated Signalling

The inventors employed a combination of data from in vivo and in vitro studies of the anti-cancer activity of 10D1F/10D1 P or seribantumab to investigate whether the mutation status of the cancer cell lines was predictive of efficacy of treatment using the antibody.

Cell lines from breast, gastric and non-small cell lung cancers, colorectal cancer & SCCHN and prostate cancers were included in the analysis. The cell lines were analysed for presence of point mutations in KRAS, PIK3CA and for PTEN loss using public databases such as COSMIC, Cellosaurus and CCLE. Cell lines were categorised as: (i) wildtype for KRAS, PIK3CA and PTEN (‘wildtype’); (ii) heterozygous for an activating mutation to KRAS or PIK3CA, or heterozygous for an inactivating mutation to PTEN (‘heterozygous’); (iii) homozygous for an activating mutation to KRAS or PIK3CA, or homozygous for an inactivating mutation to PTEN (‘homozygous’).

In scoring responses to treatment for 10D1F/10D1 P, tumor growth inhibition (TGI) data from in vivo studies using 10D1F hIgG1 were prioritised. Where such data were not available, TGI data from 10D1 P hIgG1 were used. Where in vivo data was not available for a cell line in vitro inhibition data was used. The relationship between normalised functional responses to treatment with 10D1F/10D1 P or seribantumab and mutational status was analysed using GraphPad Prism. For comparisons between wildtype, heterozygous and homozygous groups, an unpaired t-test was performed.

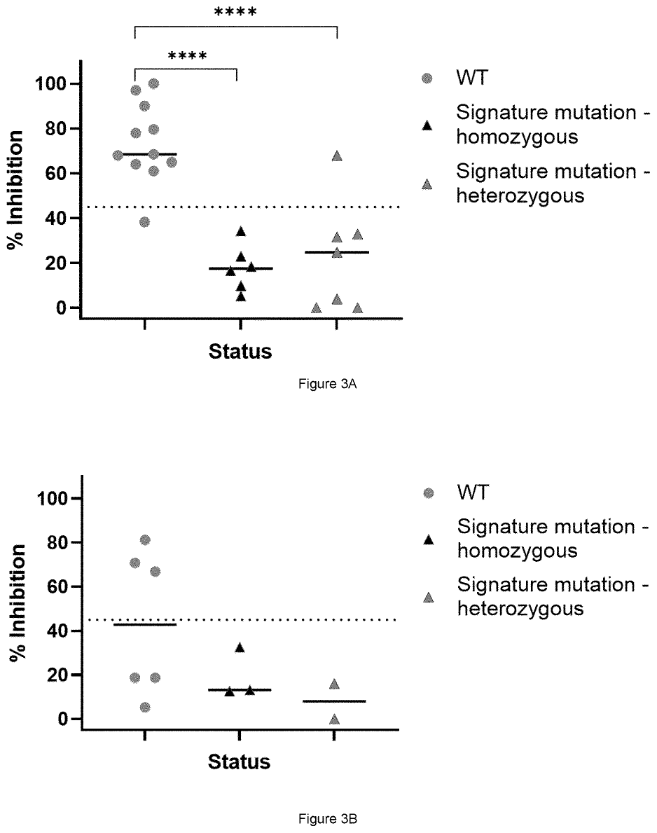

The genotypes of the cell lines for KRAS, PIK3CA and PTEN and their normalised functional responses to treatment with 10D1F/10D1 P or seribantumab (as determined by analysis in in vitro or in vivo studies as described in Example 1) are shown in the table below, and the data are represented graphically in FIGS. 2A and 2B.

| | | | | | | seribantumab |
|---|---|---|---|---|---|---|
| Cell Line | KRAS | PIK3CA | PTEN | Category | 10D1F/10D1P response | response |
| FaDu | WT/WT | WT/WT | WT/WT | Wildtype | 97 (10D1F, in vivo) | 81.2 (in vivo) |
| HCC95 | WT/WT | WT/WT | WT/WT | Wildtype | 90 (10D1P, in vivo) | 70.8 (in vitro) |
| N87 | WT/WT | WT/WT | WT/WT | Wildtype | 64 (10D1F, in vivo) | 18.7 (in vivo) |
| SNU16 | WT/WT | WT/WT | WT/WT | Wildtype | 68 (10D1P, in vivo) | 32.57 (in vitro) |
| OvCar8 | WT/WT | WT/WT | WT/WT | Wildtype | 100 (10D1F, in vivo) | 66.8 (in vivo) |
| ACHN | WT/WT | WT/WT | WT/WT | Wildtype | 61 (10D1P, in vivo) | 5.3 (in vitro) |
| BCPAP | WT/WT | WT/WT | WT/WT | Wildtype | 64.87 (10D1F, in vitro) | NT |
| BHT101 | WT/WT | WT/WT | WT/WT | Wildtype | 68.5 (10D1F, in vitro) | NT |
| LoVo | G12C/WT | WT/WT | WT/WT | Heterozygous | 31.67 (10D1F, in vivo) | NT |
| HCT15 | G12D/WT | D549N, | | Heterozygous | 32.87 (10D1F, in vivo) | NT |
| | | E545K/WT | | | | |
| HCT116 | G12D/WT | H1047R/WT | | Heterozygous | 24.73 (10D1F, in vivo) | NT |
| HT29 | WT/WT | P449T/WT | | Heterozygous | 0 (10D1P, in vivo) | 16 (in vitro) |
| DU145 | WT/WT | | KO/WT | Heterozygous | 38.3 (10D1F, in vitro) | 18.7 (in vitro) |
| 22Rv1 | | Q546R/WT | | Heterozygous | 4 (10D1F, in vivo) | 0 (in vitro) |
| H358 | G12C/WT | | | Heterozygous | 0 (10D1F, in vitro) | NT |
| PC3 ATCC | | | KO/KO | Homozygous | 23 (10D1F, in vitro) | 13.12 (in vitro) |
| LnCap | | | KO/KO | Homozygous | 9.93 (10D1F, in vitro) | 12.7 (in vitro) |
| H1373 | G12C/G12C | | | Homozygous | 16.6 (10D1F, in vitro) | NT |
| H2030 | G12C/G12C | | | Homozygous | 18.4 (10D1F, in vitro) | NT |
| H2122 | G12C/G12C | | | Homozygous | 5.3 (10D1F, in vitro) | NT |
| FTC133 | | | KO/KO | Homozygous | 34.33 (10D1F, in vitro) | NT |

NT = not tested

The analysis revealed a very striking pattern, in which the response to treatment with anti-HER3 antibody was dependent on the presence of mutations resulting in an increase in HER3-mediated signalling. Cell lines in the wildtype group were much more responsive to anti-HER3 antibody treatment compared to cell lines in the heterozygous or homozygous groups. This pattern was observed across the full range of different cancer types analysed.

### Example 3: Analysis of Efficacy of HER3-Binding Antigen-Binding Molecules to Treat Cancer Based on the Mutation Status of PTEN/KRAS/PIK3CA/BRAF and NRG1 mRNA Abundance

In further studies, the inventors employed the data from in vivo and in vitro studies of the anti-cancer activity of 10D1F/10D1P or seribantumab to investigate whether the mutation status of BRAF could be added to the triple gene signature (PTEN/KRAS/PIK3CA) described in Example 2 to predict the efficacy of anti-HER3 antibody treatment.

Cell lines from breast, gastric and non-small cell lung cancers, colorectal cancer & SCCHN and prostate cancers were included in the analysis. The cell lines were evaluated by empirical sequence analysis for the presence of point mutations in KRAS, PIK3CA and BRAF, and for PTEN loss. Cell lines were categorised as: (i) wildtype for KRAS, PIK3CA, PTEN, and BRAF (‘wildtype’); (ii) heterozygous for an activating mutation to KRAS or PIK3CA or BRAF, or heterozygous for an inactivating mutation to PTEN (‘heterozygous’); (iii) homozygous for an activating mutation to KRAS or PIK3CA or BRAF, or homozygous for an inactivating mutation to PTEN (‘homozygous’). Expression levels of NRG1 mRNA were also measured for each cell line.

In scoring responses to treatment for 1001 Ff 101 P, tumor growth inhibition (TGI) data from in vivo studies using 10D1F hIgG1 were prioritised. Where such data were not available, TGI data from 1001 P hIgG1 were used. Where in vivo data was not available for a cell line in vitro inhibition data was used.

The relationship between normalised functional responses to treatment with 10D1F/10D1P or seribantumab and mutational status was analysed using GraphPad Prism. For comparisons between wildtype, heterozygous and homozygous groups, an unpaired nest was performed.

The genotypes of the cell lines for KRAS, PIK3CA, PTEN and BRAF as determined by sequence analysis and their normalised functional responses to treatment with 101 Ff 10D1 P or seribantumab (as determined by analysis in in vitro or in vivo studies as described in Example 1) are shown in the table below, and the data are represented graphically in FIGS. 3A and 3B.

| | | | | | | | | NRG1 mRNA |
|---|---|---|---|---|---|---|---|---|
| | | | | | | | Seribantumab | expression |
| Cell line | KRAS | PIK3CA | PTEN | BRAF | Category* | 10D1F/10D1P response | response | (RPKM) |
| FaDu | WT/WT | WT/WT | WT/WT | WT/WT | Wildtype | 97 (10D1F, in vivo) | 81.2 (in vivo) | 0.75 |
| HCC95 | WT/WT | WT/WT | WT/WT | WT/WT | Wildtype | 90 (10D1P, in vivo) | 70.8 (in vitro) | 157.38 |
| N87 | WT/WT | WT/WT | WT/WT | WT/WT | Wildtype | 64 (10D1F, in vivo) | 18.7 (in vivo) | 0.07 |
| SNU16 | G12D/WT | WT/WT | WT/WT | WT/WT | Heterozygous | 68 (10D1F, in vivo) | 32.57 (in vitro) | 0.012 |
| OvCar8 | WT/WT | WT/WT | WT/WT | WT/WT | Wildtype | 100 (10D1F, in vivo) | 66.8 (in vivo) | 5.62 |
| ACHN | WT/WT | WT/WT | WT/WT | WT/WT | Wildtype | 61 (10D1P, in vivo) | 5.3 (in vitro) | 11.64 |
| BCPAP | WT/WT | WT/WT | WT/WT | V600E/WT | Heterozygous | 64.87 (10D1F, in vitro) | NT | 0.95 |
| BHT101 | WT/WT | WT/WT | WT/WT | V600E/WT | Heterozygous | 68.5 (10D1F, in vitro) | NT | 1.66 |
| LoVo | G13C/WT | WT/WT | WT/WT | WT/WT | Heterozygous | 31.67 (10D1F, in vivo) | NT | 0.0084 |
| HCT15 | G13D/WT | D549N, | WT/WT | WT/WT | Heterozygous | 32.87 (10D1F, in vivo) | NT | 0.014 |
| | | E545K/WT | | | | | | |
| HCT116 | G13D/WT | H1047R/WT | WT/WT | WT/WT | Heterozygous | 24.73 (10D1F, in vivo) | NT | 0.77 |
| HT29 | WT/WT | P449T/WT | WT/WT | V600E/WT; | Heterozygous | 0 (10D1P, in vivo) | 16 (in vitro) | 0.0038 |
| | | | | T119S/WT | | | | |
| DU145 | WT/WT | WT/WT | WT/WT | WT/WT | Wildtype | 38.3 (10D1F, in vitro) | 18.7 (in vitro) | 0.43 |
| 22Rv1 | WT/WT | Q546R/WT | WT/WT | L597R/WT | Heterozygous | 4 (10D1F, in vivo) | 0 (in vitro) | 0.012 |
| H358 | G12C/WT | WT/WT | KO/KO | WT/WT | Heterozygous | 0 (10D1F, in vitro) | NT | 3.32 |
| PC3 | WT/WT | WT/WT | KO/KO | WT/WT | Homozygous | 23 (10D1F, in vitro) | 13.12 (in vitro) | 0.022 |
| ATCC | | | | | | | | |
| LnCap | WT/WT | WT/WT | KO/KO | WT/WT | Homozygous | 9.93 (10D1F, in vitro) | 12.7 (in vitro) | 0.015 |
| H1373 | G12C/G12C | WT/WT | WT/WT | WT/WT | Homozygous | 16.6 (10D1F, in vitro) | INT | 1.44 |
| H2030 | G12C/G12C | WT/WT | WT/WT | WT/WT | Homozygous | 18.4 (10D1F, in vitro) | NT | 7.48 |
| H2122 | G12C/G12C | WT/WT | WT/WT | WT/WT | Homozygous | 5.3 (10D1F, in vitro) | NT | 0.64 |
| FTC133 | WT/WT | WT/WT | KO/KO | WT/WT | Homozygous | 34.33 (10D1F, in vitro) | NT | 2.54 |

NT = Not tested

The analysis revealed that the mutational status of BRAF in combination with PIK3CA, KRAS, and PTEN is predictive for the efficacy of anti-HER3 antibody treatment, and cell lines in the wildtype group were much more responsive to anti-HER3 antibody treatment compared with cell lines from heterozygous or homozygous groups (see FIGS. 3A and 3B). This pattern was observed across a wide range of preclinical cancer models.

The data in the table above further demonstrate a link between NRG1 expression and treatment efficacy, with increased NRG1 expression correlating with improved efficacy of anti-HER3 antibody treatment.

In conclusion, a gene signature comprising the mutational status of PIK3CA, KRAS, BRAF, and PTEN consistently and significantly predicts the efficacy of anti-HER3 antibody treatment in preclinical cancer models, and this gene signature can be combined with NRG1 mRNA abundance as an additional predictive factor.

### Example 4: Analysis of Efficacy of HER3-Binding Antigen-Binding Molecules to Treat Cancer Based on the Mutation Status of PTEN/KRAS/PIK3CA/BRAF/MET

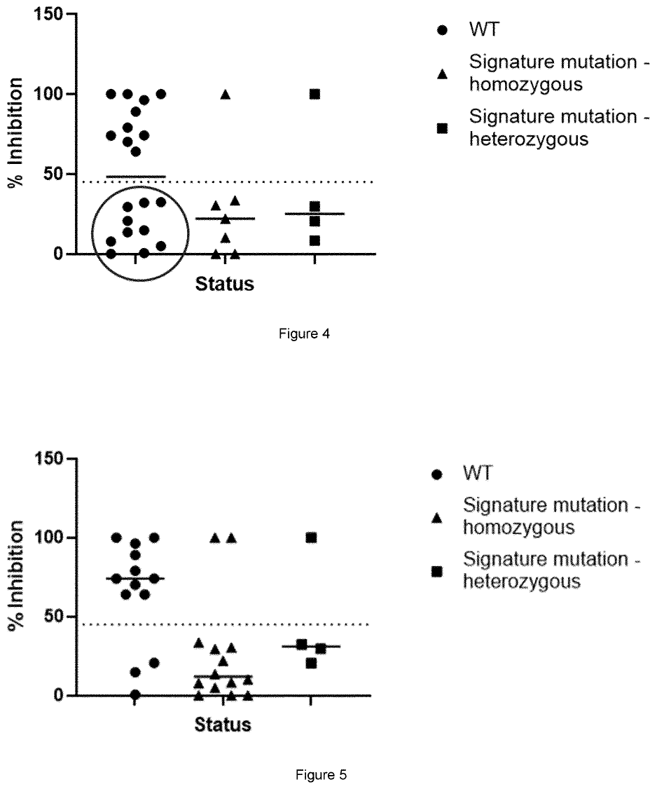

The inventors investigated cancers displaying poor response to treatment with 10D1F/10D1 P in in vivo and in vitro studies, and having a homozygous wildtype genotype for PTEN/KRAS/PIK3CA/BRAF (circled in FIG. 4). They determined that 6 out of 9 of those cancers comprised MET amplification.

In further studies, the inventors employed the data from in vivo and in vitro studies of the anti-cancer activity of 10D1F/10D1P to investigate whether the mutation status of MET could be added to the 4 gene signature (PTEN/KRAS/PIK3CA/BRAF) described in Example 3 to predict the efficacy of anti-HER3 antibody treatment.

Cell lines from various different cancers were evaluated by empirical sequence analysis for the presence of mutations in KRAS, PIK3CA and BRAF, PTEN and MET. Cell lines were categorised as: (i) wildtype for KRAS, PIK3CA, PTEN, BRAF and MET (‘wildtype’); (ii) heterozygous for an activating mutation to KRAS or PIK3CA or BRAF or MET, or heterozygous for an inactivating mutation to PTEN (‘heterozygous’); (iii) homozygous for an activating mutation to KRAS or PIK3CA or BRAF or MET, or homozygous for an inactivating mutation to PTEN (‘homozygous’).

In scoring responses to treatment for 10D1F/10D1P, tumor growth inhibition (TGI) data from in vivo studies using 10D1F hIgG1 were prioritised. Where such data were not available, TGI data from 1001 P hIgG1 were used. Where in vivo data was not available for a cell line in vitro inhibition data was used.

The relationship between normalised functional responses to treatment with 10D1F/10D1P or seribantumab and mutational status was analysed using GraphPad Prism. For comparisons between wildtype, heterozygous and homozygous groups, an unpaired t-test was performed.

The genotypes of the cell lines for KRAS, PIK3CA, PTEN and BRAF as determined by sequence analysis and their normalised functional responses to treatment with 10D1F/10D1P or seribantumab (as determined by analysis in in vitro or in vivo studies as described in Example 1) are shown in the table below.

| | | | | | | | | HMBD-001 |
|---|---|---|---|---|---|---|---|---|
| Cell line | Cancer type | KRAS | PIK3CA | PTEN | BRAF | MET | Category | response |
| CTG- | sqNSCLC | WT/WT | WT/WT | WT/WT | WT/WT | AMP/AMP | Homozygous | 114.8 |
| 2558 | | | | | | | | |
| FaDu | HNSCC | WT/WT | WT/WT | WT/WT | WT/WT | WT | Wildtype | 108.2 |
| BXPC3 | PDAC | WT/WT | WT/WT | WT/WT | V487_P492delinsA/WT | WT | Heterozygous | 101.9 |
| A549 | NSCLC | G12S/G12S | WT/WT | WT/WT | WT/WT | WT | Homozygous | 100.2 |
| OvCar8 | HGSOC | WT/WT | WT/WT | WT/WT | WT/WT | Deepdel/WT | Wildtype | 100.0 |
| LU6432 | sqNSCLC | WT/WT | WT/WT | WT/WT | WT/WT | WT | Wildtype | 96.3 |
| ESO199 | ESCC | WT/WT | WT/WT | WT/WT | WT/WT | WT | Wildtype | 89.1 |
| CTG- | sqNSCLC | WT/WT | WT/WT | WT/WT | WT/WT | WT | Wildtype | 79.1 |
| 2552 | | | | | | | | |
| Cal27 | HNSCC | WT/WT | WT/WT | WT/WT | WT/WT | WT | Wildtype | 74.2 |
| CTG- | PDAC | WT/WT | WT/WT | WT/WT | WT/WT | WT | Wildtype | 74.1 |
| 0301 | | | | | | | | |
| OE21 | ESCC | WT/WT | WT/WT | WT/WT | WT/WT | WT | Wildtype | 70.2 |
| CTG- | PDAC | WT/WT | WT/WT | WT/WT | WT/WT | WT | Wildtype | 64.1 |
| 2179 | | | | | | | | |
| KYSE- | ESCC | WT/WT | WT/WT | WT/WT | WT/WT | WT | Wildtype | 41.2 |
| 150 | | | | | | | | |
| H358 | NSCLC | G12C/G12C | WT/WT | deletion | WT/WT | WT | Homozygous | 33.6 |
| CTG- | HNSCC | WT/WT | WT/WT | WT/WT | WT/WT | H1192R/WT | Heterozygous | 32.4 |
| 2062 | | | | | | | | |
| N87 | Esophagogastric | WT/WT | WT/WT | WT/WT | WT/WT | Deepdel/WT | Wildtype | 32.0 |
| COLO201 | CRC | WT/WT | WT/WT | WT/WT | V600E | WT | Homozygous | 30.4 |
| HCT116 | CRC | G12D/WT | H1047R/WT | WT/WT | WT/WT | WT | Heterozygous | 29.7 |
| CTG- | HNSCC | WT/WT | WT/WT | WT/WT | WT/WT | AMP/R469X | Homozygous | 29.4 |
| 2352 | | | | | | and R1005Q | | |
| H1373 | NSCLC | G12C/G12C | WT/WT | WT/WT | WT/WT | WT | Homozygous | 22.0 |
| CTG- | CRC | WT/WT | WT/WT | WT/WT | WT/WT | WT | Wildtype | \|20.7 |
| 0075 | | | | | | | | |
| HCT15 | CRC | G12D/WT | D549N, | WT/WT | WT/WT | WT | Heterozygous | 20.4 |
| | | | E545K/WT | | | | | |
| HARA | sqNSCLC | WT/WT | WT/WT | WT/WT | WT/WT | WT | Wildtype | 14.8 |
| HCC15 | sqNSCLC | WT/WT | WT/WT | WT/WT | WT/WT | mt/mt | Homozygous | 13.6 |
| ME12035 | melanoma | WT/WT | WT/WT | WT/WT | V600E | WT | Homozygous | 10.2 |
| Detroit | HNSCC | WT/WT | H1047R/WT | WT/WT | WT/WT | AMP/AMP | Homozygous | 8.3 |
| 562 | | | | | | | | |
| CTG- | PDAC | WT/WT | WT/WT | WT/WT | WT/WT | AMP/AMP | Homozygous | 7.8 |
| 0851 | | | | | | | | |
| CC6658 | CCA | WT/WT | WT/WT | WT/WT | WT/WT | AMP/AMP | Homozygous | 4.9 |
| OVCAR3 | ovarian | WT/WT | WT/WT | WT/WT | WT/WT | WT | Wildtype | 0.5 |
| CTG- | ESCC | WT/WT | WT/WT | WT/WT | WT/WT | AMP/AMP | Homozygous | −11.8 |
| 1741 | | | | | | | | |
| CTG- | CRC | WT/WT | WT/WT | WT/WT | V600E | AMP | Homozygous | −12.7 |
| 1009 | | | | | | | | |
| SK-MEL- | melanoma | WT/WT | WT/WT | WT/WT | V600E | AMP | Homozygous | −14.5 |
| 5 | | | | | | | | |

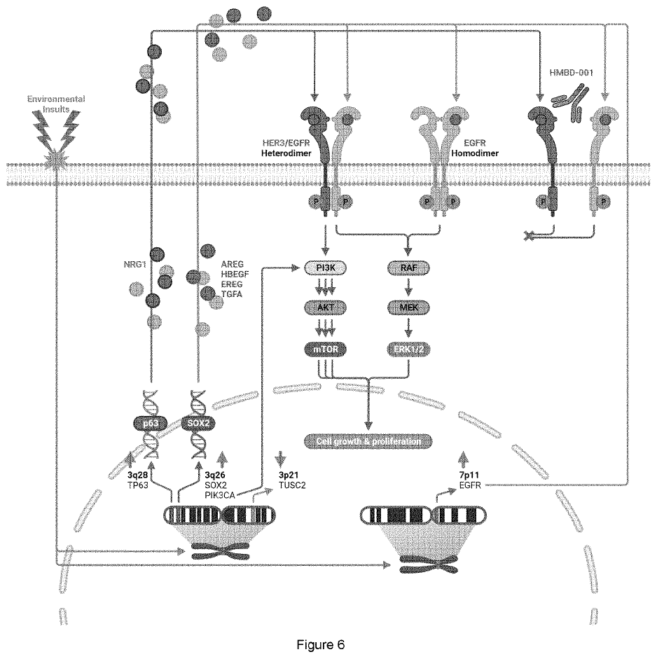

Addition of MET to the 4 gene biomarker panel improved the ability to predict positive response to treatment with anti-HER3 antibody therapy (see FIG. 5). In particular, the 5 gene panel provides for the prediction of positive response to treatment with anti-HER3 antibody therapy with greater sensitivity, relative to the 4 gene panel.

### Example 5: Analysis of Efficacy of HER3-Binding Antigen-Binding Molecules to Treat Cancer Based on the Amplification Status of Chromosomal Region 3q26 to 3q28 Genes (TP63, SOX2, PIK3CA), EGFR and TUSC2

In further studies, the inventors employed the data from in vivo and in vitro studies of the anti-cancer activity of 10D1F/10D1P to investigate whether the gene amplification status of genes in the chromosomal region 3q26 to 3q28 could be useful to predict the efficacy of anti-HER3 antibody treatment.

Cell lines from SCCHN cancers were included in the analysis. The cell lines were evaluated by empirical sequence analysis for the presence of genetic variation in the chromosome 3q26 to 3q28 genes TP63, SOX2 and PIK3CA, and also PTEN, KRAS, PIK3CA, BRAF, MET, ERBB3, NRG1 and EGFR.

In scoring responses to treatment for 10D1F/10D1P, tumor growth inhibition (TGI) data from in vivo studies using 10D1F hIgG1 were prioritised. Where such data were not available, TGI data from 1001 P hIgG1 were used. Where in vivo data was not available for a cell line in vitro inhibition data was used.

The genotypes of the cell lines as determined by sequence analysis and their normalised functional responses to treatment with 10D1F/10D1P (as determined by analysis in in vitro or in vivo studies as described in Example 1) are shown in the table below.

| | | | | | | | | | | | Response |
|---|---|---|---|---|---|---|---|---|---|---|---|
| | | PTEN/KRAS/ | | | | | | | | | to HMBD- |
| | | PIK3CA/ | | | | | | | | Response | 001 + |
| Cell | | BRAF/MET | | | | | | | | to HMBD- | cetuximab |
| line | Cancer | status | ERBB3 | NRG1 | EGFR | TP63 | SOX2 | PIK3CA | TUSC2 | 001 (TGI) | (TGI) |
| ES0199 | ESCC | WT | WT | R38Q | R521K | JAMP | Deepdel | WT | WT | 87.8% | NA |
| LU6432 | sqNSCLC | WT | WT | G58R, AMP | R521K | AMP | AMP | WT | WT | 99.8% | NA |
| OE21 | ESCC | WT | WT | WT | AMP | WT | WT | AMP | WT | 65.3% | 101.6% |
| Cal27 | HNSCC | WT | WT | Deepdel | AMP | WT | WT | WT | Deepdel | 45.9% | 108.4% |
| CTG-2552 | sqNSCLC | WT | WT | WT | AMP | AMP | AMP | AMP | Hetloss | 52.2% | 99.2% |
| CTG-2558* | sqNSCLC | MET AMP | WT | AMP | AMP | AMP | AMP | AMP | Hetloss | 102.0% | 109.0% |

WT = homozygous wildtype
Deepdel = deep deletion
AMP = amplification
Hetloss = heterozygous loss
*CTG-2558 additionally comprises SMYD3 fusion with MET.

The data indicate that cancers comprising amplification of chromosome 3q26 to 3q28 genes TP63, SOX2 and PIK3CA respond better to anti-HER3 antibody monotherapy. Furthermore, cancers comprising of TP63/SOX2/PIK3CA amplification as well as EGFR amplification and/or loss/deletion of TUSC2, respond better to anti-HER3 and anti-EGFR combination therapy relative to anti-HER3 antibody.

### Example 6: Relationship Between BRAF/KRAS/MET/PIK3CA/PTEN Signature and Clinical Response to Treatment with HRE3-Binding Antigen-Binding Molecule Therapy

Anti-HER3 monoclonal antibody HMBD-001 was administered to subjects, and responses to treatment were monitored in accordance with details of the phase I/IIa open label, dose escalation and expansion trial of HMBD-001 given intravenously as a single agent and in combination in patients with advanced HER3 positive solid tumours, described in detail in Example 16 of WO 2023/031435 A1 (which is hereby incorporated by reference in its entirety).

DNA was extracted from blood samples obtained from 20 subjects prior to commencement of treatment, to obtain circulating tumor DNA (ctDNA). The extracted DNA was analysing using next-generation sequencing technology to determine the nucleotide sequences in the ctDNA for BRAF, KRAS, MET, PIK3CA and PTEN. After sequencing was completed, the data was subjected to bioinformatics analysis to identify mutations and characterize the genomic landscape of the tumor. The bioinformatics analysis comprised computational steps such as alignment of sequencing reads to a reference genome, and variant calling to identify mutations from the sequenced samples. Preliminary mutation statuses determined for the indicated genes in the different patients at baseline are shown in the table below.

BRAF, KRAS, MET, PIK3CA and PTEN status were characterized as follows:
For BRAF, KRAS and PIK3CA: ‘no activating mutation’ (NAM)=no gain-of-function single nucleotide variants (SNVs) or insertions/deletions (INDELs) detected; ‘Mutated’=gain-of-function SNV or INDEL detected.
For MET: ‘no activating mutation’ (NAM)=no MET amplification detected; ‘Amplified’=MET amplification detected.
For PTEN: ‘no inactivating mutation’ (NIM)=no PTEN deletion (i.e. no loss of heterozygosity/homozygosity) detected; ‘LOH’=PTEN deletion (i.e. loss of heterozygosity/homozygosity) detected.

The results of the analysis are shown in the following table. 10 out of 11 (90.9%) patients that had progressive disease on treatment with HMBD-001 harboured mutation in one or more of BRAF, KRAS, MET, PIK3CA and PTEN, as defined above (i.e. had ‘Mutated’ status for BRAF, KRAS and/or PIK3CA (i.e. a gain-of-function SNV/INDEL in one or more of these genes), and/or ‘Amplified’ status for MET, and/or ‘LOH’ status for PTE/I). Conversely, 9 out of 10 (90.0%) patients that responded to treatment with HMBD-001 (i.e. having stable disease or a partial response) did not comprise mutation in any of BRAF, KRAS, MET, PIK3CA or PTEN, as defined above (i.e. had ‘no activating mutation’ (NAM) status for BRAF, KRAS and/or PIK3CA (i.e. no gain-of-function SNVs/INDELs in these genes); NAM status for MET (i.e. no MET amplification) and had ‘no inactivating mutation’ (NIM) status for PTEN (i.e. no loss of heterozygosity/homozygosity)). This translates to a OCR for subjects not comprising mutation as defined above in any of BRAF, KRAS, MET, PIK3CA or PTEN of 90.0%, versus 9.1% for patients harbouring mutation as defined above in one or more of BRAF, KRAS, MET, PIK3CA and PTEN.

| | | | Best | | | | | | |
|---|---|---|---|---|---|---|---|---|---|
| | | | overall | 5-gene | | | | | |
| Patient | | | clinical | NAM/NIM | Status of each gene at baseline | | | | |
| no. | Dose | Tumor type | response | signature? | BRAF | KRAS | MET | PIK3CA | PTEN |
| 1 | 600 mg | NSCLC | SD | Yes | NAM | NAM | NAM | NAM | NIM |
| | | (adenocarcinoma) | | | | | | | |
| 2 | 600 mg | mCRPC | PD | No | NAM | NAM | NAM | NAM* | LOH |
| 3 | 600 mg | mCRPC | PD | No | NAM | NAM | NAM | NAM | LOH |
| 4 | 1200 mg | Esophageal | SD | Yes | NAM | NAM | NAM | NAM | NIM |
| 5 | 1200 mg | mCRPC | PD | No | NAM | NAM | Amplified | NAM | LOH |
| 6 | 1200 mg | Ovarian | PD | Yes | NAM | NAM | NAM | NAM* | NIM |
| 7 | 1800 mg | Pancreatic | PD | No | NAM | Mutated | NAM | Mutated | NIM |
| 8 | 1800 mg | mCRPC | SD | Yes | NAM | NAM | NAM | NAM | NIM |
| 9 | 1800 mg | Pancreatic | SD | Yes | NAM | NAM | NAM | NAM | NIM** |
| 10 | 3000 mg | Melanoma | PD | No | NAM | NAM | NAM | NAM | LOH |
| 11 | 3000 mg | mCRPC | PD | No | NAM | NAM | NAM | NAM | LOH |
| 12 | 3000 mg | mCRC | SD | No | NAM | NAM | Amplified | Mutated | NIM |
| 13 | 1800 mg | mCRC | PD | No | NAM | Mutated | NAM | NAM | NIM |
| 14 | 1800 mg | mCRPC | SD | Yes | NAM | NAM | NAM | NAM | NIM |
| 15 | 1800 mg | HNSCC | PD | No | NAM | NAM | NAM | Mutated | NIM |
| 16 | 3000 mg | Pancreatic | PR | Yes | NAM | NAM | NAM | NAM | NIM |
| 17 | 3000 mg | HGSOC | PD | No | NAM | NAM | NAM*** | Mutated | NIM |
| 18 | 3000 mg | NSCLC | PD | No | NAM | Mutated | NAM | NAM | NIM |
| | | (adenocarcinoma) | | | | | | | |
| 19 | 3000 mg | Melanoma | SD | Yes | NAM | NAM | NAM | NAM | NIM |
| 20 | 3000 mg | NSCLC | SD | Yes | NAM | NAM | NAM | NAM | NIM** |
| | | (adenocarcinoma) | | | | | | | |
| 21 | 3000 mg | Cervical | SD | Yes | NAM | NAM | NAM | NAM | NIM |

*= amplification of PIK3CA detected
**= mutation in PTEN detected, effect on PTEN protein expression not analysed
***= mutation in MET detected, effect on MET protein expression not analysed

| | | SD/PR | PD | DCR |
|---|---|---|---|---|
| Gene | NAM/NIM | 9 | 1 | 90.0% |
| Signature | Mut | 1 | 10 | 9.1% |

1. An antigen-binding molecule that binds to HER3 for use in a method of treating or preventing a HER3-associated cancer in a subject, wherein the HER3-associated cancer does not comprise genetic variation resulting in an increase in the expression of, or an increase in the activity of a gene product of, MET.

2. Use of an antigen-binding molecule that binds to HER3 in the manufacture of a medicament for use in treating or preventing a HER3-associated cancer in a subject, wherein the HER3-associated cancer does not comprise genetic variation resulting in an increase in the expression of, or an increase in the activity of a gene product of, MET.

3. A method of treating or preventing a HER3-associated cancer in a subject, comprising administering to the subject a therapeutically- or prophylactically-effective amount of an antigen-binding molecule that binds to HER3, wherein the HER3-associated cancer does not comprise genetic variation resulting in an increase in the expression of, or an increase in the activity of a gene product of, MET.

4. The antigen-binding molecule for use according to claim 1, the use according to claim 2, or the method according to claim 3, wherein the HER3-associated cancer: (i) does not comprise genetic variation resulting in an increase in the expression of, or an increase in the activity of a gene product of, KRAS; or (ii) does not comprise genetic variation resulting in an increase in the expression of, or an increase in the activity of a gene product of, PIK3CA; or (iii) does not comprise genetic variation resulting in an increase in the expression of, or an increase in the activity of a gene product of, BRAF; or (iv) does not comprise genetic variation resulting in a decrease in the expression of, or a decrease in the activity of a gene product of, PTEN.

5. The antigen-binding molecule for use according to claim 1 or claim 4, the use according to claim 2 or claim 4, or the method according to claim 3 or claim 4, wherein the HER3-associated cancer: (i) does not comprise genetic variation resulting in an increase in the expression of, or an increase in the activity of a gene product of, KRAS; and (ii) does not comprise genetic variation resulting in an increase in the expression of, or an increase in the activity of a gene product of, PIK3CA; and (iii) does not comprise genetic variation resulting in an increase in the expression of, or an increase in the activity of a gene product of, BRAF; and (iv) does not comprise genetic variation resulting in a decrease in the expression of, or a decrease in the activity of a gene product of, PTEN.

6. The antigen-binding molecule for use according to any one of claims 1, 4 or 5, the use according to any one of claims 2, 4 or 5, or the method according to any one of claims 3 to 5, wherein the HER3-associated cancer: (i) does not comprise amplification of MET, and (ii) does not comprise an activating mutation to KRAS, and (iii) does not comprise an activating mutation to PIK3CA, and (iv) does not comprise an activating mutation to BRAF, and (iv) does not comprise deletion of PTEN.

7. The antigen-binding molecule for use according to any one of claims 1, or 4 to 6, the use according to any one of claims 2, or 4 to 6, or the method according to any one of claims 3 to 6, wherein the HER3-associated cancer comprises amplification of one or more genes located on chromosome 3q, optionally wherein the one or more genes located on chromosome 3q are located within chromosome 3q26-3q28, optionally wherein the one or more genes are selected from: TP63, SOX2 and PIK3CA.

8. The antigen-binding molecule for use according to any one of claims 1, or 4 to 7, the use according to any one of claims 2, or 4 to 7, or the method according to any one of claims 3 to 7, wherein the HER3-associated cancer comprises amplification of one or more genes located on chromosome 7p, optionally wherein the one or more genes are located within chromosome 7p11, optionally wherein the gene is EGFR.

9. The antigen-binding molecule for use according to any one of claims 1, or 4 to 7, the use according to any one of claims 2, or 4 to 7, or the method according to any one of claims 3 to 7, wherein the HER3-associated cancer comprises deletion of one or more genes located on chromosome 3p, optionally wherein the one or more genes are located within chromosome 3p21, optionally wherein the gene is TUSC2.

10. The antigen-binding molecule for use according to any one of claims 1, or 4 to 9, the use according to any one of claims 2, or 4 to 9, or the method according to any one of claims 3 to 9, wherein the HER3-associated cancer comprises genetic variation resulting in increased expression of a ligand for HER3.

11. The antigen-binding molecule for use according to any one of claims 1, or 4 to 10, the use according to any one of claims 2, or 4 to 10, or the method according to any one of claims 3 to 10, wherein the HER3-associated cancer comprises an NRG gene fusion, an NRG1 gene fusion, or an NRG2 gene fusion.

12. The antigen-binding molecule for use according to any one of claims 1, or 4 to 11, the use according to any one of claims 2, or 4 to 11, or the method according to any one of claims 3 to 11, wherein the HER3-associated cancer comprises an NRG gene fusion selected from: CLU-NRG1, CD74-NRG1, DOC4-NRG1, SLC3A2-NRG1, RBPMS-NRG1, WRN-NRG1, SDC4-NRG1, RAB21L1-NRG1, VAMP2-NRG1, KIF13B-NRG1, THAP7-NRG1, SMAD4-NRG1, MDK-NRG1, TNC-NRG1, DIP2B-NRG1, MRPL13-NRG1, PARP8-NRG1, ROCK1-NRG1, DPYSL2-NRG1, ATP1B1-NRG1, CDH6-NRG1, APP-NRG1, AKAP13-NRG1, THBS1-NRG1, FOXA1-NRG1, PDE7A-NRG1, RAB31L1-NRG1, CDK1-NRG1, BMPRIB-NRG1, TNFRSF10B-NRG1, MCPH1-NRG1, and SLC12A2-NRG2.

13. An antigen-binding molecule that binds to HER3 for use in a method of treating or preventing a HER3-associated cancer in a subject, wherein the HER3-associated cancer comprises amplification of one or more genes located on chromosome 3q, optionally wherein the one or more genes located on chromosome 3q are located within chromosome 3q26-3q28, optionally wherein the one or more genes are selected from: TP63, SOX2 and PIK3CA.

14. Use of an antigen-binding molecule that binds to HER3 in the manufacture of a medicament for use in treating or preventing a HER3-associated cancer in a subject, wherein the HER3-associated cancer comprises amplification of one or more of genes amplification of one or more genes located on chromosome 3q, optionally wherein the one or more genes located on chromosome 3q are located within chromosome 3q26-3q28, optionally wherein the one or more genes are selected from: TP63, SOX2 and PIK3CA.

15. A method of treating or preventing a HER3-associated cancer in a subject, comprising administering to the subject a therapeutically- or prophylactically-effective amount of an antigen-binding molecule that binds to HER3, wherein the HER3-associated cancer comprises amplification of one or more genes located on chromosome 3q, optionally wherein the one or more genes located on chromosome 3q are located within chromosome 3q26-3q28, optionally wherein the one or more genes are selected from: TP63, SOX2 and PIK3CA.

16. The antigen-binding molecule for use according to claim 13 or claim 16, the use according to claim 14 or claim 16, or the method according to claim 15 or claim 16, wherein the HER3-associated cancer comprises amplification of TP63, and comprises amplification of SOX2, and comprises amplification of PIK3CA.

17. An antigen-binding molecule that binds to HER3 for use in a method of treating or preventing a HER3-associated cancer in a subject, wherein the HER3-associated cancer comprises amplification of one or more genes located on chromosome 7p, optionally wherein the one or more genes are located within chromosome 7p11, optionally wherein the gene is EGFR.

18. Use of an antigen-binding molecule that binds to HER3 in the manufacture of a medicament for use in treating or preventing a HER3-associated cancer in a subject, wherein the HER3-associated cancer comprises amplification of one or more genes located on chromosome 7p, optionally wherein the one or more genes are located within chromosome 7p11, optionally wherein the gene is EGFR.

19. A method of treating or preventing a HER3-associated cancer in a subject, comprising administering to the subject a therapeutically- or prophylactically-effective amount of an antigen-binding molecule that binds to HER3, wherein the HER3-associated cancer comprises amplification of one or more genes located on chromosome 7p, optionally wherein the one or more genes are located within chromosome 7p11, optionally wherein the gene is EGFR.

20. An antigen-binding molecule that binds to HER3 for use in a method of treating or preventing a HER3-associated cancer in a subject, wherein the HER3-associated cancer comprises deletion of one or more genes located on chromosome 3p, optionally wherein the one or more genes are located within chromosome 3p21, optionally wherein the gene is TUSC2.

21. Use of an antigen-binding molecule that binds to HER3 in the manufacture of a medicament for use in treating or preventing a HER3-associated cancer in a subject, wherein the HER3-associated cancer comprises deletion of one or more genes located on chromosome 3p, optionally wherein the one or more genes are located within chromosome 3p21, optionally wherein the gene is TUSC2.

22. A method of treating or preventing a HER3-associated cancer in a subject, comprising administering to the subject a therapeutically- or prophylactically-effective amount of an antigen-binding molecule that binds to HER3, wherein the HER3-associated cancer comprises deletion of one or more genes located on chromosome 3p, optionally wherein the one or more genes are located within chromosome 3p21, optionally wherein the gene is TUSC2.

23. An antigen-binding molecule that binds to HER3 for use in a method of treating or preventing a HER3-associated cancer in a subject, wherein the HER3-associated cancer comprises genetic variation resulting in an increase in the expression of, or an increase in the activity of a gene product of, MET, and wherein the method further comprises administering an antagonist of HER3-mediated signalling.

24. Use of an antigen-binding molecule that binds to HER3 for use in the manufacture of a medicament for use in treating or preventing a HER3-associated cancer in a subject, wherein the HER3-associated cancer comprises genetic variation resulting in an increase in the expression of, or an increase in the activity of a gene product of, MET, and wherein the method further comprises administering an antagonist of HER3-mediated signalling.

25. A method of treating or preventing a HER3-associated cancer in a subject, comprising administering to the subject a therapeutically- or prophylactically-effective amount of an antigen-binding molecule that binds to HER3, wherein the HER3-associated cancer comprises genetic variation resulting in an increase in the expression of, or an increase in the activity of a gene product of, MET, and wherein the method further comprises administering an antagonist of HER3-mediated signalling.

26. The antigen-binding molecule for use according to claim 23, the use according to claim 24, or the method according to claim 25, wherein the HER3-associated cancer: (i) comprises genetic variation resulting in an increase in the expression of, or an increase in the activity of a gene product of, KRAS; or (ii) comprises genetic variation resulting in an increase in the expression of, or an increase in the activity of a gene product of, PIK3CA; or (iii) comprises genetic variation resulting in an increase in the expression of, or an increase in the activity of a gene product of, BRAF; or (iv) comprises genetic variation resulting in a decrease in the expression of, or a decrease in the activity of a gene product of, PTEN.

27. The antigen-binding molecule for use according to claim 23 or claim 26, the use according to claim 24 or claim 26, or the method according to claim 25 or claim 26, wherein the HER3-associated cancer: (i) comprises genetic variation resulting in an increase in the expression of, or an increase in the activity of a gene product of, KRAS; and (ii) comprises genetic variation resulting in an increase in the expression of, or an increase in the activity of a gene product of, PIK3CA; and (iii) comprises genetic variation resulting in an increase in the expression of, or an increase in the activity of a gene product of, BRAF; and (iv) comprises genetic variation resulting in a decrease in the expression of, or a decrease in the activity of a gene product of, PTEN.

28. A method of selecting a subject for treatment with an antigen-binding molecule that binds to HER3, comprising:
(a) analysing a subject's cancer to determine whether the cancer comprises genetic variation resulting in an increase in the expression of, or an increase in the activity of a gene product of, MET; and
(b) selecting a subject for treatment with an antigen-binding molecule that binds to HER3 where the subject's cancer is determined in step (a) not to comprise such genetic variation.

29. The method according to claim 28, wherein the method comprises:
(a)
(i) analysing a subject's cancer to determine whether the cancer comprises genetic variation resulting in an increase in the expression of, or an increase in the activity of a gene product of, KRAS; or
(ii) analysing a subject's cancer to determine whether the cancer comprises an activating mutation to PIK3CA; or
(iii) analysing a subject's cancer to determine whether the cancer comprises genetic variation resulting in an increase in the expression of, or an increase in the activity of a gene product of, BRAF; or
(iv) analysing a subject's cancer to determine whether the cancer comprises genetic variation resulting in a decrease in the expression of, or a decrease in the activity of a gene product of, PTEN; and
(b) selecting a subject for treatment with an antigen-binding molecule that binds to HER3 where the subject's cancer is determined in step (a) not to comprise such genetic variation/mutation.

30. The method according to claim 28 or claim 29, wherein the method comprises:
(a)
(i) analysing a subject's cancer to determine whether the cancer comprises genetic variation resulting in an increase in the expression of, or an increase in the activity of a gene product of, KRAS; and
(ii) analysing a subject's cancer to determine whether the cancer comprises an activating mutation to PIK3CA; and
(iii) analysing a subject's cancer to determine whether the cancer comprises genetic variation resulting in an increase in the expression of, or an increase in the activity of a gene product of, BRAF; and
(iv) analysing a subject's cancer to determine whether the cancer comprises genetic variation resulting in a decrease in the expression of, or a decrease in the activity of a gene product of, PTEN; and
(b) selecting a subject for treatment with an antigen-binding molecule that binds to HER3 where the subject's cancer is determined in step (a) not to comprise such genetic variation/mutation.

31. The method according to any one of claims 28 to 30, wherein the method further comprises:
(c) administering an antigen-binding molecule that binds to HER3 to a subject selected for treatment in step (b).

32. A method of selecting a subject for treatment with (i) an antagonist of HER3-mediated signalling and (ii) an antigen-binding molecule that binds to HER3, comprising:
(a) analysing a subject's cancer to determine whether the cancer comprises genetic variation resulting in an increase in the expression of, or an increase in the activity of a gene product of, MET; and
(b) selecting a subject for treatment with (i) an antagonist of HER3-mediated signalling and (ii) an antigen-binding molecule that binds to HER3 where the subject's cancer is determined in step (a) to comprise such genetic variation.

33. The method according to claim 32, wherein the method comprises:
(a)
(i) analysing a subject's cancer to determine whether the cancer comprises genetic variation resulting in an increase in the expression of, or an increase in the activity of a gene product of, KRAS; or
(ii) analysing a subject's cancer to determine whether the cancer comprises an activating mutation to PIK3CA; or
(iii) analysing a subject's cancer to determine whether the cancer comprises genetic variation resulting in an increase in the expression of, or an increase in the activity of a gene product of, BRAF; or
(iv) analysing a subject's cancer to determine whether the cancer comprises genetic variation resulting in a decrease in the expression of, or a decrease in the activity of a gene product of, PTEN; and
(b) selecting a subject for treatment with (i) an antagonist of HER3-mediated signalling and (ii) an antigen-binding molecule that binds to HER3 where the subject's cancer is determined in step (a) to comprise such genetic variation/mutation.

34. The method according to claim 32 or claim 33, wherein the method comprises:
(a)
(i) analysing a subject's cancer to determine whether the cancer comprises genetic variation resulting in an increase in the expression of, or an increase in the activity of a gene product of, KRAS; and
(ii) analysing a subject's cancer to determine whether the cancer comprises an activating mutation to PIK3CA; and
(iii) analysing a subject's cancer to determine whether the cancer comprises genetic variation resulting in an increase in the expression of, or an increase in the activity of a gene product of, BRAF; and
(iv) analysing a subject's cancer to determine whether the cancer comprises genetic variation resulting in a decrease in the expression of, or a decrease in the activity of a gene product of, PTEN; and
(b) selecting a subject for treatment with (i) an antagonist of HER3-mediated signalling and (ii) an antigen-binding molecule that binds to HER3 where the subject's cancer is determined in step (a) to comprise such genetic variation/mutation.

35. The method according to any one of claims 32 to 34, wherein the method further comprises:
(c) administering (i) an antagonist of HER3-mediated signalling and (ii) an antigen-binding molecule that binds to HER3 to a subject selected for treatment in step (b).

36. The antigen-binding molecule for use according to any one of claims 1, 4 to 13, 16, 17, 20, 23, 26 or 27, the use according to any one of claims 2, 4 to 12, 14, 16, 18, 21, 24, 26 or 27, or the method according to any one of claims 3 to 12, 15, 19, 22, 25, or 28 to 35, wherein the HER3-associated cancer is selected from: a solid tumor, breast cancer, breast carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck, lung cancer, non-small cell lung cancer, lung adenocarcinoma, squamous cell lung carcinoma, ovarian cancer, ovarian carcinoma, ovarian serous adenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, uterine cancer, uterine corpus endometrial carcinoma, thyroid cancer, thyroid carcinoma, pheochromocytoma, paraganglioma, bladder cancer, bladder urothelial carcinoma, prostate cancer, prostate adenocarcinoma, sarcoma and thymoma.

37. The antigen-binding molecule for use according to any one of claims 1, 4 to 13, 16, 17, 20, 23, 26, 27 or 36 the use according to any one of claims 2, 4 to 12, 14, 16, 18, 21, 24, 26, 27 or 36, or the method according to any one of claims 3 to 12, 15, 19, or 22 to 36, wherein the antigen-binding molecule that binds to HER3 is selected from: 10D1F, seribantumab, elgemtumab, patritumab, GSK2849330, lumretuzumab, CDX-3379, AV-203, barecetamab, TK-A3, TK-A4, MP-EV20, 1A5-3D4, 9F7-F11, 16D3-C1, NG33, A5, F4, huHER3-8, REGN1400 and zenocutuzumab.

38. The antigen-binding molecule for use according to any one of claims 1, 4 to 13, 16, 17, 20, 23, 26, 27, 36 or 37 the use according to any one of claims 2, 4 to 12, 14, 16, 18, 21, 24, 26, 27, 36 or 37, or the method according to any one of claims 3 to 12, 15, 19, or 22 to 37, wherein the antigen-binding molecule that binds to HER3 comprises:
(i) a heavy chain variable (VH) region incorporating the following CDRs:
HC-CDR1 having the amino acid sequence of SEQ ID NO:40
HC-CDR2 having the amino acid sequence of SEQ ID NO:43
HC-CDR3 having the amino acid sequence of SEQ ID NO:48; and
(ii) a light chain variable (VL) region incorporating the following CDRs:
LC-CDR1 having the amino acid sequence of SEQ ID NO:66
LC-CDR2 having the amino acid sequence of SEQ ID NO:69
LC-CDR3 having the amino acid sequence of SEQ ID NO:74.

39. The antigen-binding molecule for use according to any one of claims 1, 4 to 13, 16, 17, 20, 23, 26, 27, or 36 to 38 the use according to any one of claims 2, 4 to 12, 14, 16, 18, 21, 24, 26, 27, or 36 to 38, or the method according to any one of claims 3 to 12, 15, 19, or 22 to 38, wherein the antigen-binding molecule that binds to HER3 comprises:
(i) a VH region incorporating the following CDRs:
HC-CDR1 having the amino acid sequence of SEQ ID NO:38
HC-CDR2 having the amino acid sequence of SEQ ID NO:42
HC-CDR3 having the amino acid sequence of SEQ ID NO:45; and
(ii) a VL region incorporating the following CDRs:
LC-CDR1 having the amino acid sequence of SEQ ID NO:63
LC-CDR2 having the amino acid sequence of SEQ ID NO:67
LC-CDR3 having the amino acid sequence of SEQ ID NO:70.

40. The antigen-binding molecule for use according to any one of claims 1, 4 to 13, 16, 17, 20, 23, 26, 27, or 36 to 39 the use according to any one of claims 2, 4 to 12, 14, 16, 18, 21, 24, 26, 27, or 36 to 39, or the method according to any one of claims 3 to 12, 15, 19, or 22 to 39, wherein the antigen-binding molecule that binds to HER3 comprises:
a VH region comprising an amino acid sequence having at least 70% sequence identity to the amino acid sequence of SEQ ID NO:33; and
a VL region comprising an amino acid sequence having at least 70% sequence identity to the amino acid sequence of SEQ ID NO:58.

41. The antigen-binding molecule for use according to any one of claims 1, 4 to 13, 16, 17, 20, 23, 26, 27, or 36 to 40 the use according to any one of claims 2, 4 to 12, 14, 16, 18, 21, 24, 26, 27, or 36 to 40, or the method according to any one of claims 3 to 12, 15, 19, or 22 to 40, wherein the antigen-binding molecule that binds to HER3 comprises:
a polypeptide comprising, or consisting of, an amino acid sequence having at least 70% sequence identity to the amino acid sequence of SEQ ID NO:75; and
a polypeptide comprising, or consisting of, an amino acid sequence having at least 70% sequence identity to the amino acid sequence of SEQ ID NO:76.

42. The antigen-binding molecule for use according to any one of claims 1, 4 to 13, 16, 17, 20, 23, 26, 27, or 36 to 41 the use according to any one of claims 2, 4 to 12, 14, 16, 18, 21, 24, 26, 27, or 36 to 41, or the method according to any one of claims 3 to 12, 15, 19, or 22 to 41, wherein the method of treating or preventing the HER3-associated cancer further comprises administering an antagonist of EGFR to the subject.

43. The antigen-binding molecule for use according to claim 42, the use according to claim 42, or the method according to claim 42, wherein the antagonist of EGFR is antigen-binding molecule that binds to EGFR.

44. The antigen-binding molecule for use according to claim 43, the use according to claim 43, or the method according to claim 43, wherein the antigen-binding molecule that binds to EGFR comprises:
(i) a VH region incorporating the following CDRs:
HC-CDR1 having the amino acid sequence of SEQ ID NO:78
HC-CDR2 having the amino acid sequence of SEQ ID NO:79
HC-CDR3 having the amino acid sequence of SEQ ID NO:80; and
(ii) a VL region incorporating the following CDRs:
LC-CDR1 having the amino acid sequence of SEQ ID NO:82
LC-CDR2 having the amino acid sequence of SEQ ID NO:83
LC-CDR3 having the amino acid sequence of SEQ ID NO:84.

45. The antigen-binding molecule for use according to claim 43 or claim 44, the use according to claim 43 claim 44, or the method according to claim 43 claim 44, wherein the antigen-binding molecule that binds to EGFR comprises:
a VH region comprising an amino acid sequence having at least 70% sequence identity to the amino acid sequence of SEQ ID NO:77; and
a VL region comprising an amino acid sequence having at least 70% sequence identity to the amino acid sequence of SEQ ID NO:81.

46. The antigen-binding molecule for use according to any one of claims 43 to 45, the use according to any one of claims 43 to 45, or the method according to any one of claims 43 to 45, wherein the antigen-binding molecule that binds to EGFR comprises:
a polypeptide comprising, or consisting of, an amino acid sequence having at least 70% sequence identity to the amino acid sequence of SEQ ID NO:85; and
a polypeptide comprising, or consisting of, an amino acid sequence having at least 70% sequence identity to the amino acid sequence of SEQ ID NO:86.
